SEQUENCE LISTING (95 sequences)

SEQ ID NO: 1 - protein, 775 aa, synthetic construct
MRNCKKDLYVEVTPMQKELVLGIDYGGKYTGLAVVDRRNNQVLYARTVKMRDDVADILEGRREQRGIRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRDKSNKGTPLYDKNPLSIKNSLITRRWMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVEKPPEIKGKKLYTLRRGDYVTHESEDSVYRIKKLGTSPVLEAVVKGSIGRIRNSASATKLTKVSKPQKVF

SEQ ID NO: 2 - RNA, 202 nt, synthetic construct
  source 1..202: mol_type = other RNA
gttacagttaaggctctttggaaacaaagaagccttaattgtaaaacgcctatatggtaaagtgatgtacgtttgggtatatatcgccagcctgaacctctacgccagaaatggcagctttatcatgggttaggacgatatttaaaaaactttctgcggcttgcttactttagtaagctttgtggctgaggcagaattcctt

SEQ ID NO: 3 - protein, 18 aa, synthetic construct
KRTADGSEFESPKKKRKV

SEQ ID NO: 4 - protein, 16 aa, synthetic construct
KRPAATKKAGQAKKKK

SEQ ID NO: 5 - protein, 841 aa, synthetic construct
KRTADGSEFESPKKKRKVRNCKKDLYVEVTPMQKELVLGIDYGGKYTGLAVVDRRNNQVLYARTVKMRDDVADILEGRREQRGIRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRDKSNKGTPLYDKNPLSIKNSLITRRWMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVEKPPEIKGKKLYTLRRGDYVTHESEDSVYRIKKLGTSPVLEAVVKGSIGRIRNSASATKLTKVSKPQKVFSGGSSGGSSGSETPGTSESATPESSGGSSGGSSKRPAATKKAGQAKKKK

SEQ ID NO: 6 - protein, 775 aa, synthetic construct
MRNCKKDLYVEVTPMQKELVLGIDYGGKYTGLAVVDRRNNQVLYARTVKMRDDVADILEGRREQRGRRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRRKSNKGTPLYDKNPLSIKNSLITRRWMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVRKPPEIKGKKLYTLRRGDYVTHESEDSVYRIKKLGTSPVLEAVVKGSIGRIRNSASATKLTKVSKPQKVF

SEQ ID NO: 7 - protein, 775 aa, synthetic construct
MRNCKKDLYVEVTPMQKELVLGIDYGGKYTGLAVVDRRNNQVLYARTVKMRDDVARILEGRREQRGRRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRRKSNKGTPLYDKNPLSIKNSLITRRWMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVEKPPEIKGKKLYTLRRGDYVTHESEDSVYRIKKLGTSPVLEAVVKGSIGRIRNSASATKLTKVSKPQKVF

SEQ ID NO: 8 - protein, 775 aa, synthetic construct
MRNCKKDLYVEVTPMQKELVLGIDYGGKYTGLAVVDRRNNQVLYARTVKMRDDVADILEGRREQRGRRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRRKSNKGTPLYDKNPLSIKNSLITRRWMVTEIEHGKEDRIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVEKPPEIKGKKLYTLRRGDYVTHESEDSVYRIKKLGTSPVLEAVVKGSIGRIRNSASATKLTKVSKPQKVF

SEQ ID NO: 9 - protein, 775 aa, synthetic construct
MRNCKKDLYVEVTPMQKELVLGIDYGGKYTGLAVVDRRNNQVLYARTVKMRDDVARILEGRREQRGRRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRDKSNKGTPLYDKNPLSIKNSLITRRWMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVRKPPEIKGKKLYTLRRGDYVTHESEDSVYRIKKLGTSPVLEAVVKGSIGRIRNSASATKLTKVSKPQKVF

SEQ ID NO: 10 - protein, 775 aa, synthetic construct
MRNCKKDLYVEVTPMQKELVLGIDYGGKYTGLAVVDRRNNQVLYARTVKMRDDVADILEGRREQRGRRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRDKSNKGTPLYDKNPLSIKNSLITRRWMVTEIEHGKEDRIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVRKPPEIKGKKLYTLRRGDYVTHESEDSVYRIKKLGTSPVLEAVVKGSIGRIRNSASATKLTKVSKPQKVF

SEQ ID NO: 11 - protein, 775 aa, synthetic construct
MRNCKKDLYVEVTPMQKELVLGIDYGGKYTGLAVVDRRNNQVLYARTVKMRDDVADILEGRREQRGRRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRRKSNKGTPLYDKNPLSIKNSLITRRWMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYRIKNPRLLKHFVEKPPEIKGKKLYTLRRGDYVTHESEDSVYRIKKLGTSPVLEAVVKGSIGRIRNSASATKLTKVSKPQKVF

SEQ ID NO: 12 - protein, 775 aa, synthetic construct
MRNCKKDLYVEVTPMQKELVLGIDYGGKYTGLAVVDRRNNQVLYARTVKMRDDVARILEGRREQRGRRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRDKSNKGTPLYDKNPLSIKNSLITRRWMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYRIKNPRLLKHFVRKPPEIKGKKLYTLRRGDYVTHESEDSVYRIKKLGTSPVLEAVVKGSIGRIRNSASATKLTKVSKPQKVF

SEQ ID NO: 13 - protein, 775 aa, synthetic construct
MRNCKKDLYVEVTPMQKELVLGIDYGGKYTGLAVVDRRNNQVLYARTVKMRDDVARILEGRREQRGRRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRDKSNKGTPLYDKNPLSIKNSLITRRWMVTEIEHGKEDRIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVRKPPEIKGKKLYTLRRGDYVTHESEDSVYRIKKLGTSPVLEAVVKGSIGRIRNSASATKLTKVSKPQKVF

SEQ ID NO: 14 - DNA, 20 bp, synthetic construct
  source 1..20: mol_type = other DNA
ttgtgagaatggtgcgtcct

SEQ ID NO: 15 - RNA, 222 nt, synthetic construct
  source 1..222: mol_type = other RNA
ttgtgagaatggtgcgtcctgttacagttaaggctctttggaaacaaagaagccttaattgtaaaacgcctatatggtaaagtgatgtacgtttgggtatatatcgccagcctgaacctctacgccagaaatggcagctttatcatgggttaggacgatatttaaaaaactttctgcggcttgcttactttagtaagctttgtggctgaggcagaattcctt

SEQ ID NO: 16 - DNA, 20 bp, synthetic construct
  source 1..20: mol_type = other DNA
ctttgtagcctctcccgctc

SEQ ID NO: 17 - RNA, 222 nt, synthetic construct
  source 1..222: mol_type = other RNA
ctttgtagcctctcccgctcgttacagttaaggctctttggaaacaaagaagccttaattgtaaaacgcctatatggtaaagtgatgtacgtttgggtatatatcgccagcctgaacctctacgccagaaatggcagctttatcatgggttaggacgatatttaaaaaactttctgcggcttgcttactttagtaagctttgtggctgaggcagaattcctt

SEQ ID NO: 18 - DNA, 20 bp, synthetic construct
  source 1..20: mol_type = other DNA
tggatggcgacttcaggcac

SEQ ID NO: 19 - RNA, 222 nt, synthetic construct
  source 1..222: mol_type = other RNA
tggatggcgacttcaggcacgttacagttaaggctctttggaaacaaagaagccttaattgtaaaacgcctatatggtaaagtgatgtacgtttgggtatatatcgccagcctgaacctctacgccagaaatggcagctttatcatgggttaggacgatatttaaaaaactttctgcggcttgcttactttagtaagctttgtggctgaggcagaattcctt

SEQ ID NO: 20 - DNA, 20 bp, synthetic construct
  source 1..20: mol_type = other DNA
ctttagcaagggactattca

SEQ ID NO: 21 - RNA, 222 nt, synthetic construct
  source 1..222: mol_type = other RNA
ctttagcaagggactattcagttacagttaaggctctttggaaacaaagaagccttaattgtaaaacgcctatatggtaaagtgatgtacgtttgggtatatatcgccagcctgaacctctacgccagaaatggcagctttatcatgggttaggacgatatttaaaaaactttctgcggcttgcttactttagtaagctttgtggctgaggcagaattcctt

SEQ ID NO: 22 - DNA, 20 bp, synthetic construct
  source 1..20: mol_type = other DNA
ttgttgacattgtccacacc

SEQ ID NO: 23 - RNA, 222 nt, synthetic construct
  source 1..222: mol_type = other RNA
ttgttgacattgtccacaccgttacagttaaggctctttggaaacaaagaagccttaattgtaaaacgcctatatggtaaagtgatgtacgtttgggtatatatcgccagcctgaacctctacgccagaaatggcagctttatcatgggttaggacgatatttaaaaaactttctgcggcttgcttactttagtaagctttgtggctgaggcagaattcctt

SEQ ID NO: 24 - DNA, 20 bp, synthetic construct
  source 1..20: mol_type = other DNA
tttgcccatactggggacca

SEQ ID NO: 25 - RNA, 222 nt, synthetic construct
  source 1..222: mol_type = other RNA
tttgcccatactggggaccagttacagttaaggctctttggaaacaaagaagccttaattgtaaaacgcctatatggtaaagtgatgtacgtttgggtatatatcgccagcctgaacctctacgccagaaatggcagctttatcatgggttaggacgatatttaaaaaactttctgcggcttgcttactttagtaagctttgtggctgaggcagaattcctt

SEQ ID NO: 26 - RNA, 20 nt, synthetic construct
  source 1..20: mol_type = other RNA
gagaaagagaaagggagtag

SEQ ID NO: 27 - RNA, 127 nt, synthetic construct
  source 1..127: mol_type = other RNA
gttacagttaagggaaaccttaattgtaaaacgcctatatggtaaagtgatgtacgtttgggtatatatcgccagcctgaacctctacgccagaaatggcagctttatcatgggttaggacgatatt

SEQ ID NO: 28 - RNA, 118 nt, synthetic construct
  source 1..118: mol_type = other RNA
gttacagttaagggaaaccttaattgtaaaacgcctatacggtgaagggatatacgcttgggtttgtccagcctgagcctctatgccagaaatggcgccttcatcgtgggttaggaca

SEQ ID NO: 29 - RNA, 147 nt, synthetic construct
  source 1..147: mol_type = other RNA
ttgtgagaatggtgcgtcctgttacagttaagggaaaccttaattgtaaaacgcctatatggtaaagtgatgtacgtttgggtatatatcgccagcctgaacctctacgccagaaatggcagctttatcatgggttaggacgatatt

SEQ ID NO: 30 - RNA, 147 nt, synthetic construct
  source 1..147: mol_type = other RNA
ctttagcaagggactattcagttacagttaagggaaaccttaattgtaaaacgcctatatggtaaagtgatgtacgtttgggtatatatcgccagcctgaacctctacgccagaaatggcagctttatcatgggttaggacgatatt

SEQ ID NO: 31 - RNA, 138 nt, synthetic construct
  source 1..138: mol_type = other RNA
gagaaagagaaagggagtaggttacagttaagggaaaccttaattgtaaaacgcctatacggtgaagggatatacgcttgggtttgtccagcctgagcctctatgccagaaatggcgccttcatcgtgggttaggaca

SEQ ID NO: 32 - DNA, 2325 bp, synthetic construct
  source 1..2325: mol_type = other DNA
atgaggaattgtaagaaagatctatatgtagaggttaccccgatgcagaaagagttagtcctgggtatcgattacggcggtaagtataccgggctggcggtcgtggatcgtcgcaataaccaagttctgtacgcgcgtaccgtcaagatgcgtgacgatgtagctgatattttagagggtcgtcgggagcagcgtggtatccgtcgtaccggccagaccaagaagaaacgtctacgtgaattgaagacctatttgaaaaacattggcggtaagtacgacgaaagcatcggtacattcaagaacgaaccgtttatgaccgtgtacaaactgtgtcataagcgtggctatgactacgttgagatgccgaaagagaagacccgtgaagagattgaggctatgagcgcggatgagaagaagcaatgggaaaaagagaaaaaggagtgggaagagaccgaacgcaactctcgtcaccgtgacgaggtaacgaaggacgttcgtaaaatcatgagcgaacgcgacgcgaccgaacagcagatcagccatgttattaaaatcttcaacaaacaatatcgtccaaagcgttttaacaaccgtatcctgaccaagtgcaaagttgaagattgtggtgtcaataccccgctgcgtaaaaacgtgcgtgacctgctgattaaggacatcgtgcgttttctgccgctgacgcagagcgaaaaggctgaattggaggcccacgtgctggacaaagaaggccgcgagaaggttaaaaccttcttccgacgccaccgcattaacgagttcctgcgcaagcagatttatgacatcgctttcaatcagaaactttctggtcgtaccgtcttctgcaaaaagcacattctggagcacgaacaacacacgaaggaggaacgcaaggtgttccgcttggcgccaagcttgaaaaccaagatcaataacgtgttggctgtggttaaagatgagatccttccgcaatttggtattaataaagtgattatggagtccaacaattttgatatcgccgcacgtaccaagggcaaaaagagattagcaaaagacgagtatggtaagggtcataaagaagcgaaggagtccctcatcgaggtgctgctgagagaaaccaaaaaccaatgtgtttactgcggtaaggtgatcaccgttacgaccgcgaacaccgaccacatctttccgcgtaaggcgggtggtatcaacgtctttgcaaatctggttgcctgctgtcgtgagtgcaatgaaaaaaaaaaaggccgtaccccgctggagtccggtgtgttgccacaaccggaaattactgcgcacctggagaacgatctgaagaagaaaattctggaggacgctcggaacatgaatgagttgaacctgaattcgatcaaatacatgagctatgccagcatcggctggcgtcacatgcgtgaccgactccgtggtttaatcggcaacgaagaactgccgattgaacgccaaagcggcatcttgaccgcgtactttcgtcgctggtggggtttccgcaaagaaaggggcaatgacattcatcacgcactggacgcggttattttagcgtctcgcaaggattacaccgacgacggcgttgatatgacgctgaagccgaaatccagcaacggtcgcgaattcgatccggaaaaacacctgccggataccatcagttttaaacgtgacaaaagcaacaaaggcacgcctctgtacgacaagaatccgctttcgattaagaacagcttgattacccgtcgctggatggttactgaaatcgaacatggtaaggaggataccattatttcggatgagtacagaaaaaagctaaaagaagcatttaagcattggggcacgagcaaaggcaaatgcctgacggatgaagaggcgcgtcagaccggcttttatcagcgcaaaaacgggaacttcatgtcactccgctgcgttgaaaaaggtctgggctggggacaaatggtgaaaatcaataacaacgcgttcaaaaccaatgtgcataatattggtgtagcagtgtacctggatgagaagggtaagaaaaaggcgtatgagatcaagaacccgcgtctgctcaaacacttcgtggaaaaaccgccggagataaaaggcaagaagctctacacactgcgtcgtggcgattatgttacgcatgaaagtgaggacagcgtttaccgtatcaaaaagctgggtacgtccccggttctcgaggccgtggttaaaggctctattggtagaatccgtaatagcgcatcggctaccaagttgactaaggtctccaaaccgcagaaagtgttc

SEQ ID NO: 33 - DNA, 2325 bp, synthetic construct
  source 1..2325: mol_type = other DNA
atgaggaattgtaagaaagatctatatgtagaggttaccccgatgcagaaagagttagtcctgggtatcgattacggcggtaagtataccgggctggcggtcgtggatcgtcgcaataaccaagttctgtacgcgcgtaccgtcaagatgcgtgacgatgtagctgatattttagagggtcgtcgggagcagcgtggtatccgtcgtaccggccagaccaagaagaaacgtctacgtgaattgaagacctatttgaaaaacattggcggtaagtacgacgaaagcatcggtacattcaagaacgaaccgtttatgaccgtgtacaaactgtgtcataagcgtggctatgactacgttgagatgccgaaagagaagacccgtgaagagattgaggctatgagcgcggatgagaagaagcaatgggaaaaagagaaaaaggagtgggaagagaccgaacgcaactctcgtcaccgtgacgaggtaacgaaggacgttcgtaaaatcatgagcgaacgcgacgcgaccgaacagcagatcagccatgttattaaaatcttcaacaaacaatatcgtccaaagcgttttaacaaccgtatcctgaccaagtgcaaagttgaagattgtggtgtcaataccccgctgcgtaaaaacgtgcgtgacctgctgattaaggacatcgtgcgttttctgccgctgacgcagagcgaaaaggctgaattggaggcccacgtgctggacaaagaaggccgcgagaaggttaaaaccttcttccgacgccaccgcattaacgagttcctgcgcaagcagatttatgacatcgctttcaatcagaaactttctggtcgtaccgtcttctgcaaaaagcacattctggagcacgaacaacacacgaaggaggaacgcaaggtgttccgcttggcgccaagcttgaaaaccaagatcaataacgtgttggctgtggttaaagatgagatccttccgcaatttggtattaataaagtgattatggagtccaacaattttgatatcgccgcacgtaccaagggcaaaaagagattagcaaaagacgagtatggtaagggtcataaagaagcgaaggagtccctcatcgaggtgctgctgagagaaaccaaaaaccaatgtgtttactgcggtaaggtgatcaccgttacgaccgcgaacaccgcgcacatctttccgcgtaaggcgggtggtatcaacgtctttgcaaatctggttgcctgctgtcgtgagtgcaatgaaaaaaaaaaaggccgtaccccgctggagtccggtgtgttgccacaaccggaaattactgcgcacctggagaacgatctgaagaagaaaattctggaggacgctcggaacatgaatgagttgaacctgaattcgatcaaatacatgagctatgccagcatcggctggcgtcacatgcgtgaccgactccgtggtttaatcggcaacgaagaactgccgattgaacgccaaagcggcatcttgaccgcgtactttcgtcgctggtggggtttccgcaaagaaaggggcaatgacattcatcacgcactggacgcggttattttagcgtctcgcaaggattacaccgacgacggcgttgatatgacgctgaagccgaaatccagcaacggtcgcgaattcgatccggaaaaacacctgccggataccatcagttttaaacgtgacaaaagcaacaaaggcacgcctctgtacgacaagaatccgctttcgattaagaacagcttgattacccgtcgctggatggttactgaaatcgaacatggtaaggaggataccattatttcggatgagtacagaaaaaagctaaaagaagcatttaagcattggggcacgagcaaaggcaaatgcctgacggatgaagaggcgcgtcagaccggcttttatcagcgcaaaaacgggaacttcatgtcactccgctgcgttgaaaaaggtctgggctggggacaaatggtgaaaatcaataacaacgcgttcaaaaccaatgtgcataatattggtgtagcagtgtacctggatgagaagggtaagaaaaaggcgtatgagatcaagaacccgcgtctgctcaaacacttcgtggaaaaaccgccggagataaaaggcaagaagctctacacactgcgtcgtggcgattatgttacgcatgaaagtgaggacagcgtttaccgtatcaaaaagctgggtacgtccccggttctcgaggccgtggttaaaggctctattggtagaatccgtaatagcgcatcggctaccaagttgactaaggtctccaaaccgcagaaagtgttc

SEQ ID NO: 34 - protein, 775 aa, synthetic construct
MRNCKKDLYVEVTPMQKELVLGIDYGGKYTGLAVVDRRNNQVLYARTVKMRDDVADILEGRREQRGIRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTAHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRDKSNKGTPLYDKNPLSIKNSLITRRWMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVEKPPEIKGKKLYTLRRGDYVTHESEDSVYRIKKLGTSPVLEAVVKGSIGRIRNSASATKLTKVSKPQKVF

SEQ ID NO: 35 - DNA, 2325 bp, synthetic construct
  source 1..2325: mol_type = other DNA
atgaggaattgtaagaaagatctatatgtagaggttaccccgatgcagaaagagttagtcctgggtatcgattacggcggtaagtataccgggctggcggtcgtggatcgtcgcaataaccaagttctgtacgcgcgtaccgtcaagatgcgtgacgatgtagctgatattttagagggtcgtcgggagcagcgtggtatccgtcgtaccggccagaccaagaagaaacgtctacgtgaattgaagacctatttgaaaaacattggcggtaagtacgacgaaagcatcggtacattcaagaacgaaccgtttatgaccgtgtacaaactgtgtcataagcgtggctatgactacgttgagatgccgaaagagaagacccgtgaagagattgaggctatgagcgcggatgagaagaagcaatgggaaaaagagaaaaaggagtgggaagagaccgaacgcaactctcgtcaccgtgacgaggtaacgaaggacgttcgtaaaatcatgagcgaacgcgacgcgaccgaacagcagatcagccatgttattaaaatcttcaacaaacaatatcgtccaaagcgttttaacaaccgtatcctgaccaagtgcaaagttgaagattgtggtgtcaataccccgctgcgtaaaaacgtgcgtgacctgctgattaaggacatcgtgcgttttctgccgctgacgcagagcgaaaaggctgaattggaggcccacgtgctggacaaagaaggccgcgagaaggttaaaaccttcttccgacgccaccgcattaacgagttcctgcgcaagcagatttatgacatcgctttcaatcagaaactttctggtcgtaccgtcttctgcaaaaagcacattctggagcacgaacaacacacgaaggaggaacgcaaggtgttccgcttggcgccaagcttgaaaaccaagatcaataacgtgttggctgtggttaaagatgagatccttccgcaatttggtattaataaagtgattatggagtccaacaattttgatatcgccgcacgtaccaagggcaaaaagagattagcaaaagacgagtatggtaagggtcataaagaagcgaaggagtccctcatcgaggtgctgctgagagaaaccaaaaaccaatgtgtttactgcggtaaggtgatcaccgttacgaccgcgaacaccgacgcgatctttccgcgtaaggcgggtggtatcaacgtctttgcaaatctggttgcctgctgtcgtgagtgcaatgaaaaaaaaaaaggccgtaccccgctggagtccggtgtgttgccacaaccggaaattactgcgcacctggagaacgatctgaagaagaaaattctggaggacgctcggaacatgaatgagttgaacctgaattcgatcaaatacatgagctatgccagcatcggctggcgtcacatgcgtgaccgactccgtggtttaatcggcaacgaagaactgccgattgaacgccaaagcggcatcttgaccgcgtactttcgtcgctggtggggtttccgcaaagaaaggggcaatgacattcatcacgcactggacgcggttattttagcgtctcgcaaggattacaccgacgacggcgttgatatgacgctgaagccgaaatccagcaacggtcgcgaattcgatccggaaaaacacctgccggataccatcagttttaaacgtgacaaaagcaacaaaggcacgcctctgtacgacaagaatccgctttcgattaagaacagcttgattacccgtcgctggatggttactgaaatcgaacatggtaaggaggataccattatttcggatgagtacagaaaaaagctaaaagaagcatttaagcattggggcacgagcaaaggcaaatgcctgacggatgaagaggcgcgtcagaccggcttttatcagcgcaaaaacgggaacttcatgtcactccgctgcgttgaaaaaggtctgggctggggacaaatggtgaaaatcaataacaacgcgttcaaaaccaatgtgcataatattggtgtagcagtgtacctggatgagaagggtaagaaaaaggcgtatgagatcaagaacccgcgtctgctcaaacacttcgtggaaaaaccgccggagataaaaggcaagaagctctacacactgcgtcgtggcgattatgttacgcatgaaagtgaggacagcgtttaccgtatcaaaaagctgggtacgtccccggttctcgaggccgtggttaaaggctctattggtagaatccgtaatagcgcatcggctaccaagttgactaaggtctccaaaccgcagaaagtgttc

SEQ ID NO: 36 - protein, 775 aa, synthetic construct
MRNCKKDLYVEVTPMQKELVLGIDYGGKYTGLAVVDRRNNQVLYARTVKMRDDVADILEGRREQRGIRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDAIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRDKSNKGTPLYDKNPLSIKNSLITRRWMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVEKPPEIKGKKLYTLRRGDYVTHESEDSVYRIKKLGTSPVLEAVVKGSIGRIRNSASATKLTKVSKPQKVF

SEQ ID NO: 37 - DNA, 2325 bp, synthetic construct
  source 1..2325: mol_type = other DNA
atgaggaattgtaagaaagatctatatgtagaggttaccccgatgcagaaagagttagtcctgggtatcgattacggcggtaagtataccgggctggcggtcgtggatcgtcgcaataaccaagttctgtacgcgcgtaccgtcaagatgcgtgacgatgtagctgatattttagagggtcgtcgggagcagcgtggtatccgtcgtaccggccagaccaagaagaaacgtctacgtgaattgaagacctatttgaaaaacattggcggtaagtacgacgaaagcatcggtacattcaagaacgaaccgtttatgaccgtgtacaaactgtgtcataagcgtggctatgactacgttgagatgccgaaagagaagacccgtgaagagattgaggctatgagcgcggatgagaagaagcaatgggaaaaagagaaaaaggagtgggaagagaccgaacgcaactctcgtcaccgtgacgaggtaacgaaggacgttcgtaaaatcatgagcgaacgcgacgcgaccgaacagcagatcagccatgttattaaaatcttcaacaaacaatatcgtccaaagcgttttaacaaccgtatcctgaccaagtgcaaagttgaagattgtggtgtcaataccccgctgcgtaaaaacgtgcgtgacctgctgattaaggacatcgtgcgttttctgccgctgacgcagagcgaaaaggctgaattggaggcccacgtgctggacaaagaaggccgcgagaaggttaaaaccttcttccgacgccaccgcattaacgagttcctgcgcaagcagatttatgacatcgctttcaatcagaaactttctggtcgtaccgtcttctgcaaaaagcacattctggagcacgaacaacacacgaaggaggaacgcaaggtgttccgcttggcgccaagcttgaaaaccaagatcaataacgtgttggctgtggttaaagatgagatccttccgcaatttggtattaataaagtgattatggagtccaacaattttgatatcgccgcacgtaccaagggcaaaaagagattagcaaaagacgagtatggtaagggtcataaagaagcgaaggagtccctcatcgaggtgctgctgagagaaaccaaaaaccaatgtgtttactgcggtaaggtgatcaccgttacgaccgcgaacaccgacctgatctttccgcgtaaggcgggtggtatcaacgtctttgcaaatctggttgcctgctgtcgtgagtgcaatgaaaaaaaaaaaggccgtaccccgctggagtccggtgtgttgccacaaccggaaattactgcgcacctggagaacgatctgaagaagaaaattctggaggacgctcggaacatgaatgagttgaacctgaattcgatcaaatacatgagctatgccagcatcggctggcgtcacatgcgtgaccgactccgtggtttaatcggcaacgaagaactgccgattgaacgccaaagcggcatcttgaccgcgtactttcgtcgctggtggggtttccgcaaagaaaggggcaatgacattcatcacgcactggacgcggttattttagcgtctcgcaaggattacaccgacgacggcgttgatatgacgctgaagccgaaatccagcaacggtcgcgaattcgatccggaaaaacacctgccggataccatcagttttaaacgtgacaaaagcaacaaaggcacgcctctgtacgacaagaatccgctttcgattaagaacagcttgattacccgtcgctggatggttactgaaatcgaacatggtaaggaggataccattatttcggatgagtacagaaaaaagctaaaagaagcatttaagcattggggcacgagcaaaggcaaatgcctgacggatgaagaggcgcgtcagaccggcttttatcagcgcaaaaacgggaacttcatgtcactccgctgcgttgaaaaaggtctgggctggggacaaatggtgaaaatcaataacaacgcgttcaaaaccaatgtgcataatattggtgtagcagtgtacctggatgagaagggtaagaaaaaggcgtatgagatcaagaacccgcgtctgctcaaacacttcgtggaaaaaccgccggagataaaaggcaagaagctctacacactgcgtcgtggcgattatgttacgcatgaaagtgaggacagcgtttaccgtatcaaaaagctgggtacgtccccggttctcgaggccgtggttaaaggctctattggtagaatccgtaatagcgcatcggctaccaagttgactaaggtctccaaaccgcagaaagtgttc

SEQ ID NO: 38 - protein, 775 aa, synthetic construct
MRNCKKDLYVEVTPMQKELVLGIDYGGKYTGLAVVDRRNNQVLYARTVKMRDDVADILEGRREQRGIRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDLIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRDKSNKGTPLYDKNPLSIKNSLITRRWMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVEKPPEIKGKKLYTLRRGDYVTHESEDSVYRIKKLGTSPVLEAVVKGSIGRIRNSASATKLTKVSKPQKVF

SEQ ID NO: 39 - DNA, 2325 bp, synthetic construct
  source 1..2325: mol_type = other DNA
atgaggaattgtaagaaagatctatatgtagaggttaccccgatgcagaaagagttagtcctgggtatcgattacggcggtaagtataccgggctggcggtcgtggatcgtcgcaataaccaagttctgtacgcgcgtaccgtcaagatgcgtgacgatgtagctgatattttagagggtcgtcgggagcagcgtggtatccgtcgtaccggccagaccaagaagaaacgtctacgtgaattgaagacctatttgaaaaacattggcggtaagtacgacgaaagcatcggtacattcaagaacgaaccgtttatgaccgtgtacaaactgtgtcataagcgtggctatgactacgttgagatgccgaaagagaagacccgtgaagagattgaggctatgagcgcggatgagaagaagcaatgggaaaaagagaaaaaggagtgggaagagaccgaacgcaactctcgtcaccgtgacgaggtaacgaaggacgttcgtaaaatcatgagcgaacgcgacgcgaccgaacagcagatcagccatgttattaaaatcttcaacaaacaatatcgtccaaagcgttttaacaaccgtatcctgaccaagtgcaaagttgaagattgtggtgtcaataccccgctgcgtaaaaacgtgcgtgacctgctgattaaggacatcgtgcgttttctgccgctgacgcagagcgaaaaggctgaattggaggcccacgtgctggacaaagaaggccgcgagaaggttaaaaccttcttccgacgccaccgcattaacgagttcctgcgcaagcagatttatgacatcgctttcaatcagaaactttctggtcgtaccgtcttctgcaaaaagcacattctggagcacgaacaacacacgaaggaggaacgcaaggtgttccgcttggcgccaagcttgaaaaccaagatcaataacgtgttggctgtggttaaagatgagatccttccgcaatttggtattaataaagtgattatggagtccaacaattttgatatcgccgcacgtaccaagggcaaaaagagattagcaaaagacgagtatggtaagggtcataaagaagcgaaggagtccctcatcgaggtgctgctgagagaaaccaaaaaccaatgtgtttactgcggtaaggtgatcaccgttacgaccgcgaacaccgaccacatctttccgcgtaaggcgggtggtatcaacgtctttgcaaatctggttgcctgctgtcgtgagtgcgcggaaaaaaaaaaaggccgtaccccgctggagtccggtgtgttgccacaaccggaaattactgcgcacctggagaacgatctgaagaagaaaattctggaggacgctcggaacatgaatgagttgaacctgaattcgatcaaatacatgagctatgccagcatcggctggcgtcacatgcgtgaccgactccgtggtttaatcggcaacgaagaactgccgattgaacgccaaagcggcatcttgaccgcgtactttcgtcgctggtggggtttccgcaaagaaaggggcaatgacattcatcacgcactggacgcggttattttagcgtctcgcaaggattacaccgacgacggcgttgatatgacgctgaagccgaaatccagcaacggtcgcgaattcgatccggaaaaacacctgccggataccatcagttttaaacgtgacaaaagcaacaaaggcacgcctctgtacgacaagaatccgctttcgattaagaacagcttgattacccgtcgctggatggttactgaaatcgaacatggtaaggaggataccattatttcggatgagtacagaaaaaagctaaaagaagcatttaagcattggggcacgagcaaaggcaaatgcctgacggatgaagaggcgcgtcagaccggcttttatcagcgcaaaaacgggaacttcatgtcactccgctgcgttgaaaaaggtctgggctggggacaaatggtgaaaatcaataacaacgcgttcaaaaccaatgtgcataatattggtgtagcagtgtacctggatgagaagggtaagaaaaaggcgtatgagatcaagaacccgcgtctgctcaaacacttcgtggaaaaaccgccggagataaaaggcaagaagctctacacactgcgtcgtggcgattatgttacgcatgaaagtgaggacagcgtttaccgtatcaaaaagctgggtacgtccccggttctcgaggccgtggttaaaggctctattggtagaatccgtaatagcgcatcggctaccaagttgactaaggtctccaaaccgcagaaagtgttc

SEQ ID NO: 40 - protein, 775 aa, synthetic construct
MRNCKKDLYVEVTPMQKELVLGIDYGGKYTGLAVVDRRNNQVLYARTVKMRDDVADILEGRREQRGIRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECAEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRDKSNKGTPLYDKNPLSIKNSLITRRWMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVEKPPEIKGKKLYTLRRGDYVTHESEDSVYRIKKLGTSPVLEAVVKGSIGRIRNSASATKLTKVSKPQKVF

SEQ ID NO: 41 - DNA, 2325 bp, synthetic construct
  source 1..2325: mol_type = other DNA
atgaggaattgtaagaaagatctatatgtagaggttaccccgatgcagaaagagttagtcctgggtatcgcgtacggcggtaagtataccgggctggcggtcgtggatcgtcgcaataaccaagttctgtacgcgcgtaccgtcaagatgcgtgacgatgtagctgatattttagagggtcgtcgggagcagcgtggtatccgtcgtaccggccagaccaagaagaaacgtctacgtgaattgaagacctatttgaaaaacattggcggtaagtacgacgaaagcatcggtacattcaagaacgaaccgtttatgaccgtgtacaaactgtgtcataagcgtggctatgactacgttgagatgccgaaagagaagacccgtgaagagattgaggctatgagcgcggatgagaagaagcaatgggaaaaagagaaaaaggagtgggaagagaccgaacgcaactctcgtcaccgtgacgaggtaacgaaggacgttcgtaaaatcatgagcgaacgcgacgcgaccgaacagcagatcagccatgttattaaaatcttcaacaaacaatatcgtccaaagcgttttaacaaccgtatcctgaccaagtgcaaagttgaagattgtggtgtcaataccccgctgcgtaaaaacgtgcgtgacctgctgattaaggacatcgtgcgttttctgccgctgacgcagagcgaaaaggctgaattggaggcccacgtgctggacaaagaaggccgcgagaaggttaaaaccttcttccgacgccaccgcattaacgagttcctgcgcaagcagatttatgacatcgctttcaatcagaaactttctggtcgtaccgtcttctgcaaaaagcacattctggagcacgaacaacacacgaaggaggaacgcaaggtgttccgcttggcgccaagcttgaaaaccaagatcaataacgtgttggctgtggttaaagatgagatccttccgcaatttggtattaataaagtgattatggagtccaacaattttgatatcgccgcacgtaccaagggcaaaaagagattagcaaaagacgagtatggtaagggtcataaagaagcgaaggagtccctcatcgaggtgctgctgagagaaaccaaaaaccaatgtgtttactgcggtaaggtgatcaccgttacgaccgcgaacaccgaccacatctttccgcgtaaggcgggtggtatcaacgtctttgcaaatctggttgcctgctgtcgtgagtgcaatgaaaaaaaaaaaggccgtaccccgctggagtccggtgtgttgccacaaccggaaattactgcgcacctggagaacgatctgaagaagaaaattctggaggacgctcggaacatgaatgagttgaacctgaattcgatcaaatacatgagctatgccagcatcggctggcgtcacatgcgtgaccgactccgtggtttaatcggcaacgaagaactgccgattgaacgccaaagcggcatcttgaccgcgtactttcgtcgctggtggggtttccgcaaagaaaggggcaatgacattcatcacgcactggacgcggttattttagcgtctcgcaaggattacaccgacgacggcgttgatatgacgctgaagccgaaatccagcaacggtcgcgaattcgatccggaaaaacacctgccggataccatcagttttaaacgtgacaaaagcaacaaaggcacgcctctgtacgacaagaatccgctttcgattaagaacagcttgattacccgtcgctggatggttactgaaatcgaacatggtaaggaggataccattatttcggatgagtacagaaaaaagctaaaagaagcatttaagcattggggcacgagcaaaggcaaatgcctgacggatgaagaggcgcgtcagaccggcttttatcagcgcaaaaacgggaacttcatgtcactccgctgcgttgaaaaaggtctgggctggggacaaatggtgaaaatcaataacaacgcgttcaaaaccaatgtgcataatattggtgtagcagtgtacctggatgagaagggtaagaaaaaggcgtatgagatcaagaacccgcgtctgctcaaacacttcgtggaaaaaccgccggagataaaaggcaagaagctctacacactgcgtcgtggcgattatgttacgcatgaaagtgaggacagcgtttaccgtatcaaaaagctgggtacgtccccggttctcgaggccgtggttaaaggctctattggtagaatccgtaatagcgcatcggctaccaagttgactaaggtctccaaaccgcagaaagtgttc

SEQ ID NO: 42 - protein, 775 aa, synthetic construct
MRNCKKDLYVEVTPMQKELVLGIAYGGKYTGLAVVDRRNNQVLYARTVKMRDDVADILEGRREQRGIRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRDKSNKGTPLYDKNPLSIKNSLITRRWMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVEKPPEIKGKKLYTLRRGDYVTHESEDSVYRIKKLGTSPVLEAVVKGSIGRIRNSASATKLTKVSKPQKVF

SEQ ID NO: 43 - DNA, 2325 bp, synthetic construct
  source 1..2325: mol_type = other DNA
atgaggaattgtaagaaagatctatatgtagaggttaccccgatgcagaaagagttagtcctgggtatcgattacggcggtaagtataccgggctggcggtcgtggatcgtcgcaataaccaagttctgtacgcgcgtaccgtcaagatgcgtgacgatgtagctgatattttagagggtcgtcgggagcagcgtggtatccgtcgtaccggccagaccaagaagaaacgtctacgtgaattgaagacctatttgaaaaacattggcggtaagtacgacgaaagcatcggtacattcaagaacgaaccgtttatgaccgtgtacaaactgtgtcataagcgtggctatgactacgttgagatgccgaaagagaagacccgtgaagagattgaggctatgagcgcggatgagaagaagcaatgggaaaaagagaaaaaggagtgggaagagaccgaacgcaactctcgtcaccgtgacgaggtaacgaaggacgttcgtaaaatcatgagcgaacgcgacgcgaccgaacagcagatcagccatgttattaaaatcttcaacaaacaatatcgtccaaagcgttttaacaaccgtatcctgaccaagtgcaaagttgaagattgtggtgtcaataccccgctgcgtaaaaacgtgcgtgacctgctgattaaggacatcgtgcgttttctgccgctgacgcagagcgaaaaggctgaattggaggcccacgtgctggacaaagaaggccgcgagaaggttaaaaccttcttccgacgccaccgcattaacgagttcctgcgcaagcagatttatgacatcgctttcaatcagaaactttctggtcgtaccgtcttctgcaaaaagcacattctggagcacgaacaacacacgaaggaggaacgcaaggtgttccgcttggcgccaagcttgaaaaccaagatcaataacgtgttggctgtggttaaagatgagatccttccgcaatttggtattaataaagtgattatggcgtccaacaattttgatatcgccgcacgtaccaagggcaaaaagagattagcaaaagacgagtatggtaagggtcataaagaagcgaaggagtccctcatcgaggtgctgctgagagaaaccaaaaaccaatgtgtttactgcggtaaggtgatcaccgttacgaccgcgaacaccgaccacatctttccgcgtaaggcgggtggtatcaacgtctttgcaaatctggttgcctgctgtcgtgagtgcaatgaaaaaaaaaaaggccgtaccccgctggagtccggtgtgttgccacaaccggaaattactgcgcacctggagaacgatctgaagaagaaaattctggaggacgctcggaacatgaatgagttgaacctgaattcgatcaaatacatgagctatgccagcatcggctggcgtcacatgcgtgaccgactccgtggtttaatcggcaacgaagaactgccgattgaacgccaaagcggcatcttgaccgcgtactttcgtcgctggtggggtttccgcaaagaaaggggcaatgacattcatcacgcactggacgcggttattttagcgtctcgcaaggattacaccgacgacggcgttgatatgacgctgaagccgaaatccagcaacggtcgcgaattcgatccggaaaaacacctgccggataccatcagttttaaacgtgacaaaagcaacaaaggcacgcctctgtacgacaagaatccgctttcgattaagaacagcttgattacccgtcgctggatggttactgaaatcgaacatggtaaggaggataccattatttcggatgagtacagaaaaaagctaaaagaagcatttaagcattggggcacgagcaaaggcaaatgcctgacggatgaagaggcgcgtcagaccggcttttatcagcgcaaaaacgggaacttcatgtcactccgctgcgttgaaaaaggtctgggctggggacaaatggtgaaaatcaataacaacgcgttcaaaaccaatgtgcataatattggtgtagcagtgtacctggatgagaagggtaagaaaaaggcgtatgagatcaagaacccgcgtctgctcaaacacttcgtggaaaaaccgccggagataaaaggcaagaagctctacacactgcgtcgtggcgattatgttacgcatgaaagtgaggacagcgtttaccgtatcaaaaagctgggtacgtccccggttctcgaggccgtggttaaaggctctattggtagaatccgtaatagcgcatcggctaccaagttgactaaggtctccaaaccgcagaaagtgttc

SEQ ID NO: 44 - protein, 775 aa, synthetic construct
MRNCKKDLYVEVTPMQKELVLGIDYGGKYTGLAVVDRRNNQVLYARTVKMRDDVADILEGRREQRGIRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMASNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRDKSNKGTPLYDKNPLSIKNSLITRRWMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVEKPPEIKGKKLYTLRRGDYVTHESEDSVYRIKKLGTSPVLEAVVKGSIGRIRNSASATKLTKVSKPQKVF

SEQ ID NO: 45 - DNA, 2325 bp, synthetic construct
  source 1..2325: mol_type = other DNA
atgaggaattgtaagaaagatctatatgtagaggttaccccgatgcagaaagagttagtcctgggtatcgattacggcggtaagtataccgggctggcggtcgtggatcgtcgcaataaccaagttctgtacgcgcgtaccgtcaagatgcgtgacgatgtagctgatattttagagggtcgtcgggagcagcgtggtatccgtcgtaccggccagaccaagaagaaacgtctacgtgaattgaagacctatttgaaaaacattggcggtaagtacgacgaaagcatcggtacattcaagaacgaaccgtttatgaccgtgtacaaactgtgtcataagcgtggctatgactacgttgagatgccgaaagagaagacccgtgaagagattgaggctatgagcgcggatgagaagaagcaatgggaaaaagagaaaaaggagtgggaagagaccgaacgcaactctcgtcaccgtgacgaggtaacgaaggacgttcgtaaaatcatgagcgaacgcgacgcgaccgaacagcagatcagccatgttattaaaatcttcaacaaacaatatcgtccaaagcgttttaacaaccgtatcctgaccaagtgcaaagttgaagattgtggtgtcaataccccgctgcgtaaaaacgtgcgtgacctgctgattaaggacatcgtgcgttttctgccgctgacgcagagcgaaaaggctgaattggaggcccacgtgctggacaaagaaggccgcgagaaggttaaaaccttcttccgacgccaccgcattaacgagttcctgcgcaagcagatttatgacatcgctttcaatcagaaactttctggtcgtaccgtcttctgcaaaaagcacattctggagcacgaacaacacacgaaggaggaacgcaaggtgttccgcttggcgccaagcttgaaaaccaagatcaataacgtgttggctgtggttaaagatgagatccttccgcaatttggtattaataaagtgattatggagtccaacaattttgatatcgccgcacgtaccaagggcaaaaagagattagcaaaagacgagtatggtaagggtcataaagaagcgaaggagtccctcatcgaggtgctgctgagagaaaccaaaaaccaatgtgtttactgcggtaaggtgatcaccgttacgaccgcgaacaccgaccacatctttccgcgtaaggcgggtggtatcaacgtctttgcaaatctggttgcctgctgtcgtgagtgcaatgaaaaaaaaaaaggccgtaccccgctggagtccggtgtgttgccacaaccggaaattactgcgcacctggagaacgatctgaagaagaaaattctggaggacgctcggaacatgaatgagttgaacctgaattcgatcaaatacatgagctatgccagcatcggctggcgtcacatgcgtgaccgactccgtggtttaatcggcaacgaagaactgccgattgaacgccaaagcggcatcttgaccgcgtactttcgtcgctggtggggtttccgcaaagaaaggggcaatgacattcatcacgcactggcggcggttattttagcgtctcgcaaggattacaccgacgacggcgttgatatgacgctgaagccgaaatccagcaacggtcgcgaattcgatccggaaaaacacctgccggataccatcagttttaaacgtgacaaaagcaacaaaggcacgcctctgtacgacaagaatccgctttcgattaagaacagcttgattacccgtcgctggatggttactgaaatcgaacatggtaaggaggataccattatttcggatgagtacagaaaaaagctaaaagaagcatttaagcattggggcacgagcaaaggcaaatgcctgacggatgaagaggcgcgtcagaccggcttttatcagcgcaaaaacgggaacttcatgtcactccgctgcgttgaaaaaggtctgggctggggacaaatggtgaaaatcaataacaacgcgttcaaaaccaatgtgcataatattggtgtagcagtgtacctggatgagaagggtaagaaaaaggcgtatgagatcaagaacccgcgtctgctcaaacacttcgtggaaaaaccgccggagataaaaggcaagaagctctacacactgcgtcgtggcgattatgttacgcatgaaagtgaggacagcgtttaccgtatcaaaaagctgggtacgtccccggttctcgaggccgtggttaaaggctctattggtagaatccgtaatagcgcatcggctaccaagttgactaaggtctccaaaccgcagaaagtgttc

SEQ ID NO: 46 - protein, 775 aa, synthetic construct
MRNCKKDLYVEVTPMQKELVLGIDYGGKYTGLAVVDRRNNQVLYARTVKMRDDVADILEGRREQRGIRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALAAVILASRKDYTDDGVDMTLKPKSSNGREFDPEKHLPDTISFKRDKSNKGTPLYDKNPLSIKNSLITRRWMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVEKPPEIKGKKLYTLRRGDYVTHESEDSVYRIKKLGTSPVLEAVVKGSIGRIRNSASATKLTKVSKPQKVF

SEQ ID NO: 47 - RNA, 20 nt, synthetic construct
  source 1..20: mol_type = other RNA
ttgtgagaatggtgcgtcct

SEQ ID NO: 48 - RNA, 28 nt, synthetic construct
  source 1..28: mol_type = other RNA
ggctcaccttcgggtgggcctttctgcg

SEQ ID NO: 49 - RNA, 251 nt, synthetic construct
  source 1..251: mol_type = other RNA
gttgtgagaatggtgcgtcctgttacagttaaggctctttggaaacaaagaagccttaattgtaaaacgcctatatggtaaagtgatgtacgtttgggtatatatcgccagcctgaacctctacgccagaaatggcagctttatcatgggttaggacgatatttaaaaaactttctgcggcttgcttactttagtaagctttgtggctgaggcagaattccttggctcaccttcgggtgggcctttctgcg

SEQ ID NO: 50 - DNA, 10 bp, synthetic construct
  source 1..10: mol_type = other DNA
aaaactccct

SEQ ID NO: 51 - DNA, 10 bp, synthetic construct
  source 1..10: mol_type = other DNA
aggtgttcac

SEQ ID NO: 52 - DNA, 100 bp, synthetic construct
  source 1..100: mol_type = other DNA
gctcaaagctttagactggaggtaccgtagtactatgagcttaatcgcgctgagggtgatttgggtgttcgtctcatatcttcgattatcggacagtagt

SEQ ID NO: 53 - DNA, 200 bp, synthetic construct
  source 1..200: mol_type = other DNA
agtagtatcggaccggttggcgaaccacagcgtataacgagacatggactcgcattccctaccttaagcgggcggttttggagctcggaattcatgtaaactcgcccgtttaacataatctagatggatcttgcgcgcaccgcccgaacaccactcgccacaaaaaaccgccggaacgtccaaaagtacgggttttgctg

SEQ ID NO: 54 - DNA, 340 bp, synthetic construct
  source 1..340: mol_type = other DNA
gctcaaagctttagactggaggtaccgtagtactatgagcttaatcgcgctgagggtgatttgggtgttcgtctcatatcttcgattatcggacagtagtaaaactccctttgtgagaatggtgcgtcctaggtgttcacagtagtatcggaccggttggcgaaccacagcgtataacgagacatggactcgcattccctaccttaagcgggcggttttggagctcggaattcatgtaaactcgcccgtttaacataatctagatggatcttgcgcgcaccgcccgaacaccactcgccacaaaaaaccgccggaacgtccaaaagtacgggttttgctg

SEQ ID NO: 55 - DNA, 27 bp, synthetic construct
  source 1..27: mol_type = other DNA
gctcaaagctttagactggaggtaccg

SEQ ID NO: 56 - DNA, 24 bp, synthetic construct
  source 1..24: mol_type = other DNA
cagcaaaacccgtacttttggacg

SEQ ID NO: 57 - DNA, 2526 bp, synthetic construct
  source 1..2526: mol_type = other DNA
atgaaacggacagccgacggaagcgagttcgagtcaccaaagaagaagcggaaagtccggaactgcaagaaggacctgtacgtggaagtgacccctatgcagaaagagctggtgctgggcattgactacggcggaaagtacacaggcctggccgtggtggaccggagaaacaaccaggtgctgtacgccagaaccgtcaagatgagagacgacgtggccgatatcctcgaaggccggagggaacagcgaggcagaagaagaacaggccagacaaagaagaagagactgagagaactgaaaacctacctgaagaacatcggaggaaaatatgacgagtctatcggcacattcaaaaacgagcctttcatgacagtgtacaagctgtgtcacaagagaggctacgactacgtggaaatgcctaaggaaaagaccagagaagaaatcgaggccatgtctgccgacgagaaaaagcagtgggaaaaagagaaaaaggagtgggaggaaaccgagcggaactccagacaccgggatgaggtgaccaaggacgtgcggaagatcatgagcgagagagatgccaccgagcagcagatcagccacgtgatcaagatcttcaacaagcagtaccggcccaagcggttcaataatagaatcctgacaaaatgtaaggttgaggattgcggcgtgaacacacctctgagaaagaacgtcagagacctgctgatcaaggacatcgtgagattcctgcccctgacccagagcgagaaagccgagctggaagctcatgtgctggataaggaaggaagagagaaggtgaagaccttcttcagacggcacagaatcaacgagttcctgcgcaagcagatctacgacatcgcctttaaccaaaagctgagcggcagaaccgtgttctgcaagaagcacatcctggagcacgagcaacacaccaaagaggaacgcaaggtctttagactggcccctagcctgaaaactaagattaacaacgtgctggctgtggttaaggacgagatcctgccccagttcggcatcaacaaggtcatcatggaatccaacaacttcgatattgccgctagaaccaagggcaagaagcggctggccaaggatgagtacggcaagggccacaaggaggccaaagagagcctgatcgaggtgctgctgcgggaaaccaagaaccagtgcgtgtactgcggaaaggtgatcaccgtgaccaccgccaataccgaccacatcttccccagaaaggcgggcggcatcaacgttttcgccaacctggtggcctgttgtagagagtgcaacgagaagaagaagggcagaacacctctggaatccggcgtgcttccacagcctgagatcaccgcccacctggaaaacgacctcaaaaagaagatccttgaggacgccagaaatatgaacgagctgaatctgaacagcatcaagtacatgagctacgccagtatcggatggcggcacatgagagatagactgaggggccttatcggtaatgaagaactgcctatcgagcgccaaagcggcatcctgacagcttacttcagacggtggtggggatttcggaaggaacggggaaatgacattcaccacgccctggacgctgtgatcctggccagcagaaaagactatacagatgacggcgtggatatgaccctgaagcccaaaagcagcaacggccgagaattcgaccccgagaaacatctgcctgatacaatcagcttcaaaagaagaaagtccaacaaaggaacccctctgtatgataagaacccattgtctatcaagaattctctgatcacaagaaggcggatggtgacagagatcgaacacggcaaggaggacaccatcatcagcgacgagtacagaaagaagctgaaggaggctttcaaacactggggcaccagcaagggcaagtgcctgaccgacgaggaagccagacagaccggcttctaccagagaaagaatggcaacttcatgagcctgcggtgcgtggagaagggcctgggctggggccagatggtgaagatcaataacaacgcctttaagacgaacgtgcacaacatcggggtggctgtttacctggacgagaagggcaagaagaaggcctacgaaattaagaaccccagactgctgaaacatttcgtgagaaagccacctgagatcaagggaaagaagctgtacaccctgcggagaggcgactacgtgacccacgagagcgaggactccgtgtacagaatcaaaaaactgggcacaagccctgtgctggaagctgtggtcaagggctctatcggccgcatccggaacagcgccagcgccacaaaactgaccaaggtgtccaagcctcagaaggtgttttctggaggatctagcggaggatcctctggcagcgagacaccaggaacaagcgagtcagcaacaccagagagcagtggcggcagcagcggcggcagcagcaaaaggccggcggccacgaaaaaggccggccaggcaaaaaagaaaaag

SEQ ID NO: 58 - protein, 842 aa, synthetic construct
MKRTADGSEFESPKKKRKVRNCKKDLYVEVTPMQKELVLGIDYGGKYTGLAVVDRRNNQVLYARTVKMRDDVADILEGRREQRGRRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRRKSNKGTPLYDKNPLSIKNSLITRRRMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVRKPPEIKGKKLYTLRRGDYVTHESEDSVYRIKKLGTSPVLEAVVKGSIGRIRNSASATKLTKVSKPQKVFSGGSSGGSSGSETPGTSESATPESSGGSSGGSSKRPAATKKAGQAKKKK

SEQ ID NO: 59 - DNA, 2484 bp, synthetic construct
  source 1..2484: mol_type = other DNA
atgaaacggacagccgacggaagcgagttcgagtcaccaaagaagaagcggaaagtccagaaagagctggtgctgggcattgactacggcggaaagtacacaggcctggccgtggtggaccggagaaacaaccaggtgctgtacgccagaaccgtcaagatgagagacgacgtggccgatatcctcgaaggccggagggaacagcgaggcattagaagaacaggccagacaaagaagaagagactgagagaactgaaaacctacctgaagaacatcggaggaaaatatgacgagtctatcggcacattcaaaaacgagcctttcatgacagtgtacaagctgtgtcacaagagaggctacgactacgtggaaatgcctaaggaaaagaccagagaagaaatcgaggccatgtctgccgacgagaaaaagcagtgggaaaaagagaaaaaggagtgggaggaaaccgagcggaactccagacaccgggatgaggtgaccaaggacgtgcggaagatcatgagcgagagagatgccaccgagcagcagatcagccacgtgatcaagatcttcaacaagcagtaccggcccaagcggttcaataatagaatcctgacaaaatgtaaggttgaggattgcggcgtgaacacacctctgagaaagaacgtcagagacctgctgatcaaggacatcgtgagattcctgcccctgacccagagcgagaaagccgagctggaagctcatgtgctggataaggaaggaagagagaaggtgaagaccttcttcagacggcacagaatcaacgagttcctgcgcaagcagatctacgacatcgcctttaaccaaaagctgagcggcagaaccgtgttctgcaagaagcacatcctggagcacgagcaacacaccaaagaggaacgcaaggtctttagactggcccctagcctgaaaactaagattaacaacgtgctggctgtggttaaggacgagatcctgccccagttcggcatcaacaaggtcatcatggaatccaacaacttcgatattgccgctagaaccaagggcaagaagcggctggccaaggatgagtacggcaagggccacaaggaggccaaagagagcctgatcgaggtgctgctgcgggaaaccaagaaccagtgcgtgtactgcggaaaggtgatcaccgtgaccaccgccaataccgaccacatcttccccagaaaggcgggcggcatcaacgttttcgccaacctggtggcctgttgtagagagtgcaacgagaagaagaagggcagaacacctctggaatccggcgtgcttccacagcctgagatcaccgcccacctggaaaacgacctcaaaaagaagatccttgaggacgccagaaatatgaacgagctgaatctgaacagcatcaagtacatgagctacgccagtatcggatggcggcacatgagagatagactgaggggccttatcggtaatgaagaactgcctatcgagcgccaaagcggcatcctgacagcttacttcagacggtggtggggatttcggaaggaacggggaaatgacattcaccacgccctggacgctgtgatcctggccagcagaaaagactatacagatgacggcgtggatatgaccctgaagcccaaaagcagcaacggccgagaattcgaccccgagaaacatctgcctgatacaatcagcttcaaaagagataagtccaacaaaggaacccctctgtatgataagaacccattgtctatcaagaattctctgatcacaagaaggtggatggtgacagagatcgaacacggcaaggaggacaccatcatcagcgacgagtacagaaagaagctgaaggaggctttcaaacactggggcaccagcaagggcaagtgcctgaccgacgaggaagccagacagaccggcttctaccagagaaagaatggcaacttcatgagcctgcggtgcgtggagaagggcctgggctggggccagatggtgaagatcaataacaacgcctttaagacgaacgtgcacaacatcggggtggctgtttacctggacgagaagggcaagaagaaggcctacgaaattaagaaccccagactgctgaaacatttcgtggaaaagccacctgagatcaagggaaagaagctgtacaccctgcggagaggcgactacgtgacccacgagagcgaggactccgtgtacagaatcaaaaaactgggcacaagccctgtgctggaagctgtggtcaagggctctatcggccgcatccggaacagcgccagcgccacaaaactgaccaaggtgtccaagcctcagaaggtgttttctggaggatctagcggaggatcctctggcagcgagacaccaggaacaagcgagtcagcaacaccagagagcagtggcggcagcagcggcggcagcagcaaaaggccggcggccacgaaaaaggccggccaggcaaaaaagaaaaag

SEQ ID NO: 60 - protein, 828 aa, synthetic construct
MKRTADGSEFESPKKKRKVQKELVLGIDYGGKYTGLAVVDRRNNQVLYARTVKMRDDVADILEGRREQRGIRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRDKSNKGTPLYDKNPLSIKNSLITRRWMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVEKPPEIKGKKLYTLRRGDYVTHESEDSVYRIKKLGTSPVLEAVVKGSIGRIRNSASATKLTKVSKPQKVFSGGSSGGSSGSETPGTSESATPESSGGSSGGSSKRPAATKKAGQAKKKK

SEQ ID NO: 61 - DNA, 2484 bp, synthetic construct
  source 1..2484: mol_type = other DNA
atgaaacggacagccgacggaagcgagttcgagtcaccaaagaagaagcggaaagtccagaaagagctggtgctgggcattgactacggcggaaagtacacaggcctggccgtggtggaccggagaaacaaccaggtgctgtacgccagaaccgtcaagatgagagacgacgtggccgatatcctcgaaggccggagggaacagcgaggcagaagaagaacaggccagacaaagaagaagagactgagagaactgaaaacctacctgaagaacatcggaggaaaatatgacgagtctatcggcacattcaaaaacgagcctttcatgacagtgtacaagctgtgtcacaagagaggctacgactacgtggaaatgcctaaggaaaagaccagagaagaaatcgaggccatgtctgccgacgagaaaaagcagtgggaaaaagagaaaaaggagtgggaggaaaccgagcggaactccagacaccgggatgaggtgaccaaggacgtgcggaagatcatgagcgagagagatgccaccgagcagcagatcagccacgtgatcaagatcttcaacaagcagtaccggcccaagcggttcaataatagaatcctgacaaaatgtaaggttgaggattgcggcgtgaacacacctctgagaaagaacgtcagagacctgctgatcaaggacatcgtgagattcctgcccctgacccagagcgagaaagccgagctggaagctcatgtgctggataaggaaggaagagagaaggtgaagaccttcttcagacggcacagaatcaacgagttcctgcgcaagcagatctacgacatcgcctttaaccaaaagctgagcggcagaaccgtgttctgcaagaagcacatcctggagcacgagcaacacaccaaagaggaacgcaaggtctttagactggcccctagcctgaaaactaagattaacaacgtgctggctgtggttaaggacgagatcctgccccagttcggcatcaacaaggtcatcatggaatccaacaacttcgatattgccgctagaaccaagggcaagaagcggctggccaaggatgagtacggcaagggccacaaggaggccaaagagagcctgatcgaggtgctgctgcgggaaaccaagaaccagtgcgtgtactgcggaaaggtgatcaccgtgaccaccgccaataccgaccacatcttccccagaaaggcgggcggcatcaacgttttcgccaacctggtggcctgttgtagagagtgcaacgagaagaagaagggcagaacacctctggaatccggcgtgcttccacagcctgagatcaccgcccacctggaaaacgacctcaaaaagaagatccttgaggacgccagaaatatgaacgagctgaatctgaacagcatcaagtacatgagctacgccagtatcggatggcggcacatgagagatagactgaggggccttatcggtaatgaagaactgcctatcgagcgccaaagcggcatcctgacagcttacttcagacggtggtggggatttcggaaggaacggggaaatgacattcaccacgccctggacgctgtgatcctggccagcagaaaagactatacagatgacggcgtggatatgaccctgaagcccaaaagcagcaacggccgagaattcgaccccgagaaacatctgcctgatacaatcagcttcaaaagaagaaagtccaacaaaggaacccctctgtatgataagaacccattgtctatcaagaattctctgatcacaagaaggtggatggtgacagagatcgaacacggcaaggaggacaccatcatcagcgacgagtacagaaagaagctgaaggaggctttcaaacactggggcaccagcaagggcaagtgcctgaccgacgaggaagccagacagaccggcttctaccagagaaagaatggcaacttcatgagcctgcggtgcgtggagaagggcctgggctggggccagatggtgaagatcaataacaacgcctttaagacgaacgtgcacaacatcggggtggctgtttacctggacgagaagggcaagaagaaggcctacgaaattaagaaccccagactgctgaaacatttcgtgagaaagccacctgagatcaagggaaagaagctgtacaccctgcggagaggcgactacgtgacccacgagagcgaggactccgtgtacagaatcaaaaaactgggcacaagccctgtgctggaagctgtggtcaagggctctatcggccgcatccggaacagcgccagcgccacaaaactgaccaaggtgtccaagcctcagaaggtgttttctggaggatctagcggaggatcctctggcagcgagacaccaggaacaagcgagtcagcaacaccagagagcagtggcggcagcagcggcggcagcagcaaaaggccggcggccacgaaaaaggccggccaggcaaaaaagaaaaag

SEQ ID NO: 62 - protein, 828 aa, synthetic construct
MKRTADGSEFESPKKKRKVQKELVLGIDYGGKYTGLAVVDRRNNQVLYARTVKMRDDVADILEGRREQRGRRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRRKSNKGTPLYDKNPLSIKNSLITRRWMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVRKPPEIKGKKLYTLRRGDYVTHESEDSVYRIKKLGTSPVLEAVVKGSIGRIRNSASATKLTKVSKPQKVFSGGSSGGSSGSETPGTSESATPESSGGSSGGSSKRPAATKKAGQAKKKK

SEQ ID NO: 63 - DNA, 2484 bp, synthetic construct
  source 1..2484: mol_type = other DNA
atgaaacggacagccgacggaagcgagttcgagtcaccaaagaagaagcggaaagtccagaaagagctggtgctgggcattgactacggcggaaagtacacaggcctggccgtggtggaccggagaaacaaccaggtgctgtacgccagaaccgtcaagatgagagacgacgtggccgatatcctcgaaggccggagggaacagcgaggcagaagaagaacaggccagacaaagaagaagagactgagagaactgaaaacctacctgaagaacatcggaggaaaatatgacgagtctatcggcacattcaaaaacgagcctttcatgacagtgtacaagctgtgtcacaagagaggctacgactacgtggaaatgcctaaggaaaagaccagagaagaaatcgaggccatgtctgccgacgagaaaaagcagtgggaaaaagagaaaaaggagtgggaggaaaccgagcggaactccagacaccgggatgaggtgaccaaggacgtgcggaagatcatgagcgagagagatgccaccgagcagcagatcagccacgtgatcaagatcttcaacaagcagtaccggcccaagcggttcaataatagaatcctgacaaaatgtaaggttgaggattgcggcgtgaacacacctctgagaaagaacgtcagagacctgctgatcaaggacatcgtgagattcctgcccctgacccagagcgagaaagccgagctggaagctcatgtgctggataaggaaggaagagagaaggtgaagaccttcttcagacggcacagaatcaacgagttcctgcgcaagcagatctacgacatcgcctttaaccaaaagctgagcggcagaaccgtgttctgcaagaagcacatcctggagcacgagcaacacaccaaagaggaacgcaaggtctttagactggcccctagcctgaaaactaagattaacaacgtgctggctgtggttaaggacgagatcctgccccagttcggcatcaacaaggtcatcatggaatccaacaacttcgatattgccgctagaaccaagggcaagaagcggctggccaaggatgagtacggcaagggccacaaggaggccaaagagagcctgatcgaggtgctgctgcgggaaaccaagaaccagtgcgtgtactgcggaaaggtgatcaccgtgaccaccgccaataccgaccacatcttccccagaaaggcgggcggcatcaacgttttcgccaacctggtggcctgttgtagagagtgcaacgagaagaagaagggcagaacacctctggaatccggcgtgcttccacagcctgagatcaccgcccacctggaaaacgacctcaaaaagaagatccttgaggacgccagaaatatgaacgagctgaatctgaacagcatcaagtacatgagctacgccagtatcggatggcggcacatgagagatagactgaggggccttatcggtaatgaagaactgcctatcgagcgccaaagcggcatcctgacagcttacttcagacggtggtggggatttcggaaggaacggggaaatgacattcaccacgccctggacgctgtgatcctggccagcagaaaagactatacagatgacggcgtggatatgaccctgaagcccaaaagcagcaacggccgagaattcgaccccgagaaacatctgcctgatacaatcagcttcaaaagaagaaagtccaacaaaggaacccctctgtatgataagaacccattgtctatcaagaattctctgatcacaagaaggcggatggtgacagagatcgaacacggcaaggaggacaccatcatcagcgacgagtacagaaagaagctgaaggaggctttcaaacactggggcaccagcaagggcaagtgcctgaccgacgaggaagccagacagaccggcttctaccagagaaagaatggcaacttcatgagcctgcggtgcgtggagaagggcctgggctggggccagatggtgaagatcaataacaacgcctttaagacgaacgtgcacaacatcggggtggctgtttacctggacgagaagggcaagaagaaggcctacgaaattaagaaccccagactgctgaaacatttcgtgagaaagccacctgagatcaagggaaagaagctgtacaccctgcggagaggcgactacgtgacccacgagagcgaggactccgtgtacagaatcaaaaaactgggcacaagccctgtgctggaagctgtggtcaagggctctatcggccgcatccggaacagcgccagcgccacaaaactgaccaaggtgtccaagcctcagaaggtgttttctggaggatctagcggaggatcctctggcagcgagacaccaggaacaagcgagtcagcaacaccagagagcagtggcggcagcagcggcggcagcagcaaaaggccggcggccacgaaaaaggccggccaggcaaaaaagaaaaag

SEQ ID NO: 64 - protein, 828 aa, synthetic construct
MKRTADGSEFESPKKKRKVQKELVLGIDYGGKYTGLAVVDRRNNQVLYARTVKMRDDVADILEGRREQRGRRRTGQTKKKRLRELKTYLKNIGGKYDESIGTFKNEPFMTVYKLCHKRGYDYVEMPKEKTREEIEAMSADEKKQWEKEKKEWEETERNSRHRDEVTKDVRKIMSERDATEQQISHVIKIFNKQYRPKRFNNRILTKCKVEDCGVNTPLRKNVRDLLIKDIVRFLPLTQSEKAELEAHVLDKEGREKVKTFFRRHRINEFLRKQIYDIAFNQKLSGRTVFCKKHILEHEQHTKEERKVFRLAPSLKTKINNVLAVVKDEILPQFGINKVIMESNNFDIAARTKGKKRLAKDEYGKGHKEAKESLIEVLLRETKNQCVYCGKVITVTTANTDHIFPRKAGGINVFANLVACCRECNEKKKGRTPLESGVLPQPEITAHLENDLKKKILEDARNMNELNLNSIKYMSYASIGWRHMRDRLRGLIGNEELPIERQSGILTAYFRRWWGFRKERGNDIHHALDAVILASRKDYTDDGVDMTLKPKSSNGREFDPEKHLPDTISFKRRKSNKGTPLYDKNPLSIKNSLITRRRMVTEIEHGKEDTIISDEYRKKLKEAFKHWGTSKGKCLTDEEARQTGFYQRKNGNFMSLRCVEKGLGWGQMVKINNNAFKTNVHNIGVAVYLDEKGKKKAYEIKNPRLLKHFVRKPPEIKGKKLYTLRRGDYVTHESEDSVYRIKKLGTSPVLEAVVKGSIGRIRNSASATKLTKVSKPQKVFSGGSSGGSSGSETPGTSESATPESSGGSSGGSSKRPAATKKAGQAKKKK

SEQ ID NO: 65 - protein, 747 aa, synthetic construct
MERELVLGIDYGGKYTGLAVVDRRHNQVLYANRLKMRDDVAGILKDRRKQRGIRRTAQTKKKRLRELKNYLKSIGYNESTATFETVYSLAHKRGYDYADMPEEKTSEEIEAMDVEERKQWEKEKQEWEETKRNSRHRKEVVKDVHKAMIEGRATEEQIKRVERIFNKQYRPKRFNNRILTKCKVEDCGVNTPLRKNVRDLLIENIVRFFPIEQSEKDNLKDAVLDKNRREEVKSFFRKHKTDEHIRKQVYDIADNKLSGRTVFCKEHILEHTEHSKEERKVFRLAPSLKTKIENVLAVIKDEILPKFTVNKVVMESNNFDIAAKTQGKKRLAKEEYGKGPREGKETRKEALLRETDGRCIYCGKSIDISNAHDDHIFPRKAGGLNIFANLVACCAVCNENKKGRTPLESGISPKPEIIAFMKNDLKKKILEDARNINTVDFNKYMSHASIGWRYMRDRLRESAGNKKLPIERQSGIYTAYFRRWWGFKKERGNTLHHALDAVILASRKGYSDDGLVDMTLKPKYNKGGGFDSEKHLPEPIEFKRDKGSKGSALHDRNPLSYKKGIITRRFMVTEIECGKEDDVISETYREKLKEAFKRFDTKKGKCLTDKEAKEAGFCIKKNELVMSLKCSIKGTGPGQMIRINNNVFKTNVHNVGVDVYLDEKGKKKAYERKNPRLSKHFIEPPPQPNGRVSFTLKRRDMVTVEGEDAIYRIKKLGTSPTIEAVVGSDGKTRTVSATKLTKANSAE

SEQ ID NO: 66 - protein, 781 aa, synthetic construct
MKRTADGSEFESPKKKRKVERELVLGIDYGGKYTGLAVVDRRHNQVLYANRLKMRDDVAGILKDRRKQRGIRRTAQTKKKRLRELKNYLKSIGYNESTATFETVYSLAHKRGYDYADMPEEKTSEEIEAMDVEERKQWEKEKQEWEETKRNSRHRKEVVKDVHKAMIEGRATEEQIKRVERIFNKQYRPKRFNNRILTKCKVEDCGVNTPLRKNVRDLLIENIVRFFPIEQSEKDNLKDAVLDKNRREEVKSFFRKHKTDEHIRKQVYDIADNKLSGRTVFCKEHILEHTEHSKEERKVFRLAPSLKTKIENVLAVIKDEILPKFTVNKVVMESNNFDIAAKTQGKKRLAKEEYGKGPREGKETRKEALLRETDGRCIYCGKSIDISNAHDDHIFPRKAGGLNIFANLVACCAVCNENKKGRTPLESGISPKPEIIAFMKNDLKKKILEDARNINTVDFNKYMSHASIGWRYMRDRLRESAGNKKLPIERQSGIYTAYFRRWWGFKKERGNTLHHALDAVILASRKGYSDDGLVDMTLKPKYNKGGGFDSEKHLPEPIEFKRDKGSKGSALHDRNPLSYKKGIITRRFMVTEIECGKEDDVISETYREKLKEAFKRFDTKKGKCLTDKEAKEAGFCIKKNELVMSLKCSIKGTGPGQMIRINNNVFKTNVHNVGVDVYLDEKGKKKAYERKNPRLSKHFIEPPPQPNGRVSFTLKRRDMVTVEGEDAIYRIKKLGTSPTIEAVVGSDGKTRTVSATKLTKANSAEKRPAATKKAGQAKKKK

SEQ ID NO: 67 - DNA, 20 bp, synthetic construct
  source 1..20: mol_type = other DNA
ttgtgagaatggtgcgtcct

SEQ ID NO: 68 - RNA, 199 nt, synthetic construct
  source 1..199: mol_type = other RNA
ttgtgagaatggtgcgtcctgttacagttaaggctctgaaaagagccttaattgtaaaacgcctatacagtgaagggatatacgcttgggtttgtccagcctgagcctctatgccagaaatggcgccttcatcgtgggttaggacatttaaatttaaaaactattcagcactgtttgctcttgtcagcttggtggcaga

SEQ ID NO: 69 - DNA, 20 bp, synthetic construct
  source 1..20: mol_type = other DNA
ctttgtagcctctcccgctc

SEQ ID NO: 70 - RNA, 199 nt, synthetic construct
  source 1..199: mol_type = other RNA
ctttgtagcctctcccgctcgttacagttaaggctctgaaaagagccttaattgtaaaacgcctatacagtgaagggatatacgcttgggtttgtccagcctgagcctctatgccagaaatggcgccttcatcgtgggttaggacatttaaatttaaaaactattcagcactgtttgctcttgtcagcttggtggcaga

SEQ ID NO: 71 - DNA, 20 bp, synthetic construct
  source 1..20: mol_type = other DNA
tggatggcgacttcaggcac

SEQ ID NO: 72 - RNA, 199 nt, synthetic construct
  source 1..199: mol_type = other RNA
tggatggcgacttcaggcacgttacagttaaggctctgaaaagagccttaattgtaaaacgcctatacagtgaagggatatacgcttgggtttgtccagcctgagcctctatgccagaaatggcgccttcatcgtgggttaggacatttaaatttaaaaactattcagcactgtttgctcttgtcagcttggtggcaga

SEQ ID NO: 73 - DNA, 20 bp, synthetic construct
  source 1..20: mol_type = other DNA
ctttagcaagggactattca

SEQ ID NO: 74 - RNA, 199 nt, synthetic construct
  source 1..199: mol_type = other RNA
ctttagcaagggactattcagttacagttaaggctctgaaaagagccttaattgtaaaacgcctatacagtgaagggatatacgcttgggtttgtccagcctgagcctctatgccagaaatggcgccttcatcgtgggttaggacatttaaatttaaaaactattcagcactgtttgctcttgtcagcttggtggcaga

SEQ ID NO: 75 - DNA, 20 bp, synthetic construct
  source 1..20: mol_type = other DNA
ttgttgacattgtccacacc

SEQ ID NO: 76 - RNA, 199 nt, synthetic construct
  source 1..199: mol_type = other RNA
ttgttgacattgtccacaccgttacagttaaggctctgaaaagagccttaattgtaaaacgcctatacagtgaagggatatacgcttgggtttgtccagcctgagcctctatgccagaaatggcgccttcatcgtgggttaggacatttaaatttaaaaactattcagcactgtttgctcttgtcagcttggtggcaga

SEQ ID NO: 77 - DNA, 20 bp, synthetic construct
  source 1..20: mol_type = other DNA
tttgcccatactggggacca

SEQ ID NO: 78 - RNA, 199 nt, synthetic construct
  source 1..199: mol_type = other RNA
tttgcccatactggggaccagttacagttaaggctctgaaaagagccttaattgtaaaacgcctatacagtgaagggatatacgcttgggtttgtccagcctgagcctctatgccagaaatggcgccttcatcgtgggttaggacatttaaatttaaaaactattcagcactgtttgctcttgtcagcttggtggcaga

SEQ ID NO: 79 - RNA, 179 nt, synthetic construct
  source 1..179: mol_type = other RNA
gttacagttaaggctctgaaaagagccttaattgtaaaacgcctatacagtgaagggatatacgcttgggtttgtccagcctgagcctctatgccagaaatggcgccttcatcgtgggttaggacatttaaatttaaaaactattcagcactgtttgctcttgtcagcttggtggcaga

SEQ ID NO: 80 - protein, 497 aa, synthetic construct
MVYVISKNGKPLMPTKRHGLVRILLKEKKARVVQRKPFTIQLLYDSTTYTQDITAGFDTGRAYQSITAVNARTGEVLYSSVLETRNKEVPKLMKSRKMYRAIRRHNRRMKKVRRAVRNKTYFRAPKKVVWPGAKEPITAKYIKPKEARFNNRKRPEGWLTPTAVHLLRTHLNYFKKVRKILPIKTLVLEYGKFDIQKLENPDIKGAEYQRGKLYGYNNLREYVIAVQQGKCLLCEKKPIEHLHHVMPGSKEGSDTYKNIAGLCSKCHAKVHTSPKAKEKLAEKAAGTAKEYADTSILNIIMPYLYSELKSMLGTENIALCYGYETEAARKSFGLAKSHSNDSYAMALMAIGQASRIEKIEPYRYKQYRRHNRAFCDAQRDRLYKKDGKIAARNRRRRTEQEGISLAEYRSELITALGKKIATREISKLKVYRAVKKKSTSIKDVLFPPGCAVNYKGQRAVVKSFFNKGSSLILEGISGYVPAKDCQLITKKAGIVCL

SEQ ID NO: 81 - protein, 564 aa, synthetic construct
MKRTADGSEFESPKKKRKVVYVISKNGKPLMPTKRHGLVRILLKEKKARVVQRKPFTIQLLYDSTTYTQDITAGFDTGRAYQSITAVNARTGEVLYSSVLETRNKEVPKLMKSRKMYRAIRRHNRRMKKVRRAVRNKTYFRAPKKVVWPGAKEPITAKYIKPKEARFNNRKRPEGWLTPTAVHLLRTHLNYFKKVRKILPIKTLVLEYGKFDIQKLENPDIKGAEYQRGKLYGYNNLREYVIAVQQGKCLLCEKKPIEHLHHVMPGSKEGSDTYKNIAGLCSKCHAKVHTSPKAKEKLAEKAAGTAKEYADTSILNIIMPYLYSELKSMLGTENIALCYGYETEAARKSFGLAKSHSNDSYAMALMAIGQASRIEKIEPYRYKQYRRHNRAFCDAQRDRLYKKDGKIAARNRRRRTEQEGISLAEYRSELITALGKKIATREISKLKVYRAVKKKSTSIKDVLFPPGCAVNYKGQRAVVKSFFNKGSSLILEGISGYVPAKDCQLITKKAGIVCLSGGSSGGSSGSETPGTSESATPESSGGSSGGSSKRPAATKKAGQAKKKK

SEQ ID NO: 82 - DNA, 20 bp, synthetic construct
  source 1..20: mol_type = other DNA
gctgccaaagccagggccag

SEQ ID NO: 83 - RNA, 236 nt, synthetic construct
  source 1..236: mol_type = other RNA
gctgccaaagccagggccaggtcaactacccccgtctaaagacggaggcatgaggtttcgtaaccaagtgttgtacctgcgggtacagtagttgaacaggcggcgatgcggctgggcactccaggatgccactcccagtcccggacactgccgacgagccgcatcaagccgggggagaccaaccggctaacgatagccgagcaattacctaaaaagaggtgcaaaggaaatggtat

SEQ ID NO: 84 - DNA, 20 bp, synthetic construct
  source 1..20: mol_type = other DNA
caaagctggggccttcggct

SEQ ID NO: 85 - RNA, 236 nt, synthetic construct
  source 1..236: mol_type = other RNA
caaagctggggccttcggctgtcaactacccccgtctaaagacggaggcatgaggtttcgtaaccaagtgttgtacctgcgggtacagtagttgaacaggcggcgatgcggctgggcactccaggatgccactcccagtcccggacactgccgacgagccgcatcaagccgggggagaccaaccggctaacgatagccgagcaattacctaaaaagaggtgcaaaggaaatggtat

SEQ ID NO: 86 - DNA, 20 bp, synthetic construct
  source 1..20: mol_type = other DNA
agtggtgcctggaaaataaa

SEQ ID NO: 87 - RNA, 236 nt, synthetic construct
  source 1..236: mol_type = other RNA
agtggtgcctggaaaataaagtcaactacccccgtctaaagacggaggcatgaggtttcgtaaccaagtgttgtacctgcgggtacagtagttgaacaggcggcgatgcggctgggcactccaggatgccactcccagtcccggacactgccgacgagccgcatcaagccgggggagaccaaccggctaacgatagccgagcaattacctaaaaagaggtgcaaaggaaatggtat

SEQ ID NO: 88 - DNA, 20 bp, synthetic construct
  source 1..20: mol_type = other DNA
cttgctaaagaaacatgtgt

SEQ ID NO: 89 - RNA, 236 nt, synthetic construct
  source 1..236: mol_type = other RNA
cttgctaaagaaacatgtgtgtcaactacccccgtctaaagacggaggcatgaggtttcgtaaccaagtgttgtacctgcgggtacagtagttgaacaggcggcgatgcggctgggcactccaggatgccactcccagtcccggacactgccgacgagccgcatcaagccgggggagaccaaccggctaacgatagccgagcaattacctaaaaagaggtgcaaaggaaatggtat

SEQ ID NO: 90 - DNA, 20 bp, synthetic construct
  source 1..20: mol_type = other DNA
tcaccatgactggtggagga

SEQ ID NO: 91 - RNA, 236 nt, synthetic construct
  source 1..236: mol_type = other RNA
tcaccatgactggtggaggagtcaactacccccgtctaaagacggaggcatgaggtttcgtaaccaagtgttgtacctgcgggtacagtagttgaacaggcggcgatgcggctgggcactccaggatgccactcccagtcccggacactgccgacgagccgcatcaagccgggggagaccaaccggctaacgatagccgagcaattacctaaaaagaggtgcaaaggaaatggtat

SEQ ID NO: 92 - DNA, 20 bp, synthetic construct
  source 1..20: mol_type = other DNA
gagcctggtccatggagagt

SEQ ID NO: 93 - RNA, 236 nt, synthetic construct
  source 1..236: mol_type = other RNA
gagcctggtccatggagagtgtcaactacccccgtctaaagacggaggcatgaggtttcgtaaccaagtgttgtacctgcgggtacagtagttgaacaggcggcgatgcggctgggcactccaggatgccactcccagtcccggacactgccgacgagccgcatcaagccgggggagaccaaccggctaacgatagccgagcaattacctaaaaagaggtgcaaaggaaatggtat

SEQ ID NO: 94 - RNA, 216 nt, synthetic construct
  source 1..216: mol_type = other RNA
gtcaactacccccgtctaaagacggaggcatgaggtttcgtaaccaagtgttgtacctgcgggtacagtagttgaacaggcggcgatgcggctgggcactccaggatgccactcccagtcccggacactgccgacgagccgcatcaagccgggggagaccaaccggctaacgatagccgagcaattacctaaaaagaggtgcaaaggaaatggtat

SEQ ID NO: 95 - protein, 33 aa, synthetic construct
SGGSSGGSSGSETPGTSESATPESSGGSSGGSS

(19) United States
(12) Patent Application Publication
(10) Pub. No.: US 2026/0275311 A1
(43) Pub. Date: Sep. 17, 2026

(54) CRISPR NUCLEASE POLYPEPTIDES AND GENE EDITING SYSTEMS COMPRISING SUCH

(71) Applicant: ARBOR BIOTECHNOLOGIES, INC., Cambridge, MA (US)

(72) Inventors: Lauren E. Alfonse, Cambridge, MA (US); Zachary J. Maben, Cambridge, MA (US); Chad D. Torgerson, Cambridge, MA (US); Kyle E. Watters, Cambridge, MA (US)

(21) Appl. No.: 19/469,502

(22) PCT Filed: Mar. 29, 2024
(86) PCT No.: PCT/US2024/022145

§ 371 Date: Sep. 26, 2025

Related U.S. Application Data
(60) Provisional application No. 63/493,363, filed on Mar. 31, 2023.
(60) Provisional application No. 63/493,360, filed on Mar. 31, 2023.
(60) Provisional application No. 63/493,355, filed on Mar. 31, 2023.
(60) Provisional application No. 63/516,246, filed on Jul. 28, 2023.
(60) Provisional application No. 63/562,132, filed on Mar. 6, 2024.
(60) Provisional application No. 63/566,661, filed on Mar. 18, 2024.

(51) Int. Cl. C12N 9/22; C12N 15/11; C12N 15/86; C12N 15/88

(52) U.S. Cl. CPC C12N 9/226; C12N 15/111; C12N 15/86; C12N 15/88; C07K 2319/09; C12N 2310/20

(57) ABSTRACT

A nuclease polypeptide such as a CRISPR nuclease polypeptide derived from a reference nuclease, which can be Nuclease A, Nuclease K, or Nuclease M, the nuclease polypeptide comprising a RuvC nuclease domain and an HNH nuclease domain. Also provided herein are gene editing systems comprising such a nuclease polypeptide and gene editing methods using the gene editing system.

### CROSS REFERENCE TO RELATED APPLICATIONS

This application claims the benefit of the filing dates of U.S. Provisional Application No. 63/493,360, filed Mar. 31, 2023, U.S. Provisional Application No. 63/516,246, filed Jul. 28, 2023; U.S. Provisional Application No. 63/566,661, filed Mar. 18, 2024, U.S. Provisional Application No. 63/493,355, filed Mar. 31, 2023, U.S. Provisional Application No. 63/562,132, filed Mar. 6, 2024, and U.S. Provisional Application No. 63/493,363, filed Mar. 31, 2023. Each of the priority applications is incorporated by reference herein in their entities.

### SEQUENCE LISTING

The instant application contains a Sequence Listing which has been filed electronically in XML format and is hereby incorporated by reference in its entirety. Said XML copy, created on Mar. 27, 2024, is named 063586-514001WO_SeqList_ST26.xml and is 137,220 bytes in size.

### BACKGROUND

Clustered Regularly Interspaced Short Palindromic Repeats (CRISPR) and CRISPR-associated (Cas) genes, collectively known as CRISPR-Cas or CRISPR/Cas systems, are adaptive immune systems in archaea and bacteria that defend particular species against foreign genetic elements.

A CRISPR-Cas system typically comprises a CRISPR nuclease and one or more RNA components that direct the CRISPR nuclease to a target genomic site for gene editing. It is of interest to develop efficient CRISPR nucleases to improve gene editing efficiency.

### SUMMARY OF THE INVENTION

The present disclosure provides CRISPR nucleases that exhibit advantageous enzymatic activities (e.g., nickase activity, high indel activities, high binding activities to the scaffold of a cognate guide RNA, and/or high DNA cleavage activity). Accordingly, the CRISPR nucleases provided herein would be expected to show superior effectiveness when used in gene editing.

Accordingly, provided herein are CRISPR nuclease polypeptides derived from a reference CRISPR nuclease, including Nuclease A (SEQ ID NO: 1), Nuclease K (SEQ ID NO: 65) or Nuclease M (SEQ ID NO: 80). Such CRISPR nucleases comprise a RuvC nuclease domain and an HNH nuclease domain. In some instances, the CRISPR nuclease polypeptides provided herein share an amino acid sequence at least 85% (e.g., at least 90%) identical to the reference CRISPR nuclease provided herein. In some instances, the CRISPR nuclease polypeptides disclosed herein comprise a variant CRISPR nuclease (a.k.a., engineered CRISPR nuclease), which comprises at least one mutation relative to the reference CRISPR nuclease.

In some aspects, the present disclosure provides CRISPR nuclease polypeptides derived from reference Nuclease A, gene editing systems comprising the CRISPR nuclease polypeptide derived from Nuclease A and a guide RNA targeting a genomic site of interest, and uses of the gene editing system for modifying the genomic site of interest in host cells.

In some embodiments, the CRISPR nuclease polypeptide is an engineered variant of reference Nuclease A (SEQ ID NO: 1), comprising at least one mutation relative to Nuclease A. In some instances, the at least one mutation may (a) comprise one or more arginine and/or lysine substitutions (e.g., arginine substitutions) relative to Nuclease A, (b) comprise one or more nickase mutations in the HNH nuclease domain or in the RuvC nuclease domain of Nuclease A, (c) comprise an N-terminal truncation relative to Nuclease A, or any combination of (a), (b) and (c).

Like Nuclease A, the CRISPR nuclease polypeptide derived from Nuclease A may comprise a bridge helix (BH) domain, a phosphate lock loop (PLL) domain, a wedge (WED) domain, and a PAM-interacting (PID) domain. In some examples, the variant of Nuclease A provided herein may comprise one or more arginine and/or lysine substitutions (e.g., arginine substitutions). In some instances, the one or more arginine and/or lysine substitutions (e.g., arginine substitutions) are located in the BH domain, in the PLL domain, in the WED domain, in the PID domain, or a combination thereof. In some instances, the one or more arginine and/or lysine substitutions (e.g., arginine substitutions) may be located at one or more of positions D56, E59, G60, E63, 167, G71, S564, D568, W593, T605, E655, E694, and E706 in SEQ ID NO: 1. In some specific examples, the engineered variant of Nuclease A comprises arginine and/or lysine substitutions (e.g., arginine substitutions) at the following positions relative to SEQ ID NO: 1: a) I67, D568, and E706; b) 167, D56, and D568; c) I67, T605, and D568; d) I67, D56, and E706; e) I67, T605, and E706; 1) 167, D568, and E694; g) I67, D56, E694, and E706; h) 167, D56, T605, and E706; or i) 167, D568, W593, and E706. In one specific example, the engineered variant of Nuclease A comprises arginine and/or lysine substitutions at 167, D568, and E706.

In some examples, the engineered variant nuclease polypeptide of Nuclease A contains the arginine substitutions of (a) I67R, D568R, and E706R. In some examples, the engineered CRISPR nuclease polypeptide contains the arginine substitutions of (b) I67R, D56R, and D568R. In some examples, the engineered CRISPR nuclease polypeptide contains the arginine substitutions of (c) 167R, T605R, and D568R. In some examples, the engineered CRISPR nuclease polypeptide contains the arginine substitutions of (d) I67R, D56R, and E706R. In some examples, the engineered CRISPR nuclease polypeptide contains the arginine substitutions of (e) I67R, T605R, and E706R. In some examples, the engineered CRISPR nuclease polypeptide contains the arginine substitutions of (f) I67R, D568R, and E694R. In some examples, the engineered CRISPR nuclease polypeptide contains the arginine substitutions of (g) I67R, D56R, E694R, and E706R. In some examples, the engineered CRISPR nuclease polypeptide contains the arginine substitutions of (h) I67R, D56R, T605R, and E706R. In some examples, the engineered CRISPR nuclease polypeptide contains the arginine substitutions of (i) I67R, D568R, W593R, and E706R. In some specific examples, the engineered CRISPR nuclease polypeptide contains the arginine substitutions of I67R, D568R, and E706R.

Any of the engineered variant of Nuclease A disclosed herein may contain up to 20 arginine and/or lysine substitutions (e.g., arginine substitutions), for example, up to 15 arginine and/or lysine substitutions (e.g., arginine substitutions).

Alternatively or in addition, the engineered variant of Nuclease A may have an N-terminal truncation relative to Nuclease A (SEQ ID NO: 1). In some examples, the N-terminal truncation is a deletion within residues 1-15 of SEQ ID NO: 1. In some specific examples, the N-terminal truncation is a deletion of residues 1-14. In another specific example, the N-terminal truncation is a deletion of residues 1-15 of SEQ ID NO: 1.

In addition, the CRISPR nuclease polypeptide disclosed herein may be a nickase variant of Nuclease A, which exhibit nickase activity. Such a nickase variant may comprise one or more mutations at position H397, D396, N420, D24, E337, and/or D524 of SEQ ID NO: 1. In some examples, the nickase variant may comprise a mutation at position H397. In some specific examples, the mutation is amino acid substitution of H397A or H397L.

In some embodiments, an engineered variant of Nuclease A as disclosed herein may comprise a combination of any of the mutations disclosed herein, e.g., one or more arginine/lysine substitutions, one or more nickase mutations, and/or N-terminal truncation. For example, the variant may comprise: (a) the one or more nickase mutations in the HNH nuclease domain (e.g., at positions D396, H397, and/or N420 relative to SEQ ID NO: 1); and the one or more arginine and/or lysine substitutions. In specific examples, the nickase mutation is at position H397 and the arginine/lysine substitutions are at positions I67, D568, and E706 of SEQ ID NO: 1.

In another example, the engineered variant of Nuclease A may comprise: (a) the one or more nickase mutations in the HNH nuclease domain (e.g., at positions D396, H397, and/or N420 relative to SEQ ID NO: 1); (b) the one or more arginine and/or lysine substitutions (e.g., at positions 167, D568, and E706); and (c) the N-terminal truncation within residues 1-15 of SEQ ID NO: 1 (e.g., deletion of residues 1-14 or 1-15 of SEQ ID NO: 1). Alternatively or in addition, the engineered variant of Nuclease A may comprise a C-terminal truncation relative to Nuclease A (e.g., within the C-terminal 1-5 residues of SEQ ID NO: 1).

In yet another example, the variant of Nuclease A comprises: (a) the nickase mutation of H397A; (b) the arginine substitutions of I67R, D568R, and E706R; and (c) the N-terminal truncation comprising deletion of residues 1-14 or 1-15 of SEQ ID NO: 1.

Any of the engineered variants of Nuclease A disclosed herein may comprise an amino acid sequence at least 95% identical to SEQ ID NO: 1. In some examples, the engineered variant may comprise an amino acid sequence at least 98% identical to SEQ ID NO: 1.

Exemplary CRISPR nuclease polypeptides derived from Nuclease A as provided herein are listed in Table 1, Table 11, or Table 14, each of which is within the scope of the present disclosure.

Any of the CRISPR nuclease polypeptides derived from Nuclease A disclosed herein may be a fusion polypeptide, which may further comprise one or more functional fragments. In some embodiments, the one or more functional fragments may comprise one or more nuclear localization signals (NLSs), one or more peptide linkers, or a combination thereof. The one or more NLS may be located at the N-terminus, at the C-terminus, or both.

Also provided herein is a nucleic acid, comprising a nucleotide sequence encoding any of the CRISPR nuclease polypeptides derived from Nuclease A as disclosed herein. In some instances, the nucleic acid is an expression vector, in which the nucleotide sequence encoding the CRISPR nuclease polypeptide is in operable linkage to a promoter. Further, the present disclosure provides a host cell comprising the nucleic acid coding for the CRISPR nuclease polypeptide as disclosed herein.

In some embodiments, the present disclosure features a gene editing system, comprising: (a) any CRISPR nuclease polypeptide derived from Nuclease A as disclosed herein or a first nucleic acid encoding the CRISPR nuclease polypeptide; and (b) a guide RNA (gRNA) or a second nucleic acid encoding the gRNA. The gRNA comprises a scaffold sequence recognizable by the CRISPR nuclease polypeptide and a spacer sequence specific to a target sequence in a genomic site of interest. The target sequence is adjacent to a protospacer adjacent motif (PAM). In some embodiments, the target sequence is upstream (5′-) to a protospacer adjacent motif (PAM) of 5′-NGG-3′, in which N represents any nucleotide.

In some instances, the scaffold sequence capable of being utilized by a CRISPR nuclease polypeptide derived from Nuclease A as disclosed herein may comprise a nucleotide sequence at least 85% identical to SEQ ID NO: 2. In some instances, the scaffold sequence comprises one or more deletions, one or more nucleotide substitutions, or a combination thereof, as compared with SEQ ID NO: 2.

In some instances, the scaffold sequence is a truncated variant of SEQ ID NO: 2. Such a truncation variant may be of about 110-140-nt in length. In some examples, the truncated variant may have a 3′ truncation relative to SEQ ID NO: 2. For example, the 3′ truncation may comprise deletions within residues 143-202 of SEQ ID NO: 2. In other examples, the truncated variant of SEQ ID NO: 2 may have an internal truncation relative to SEQ ID NO: 2. For example, the variant may comprise one or more deletions within residues 10-40 of SEQ ID NO: 2. In specific examples, the deletions may comprise residues 14-20 and/or 25-32 of SEQ ID NO: 2. In yet other examples, the truncated variant of SEQ ID NO: 2 may have a 3′ truncation (e.g., those disclosed herein) and an internal truncation relative to SEQ ID NO: 2 (e.g., those disclosed herein). Alternatively or in addition, the truncated variant may further comprise one or more mutations relative to SEQ ID NO: 2, for example, a deletion and/or substitution within residues 81-85 of SEQ ID NO: 2. In one specific examples, the scaffold sequence comprises (e.g., consists of) the nucleotide sequence of SEQ ID NO: 27. In another specific example, the scaffold sequence comprises (e.g., consists of) SEQ ID NO: 28. Such a scaffold sequence may be about 115-130-nt in length.

Guide RNAs comprising a spacer sequence as defined herein and any of the variant scaffold sequence relative to SEQ ID NO: 2 are also within the scope of the present disclosure.

In some instances, the CRISPR nuclease polypeptide comprised in the gene editing system disclosed herein is the reference CRISPR nuclease of SEQ ID NO: 1 and the scaffold sequence comprises the nucleotide sequence of SEQ ID NO: 27. Alternatively, the CRISPR nuclease polypeptide in the gene editing system can be a variant of SEQ ID NO: 1, e.g., comprising mutations at positions 167, D568, and E706 (e.g., arginine substitutions I67R, D568R, and E706R) and the scaffold sequence comprises the nucleotide sequence of SEQ ID NO: 28.

In other aspects, provided herein are CRISPR nuclease polypeptides derived from CRISPR nuclease K (SEQ ID NO: 65), which comprises a RuvC nuclease domain and an HNH nuclease domain. The CRISPR nuclease polypeptides derived from Nuclease K may comprise an amino acid sequence at least 90% identical to SEQ ID NO: 65. In addition to the RuvC and HNH nuclease domains, the CRISPR nuclease polypeptide may also comprise a bridge helix (BH) domain, a wedge (WED) domain, and a PAM-interacting (PID) domain.

In some embodiments, the CRISPR nuclease polypeptide may be a variant of Nuclease K, comprising at least one mutation relative to SEQ ID NO: 65. Such a variant CRISPR nuclease polypeptide may have enhanced enzymatic activity as compared with the reference CRISPR Nuclease K. In some instances, the at least one mutation may comprise (a) one or more arginine and/or lysine substitutions, optionally one or more arginine substitutions, relative to SEQ ID NO: 65; (b) one or more nickase mutations in the HNH nuclease domain or in the RuvC nuclease domain of SEQ ID NO: 65; or (c) a combination of (a) and (b).

In some examples, the variant of Nuclease K may comprise the one or more arginine and/or lysine substitutions of (a) (e.g., arginine substitutions). Any of the variant CRISPR nuclease polypeptides disclosed herein may contain up to 20 arginine and/or lysine substitutions (e.g., up to 20 arginine substitutions), for example, up to 15 arginine and/or lysine substitutions (e.g., up to 15 arginine substitutions). In some instances, the one or more arginine and/or lysine substitutions are located in the BH domain, in the WED domain, in the PID domain, or a combination thereof. In specific examples, the one or more arginine and/or lysine substitutions (e.g., arginine substitutions) are located at one or more of positions G42, D46, I53, F83, E128, E541, F570, E576, D582, C630, 1631, E683, S719, and T734 in SEQ ID NO: 65.

In some instances, the variant of Nuclease K may comprise a combination of arginine and/or lysine substitutions. For example, the variant may comprise the arginine and/or lysine substitutions at positions G42 and D582 of SEQ ID NO: 65 (e.g., G42R and D582R). In another example, the variant may comprise the arginine and/or lysine substitutions at positions 153, D582, and T734 of SEQ ID NO: 65 (e.g., 153R, D582R, and T734R).

In some embodiments, the engineered CRISPR nuclease polypeptide derived from Nuclease K provided herein may comprise one or more mutations leading to nickase activity, for example, one or more mutations at position D374, H375, and/or N398 in SEQ ID NO: 65. In some examples, the nickase variant may comprise a mutation at position H375; optionally wherein the mutation is an amino acid substitution of H375A. In specific examples, the engineered CRISPR nuclease polypeptide may comprise arginine and/or lysine substitutions (e.g., arginine substitutions) at positions 153, D582, and T734 of SEQ ID NO: 65 and further comprise the amino acid substitution of H375A.

In some embodiments, the engineered CRISPR nuclease polypeptide derived from Nuclease K disclosed herein may comprise an amino acid sequence at least 90% identical to SEQ ID NO: 65. In some examples, the engineered CRISPR nuclease derived from Nuclease K may comprise an amino acid sequence at least 95% identical to SEQ ID NO: 65. In specific examples, the engineered CRISPR nuclease derived from Nuclease K may comprise an amino acid sequence at least 98% identical to SEQ ID NO: 65.

In some embodiments, the engineered CRISPR nuclease polypeptide derived from Nuclease K as disclosed herein may be a fusion polypeptide comprising the CRISPR nuclease and one or more additional fragments, which are heterologous to the CRISPR nuclease. In some embodiments, the one or more functional fragments may comprise one or more NLS, one or more peptide linkers, or a combination thereof. The one or more NLS may be located at the N-terminus, at the C-terminus, or both.

Also provided herein is a nucleic acid, comprising a nucleotide sequence encoding any of the engineered CRISPR nuclease polypeptides derived from Nuclease K disclosed herein. In some instances, the nucleic acid is an expression vector, in which the nucleotide sequence encoding the engineered CRISPR nuclease polypeptide is in operable linkage to a promoter. Further, the present disclosure provides a host cell comprising the nucleic acid coding for the engineered CRISPR nuclease polypeptide as disclosed herein.

In another aspect, the present disclosure features a gene editing system, comprising: (a) any of the engineered CRISPR nuclease polypeptides derived from Nuclease K disclosed herein or a first nucleic acid encoding the CRISPR nuclease polypeptide; and (b) a guide RNA (gRNA), which comprises a scaffold sequence recognizable by the engineered CRISPR nuclease polypeptide and a spacer, or a second nucleic acid encoding the gRNA. The gRNA comprises a scaffold sequence recognizable by the CRISPR nuclease polypeptide and a spacer sequence specific to a target sequence in a genomic site of interest. The target sequence is adjacent to a protospacer adjacent motif (PAM). In some embodiments, the target sequence is upstream (5′-) to a protospacer adjacent motif (PAM) of 5′-NGG-3′, in which N represents any nucleotide.

In some embodiments, the scaffold capable of being utilized by a CRISPR nuclease polypeptide derived from Nuclease A as disclosed herein may comprise a nucleotide sequence at least 85% identical to SEQ ID NO: 79. Alternatively or in addition, the scaffold may be a fragment of SEQ ID NO: 79. In some instances, the scaffold comprises one or more deletions, one or more nucleotide substitutions, or a combination thereof, as compared with SEQ ID NO: 79.

In yet another aspect, the present disclosure provides nuclease polypeptides derived from reference Nuclease M (SEQ ID NO: 80), which comprises a RuvC nuclease domain and an HNH nuclease domain. Such a nuclease polypeptide may comprise an amino acid sequence at least 90% identical to SEQ ID NO: 80 (referring to the reference Nuclease M disclosed herein). In some embodiments, the nuclease polypeptide derived from Nuclease M may be a variant of Nuclease M comprising at least one mutation relative to Nuclease M. Such a variant of Nuclease M may have enhanced enzymatic activity as compared with Nuclease M.

In some instances, the at least one mutation in a variant of Nuclease M may comprise (a) one or more arginine and/or lysine substitutions (e.g., arginine substitutions) relative to Nuclease M (SEQ ID NO: 80), (b) one or more nickase mutations in the HNH nuclease domain or in the RuvC nuclease domain of SEQ ID NO: 80; or (c) a combination of (a) and (b).

In some examples, a variant of Nuclease M may comprise the one or more one or more arginine and/or lysine substitutions (e.g., arginine substitutions), which may be located in one or more of a bridge helix (BH) domain, in a nucleic acid recognition (REC) domain, in a phosphate lock loop (PLL) domain, in a wedge (WED) domain, in a PAM-interacting (PID) domain, in a nuclease domain, or a combination thereof. In some instances, the variant nuclease polypeptide of Nuclease M as disclosed herein may contain up to 20 arginine and/or lysine substitutions (e.g., arginine substitutions) relative to the reference Nuclease M. For example, the variant nuclease polypeptide may contain up to 15 arginine and/or lysine substitutions (e.g., up to 12 arginine/lysine substitutions) such as up to 15 arginine substitutions or up to 12 arginine substitutions relative to the reference Nuclease M.

In specific examples, the one or more arginine and/or lysine substitutions (e.g., arginine substitutions) can be located are at positions E88, S95, L92, E401, E83, N371, P481, and/or A373 in SEQ ID NO: 80.

Alternatively or in addition, the variant nuclease polypeptide of Nuclease M may comprise one or more mutations of leading to nickase activity. In some embodiments, the one or more nickase mutations are at one or more of positions D58, E189, D341, H243, H244, H267, R329, and/or H338 in SEQ ID NO: 80.

Any of the variants of Nuclease M provided herein may comprise an amino acid sequence at least 90% identical to SEQ ID NO: 80. In some examples, the nuclease polypeptide derived from Nuclease M may comprise an amino acid sequence at least 95% identical to SEQ ID NO: 80. In other examples, the nuclease polypeptide derived Nuclease M may comprise an amino acid sequence at least 98% identical to SEQ ID NO: 80.

In some embodiments, the nuclease polypeptide derived from Nuclease M may be a fusion polypeptide, which may further comprise one or more functional fragments. In some instances, the one or more functional fragment can be heterologous to the nuclease moiety in the fusion polypeptide. In some instances, the one or more functional fragments comprise one or more NLSs, which may be located at the N-terminus, at the C-terminus, or both, one or more peptide linkers, or a combination thereof.

Also provided herein is a nucleic acid, comprising a nucleotide sequence encoding any of the nuclease polypeptides derived from Nuclease M as disclosed herein. In some instances, the nucleic acid is an expression vector, in which the nucleotide sequence encoding the nuclease polypeptide is in operable linkage to a promoter. Alternatively, the nucleic acid can be a messenger RNA (mRNA) molecule. Further, the present disclosure provides a host cell comprising the nucleic acid coding for the nuclease polypeptide derived from Nuclease M as disclosed herein.

In addition, the present disclosure features a gene editing system, comprising: (a) any of the nuclease polypeptides derived from Nuclease M as disclosed herein or a first nucleic acid encoding such; and (b) a guide RNA (gRNA) or a second nucleic acid encoding the gRNA, which comprises a scaffold sequence recognizable by the nuclease polypeptide derived from Nuclease M and a spacer, which is specific to a target sequence within a genomic site, the target sequence being adjacent to a protospacer adjacent motif (PAM). In some embodiments, the PAM is 5′-WTAAH-3′, in which W is A or T and His A, C, or T. In one example, the PAM is 5′-TTAAA-3′.

In some embodiments, the scaffold sequence capable of being utilized by a nuclease polypeptide derived from Nuclease M as disclosed herein may comprise a nucleotide sequence at least 85% identical to SEQ ID NO: 94. Alternatively or in addition, the scaffold sequence may be a fragment of SEQ ID NO: 94. In some instances, the scaffold sequence comprises one or more deletions, one or more nucleotide substitutions, or a combination thereof, as compared with SEQ ID NO: 94.

Any of the gene editing systems disclosed herein may further comprise one or more lipid excipients associated with the element (a) and/or element (b) of the gene editing system. In some examples, the one or more lipid excipients form lipid nanoparticles, which are associated with or encapsulate the element (a) and/or element (b) of the gene editing system.

Alternatively, any of the gene editing systems disclosed herein may comprise a viral vector such as an adeno-associated viral (AAV) vector comprising coding sequences for both the CRISPR nuclease polypeptide and the gRNA therein.

Further, provided herein is a gene editing method, comprising delivering any of the gene editing systems as disclosed herein to a host cell to edit a genomic site targeted by the gRNA of the gene editing system. In some instances, the host cell is cultured in vitro. In other instances, the host cell is located in a subject who needs the gene editing.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a diagram showing percentages of NGS reads comprising indels at six genetic loci as indicated in the presence or absence of the CRISPR nuclease of SEQ ID NO: 1 (Nuclease A).

FIGS. 2A-2D are gel images showing in vitro cleavage of target or non-target strand of a target DNA substrate and a bar graph showing quantification of nuclease activity by Nuclease A and variants thereof. FIG. 2A is a gel image captured using an 800 nm channel showing in vitro cleavage of the target strand (labelled on the 5′ end with an IR800 dye) of the target DNA substrate by the reference CRISPR nuclease, putative HNH-knockout nickases, or putative RuvC-knockout nickases. FIG. 2B is a gel image captured using a 700 nm channel showing in vitro cleavage of the non-target strand (labelled on the 5′ end with an IR700 dye) of a target DNA substrate by the reference CRISPR nuclease, putative HNH-knockout nickases, or putative RuvC-knockout nickases. FIG. 2C is an overlaid image captured using 800 nm and 700 nm channels of FIG. 2A and FIG. 2B. FIG. 2D is a bar graph showing quantification of the percent of cleaved target and non-target DNA generated by the reference CRISPR nuclease, the putative HNH-knockout nickases, and the putative RuvC-knockout nickases tested.

FIGS. 3A-3C are schematic illustrations depicting predicted secondary structures of scaffold sequences. FIG. 3A: Reference Scaffold (SEQ ID NO: 2). FIG. 3B: Truncated Reference Scaffold (SEQ ID NO: 27). FIG. 3C: Scaffold 2 (SEQ ID NO: 28).

FIG. 4 is a diagram showing percentages of NGS reads comprising indels at six genetic loci as indicated in the presence or absence of the CRISPR nuclease of SEQ ID NO: 65 (Nuclease K).

FIG. 5 is a diagram showing percentages of NGS reads comprising indels at six genetic loci as indicated in the presence or absence of the nuclease of SEQ ID NO: 80 (Nuclease M).

### DETAILED DESCRIPTION OF THE INVENTION

Provided herein are nuclease polypeptides such as CRISPR nuclease polypeptides derived from the reference Nuclease A (SEQ ID NO: 1), Nuclease K (SEQ ID NO: 65), or Nuclease M (SEQ ID NO: 80). Such a CRISPR nuclease polypeptide may comprise the reference nuclease or a variant thereof as disclosed herein. In some instances, the CRISPR nuclease polypeptides provided herein may be a variant nuclease of the reference nuclease comprising at least one mutation relative to the reference nuclease.

In some embodiments, the variant CRISPR nuclease polypeptides may comprise one or more mutations (e.g., arginine substitutions, lysine substitutions, or a combination thereof) relative to the reference CRISPR nuclease. Alternatively or in addition, the variant CRISPR nuclease polypeptide may comprise one or more mutations in either the RuvC nuclease domain or the HNH nuclease domain. Such mutations (i.e., nickase mutations) may reduce or eliminate the nuclease activity of either the RuvC or the HNH nuclease domain, leading to a variant exhibiting nickase activity. As used herein, the term “nickase” refers to an enzyme that cuts one strand of a double-stranded DNA at a specific recognition nucleotide sequence (e.g., the target sequence disclosed herein). A nickase may interact with one strand of the DNA duplex to produce DNA molecules that are cut at one strand (a.k.a., nicked). In some embodiments, a nickase is a variant of a CRISPR nuclease that comprises a deactivated HNH domain. In some embodiments, a nickase is a variant of a CRISPR nuclease that comprises a deactivated RuvC domain.

Any of the variant CRISPR nuclease polypeptides provided herein may share a high sequence homology relative to the reference CRISPR Nuclease A (SEQ ID NO: 1), Nuclease K (SEQ ID NO: 65), or Nuclease M (SEQ ID NO: 80) (e.g., at least 85% sequence identity, for example, at least 90%, at least 95%, at least 97%, at least 98%, at least 99% or higher sequence identity).

The variant CRISPR nuclease polypeptides provided herein are expected to possess advantageous features relative to the reference CRISPR nuclease, for example, increased binding to a cognate guide RNA, higher nuclease activity, etc. As such, the variant CRISPR nuclease polypeptides disclosed herein would be expected to exhibit better activities in gene editing relative to the reference CRISPR nuclease, e.g., nickase activity, higher efficiency and accuracy in gene editing involving strand replacement.

Alternatively or in addition, the variant CRISPR nuclease polypeptides may be fusion polypeptides comprising a CRISPR nuclease (e.g., the reference Nuclease A, Nuclease K, or Nuclease M, or a variant of the reference nuclease as provided herein) (nuclease moiety) and one or more additional functional fragments such as those described herein (e.g., NLSs and/or peptide linkers). In addition to the advantageous features noted above, the fusion polypeptides possess additional functions attributable to the fusion partners.

Accordingly, the present disclosure provides CRISPR nuclease polypeptides derived from the reference CRISPR nuclease (Nuclease A, Nuclease K, or Nuclease M), gene editing systems comprising such, and gene editing methods using such.

### I. CRISPR Nuclease Polypeptides

As used herein, the term “CRISPR nuclease” refers to an RNA-guided effector that is capable of binding a nucleic acid and introducing a single-stranded break or double-stranded break. A CRISPR nuclease typically comprises multiple functional domains, e.g., nuclease domains (e.g., RuvC and/or HNH), PLMP domain, bridge helix (BH) domain, nucleic acid recognition (REC) domain, phosphate lock loop (PLL), wedge domain (WED), PAM-interacting domain (PID), or a combination thereof. As used herein, the term “domain” refers to a distinct functional and/or structural unit of a polypeptide. In some instances, a functional domain may be linear. In other instances, a functional domain can be discontinuous and conformational. In some embodiments, a domain may comprise a conserved amino acid sequence.

As used herein, the term “variant CRISPR nuclease polypeptide” refers to a CRISPR nuclease polypeptide comprising an alteration, e.g., a substitution, insertion, deletion and/or fusion, at one or more residue positions, compared to the reference CRISPR nuclease (Nuclease A, Nuclease K, or Nuclease M).

The variant CRISPR nuclease polypeptides provided herein are expected to exhibit one or more modulated activities (e.g., enhanced or reduced) relative to the reference CRISPR nuclease. As used herein, the term “activity” refers to a biological activity. In some embodiments, activity includes enzymatic activity, e.g., catalytic ability of an effector. For example, activity can include nuclease activity. In some embodiments, activity includes binding activity, e.g., binding of an effector (e.g., a CRISPR nuclease) to an RNA guide and/or target nucleic acid. In some examples, the variant CRISPR nuclease polypeptides disclosed herein have an enhanced binding to a cognate guide RNA (gRNA) as compared with the reference CRISPR nuclease, e.g., having a binding activity at least 20%, 30%, 40%, 50%, 60%, 70%, 80%, 90%, 100%, 2-fold, 2-fold, 5-fold, 10-fold, or greater than that of the reference CRISPR nuclease. A cognate gRNA refers to a gRNA having a scaffold recognizable by the CRISPR nuclease.

In some examples, the variant CRISPR nuclease polypeptides disclosed herein have an enhanced enzymatic activity relative to the reference CRISPR nuclease, e.g., having an enzymatic activity at least 20%, 30%, 40%, 50%, 60%, 70%, 80%, 90%, 100%, 2-fold, 2-fold, 5-fold, 10-fold, or greater than that of the reference CRISPR nuclease. In other examples, the variant CRISPR nuclease polypeptides disclosed herein have a decreased enzymatic activity relative to the reference CRISPR nuclease, e.g., having an enzymatic activity at least 20%, 30%, 40%, 50%, 60%, or 70% lower than that of the reference CRISPR nuclease. In some instances, the decreased enzymatic activity is achieved by reducing or diminishing the nuclease activity of the RuvC domain. In some instances, the decreased enzymatic activity is achieved by reducing or diminishing the nuclease activity of the HNH domain.

In some instances, the variant CRISPR nuclease polypeptides disclosed herein have enhanced indel activity relative to the reference CRISPR nuclease. As used herein, the term “indel activity” refers to the ability of a CRISPR nuclease to introduce an indel (insertion/deletion) into a sequence (e.g., a genomic target).

In some embodiments, the variant CRISPR nuclease polypeptide provided herein share a high sequence homology relative to the reference CRISPR nuclease. For example, the variant CRISPR nuclease polypeptide may comprise an amino acid sequence at least 70% (e.g., at least 80%, 85%, 90%, 95%, or higher) identical to SEQ ID NO: 1, SEQ ID NO: 65, or SEQ ID NO: 80. In some instances, the variant CRISPR nuclease polypeptide may comprise an amino acid sequence at least 90% identical to SEQ ID NO: 1, SEQ ID NO: 65, or SEQ ID NO: 80. In some instances, the variant CRISPR nuclease polypeptide may comprise an amino acid sequence at least 95% identical to SEQ ID NO: 1, SEQ ID NO: 65, or SEQ ID NO: 80. In other instances, the variant CRISPR nuclease polypeptide may comprise an amino acid sequence at least 97% (e.g., 98%, 99%, 99.5%, or greater) identical to SEQ ID NO: 1, SEQ ID NO: 65, or SEQ ID NO: 80.

The “percent identity” (a.k.a., sequence identity) of two nucleic acids or of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST nucleotide searches can be performed with the NBLAST program, score=100, wordlength-12 to obtain nucleotide sequences homologous to the nucleic acid molecules of the invention. BLAST protein searches can be performed with the XBLAST program, score=50, word length=3 to obtain amino acid sequences homologous to the protein molecules of the invention. Where gaps exist between two sequences, Gapped BLAST can be utilized as described in Altschul et al., Nucleic Acids Res. 25 (17): 3389-3402, 1997. When utilizing BLAST and Gapped BLAST programs, the default parameters of the respective programs (e.g., XBLAST and NBLAST) can be used.

In some instances, the variant CRISPR nuclease polypeptide disclosed herein may comprise one or more arginine and/or lysine substitutions relative to the reference Nuclease A, Nuclease K, or Nuclease M provided herein. “Arginine substitutions” and/or “lysine substitutions” refers to the replacement of a non-arginine or non-lysine residue in SEQ ID NO: 1 (Nuclease A), SEQ ID NO: 65 (Nuclease K), or SEQ ID NO: 80 (Nuclease M) with an arginine or lysine residue.

In some instances, one or more of the substituting arginine residues may be replaced by a conservative amino acid residue such as lysine or histidine. In some embodiments, the variant CRISPR nuclease polypeptide provided herein may comprise one or more arginine substitutions, one or more lysine substitutions, or a combination thereof.

In some instances, a variant CRISPR nuclease polypeptide provided herein may contain one or more conservative amino acid residue substitutions, either taken alone or in combination with the other types of mutations disclosed herein.

As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

### (A) Nuclease A and Engineered Variants Thereof

In some embodiments, the CRISPR nuclease polypeptide is derived from Nuclease A, including both the wild-type Nuclease A (SEQ ID NO: 1), a variant thereof such as those disclosed herein, or a fusion polypeptide comprising such. The variant CRISPR nuclease polypeptides of Nuclease A may be produced via introducing one or more mutations to the reference CRISPR nuclease to modulate (e.g., enhance or reduce) one or more activities of the nuclease.

The reference CRISPR nuclease of Nuclease A (SEQ ID NO: 1) (see Table 1 below) is a CRISPR nuclease that comprises both a RuvC nuclease domain (located at residues 15-53, 308-338, and 472-566 of SEQ ID NO: 1) and a HNH domain (located at residues 339-471 of SEQ ID NO: 1). The RuvC nuclease domain and the HNH nuclease domain coordinate cleavage of the DNA strand adjacent to the 5′-NGG-3′ PAM motif, in which N represents any nucleotide. Positions D24, E335 and D524 are deemed the active sites in the RuvC domain, and positions D396, H397, and N420 are deemed the active sites in the HNH domain. R506 and H521 may also be important for the nuclease activity of the RuvC domain. In addition to the nuclease domains, the reference CRISPR nuclease of SEQ ID NO: 1 also includes a BH domain (residues 54-89 of SEQ ID NO: 1), a REC domain (residues 90-307 of SEQ ID NO: 1), a PLL domain (residues 567-580 of SEQ ID NO: 1), a WED domain (residues 581-672 of SEQ ID NO: 1), and a PID domain (residues 673-776 of SEQ ID NO: 1).

Compared to the CRISPR-Cas9 nuclease from Streptococcus pyogenes, the reference CRISPR nuclease of SEQ ID NO: 1 disclosed herein is smaller. The scaffold utilized by the reference CRISPR nuclease of SEQ ID NO: 1 and variants thereof can be miniaturized. This scaffold comprises a distinctive structure compared to the SpCas9 nuclease scaffold. The distinctive scaffold is expected to allow for decreased size of some domains (e.g., the REC domain) and thus contribute to the smaller CRISPR nuclease size. These features would be beneficial for delivery. Arginine and/or lysine substitutions (e.g., arginine substitutions) can be introduced into the CRISPR nuclease of SEQ ID NO: 1 to increase indel activity.

Further, the RuvC domain of Nuclease A (SEQ ID NO: 1) cleaves the non-target strand of a target nucleic acid within 4 to 8 nucleotides upstream of the PAM, and the HNH domain cleaves the target strand between 3 and 4 nucleotides upstream of the PAM, each leading to a cut site with an overhang of 0-5 nucleotides (most commonly a 3-5 nucleotide overhang). In contrast, the RuvC domain of SpCas9 cleaves the non-target strand of a target nucleic acid within 3 to 5 nucleotides upstream of the cognate PAM (5′-NGG-3′, in which N is any nucleotide), and the HNH domain cleaves the target strand between 3 and 4 nucleotides upstream of the PAM, each leading to an overhang of 0-3 nucleotides (mostly commonly a blunt cut).

Additionally, use of gene editing systems comprising Nuclease A (SEQ ID NO: 1) or variants thereof can result in the introduction of indels into a target nucleic acid that are larger than those capable of being introduced by SpCas9. For example, insertions induced by use of Nuclease A or variants thereof can range from about 1-nucleotide to about 7-nucleotides (most commonly about 4-nucleotides). Deletions induced by use of Nuclease A or variants thereof can range from about 1-nucleotide to about 25-nucleotides or larger (most commonly about 11-nucleotides). Further, since the reference CRISPR nuclease of SEQ ID NO: 1 comprises a RuvC domain and an HNH domain, nickase variants can be engineered, e.g., via disrupting nuclease activity of either the RuvC domain or the HNH domain.

The variant CRISPR nuclease polypeptide of Nuclease A as provided herein can contain one or more alterations relative to Nuclease A (SEQ ID NO: 1), e.g., one or more amino acid residue substitutions, one or more deletions, one or more insertions, one or more fusions, or a combination thereof. In some instances, the alterations may be introduced into the BH domain, the PLL domain, the WED domain, the PID domain, or a combination thereof. In some instances, no alterations are introduced into the RuvC and/or the HNH nuclease domains, or at the active sites and/or sites involved in activity in these domains as provided herein. Alternatively, conservative amino acid substitutions may be introduced into SEQ ID NO: 1, including in the RuvC and/or the HNH nuclease domains.

In some embodiments, the variant CRISPR nuclease polypeptide of Nuclease A may comprise one or more arginine substitutions, one or more lysine substitutions, or a combination thereof relative to SEQ ID NO: 1. In some examples, the variant CRISPR nuclease polypeptide may contain up to 20 arginine and/or lysine substitutions (e.g., up to 20 arginine substitutions, up to 20 lysine substitutions, or a combination thereof), e.g., up to 19, 18, 17, 16, 15, 14, 13, 12, 11, 10, 9, 8, 7, 6, 5, 4, 3, or 2 arginine substitutions, lysine substitutions, or a combination thereof. In specific examples, the variant CRISPR nuclease polypeptide may contain 12, 11, 10, 9, 8, 7, 6, 5, 4, 3, or 2 arginine substitutions, lysine substitutions, or a combination thereof. In some examples, the variant CRISPR nuclease polypeptide provided herein contains arginine substitutions.

In some instances, the arginine and/or lysine substitutions may be located in the BH domain, in the PLL domain, in the WED domain, in the PID domain, or in any of the combination thereof of Nuclease A. In some examples, the variant CRISPR nuclease polypeptide derived from Nuclease A may contain one or more of the arginine and/or lysine substitutions (e.g., arginine substitutions) at one or more of the following positions in SEQ ID NO: 1: D56, E59, G60, E63, 167, G71, S564, D568, T605, E655, E694, and E706. In some examples, the variant CRISPR nuclease polypeptide derived from Nuclease A comprises one or more of the following arginine substitutions relative to SEQ ID NO: 1: D56R, E59R, G60R, E63R, I67R, G71R, S564R, D568R, T605R, E655R, E694R, and E706R.

In some instances, the arginine and/or lysine substitution may be located within the RuvC and/or the HNH nuclease domains of Nuclease A. In some examples, the arginine and/or lysine substitutions may be within the RuvC nuclease domain of Nuclease A to reduce or inactivate the RuvC domain (e.g., at positions D24, E335, R506, H521, and/or D524 in the RuvC domain of SEQ ID NO: 1). In other examples, the arginine and/or lysine substitutions may be within the HNH domain of Nuclease A, e.g., at positions D396, H397, and/or N420 in the HNH domain of SEQ ID NO: 1. In yet other examples, the arginine and/or lysine substitutions may be within both the RuvC domain and the HNH domain of Nuclease A to reduce or diminish the nuclease enzymatic activity.

Alternatively, the arginine and/or lysine substitution may not be at the active sites and/or sites involved in activity in the RuvC and/or the HNH nuclease domains of Nuclease A (e.g., not at positions D24, E335, R506, H521, and/or D524 in the RuvC domain and/or at positions D396, H397, and/or N420 in the HNH domain of SEQ ID NO: 1). In some examples, the arginine and/or lysine substitution may not be in the RuvC and/or HNH domains of Nuclease A.

It is reported herein that arginine substitutions at the following positions in SEQ ID NO: 1 led to diminished or no nuclease activity, indicating that these positions are not tolerable to mutations with respect to nuclease activity: L21, G22, 123, D24, G26, G27, T30, G31, L32, A33, V34, V35, V42, V48, M50, L85, V107, Y108, C111, G115, M169, V182, F186, I270, C286, H289, S309, L310, V317, V321, M336, 1343, V334, E337, S338, N339, F341, T347, Y358, T377, V382, Y383, C384, T389, A393, D396, H397, I398, F399, P400, I406, N411, V413, A414, C415, C416, N420, K423, K450, L452, A455, 1466, M469, S470, A472, S473, 1474, G475, L483, G499, T502, W509, F511, H521, L523, D524, A525, V526, 1527, L528, A529, P581, V595, T596, 1606, Y611, L615, L630, A635, F640, Y641, L651, G657, L658, G659, Q662, M663, V664, K672, T673, N674, V675, Y683, L700, V725, I736, L739, P743, L745, and L765 in SEQ ID NO: 1.

In some embodiments, a variant of CRISPR Nuclease A provided herein exhibits nuclease activity and may not have mutations at the above positions. Alternatively, a variant CRISPR nuclease may be a dead nuclease (e.g., for use in base editing) having mutations at one or more of these positions.

In some instances, a variant CRISPR nuclease comprising arginine and/or lysine substitutions at a combination of 2 or more (e.g., 3 or 4) positions, for example, of D56, 167, D568, T605, E694, and E706 of SEQ ID NO: 1. Examples include, but are not limited to, (a) I67, D568, and E706; (b) D56, 167, and D568; (c) I67, D568, and T605; (d) D56; 167, and E706; (e) I67, T605, and E706; (f) I67, D568, and E694; (g) D56, 167, E694, and E706; (h) D56, 167, T605, and E706; and (i) I67, D568, W593, and E706. In some specific examples, the engineered CRISPR nuclease polypeptide comprises the arginine substitutions of 167, D568, and E706. In some examples, the variant CRISPR nuclease comprise arginine substitutions at the combined positions listed herein.

Addition exemplary combinations of arginine and/or lysine substitutions (e.g., arginine substitutions) include (relative to SEQ ID NO: 1): (i) at least two positions of D56, E59, G60, E63, 167, G71, S564, D568, T605, E655, E694, and E706; (ii) at least two positions D56, 167, T605, E694, and E706; (iii) at least two positions of D56, 167, D568, T605, E694, and E706; (iv) at least two positions of D56, E63, G71, D568, T605, E655, E694, and E706; and (v) at least two positions of D56, 167, G71, D568, T605, E655, E694, and E706.

Alternatively or in addition, the engineered variant CRISPR nuclease polypeptide of Nuclease A disclosed herein may have an N-terminal truncation relative to Nuclease A of SEQ ID NO: 1. Such an engineered variant CRISPR nuclease polypeptide has a fragment at the N-terminus of Nuclease A deleted. In some instances, the deleted N-terminal fragment has up to 80 amino acid residues, for example, up to 70 amino acid residues, up to 60 amino acid residues, up to 50 amino acid residues, up to 40 amino acid residues, up to 30 amino acid residues, or up to 20 amino acid residues. In some examples, the N-terminal truncation is a deletion within the residues 1-15 of SEQ ID NO: 1. In one specific example, the N-terminal truncation is a deletion of residues 1-14 of SEQ ID NO: 1. In another specific example, the N-terminal truncation is a deletion of residues 1-15 of SEQ ID NO: 1.

Alternatively or in addition, the engineered CRISPR nuclease polypeptide disclosed herein may have a C-terminal truncation relative to the reference CRISPR nuclease A of SEQ ID NO: 1. Such an engineered CRISPR nuclease polypeptide has a fragment at the C-terminus of the reference CRISPR nuclease deleted. In some instances, the deleted C-terminal fragment has up to 10 amino acid residues, for example, up to 5 amino acid residues, up to 4 amino acid residues, up to 3 amino acid residues, up to 2 amino acid residues, or up to 1 amino acid residues. In some examples, the C-terminal truncation is a deletion of the final residue, the final two residues, the final three residues, the final four residues, or the final five residues of SEQ ID NO: 1. In some instances, the C-terminal truncated variant may further comprise one or more of the mutations disclosed herein, for example, one or more arginine/lysine substitutions, one or more mutations resulting in nickase activity, or a combination thereof. In some specific examples, the C-terminal truncated variant further comprises arginine substitutions of I67R, D568R, E706R relative to SEQ ID NO: 1. In other specific examples, the C-terminal truncated variant further comprises arginine substitutions of I67R, D568R, W593R, and E706R relative to SEQ ID NO: 1. In some examples, the C-terminal truncated variant further comprises an N-terminal truncation. In some examples, the C-terminal truncated variant further comprises a deletion of residues 1-14 or 1-15 of SEQ ID NO: 1.

In some instances, the truncated variant may further comprise one or more of the mutations disclosed herein, for example, one or more arginine/lysine substitutions, one or more mutations resulting in nickase activity (nickase mutations), or a combination thereof. In some specific examples, the truncated variant may further comprise arginine/lysine substitutions at positions 167, D568, and E706 of SEQ ID NO: 1 (e.g., arginine substitutions I67R, D568R, and E706R). In other specific examples, the truncated variant further comprises arginine/lysine substitutions at positions 167, D568, W593, and E706 of SEQ ID NO: 1 (e.g., arginine substitutions of I67R, D568R, W593R, and E706R).

Alternatively or in addition, any of the variant CRISPR nuclease polypeptides of Nuclease A provided herein may comprise one or more nickase mutations within either the RuvC or the HNH nuclease domain to reduce or eliminate the nuclease activity of the target domain, thereby producing a variant with nickase activity. Such mutations may be deletions, insertions, amino acid substitutions, or a combination thereof. In some embodiments, the mutations within either the RuvC or the HNH nuclease domain are amino acid substitutions, of which the substituting amino acid residue is not a conservative substitution of the native amino acid residue at the position of the mutation. For example, if the native amino acid residue is R, the substituting residue can be any amino acid residue except for K. Similarly, if the native amino acid residue is K, the substituting residue can be any amino acid residue except for R. Groups of conservative amino acid residue substitutions are provided herein.

Positions D24, E337, and D524 are identified as putative catalytic residues in the RuvC domain of Nuclease A, and positions D396, H397, and N420 are identified as putative catalytic residues in the HNH domain of Nuclease A of SEQ ID NO: 1. In some examples, the one or more mutations may be within the RuvC nuclease domain to reduce or inactivate the RuvC domain (e.g., at positions D24, E337, and/or D524 in the RuvC domain). In some specific examples, the variant CRISPR nuclease polypeptide derived from Nuclease A may contain substitution(s) of D24A, E337A, and/or D524A. In other examples, any of D24, E337, and D524 may be substituted by an amino acid residue similar to A, for example, G, S, or L.

In some examples, the one or more mutations may be within the HNH domain to reduce or inactivate the HNH domain (e.g., at positions D396, H397, and/or N420 in the HNH domain). In some specific examples, the variant CRISPR nuclease polypeptide may contain substitutions of H397A, D396A, and/or N420A. Alternatively, any of H397, D396, and N420 may be replaced with an amino acid residue similar to A, for example, G, L, or S. In one specific example, the variant CRISPR nuclease polypeptide may contain substitution of H397A or H397L.

In yet other examples, the one or more mutations may be within both the RuvC domain and the HNH domain of Nuclease A to reduce or diminish the nuclease enzymatic activity.

In some embodiments, the variant CRISPR nuclease polypeptide provided herein may comprise both arginine/lysine substitutions (e.g., arginine substitutions), e.g., at one or more positions provided herein, and nickase mutations. For example, the variant CRISPR nuclease polypeptide may comprise arginine/lysine substitutions (e.g., arginine substitutions) at positions I67, D568, and/or E706, and further comprise an amino acid substitution at H397 (e.g., H1397A or H397L) in SEQ ID NO: 1. In some instances, such a variant may further comprise an N-terminal truncation as disclosed herein, e.g., a deletion within residues 1-15 of SEQ ID NO: 1 such as the deletion of 1-14 aa or 1-15 aa of SEQ ID NO: 1. In some instances, such a variant may further comprise a C-terminal truncation as disclosed herein, e.g., a deletion within the final five residues of SEQ ID NO: 1.

In some examples, the variant CRISPR nuclease polypeptide provided herein may share a sequence identity at least 90% (e.g., 95%, 97%, 98%, 99%, 99.5%, or greater) with SEQ ID NO: 1. Exemplary engineered variant CRISPR nuclease polypeptide of Nuclease A is listed in Table 1, Table 11, or Table 14, each of which is within the scope of the present disclosure. In specific examples, the variant CRISPR nuclease polypeptide may comprise (e.g., consist of) an amino acid sequence of any one of SEQ ID NOs: 6-13, 34, 36, 38, 40, 42, 44, 46, 58, 62, and 64 (e.g., SEQ ID NO: 6 or SEQ ID NO: 36).

### (B) Nuclease K and Engineered Variants Thereof

In some embodiments, the CRISPR nuclease polypeptide is derived from Nuclease K, including both the wild-type Nuclease K (SEQ ID NO: 65), a variant thereof such as those disclosed herein, or a fusion polypeptide comprising such. The variant CRISPR nuclease polypeptides of Nuclease K may be produced via introducing one or more mutations to the reference CRISPR nuclease to modulate (e.g., enhance or reduce) one or more activities of the nuclease.

The reference CRISPR Nuclease K (SEQ ID NO: 65; see Table 15 below) is a CRISPR nuclease that comprises both a RuvC nuclease domain (located at residues 1-39, 286-320, and 448-543 of SEQ ID NO: 1) and a HNH domain (located at residues 321-447 of SEQ ID NO: 1). The RuvC nuclease domain and the HNH nuclease domain coordinate cleavage of the DNA strand adjacent to the 5′-NGG-3′ PAM motif, in which N represents any nucleotide. Positions D10, E315 and D500 are deemed the active sites in the RuvC domain and positions D374, H375, and N398 are deemed the active sites in the HNH domain. R482 and H497 may also be important for the nuclease activity of the RuvC domain. In addition to the nuclease domains, the reference CRISPR nuclease of SEQ ID NO: 1 also includes a BH domain (residues 40-77 of SEQ ID NO: 1), a REC domain (residues 78-285 of SEQ ID NO: 1), a PLL domain (residues 544-556), a WED domain (residues 557-648), and a PID domain (residues 649-747).

Compared to the CRISPR-Cas9 nuclease from Streptococcus pyogenes, the reference CRISPR Nuclease K of SEQ ID NO: 65 disclosed herein is smaller. The scaffold utilized by the reference CRISPR nuclease of SEQ ID NO: 65 and variants thereof can be miniaturized. This scaffold comprises a distinctive structure compared to the SpCas9 nuclease scaffold. The distinctive scaffold is expected to allow for decreased size of some domains (e.g., the REC domain) and thus contribute to the smaller CRISPR nuclease size. These features would be beneficial for delivery. Arginine and/or lysine substitutions can be introduced into the CRISPR nuclease of SEQ ID NO: 65 to increase indel activity.

Further, the RuvC domain of Nuclease K cleaves the non-target strand within 4 to 6 nucleotides upstream of the PAM and the HNH domain cleaves the target strand between 3 and 4 nucleotides upstream of the PAM, each leading to a cut site with an overhang of 0-3 nucleotides (most commonly a 3-nucleotide overhang). This cutting pattern is different from that of SpCas9 as described above. Additionally, use of gene editing systems comprising Nuclease K or variants thereof can result in the introduction of indels into a target nucleic acid that are larger than those capable of being introduced by SpCas9. For example, insertions induced by use of Nuclease K or variants thereof can range from about 1-nucleotide to about 10-nucleotides (most commonly about 4-nucleotides). Deletions induced by use of Nuclease A or variants thereof can range from about 1-nucleotide to about 25-nucleotides or larger (most commonly about 11-nucleotides). Further, since Nuclease K comprises a RuvC domain and an HNH domain, nickase variants can be engineered, e.g., via disrupting nuclease activity of either the RuvC domain or the HNH domain.

The variant of Nuclease K polypeptide provided herein can contain one or more mutations relative to Nuclease K (SEQ ID NO: 65), e.g., one or more amino acid residue substitutions, one or more deletions, one or more insertions, one or more fusions, or a combination thereof. In some instances, the alterations may be introduced into the BH domain, the WED, the PID, or a combination thereof of Nuclease K. In some instances, no alterations are introduced into the RuvC and/or the HNH nuclease domains of Nuclease K, or at the active sites and/or sites involved in activity in these domains as provided herein. Alternatively, conservative amino acid substitutions may be introduced into SEQ ID NO: 65, including in the RuvC and/or the HNH nuclease domains of Nuclease K.

In some embodiments, the variant CRISPR nuclease polypeptide derived from Nuclease K provided herein may comprise one or more arginine substitutions, one or more lysine substitutions, or a combination thereof relative to SEQ ID NO: 65. In some examples, the variant CRISPR nuclease polypeptide derived from Nuclease K may contain up to 20 arginine and/or lysine substitutions (e.g., up to 20 arginine substitutions, up to 20 lysine substitutions, or a combination thereof), e.g., up to 19, 18, 17, 16, 15, 14, 13, 12, 11, 10, 9, 8, 7, 6, 5, 4, 3, or 2 arginine substitutions, lysine substitutions, or a combination thereof. In specific examples, the variant CRISPR nuclease polypeptide derived from Nuclease K may contain 12, 11, 10, 9, 8, 7, 6, 5, 4, 3, or 2 arginine substitutions, lysine substitutions, or a combination thereof. In some examples, the variant CRISPR nuclease polypeptide derived from Nuclease K provided herein contains arginine substitutions.

In some instances, the arginine and/or lysine substitutions may be located in the BH domain, in the WED domain, in the PID domain, or in any of the combinations thereof of Nuclease K. For example, the arginine and/or lysine substitution may be introduced into one or more of the following positions: G42, D46, 153, F83, E128, E541, F570, E576, D582, C630, I631, E683, S719, and T734 of SEQ ID NO: 65. In some instances, the arginine and/or lysine substitutions may be at positions G42 and D582 of SEQ ID NO: 65. In other instances, the arginine and/or lysine substitutions may be at positions 153, D582, and T734 of SEQ ID NO: 65.

In some examples, the variant CRISPR nuclease polypeptide derived from Nuclease K (SEQ ID NO: 65) may contain one or more of the following arginine substitutions: G42R, D46R, I53R, E128R, D582R, F570R, E576R, C630R, I631R, E683R, T734R, and S719R. In other examples, the variant CRISPR nuclease polypeptide derived from Nuclease K may contain one or more of the following arginine substitutions: G42R, D46R, I53R, F83R, E541R, F570R, D582R, E683R, and S719R. In some specific examples, the variant CRISPR nuclease polypeptide derived from Nuclease K may contain arginine substitutions of D582R and G42R. In other specific examples, the variant CRISPR nuclease polypeptide may contain arginine substitutions of D582R, 153R, and T734R.

In some instances, the arginine and/or lysine substitution may be located within the RuvC and/or the HNH nuclease domains of Nuclease K. In some examples, the arginine and/or lysine substitutions may be within the RuvC nuclease domain of Nuclease K to reduce or inactivate the RuvC domain (e.g., at positions D10, E315, R482, H497, and/or D500 in the RuvC domain of SEQ ID NO: 65). In other examples, the arginine and/or lysine substitutions may be within the HNH domain of Nuclease K, e.g., at positions D374, H375, and/or N398 in HNH domain of SEQ ID NO: 65. In yet other examples, the arginine and/or lysine substitutions may be within both the RuvC domain and the HNH domain of Nuclease K to reduce or diminish the nuclease enzymatic activity.

Alternatively, the arginine and/or lysine substitution may not be at the active sites and/or sites involved in the activity of the RuvC and/or the HNH nuclease domains of Nuclease K (e.g., not at positions D10, E315, R482, H497, and/or D500 in the RuvC domain and/or at positions D374, H375, and/or N398 in HNH domain). In some examples, the arginine and/or lysine substitution may not be in the RuvC and/or HNH domains of Nuclease K.

Alternatively or in addition, the variant CRISPR nuclease polypeptide derived from Nuclease K provided herein may comprise one or more mutations (i.e., nickase mutations) within either the RuvC or the HNH nuclease domain to reduce or eliminate the nuclease activity of the target domain, thereby producing a variant with nickase activity. Such mutations may be deletions, insertions, amino acid substitutions, or a combination thereof. In some embodiments, the mutations within either the RuvC or the HNH nuclease domain of Nuclease K are amino acid substitutions, of which the substituting amino acid residue is not a conservative substitution of the native amino acid residue at the position of the mutation. For example, if the native amino acid residue is R, the substituting residue can be any amino acid residue except for K. Similarly, if the native amino acid residue is K, the substituting residue can be any amino acid residue except for R. Groups of conservative amino acid residue substitutions are provided herein.

Positions D10, E315, and D500 are deemed the active sites in the RuvC domain of Nuclease K (SEQ ID NO: 65), and positions D374, H375, and N398 are deemed the active sites in the HNH domain. R482 and H497 may also be important for nuclease activity of the RuvC domain of Nuclease K. In some examples, the one or more mutations may be within the RuvC nuclease domain to reduce or inactivate the RuvC domain (e.g., at positions D10, E315, R482, H497, and/or D500 in the RuvC domain). In some specific examples, the variant CRISPR nuclease polypeptide may contain substitution(s) of D10A, E315A, and/or D500A. In other examples, any of D10, E315, and D500 may be substituted by an amino acid residue similar to A, for example, G, S, or L.

In some examples, the one or more mutations may be within the HNH domain (e.g., at positions H375, D374, and/or N398 in the HNH domain of SEQ ID NO: 65). In some specific examples, the variant CRISPR nuclease polypeptide derived from Nuclease K may contain substitutions of H375A, D374A, and/or N398A. Alternatively, any of H375, D374, and N398 may be replaced with an amino acid residue similar to A, for example, G, L, or S. In one specific example, the variant CRISPR nuclease polypeptide derived from Nuclease K may contain substitution of H375A or H375L.

In yet other examples, the one or more mutations may be within both the RuvC domain and the HNH domain of Nuclease K to reduce or diminish the nuclease enzymatic activity.

In some embodiments, the variant CRISPR nuclease polypeptide derived from Nuclease K provided herein may comprise both arginine/lysine substitutions (e.g., arginine substitutions), e.g., at one or more positions provided herein, and nickase mutations, e.g., at one or more of the positions also provided herein. In some instances, the variant CRISPR nuclease polypeptide of Nuclease K may comprise arginine/lysine substitutions (e.g., arginine substitutions) listed in Table 18 and Table 20. Such variant CRISPR nuclease polypeptide may further comprise one or more mutations that reduce or inactivate the RuvC and/or HNH nuclease domain (e.g., one or more mutations within the RuvC and/or HNH nuclease domain). For example, the variant CRISPR nuclease polypeptide derived from Nuclease K may comprise arginine/lysine substitutions (e.g., arginine substitutions) at positions D582, 153, and T734, and an amino acid substitution at H375 (e.g., H375A) in SEQ ID NO:65.

### (C) Nuclease M and Engineered Variants Thereof

In some embodiments, the nuclease polypeptide is derived from Nuclease M, including both the wild-type Nuclease M (SEQ ID NO: 80), a variant thereof such as those disclosed herein, or a fusion polypeptide comprising such. The variant nuclease polypeptides of Nuclease M may be produced via introducing one or more mutations to Nuclease M to modulate (e.g., enhance or reduce) one or more activities of the nuclease.

The reference Nuclease M of SEQ ID NO: 80 (see Table 21 below) is an RNA-guided nuclease that comprises both a RuvC nuclease domain (located at residues 52-84, 160-192, and 289-367 of SEQ ID NO: 80) and a HNH domain (located at residues 193-288 of SEQ ID NO: 1). The RuvC nuclease domain and the HNH nuclease domain coordinate cleavage of the DNA strand adjacent to the 5′-WTAAH-3′ PAM motif, in which W is A or T and His A, C, or T. In one example, the PAM motif is 5′-TTAAA-3′.

Positions D58, E189, and D341 are deemed the active sites in the RuvC domain, and positions H243, H244, and H267 are deemed the active sites in the HNH domain. R329 and H338 may also be important for the nuclease activity of the RuvC domain. In addition to the nuclease domains, Nuclease M of SEQ ID NO: 80 also includes a PLMP domain (residues 1-51 of SEQ ID NO: 1), a BH domain (residues 85-117 of SEQ ID NO:1), a REC domain (residues 118-159 of SEQ ID NO:1), a PLL domain (residues 368-380), a WED domain (residues 381-427), and a PID domain (residues 428-497).

Compared to the CRISPR-Cas9 nuclease from Streptococcus pyogenes, the reference Nuclease M of SEQ ID NO: 80 disclosed herein is smaller. The scaffold utilized by the reference CRISPR nuclease of SEQ ID NO: 80 and variants thereof can be miniaturized. This scaffold comprises a distinctive structure compared to the SpCas9 nuclease scaffold. The distinctive scaffold is expected to allow for decreased size of some domains (e.g., the REC domain) and thus contribute to the smaller nuclease size. These features would be beneficial for delivery. Arginine substitutions can be introduced into Nuclease M SEQ ID NO: 80 to increase indel activity. Nuclease M recognizes different PAM motifs in genomic targets as compared with SpCas9, allowing for additional gene targets to be edited, allowing for additional gene targets to be edited.

Further, the RuvC domain of Nuclease M cleaves the non-target strand within 3 to 12 nucleotides upstream of the PAM, and the HNH domain cleaves the target strand between 3 and 4 nucleotides upstream of the PAM, each leading to a cute site with an overhang of 0-9 nucleotides (most commonly a 5-nucleotide overhang). This cutting pattern is different from that of SpCas9 as described above. Also, unlike SpCas9, the RNA-guided nuclease of SEQ ID NO: 80 comprises a PLMP domain. The PLMP domain is expected to bind a helix on the 3′ end of the scaffold and provide an increased binding affinity of the RNA-guided nuclease to its cognate scaffold.

Additionally, since Nuclease M of SEQ ID NO: 80 comprises a RuvC domain and an HNH domain, nickase variants can be engineered, e.g., via disrupting the nuclease activity of either the RuvC domain or the HNH domain.

The variant nuclease polypeptides of Nuclease M may contain one or more mutations relative to Nuclease M (SEQ ID NO: 80) to modulate (e.g., enhance or reduce) one or more activities of the nuclease, for example, one or more amino acid residue substitutions, one or more deletions, one or more insertions, one or more fusions, or a combination thereof. In some instances, the mutations may be introduced into the BH domain, the REC domain, the PLL domain, the WED domain, the PID domain, or a combination thereof. In some instances, no mutations are introduced into the RuvC and/or the HNH nuclease domains, or at the active sites and/or sites involved in activity of these domains as provided herein. Alternatively, conservative amino acid substitutions may be introduced into SEQ ID NO: 80, including in the RuvC and/or the HNH nuclease domains.

In some embodiments, the variant nuclease polypeptide of Nuclease M provided herein may comprise one or more arginine substitutions, one or more lysine substitutions, or a combination thereof, relative to SEQ ID NO: 80. In some examples, the variant nuclease polypeptide derived from Nuclease M may contain up to 20 arginine substitutions, up to 20 lysine substitutions, or a combination thereof, e.g., up to 19, 18, 17, 16, 15, 14, 13, 12, 11, 10, 9, 8, 7, 6, 5, 4, 3, or 2 arginine substitutions, lysine substitutions, or a combination thereof. In specific examples, the variant nuclease polypeptide derived from Nuclease M may contain 12, 11, 10, 9, 8, 7, 6, 5, 4, 3, or 2 arginine substitutions, lysine substitutions, or a combination thereof. In some specific examples, the variant nuclease polypeptide derived from Nuclease M comprises arginine substitutions relative to SEQ ID NO: 80.

In some examples, the variant nuclease polypeptide of Nuclease M may comprise one or more arginine and/or lysine substitutions (e.g., arginine substitutions) at positions E88, S95, L92, E401, E83, N371, P481, and/or A373 in SEQ ID NO: 80. In some instances, the variant nuclease polypeptide derived from Nuclease M may comprise at least two arginine and/or lysine substitutions such as at least two arginine substitutions at these positions.

In some instances, the arginine and/or lysine substitutions (e.g., arginine substitutions) may be located in the BH domain, in the REC domain, in the PLL domain, in the WED domain, in the PID domain, or in any of the combination thereof in Nuclease M. Alternatively or in addition, the arginine and/or lysine substitutions (e.g., arginine substitutions) may be located within the RuvC and/or the HNH nuclease domains of Nuclease M. In some examples, the arginine and/or lysine substitutions (e.g., arginine substitutions) may be within the RuvC nuclease domain of Nuclease M to reduce or inactivate the RuvC domain of Nuclease M (e.g., at positions D58, E189, R329, H338 and/or D341 in the RuvC domain of SEQ ID NO: 80). In other examples, the arginine and/or lysine substitutions (e.g., arginine substitutions) may be within the HNH domain of Nuclease M (SEQ ID NO: 80), e.g., at positions H243, H244, and/or H267 in HNH domain. In yet other examples, the arginine and/or lysine substitutions (e.g., arginine substitutions) may be within both the RuvC domain and the HNH domain of Nuclease M to reduce or diminish the nuclease enzymatic activity.

Alternatively, the arginine and/or lysine substitutions (e.g., arginine substitutions) may not be at the active sites and/or sites involved in the activity of the RuvC and/or the HNH nuclease domains of Nuclease M (e.g., not at positions D58, E189, R329, H338 and/or D341 in the RuvC domain and/or at positions H243, H244, and/or H267 in HNH domain of SEQ ID NO: 80). In some examples, the arginine and/or lysine substitutions (e.g., arginine substitutions) may not be in the RuvC and/or HNH domains of Nuclease M.

Exemplary arginine substitution variants of Nuclease M are listed in Table 24 below, each of which is within the scope of the present disclosure.

Alternatively or in addition, the variant nuclease polypeptide of Nuclease M provided herein may comprise one or more mutations (i.e., nickase mutations) within either the RuvC or the HNH nuclease domain to reduce or eliminate the nuclease activity of the target domain, thereby producing a variant with nickase activity. Such mutations may be deletions, insertions, amino acid substitutions, or a combination thereof. In some embodiments, the mutations within either the RuvC or the HNH nuclease domain are amino acid substitutions, of which the substituting amino acid residue is not a conservative substitution of the native amino acid residue at the position of the mutation. For example, if the native amino acid residue is R, the substituting residue can be any amino acid residue except for K. Similarly, if the native amino acid residue is K, the substituting residue can be any amino acid residue except for R. Groups of conservative amino acid residue substitutions are provided herein.

In some examples, the one or more mutations may be within the RuvC nuclease domain to reduce or inactivate the RuvC domain of Nuclease M (e.g., at positions D58, E189, R329, H338, and/or D341 in the RuvC domain of SEQ ID NO: 80). Alternatively, the one or more nickase mutations may be within the HNH domain of Nuclease M (e.g., at positions H243, H244, and/or H267 in the HNH domain of SEQ ID NO:80). In specific examples, the variant nuclease polypeptide of Nuclease M may comprise one or more nickase mutations are at one or more of positions D58, E189, D341, H243, H244, H267, R329, and/or H338 in SEQ ID NO: 80. In some instances, one or more of the original residues in either the RuvC domain or the HNH domain of Nuclease M can be replaced with Alanine (A). Alternatively, one or more of the original residues in either the RuvC domain or the HNH domain may be substituted by an amino acid residue similar to A, for example, G, S, or L.

In yet other examples, the one or more mutations may be within both the RuvC domain and the HNH domain of Nuclease M to reduce or diminish the nuclease enzymatic activity.

In some embodiments, the variant nuclease polypeptide derived from Nuclease M provided herein may comprise both arginine/lysine substitutions (e.g., arginine substitutions), e.g., at one or more positions provided herein, and nickase mutations, e.g., at one or more of the positions also provided herein.

In some examples, the variant nuclease polypeptide derived from Nuclease M may share a sequence identity at least 90% (e.g., 95%, 97%, 98%, 99%, 99.5%, or greater) with SEQ ID NO: 80.

In some embodiments, the CRISPR nuclease polypeptide derived from Nuclease A, Nuclease K, or Nuclease M as provided herein may be a fusion polypeptide comprising a CRISPR nuclease moiety as disclosed herein and one or more additional functional elements. In some instances, the one or more additional functional elements may be heterologous to the CRISPR nuclease moiety.

As used herein, the terms “fusion” and “fused” refer to the joining of at least two nucleotide or protein molecules. For example, “fusion” and “fused” can refer to the joining of at least two polypeptide domains that are encoded by separate genes in nature. The fusion can be an N-terminal fusion, a C-terminal fusion, or an intramolecular fusion. In some aspects, the domains are transcribed and translated to produce a single polypeptide.

In some instances, the CRISPR nuclease moiety in the fusion polypeptide may be the reference CRISPR Nuclease A (SEQ ID NO: 1), Nuclease K (SEQ ID NO: 65), or Nuclease M (SEQ ID NO: 80). Alternatively, the CRISPR nuclease portion in the fusion polypeptide may be a variant of any of the reference nucleases as disclosed herein. Exemplary additional functional moieties to include in the fusion polypeptide include a peptide tag, a fluorescent protein, a base-editing domain, a DNA methylation domain, a histone residue modification domain, a localization factor, a transcription modification factor, a light-gated control factor, a chemically inducible factor, a chromatin visualization factor, or a combination thereof.

In some embodiments, the additional functional moiety may comprise a nuclear localization signal (NLS), a nuclear export signal (NES), or a combination thereof. In some examples, the fusion polypeptide may comprise an NLS, which may be located at either the N-terminus or the C-terminus. In specific examples, the fusion polypeptide may comprise a first NLS located at the N-terminus and a second NLS located at the C-terminus. The first and second NLS fragments may be identical. Alternatively, the two NLS fragments may be different. In some embodiments, the fusion polypeptide may comprise an NLS near the N-terminus and/or near the C-terminus (e.g., within about 1, 2, 3, 4, or 5 of the first amino acid or last amino acid of the CRISPR nuclease). In some embodiments, the fusion polypeptide may comprise an NLS within a flexible loop of the CRISPR nuclease. Exemplary fusion CRISPR nuclease polypeptides comprising one or more NLS signals are provided in Tables 1, 15, and 21.

In some embodiments, the additional functional moiety may be a flexible peptide linker, for example, an XTEN peptide linker, or a G/S rich peptide linker. Examples of such peptide linkers are provided in Example 1 below, which may be applicable to any of the CRISPR nuclease polypeptides disclosed herein.

### B. Preparation of CRISPR Nuclease Polypeptides

The CRISPR nuclease polypeptides as disclosed herein may be prepared by conventional methods or the methods disclosed herein. For example, the CRISPR nuclease polypeptides can be prepared by culturing host cells such as bacteria cells or mammalian cells, capable of producing the nuclease polypeptides, isolating the nuclease polypeptides thus produced, and optionally, purifying the nuclease polypeptides. The CRISPR nuclease polypeptides thus prepared may be complexed with a gRNA.

The CRISPR nuclease polypeptides can be also prepared by an in vitro coupled transcription-translation system and optionally complexes with gRNA. Bacteria that can be used for preparation of the CRISPR nuclease polypeptides are not particularly limited as long as they can produce the CRISPR nuclease polypeptides. Some nonlimiting examples of the bacteria include E. coli cells described herein.

Unless otherwise noted, all compositions and complexes and polypeptides provided herein are made in reference to the active level of that composition or complex or polypeptide, and are exclusive of impurities, for example, residual solvents or by-products, which may be present in commercially available sources. Enzymatic component weights are based on total active protein. All percentages and ratios are calculated by weight unless otherwise indicated. All percentages and ratios are calculated based on the total composition unless otherwise indicated. In the exemplified composition, the enzymatic levels are expressed by pure enzyme by weight of the total composition and unless otherwise specified, the ingredients are expressed by weight of the total compositions.

### (i) Vectors

The present disclosure provides vectors for expressing the CRISPR nuclease polypeptides. In some embodiments, a vector disclosed herein includes a nucleotide sequence encoding a CRISPR nuclease polypeptide as provided herein. In some embodiments, the vector comprises a Pol II promoter or a Pol III promoter.

Expression of natural or synthetic polynucleotides is typically achieved by operably linking a polynucleotide encoding the CRISPR nuclease polypeptide to a promoter and incorporating the construct into an expression vector. The expression vector is not particularly limited as long as it includes a polynucleotide encoding the CRISPR nuclease polypeptides and can be suitable for replication and integration in eukaryotic cells.

Typical expression vectors include transcription and translation terminators, initiation sequences, and promoters useful for expression of the desired polynucleotide. For example, plasmid vectors carrying a recognition sequence for RNA polymerase (pSP64, pBluescript, etc.). may be used. Vectors including those derived from retroviruses such as lentivirus are suitable tools to achieve long-term gene transfer since they allow long-term, stable integration of a transgene and its propagation in daughter cells. Examples of vectors include expression vectors, replication vectors, probe generation vectors, and sequencing vectors. The expression vector may be provided to a cell in the form of a viral vector.

Viral vector technology is well known in the art and described in a variety of virology and molecular biology manuals. Viruses useful as vectors include, but are not limited to phage viruses, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers.

The kind of the vector is not particularly limited, and a vector that can be expressed in host cells can be appropriately selected. To be more specific, depending on the kind of the host cell, a promoter sequence to ensure the expression of the polypeptide(s) from the polynucleotide is appropriately selected, and this promoter sequence and the polynucleotide are inserted into any of various plasmids etc. for preparation of the expression vector.

Additional promoter elements, e.g., enhancing sequences, regulate the frequency of transcriptional initiation. Typically, these are located in the region 30-110 bp upstream of the start site, although a number of promoters have recently been shown to contain functional elements downstream of the start site as well. Depending on the promoter, it appears that individual elements can function either cooperatively or independently to activate transcription.

Further, the disclosure should not be limited to the use of constitutive promoters. Inducible promoters are also contemplated as part of the disclosure. The use of an inducible promoter provides a molecular switch capable of turning on expression of the polynucleotide sequence which it is operatively linked when such expression is desired or turning off the expression when expression is not desired. Examples of inducible promoters include, but are not limited to a metallothionine promoter, a glucocorticoid promoter, a progesterone promoter, and a tetracycline promoter.

The expression vector to be introduced can Also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate transcriptional control sequences to enable expression in the host cells. Examples of such a marker include a dihydrofolate reductase gene and a neomycin resistance gene for eukaryotic cell culture; and a tetracycline resistance gene and an ampicillin resistance gene for culture of E. coli and other bacteria. By use of such a selection marker, it can be confirmed whether the polynucleotide encoding the polypeptide(s) of the present invention has been transferred into the host cells and then expressed without fail.

The preparation method using recombinant expression vectors is not particularly limited, and examples thereof include methods using a plasmid, a phage or a cosmid.

### (ii) Methods of Expression

The present disclosure includes a method for protein expression, comprising translating the CRISPR nuclease polypeptides described herein.

In some embodiments, a host cell described herein is used to express the CRISPR nuclease polypeptides. The host cell is not particularly limited, and various known cells can be preferably used. Specific examples of the host cell include bacteria such as E. coli, yeasts (budding yeast, Saccharomyces cerevisiae, and fission yeast, Schizosaccharomyces pombe), nematodes (Caenorhabditis elegans), Xenopus laevis oocytes, and animal cells (for example, CHO cells, COS cells and HEK293 cells). The method for transferring the expression vector described above into host cells, i.e., the transformation method, is not particularly limited, and known methods such as electroporation, the calcium phosphate method, the liposome method and the DEAE dextran method can be used.

After a host is transformed with the expression vector, the host cells may be cultured, cultivated or bred, for production the CRISPR nuclease polypeptides. After expression, the host cells can be collected and CRISPR nuclease polypeptides purified from the cultures etc. according to conventional methods (for example, filtration, centrifugation, cell disruption, gel filtration chromatography, ion exchange chromatography, etc.).

A variety of methods can be used to determine the level of production of a mature CRISPR nuclease polypeptide in a host cell. Such methods include, but are not limited to, for example, methods that utilize either polyclonal or monoclonal antibodies specific for the proteins or a labeling tag as described elsewhere herein. Exemplary methods include, but are not limited to, enzyme-linked immunosorbent assays (ELISA), radioimmunoassays (MA), fluorescent immunoassays (FIA), and fluorescent activated cell sorting (FACS). These and other assays are well known in the art (See, e.g., Maddox et al., J. Exp. Med. 158:1211 [1983]).

The present disclosure provides methods of in vivo expression of CRISPR nuclease polypeptides (and optionally the gRNA in the gene editing system disclosed herein). Such a method may comprise providing a polyribonucleotide encoding the CRISPR nuclease polypeptide to a host cell in a subject (e.g., a human subject) wherein the polyribonucleotide encodes the CRISPR nuclease polypeptide expressing the CRISPR nuclease polypeptide from the cell.

### II. Gene Editing System

In some aspects, the present disclosure provides gene editing systems with enhanced gene editing efficiencies. The gene editing system comprises any of the CRISPR nuclease polypeptides derived from Nuclease A, Nuclease K, or Nuclease M as disclosed herein or a nucleic encoding the CRISPR nuclease and one or more guide RNAs (gRNAs) or nucleic acid(s) encoding the gRNAs.

### CRISPR Nuclease

In some embodiments, the gene editing system disclosed herein comprises a CRISPR nuclease polypeptide as provided herein, e.g., any of the reference CRISPR Nuclease A, Nuclease K, or Nuclease M or a variant thereof, e.g., comprising one or more arginine and/or lysine substitutions (e.g., arginine substitutions), one or more nickase mutations, additional mutations such as N-terminal truncation where applicable, or a combination thereof. See above disclosures. Such a protein component may form a complex with the gRNA(s) in the same gene editing system. Alternatively, the gene editing system comprises a nucleic acid encoding the CRISPR nuclease polypeptide. In some instances, the nucleic acid can be an expression vector (e.g., a viral vector) for producing the encoded nuclease polypeptide in host cells. In some instances, the expression vector may further comprise a coding sequence for producing one or more gRNAs of the gene editing system.

### Guide RNAs

The gene editing system disclosed herein further comprises one or more gRNAs or nuclei acid(s) encoding such. As used herein, the terms “RNA guide”, “RNA guide sequence,” or “guide RNA (gRNA)” refer to an RNA molecule or a modified RNA molecule that facilitates the targeting of a CRISPR nuclease described herein to a genomic site of interest. For example, an RNA guide can be a molecule that comprises a spacer sequence and a scaffold sequence. The spacer sequence recognizes (e.g., binds to) a site in a non-PAM strand that is complementary to a target sequence in the PAM strand, e.g., designed to be complementary to a specific nucleic acid sequence. The scaffold sequence contains a nuclease binding sequence for binding to the CRISPR nuclease. In some embodiments, the scaffold is an RNA sequence. In some instances, the gRNA disclosed herein may further comprise a linker sequence, a 5′ end and/or 3′ end protection fragment, or a combination thereof.

### (i) Spacer Sequences

As used herein, the term “spacer” and “spacer sequence” (a.k.a., a DNA-binding sequence) is a portion in an RNA guide that is the RNA equivalent of the target sequence (a DNA sequence). The spacer contains a sequence capable of binding to the non-PAM strand via base-pairing at the site complementary to the target sequence (which is in the PAM strand). Such a spacer is also known as specific to the target sequence. In some instances, the spacer may be at least 75% identical to the target sequence (e.g., at least 80%, at least 85%, at least 90%, at least 95%, at least 98%, or at least 99%), except for the RNA-DNA sequence difference. In some instances, the spacer may be 100% identical to the target sequence except for the RNA-DNA sequence difference.

The gene editing system disclosed herein comprises one or more gRNAs, each comprising a spacer sequence specific to a target sequence in a genomic site of interest and a scaffold sequence, which is recognizable by the CRISPR nuclease polypeptide contained in the gene editing system.

In association with Nuclease A or a variant thereof as disclosed herein, the target sequence can be adjacent to (e.g., upstream to or 5′ to) PAM of 5′-NGG-3′, in which N represents any nucleotide.

In association with Nuclease K or a variant thereof as disclosed herein, the target sequence can be adjacent to (e.g., upstream to or 5′ to) a PAM of 5′-NGG-3′, in which N represents any nucleotide.

In association with Nuclease M, the target sequence can be adjacent to (e.g., upstream to or 5′ to) a PAM of 5′-WTAAH-3′, in which W is A or T and His A, C, or T. In some examples, the PAM can be 5′-TTAAA-3′.

As used herein, the term “protospacer adjacent motif” or “PAM sequence” refers to a DNA sequence adjacent to a target sequence. In some embodiments, a PAM sequence is required for binding of the CRISPR nuclease and/or indel activity. In a double-stranded DNA molecule, the strand containing the PAM motif is called the “PAM-strand” and the complementary strand is called the “non-PAM strand.” The gRNA binds to a site in the non-PAM strand that is complementary to a target sequence disclosed herein, and the PAM sequence as described herein is present in the PAM-strand. The PAM motif can be located upstream to the target sequence.

As used herein, the term “adjacent to” refers to a nucleotide or amino acid sequence in close proximity to another nucleotide or amino acid sequence. In some embodiments, a nucleotide sequence is adjacent to another nucleotide sequence if no nucleotides separate the two sequences (i.e., immediately adjacent). In some embodiments, a nucleotide sequence is adjacent to another nucleotide sequence if a small number of nucleotides separate the two sequences (e.g., about 1, 2, 3, 4, 5, 6, 7, 8, 9, 10, 11, 12, 13, 14, 15, 16, 17, 18, 19, or 20 nucleotides). In some embodiments, a first sequence is adjacent to a second sequence if the two sequences are separated by about 5, 6, 7, 8, 9, 10, 11, 12, 13, 14, or 15 nucleotides. In some embodiments, a first sequence is adjacent to a second sequence if the two sequences are separated by up to 2 nucleotides, up to 5 nucleotides, up to 8 nucleotides, up to 10 nucleotides, up to 12 nucleotides, or up to 15 nucleotides. In some embodiments, a first sequence is adjacent to a second sequence if the two sequences are separated by 2-5 nucleotides, 4-6 nucleotides, 4-8 nucleotides, 4-10 nucleotides, 6-8 nucleotides, 6-10 nucleotides, 6-12 nucleotides, 8-10 nucleotides, 8-12 nucleotides, 10-12 nucleotides, 10-15 nucleotides, or 12-15 nucleotides.

In specific examples, the spacer is specific to a target sequence in a genomic site of interest, the target sequence being immediately adjacent to the PAM motif. In other specific examples, the target sequence and the PAM motif may have a small gap of less than 5 (e.g., 1, 2, 3, 4, or 5) nucleotides.

A spacer sequence as disclosed herein may have a length of from about 15 nucleotides to about 30 nucleotides. For example, the spacer can have a length of from about 15 nucleotides to about 20 nucleotides, from about 15 nucleotides to about 25 nucleotides, from about 20 nucleotides to about 25 nucleotides, or from about 20 nucleotides to about 30 nucleotides. In some embodiments, the spacer in the gRNA may be generally designed to have a length of between 15 and 25 nucleotides (e.g., 15, 16, 17, 18, 19, 20, 21, 22, 23, 24, and 25) and be complementary to a specific target sequence. In some embodiments, the spacer sequence may be designed to have a length of between 18-22 nucleotides (e.g., 20 nucleotides).

In some embodiments, the spacer sequence may have at least about 60%, at least about 65%, at least about 70%, at least about 75%, at least about 80%, at least about 85%, at least about 90%, at least about 91%, at least about 92%, at least about 93%, at least about 94%, at least about 95%, at least about 96%, at least about 97%, at least about 98%, at least about 99%, or at least about 99.5% sequence identity to a target sequence as described herein and is capable of binding to the complementary region of the target sequence via base-pairing.

In some embodiments, the spacer sequence comprises only RNA bases. In some embodiments, the spacer sequence comprises a DNA base (e.g., the spacer comprises at least one thymine). In some embodiments, the spacer sequence comprises RNA bases and DNA bases (e.g., the DNA-binding sequence comprises at least one thymine and at least one uracil).

### (ii) Scaffold Sequence

The scaffold sequence in the gRNA is recognizable by the CRISPR nuclease polypeptide also in the gene editing system.

In some instances, the scaffold sequence is recognizable by Nuclease A (SEQ ID NO: 1) or any engineered variants thereof as disclosed herein. Such a scaffold sequence may comprise SEQ ID NO: 2 below.

(SEQ ID NO: 2)
GUUACAGUUAAGGCUCUUUGGAAACAAAGAAGCCUUAAUUGUAAAACG
CCUAUAUGGUAAAGUGAUGUACGUUUGGGUAUAUAUCGCCAGCCUGAA
CCUCUACGCCAGAAAUGGCAGCUUUAUCAUGGGUUAGGACGAUAUUUA
AAAAACUUUCUGCGGCUUGCUUACUUUAGUAAGCUUUGUGGCUGAGGC
AGAAUUCCUU

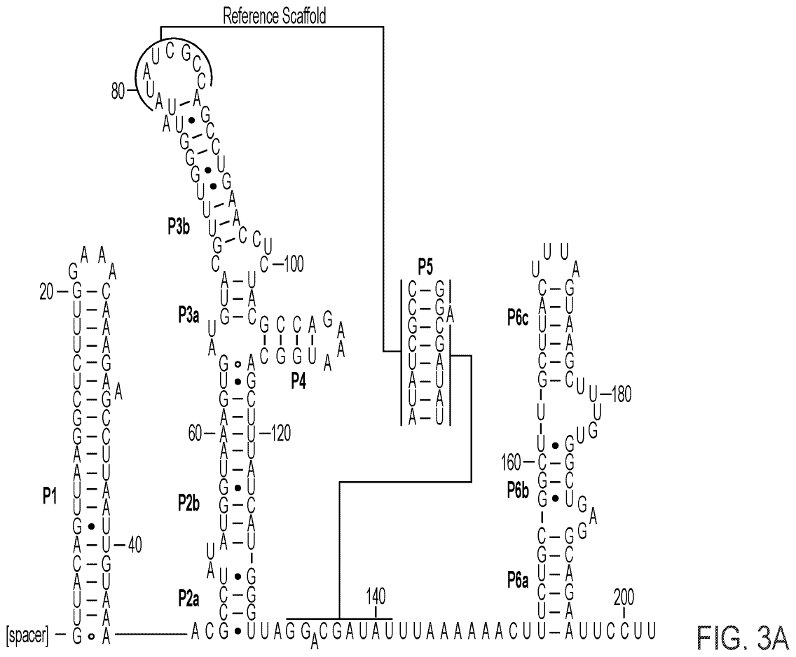

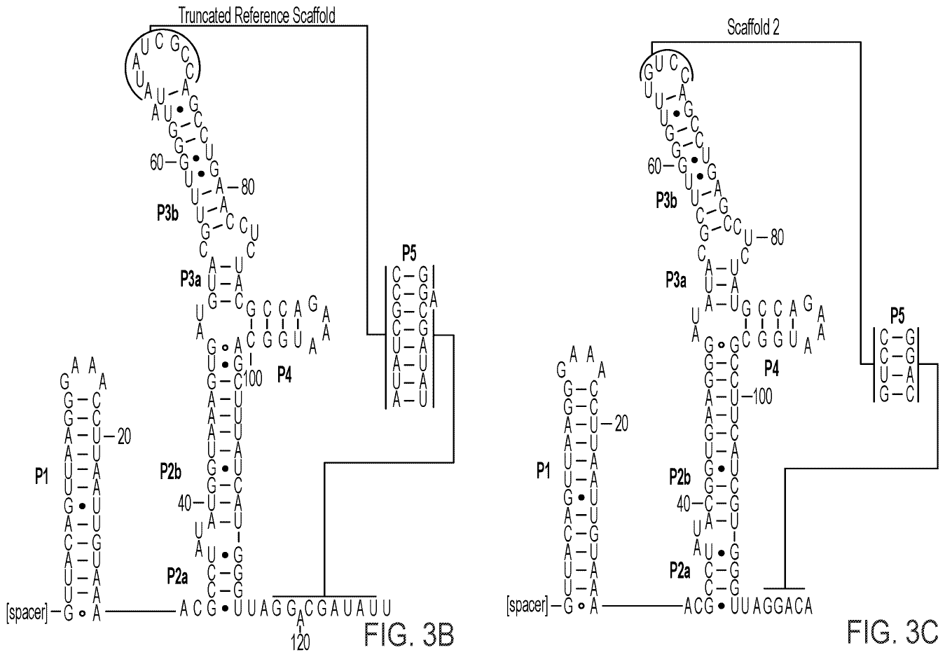

The predicted secondary structure of the reference scaffold sequence of SEQ ID NO: 2 is provided in FIG. 3A.

In other instances, the scaffold sequence recognizable by Nuclease A (SEQ ID NO: 1) or any engineered variants thereof may be a variant derived from SEQ ID NO: 2. Such a variant scaffold sequence may comprise a nucleotide sequence at least 80% (e.g., at least 85%, 90%, 95%, 98%, or greater) identical to SEQ ID NO: 2. Alternatively or in addition, the variant scaffold sequence may comprise deletions, nucleotide substitutions, or a combination thereof. The variant CRISPR nuclease polypeptide may have increased binding to the variant scaffold sequence as compared with the scaffold of SEQ ID NO: 2. In some examples, the variant scaffold may be a fragment of SEQ ID NO: 2 or a variant thereof as disclosed herein. For example, the variant scaffold for use in the gRNAs provided herein may have a length ranging from 100-150.

In some embodiments, the scaffold sequence recognizable by Nuclease A (SEQ ID NO: 1) or any engineered variants thereof can be a truncated variant of SEQ ID NO: 2. Any of the truncation variants provided herein may be about 110-140-nucleotide in length.

In some examples, the truncated variant scaffold sequence recognizable by Nuclease A (SEQ ID NO: 1) or any engineered variants thereof may have a 3′ truncation relative to SEQ ID NO: 2. Such a 3′ truncation may remove one or more of the stem structures P6a, P6b, and P6c depicted in FIG. 3A. In specific examples, the 3′ truncation may comprise a deletion within residues 143-202 of SEQ ID NO: 2, e.g., deletion of the 143-202 fragment of SEQ ID NO: 2. See, FIG. 3B.

Alternatively or in addition, the variant scaffold sequence can be a truncated variant of SEQ ID NO: 2 having an internal truncation relative to SEQ ID NO: 2. For example, such a truncation variant may comprise a deletion of whole or part of the stem structure P1 depicted in FIG. 3A. In some examples, the truncation variant may comprise a deletion within residues 14-20 of SEQ ID NO: 2, within residues 25-32 of SEQ ID NO: 2, or a combination thereof. In specific examples, the truncation variant may comprise deletions of the 14-20 fragment and 25-32 fragment of SEQ ID NO: 2. See, e.g., FIGS. 3B and 3C. In some instances, the truncation variant may comprise a deletion in the loop consisting of residues 77-86 of SEQ ID NO: 2 (see, FIG. 3C), which may result in shortening of the stem structure P5 depicted in FIG. 3A. In some examples, the truncation variant may comprise a deletion within residues 81-85 (e.g., deletion of the 81-85 fragment) of SEQ ID NO: 2.

In some examples, the variant scaffold sequence recognizable by Nuclease A (SEQ ID NO: 1) or any engineered variants thereof may comprise a combination of the 3′ truncation and one or more of the internal truncations as disclosed herein. Alternatively or in addition, the variant scaffold sequence may comprise one or more nucleotide variations relative to the corresponding residues in SEQ ID NO: 2.

In one specific examples, the scaffold sequence recognizable by Nuclease A (SEQ ID NO: 1) or any engineered variants thereof comprises (e.g., consists of) the nucleotide sequence of SEQ ID NO: 27. In another specific example, the scaffold sequence comprises (e.g., consists of) SEQ ID NO: 28. Such a scaffold sequence may be about 115-130-nt in length.

In some instances, the scaffold sequence is recognizable by Nuclease K (SEQ ID NO: 65) or any engineered variants thereof as disclosed herein. Such a scaffold sequence may comprise SEQ ID NO: 79 below.

(SEQ ID NO: 79)
GUUACAGUUAAGGCUCUGAAAAGAGCCUUAAUUGUAAAACGCCUAUAC
AGUGAAGGGAUAUACGCUUGGGUUUGUCCAGCCUGAGCCUCUAUGCCA
GAAAUGGCGCCUUCAUCGUGGGUUAGGACAUUUAAAUUUAAAAACUAU
UCAGCACUGUUUGCUCUUGUCAGCUUGGUGGCAGA

In other instances, the scaffold sequence recognizable by Nuclease K (SEQ ID NO: 65) or any engineered variants thereof may be a variant derived from SEQ ID NO: 79. Such a variant scaffold sequence may comprise a nucleotide sequence at least 80% (e.g., at least 85%, 90%, 95%, 98%, or greater) identical to SEQ ID NO: 79. Alternatively or in addition, the variant scaffold sequence may comprise deletions, nucleotide substitutions, or a combination thereof. The variant CRISPR nuclease polypeptide may have increased binding to the variant scaffold sequence as compared with the scaffold of SEQ ID NO: 79. In some examples, the variant scaffold may be a fragment of SEQ ID NO: 79 or a variant thereof as disclosed herein. For example, the variant scaffold sequence for use in the gRNAs provided herein may have a length ranging from 100-150 nt.

In some instances, the scaffold sequence is recognizable by Nuclease M (SEQ ID NO: 80) or any engineered variants thereof as disclosed herein. Such a scaffold sequence may comprise SEQ ID NO: 94 below.

(SEQ ID NO: 94)
GUCAACUACCCCCGUCUAAAGACGGAGGCAUGAGGUUUCGUAACCAAG
UGUUGUACCUGCGGGUACAGUAGUUGAACAGGCGGCGAUGCGGCUGGG
CACUCCAGGAUGCCACUCCCAGUCCCGGACACUGCCGACGAGCCGCAUC
AAGCCGGGGGAGACCAACCGGCUAACGAUAGCCGAGCAAUUACCUAAA
AAGAGGUGCAAAGGAAAUGGUAU

In other instances, the scaffold sequence recognizable by Nuclease M (SEQ ID NO: 80) or any engineered variants thereof may be a variant derived from SEQ ID NO: 94. Such a variant scaffold sequence may comprise a nucleotide sequence at least 80% (e.g., at least 85%, 90%, 95%, 98%, or greater) identical to SEQ ID NO: 94. Alternatively or in addition, the variant scaffold sequence may comprise deletions, nucleotide substitutions, or a combination thereof. The variant CRISPR nuclease polypeptide may have increased binding to the variant scaffold sequence as compared with the scaffold of SEQ ID NO: 94. In some examples, the variant scaffold may be a fragment of SEQ ID NO: 94 or a variant thereof as disclosed herein. For example, the variant scaffold for use in the gRNAs provided herein may have a length ranging from 150-200 nt.

In any of the gRNAs disclosed herein, the scaffold sequence may be located at the 3′ end of the spacer sequence. In some instances, the scaffold sequence and spacer sequence are connected directly. In other instances, the scaffold sequence and spacer sequence may be connected via a nucleotide linker.

### Modification of Nucleic Acids

Any of the guide RNAs or encoding nucleic acids (e.g., mRNA encoding the CRISPR nuclease polypeptide) in a gene editing system as disclosed herein may include one or more modifications.

Exemplary modifications can include any modification to the sugar, the nucleobase, the internucleoside linkage (e.g., to a linking phosphate/to a phosphodiester linkage/to the phosphodiester backbone), and any combination thereof. Some of the exemplary modifications provided herein are described in detail below.

The gRNA or any of the nucleic acid sequences encoding components of the composition may include any useful modification, such as to the sugar, the nucleobase, or the internucleoside linkage (e.g., to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). One or more atoms of a purine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. In some embodiments, the gRNA or any of the nucleic acid sequences encoding components of the composition may comprise an abasic site (i.e., a location that does not have a purine or a pyrimidine). Modifications may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof). Additional modifications are described herein.

In some embodiments, the modification may include a chemical or cellular induced modification. For example, some nonlimiting examples of intracellular RNA modifications are described by Lewis and Pan in “RNA modifications and structures cooperate to guide RNA-protein interactions” from Nat Reviews Mol Cell Biol, 2017, 18:202-210.

Different sugar modifications, nucleotide modifications, and/or internucleoside linkages (e.g., backbone structures) may exist at various positions in the sequence. One of ordinary skill in the art will appreciate that the nucleotide analogs or other modification(s) may be located at any position(s) of the sequence, such that the function of the sequence is not substantially decreased. The sequence may include from about 1% to about 100% modified nucleotides (either in relation to overall nucleotide content, or in relation to one or more types of nucleotide, i.e. any one or more of A, G, U or C) or any intervening percentage (e.g., from 1% to 20%>, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, sugar modifications (e.g., at the 2′ position or 4′ position) or replacement of the sugar at one or more ribonucleotides of the sequence may, as well as backbone modifications, include modification or replacement of the phosphodiester linkages. Specific examples of a sequence include, but are not limited to, sequences including modified backbones or no natural internucleoside linkages such as internucleoside modifications, including modification or replacement of the phosphodiester linkages. Sequences having modified backbones include, among others, those that do not have a phosphorus atom in the backbone. For the purposes of this application, and as sometimes referenced in the art, modified RNAs that do not have a phosphorus atom in their internucleoside backbone can also be considered to be oligonucleosides. In particular embodiments, a sequence will include ribonucleotides with a phosphorus atom in its internucleoside backbone.

Modified sequence backbones may include, for example, phosphorothioates, chiral phosphorothioates, phosphorodithioates, phosphotriesters, aminoalkylphosphotriesters, methyl and other alkyl phosphonates such as 3′-alkylene phosphonates and chiral phosphonates, phosphinates, phosphoramidates such as 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included. In some embodiments, the sequence may be negatively or positively charged.

The modified nucleotides, which may be incorporated into the sequence, can be modified on the internucleoside linkage (e.g., phosphate backbone). Herein, in the context of the polynucleotide backbone, the phrases “phosphate” and “phosphodiester” are used interchangeably. Backbone phosphate groups can be modified by replacing one or more of the oxygen atoms with a different substituent. Further, the modified nucleosides and nucleotides can include the wholesale replacement of an unmodified phosphate moiety with another internucleoside linkage as described herein. Examples of modified phosphate groups include, but are not limited to, phosphorothioate, phosphoroselenates, boranophosphates, boranophosphate esters, hydrogen phosphonates, phosphoramidates, phosphorodiamidates, alkyl or aryl phosphonates, and phosphotriesters. Phosphorodithioates have both non-linking oxygens replaced by sulfur. The phosphate linker can also be modified by the replacement of a linking oxygen with nitrogen (bridged phosphoramidates), sulfur (bridged phosphorothioates), and carbon (bridged methylene-phosphonates).

The α-thio substituted phosphate moiety is provided to confer stability to RNA and DNA polymers through the unnatural phosphorothioate backbone linkages. Phosphorothioate DNA and RNA have increased nuclease resistance and subsequently a longer half-life in a cellular environment.

In specific embodiments, a modified nucleoside includes an alpha-thio-nucleoside (e.g., 5′-O-(1-thiophosphate)-adenosine, 5′-O-(1-thiophosphate)-cytidine (α-thio-cytidine), 5′-O-(1-thiophosphate)-guanosine, 5′-O-(1-thiophosphate)-uridine, or 5′-O-(1-thiophosphate)-pseudouridine).

Other internucleoside linkages that may be employed according to the present invention, including internucleoside linkages which do not contain a phosphorous atom, are described herein.

In some embodiments, the sequence may include one or more cytotoxic nucleosides. For example, cytotoxic nucleosides may be incorporated into sequence, such as bifunctional modification. Cytotoxic nucleoside may include, but are not limited to, adenosine arabinoside, 5-azacytidine, 4′-thio-aracytidine, cyclopentenylcytosine, cladribine, clofarabine, cytarabine, cytosine arabinoside, 1-(2-C-cyano-2-deoxy-beta-D-arabino-pentofuranosyl)-cytosine, decitabine, 5-fluorouracil, fludarabine, floxuridine, gemcitabine, a combination of tegafur and uracil, tegafur ((RS)-5-fluoro-1-(tetrahydrofuran-2-yl)pyrimidine-2,4 (1H,3H)-dione), troxacitabine, tezacitabine, 2′-deoxy-2′-methylidenecytidine (DMDC), and 6-mercaptopurine. Additional examples include fludarabine phosphate, N4-behenoyl-1-beta-D-arabinofuranosylcytosine, N4-octadecyl-1-beta-D-arabinofuranosylcytosine, N4-palmitoyl-1-(2-C-cyano-2-deoxy-beta-D-arabino-pentofuranosyl) cytosine, and P-4055 (cytarabine 5′-elaidic acid ester).

In some embodiments, the sequence includes one or more post-transcriptional modifications (e.g., capping, cleavage, polyadenylation, splicing, poly-A sequence, methylation, acylation, phosphorylation, methylation of lysine and arginine residues, acetylation, and nitrosylation of thiol groups and tyrosine residues, etc.). The one or more post-transcriptional modifications can be any post-transcriptional modification, such as any of the more than one hundred different nucleoside modifications that have been identified in RNA (Rozenski, J, Crain, P, and McCloskey, J. (1999). The RNA Modification Database: 1999 update. Nucl Acids Res 27:196-197) In some embodiments, the first isolated nucleic acid comprises messenger RNA (mRNA). In some embodiments, the mRNA comprises at least one nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine. In some embodiments, the mRNA comprises at least one nucleoside selected from the group consisting of 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine. In some embodiments, the mRNA comprises at least one nucleoside selected from the group consisting of 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl) adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine. In some embodiments, mRNA comprises at least one nucleoside selected from the group consisting of inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

The sequence may or may not be uniformly modified along the entire length of the molecule. For example, one or more or all types of nucleotides (e.g., naturally-occurring nucleotides, purine or pyrimidine, or any one or more or all of A, G, U, C, I, pU) may or may not be uniformly modified in the sequence, or in a given predetermined sequence region thereof. In some embodiments, the sequence includes a pseudouridine. In some embodiments, the sequence includes an inosine, which may aid in the immune system characterizing the sequence as endogenous versus viral RNAs. The incorporation of inosine may also mediate improved RNA stability/reduced degradation. See for example, Yu, Z. et al. (2015) RNA editing by ADAR1 marks dsRNA as “self”. Cell Res. 25, 1283-1284, which is incorporated by reference in its entirety.

In some embodiments, any RNA sequence described herein may comprise an end modification (e.g., a 5′ end modification or a 3′ end modification). In some embodiments, the end modification is a chemical modification. In some embodiments, the end modification is a structural modification. See disclosures herein.

When a gene editing system disclosed herein comprises nucleic acids encoding the CRISPR nuclease, such nucleic acid molecules may contain any of the modifications disclosed herein, where applicable.

### III. Gene Editing Methods

Any of the gene editing systems can be used to genetically modify (edit) a target nucleic acid, which can be a genetic site of interest, e.g., a genetic site where genetic editing is needed, for example, to fix a genetic mutation, to introduce a protective mutation, to introduce modifications for modulating expression of a gene, etc.

The gene editing systems and compositions disclosed herein are applicable for editing and introducing edits into a variety of target sequences. In some embodiments, the target sequence is a DNA molecule, such as a DNA locus (referred to herein as a target sequence or an on-target sequence).

For gene editing systems comprising Nuclease A (SEQ ID NO: 1) or an engineered variant thereof, the target sequence is adjacent to the PAM motif of 5′-NGG-3′, in which N refers to any nucleotide. In some instances, the PAM motif is 3′ (downstream) to the target sequence.

For gene editing systems comprising Nuclease K (SEQ ID NO: 65) or an engineered variant thereof, the target sequence is adjacent to the PAM motif of 5′-NGG-3′, in which N refers to any nucleotide. In some instances, the PAM motif is 3′ (downstream) to the target sequence.

For gene editing systems comprising Nuclease M (SEQ ID NO: 80) or an engineered variant thereof, the target sequence is adjacent to the PAM motif of 5′-WTAAH-3′, in which W is A or T and His A, C, or T. In one example, the PAM is 5′-TTAAA-3′. In some instances, the PAM motif is 3′ (downstream) to the target sequence.

In some embodiments, the target nucleic acid is a genomic site in a cell. In some instances, the target nucleic acid where the genetic edit would occur can be in a protein-coding region. Alternatively, the target nucleic acid may be in a regulatory region, such as a promoter, enhancer, a 5′ or 3′ untranslated region. In other instances, the target nucleic acid can be in a non-coding gene, such as transposon, miRNA, tRNA, ribosomal RNA, ribozyme, or lincRNA.

### A. Gene Edits

Any of the gene editing systems disclosed herein may be used to edit a target gene of interest, e.g., a gene involved in a disease (e.g., a genetic disease). In some embodiments, the target gene can be one that is involved in an immune response in a subject. For example, the target gene can be an immune checkpoint gene or a tumor necrosis factor receptor superfamily member. The gene edit may occur in an exon (e.g., in a coding region). Alternatively, the gene editing may occur in an intron or in a regulatory element (e.g., promoter, enhancer, inhibitory element, etc.). In some instances, the gene edit may result in reducing or eliminating the expression of the target gene. In other instances, the gene edit may result in enhancing expression of the target gene (e.g., disrupting an inhibitory factor).

In some aspects, provided herein are methods for introducing at least one edit into a target nucleic acid (e.g., a genomic site of interest such as in any of the target genes disclosed herein) using the gene editing system described herein.

As used herein, the term “edit” refers to one or more modifications introduced into a nucleotide sequence in a target nucleic acid such as in a genomic site of interest. The edit may occur within a target sequence as defined herein. Alternatively, the edit may occur outside the target sequence (e.g., adjacent to the target sequence). The edit can be one or more substitutions, one or more insertions, one or more deletions, or a combination thereof.

Deletion refers to a loss of a nucleotide or nucleotides in a nucleic acid sequence, relative to a reference sequence. No particular process is implied in how to make a sequence comprising a deletion. For instance, a sequence comprising a deletion can be synthesized directly from individual nucleotides. In other embodiments, a deletion is made by providing and then altering a reference sequence. The nucleic acid sequence can be in a genome of an organism. The nucleic acid sequence can be in a cell. The nucleic acid sequence can be a DNA sequence. The deletion can be a frameshift mutation or a non-frameshift mutation. A deletion described herein refers to an insertion of up to several kilobases.

Insertion refers to a gain of a nucleotide or nucleotides in a nucleic acid sequence, relative to a reference sequence. No particular process is implied in how to make a sequence comprising an insertion. For instance, a sequence comprising an insertion can be synthesized directly from individual nucleotides. In other embodiments, an insertion is made by providing and then altering a reference sequence. The nucleic acid sequence can be in a genome of an organism. The nucleic acid sequence can be in a cell. The nucleic acid sequence can be a DNA sequence. The insertion can be a frameshift mutation or a non-frameshift mutation. An insertion described herein refers to an insertion of up to several kilobases.

In some embodiments, the gene editing methods disclosed herein may introduce edits, including a substitution, an insertion, a deletion, or a combination thereof, into the target nucleic acid.

In some examples, the edits can include at least one substitution, at least one insertion, and/or at least one deletion. In some embodiments, the edit comprises at least one substitution, insertion, or deletion. In some embodiments, the substitution, insertion, or deletion is at least 1-500 nucleotides (e.g., 1-10 nucleotides, 10-30 nucleotides, 30-50 nucleotides, 50-100 nucleotides, 100-200 nucleotides, 200-300 nucleotides, 300-400 nucleotides, or 400-500 nucleotides).

In some examples, the edit may occur within about 500 nucleotides of any of the PAM sequences disclosed herein (e.g., a PAM sequence associated with Nuclease A, Nuclease K, or Nuclease M as disclosed herein). In some embodiments, the edit occurs adjacent to the PAM sequence, e.g., within about 1-500 nucleotides upstream or downstream of the PAM sequence. In some embodiments, the edit may occur within about 1-10 nucleotides, 10-30 nucleotides, 30-50 nucleotides, 50-100 nucleotides, 100-200 nucleotides, 200-300 nucleotides, 300-400 nucleotides, or 400-500 nucleotides upstream of the PAM sequence. Alternatively, or in addition, the edit may occur within about 1-10 nucleotides, 10-30 nucleotides, 30-50 nucleotides, 50-100 nucleotides, 100-200 nucleotides, 200-300 nucleotides, 300-400 nucleotides, or 400-500 nucleotides downstream of the PAM sequence.

In some embodiments, the edit starts at the PAM sequence. In some embodiments, the edit may start within about 1-30 nucleotide downstream of the PAM. Alternatively, the edits may start within about 1-30 nucleotide upstream of the PAM.

In some embodiments, the edit may end within about 1-300 nucleotides upstream of the PAM sequence, for example, ends within about 1-10 nucleotides, about 10-30 nucleotides, about 30-50 nucleotides, about 50-100 nucleotides, about 100-200 nucleotides, or about 200-300 nucleotides upstream of the PAM sequence. Alternatively, the edit may end within about 1-300 nucleotides downstream of the PAM sequence, for example, ends within about 1-10 nucleotides, about 10-30 nucleotides, about 30-50 nucleotides, about 50-100 nucleotides, about 100-200 nucleotides, or about 200-300 nucleotides downstream of the PAM sequence.

In some embodiments, the edit may end at the PAM sequence. In some embodiments, the edit ends within about 1-30 nucleotide downstream of the PAM. In other embodiments, the edit may end within about 1-30 nucleotide upstream of the PAM.

### B. Gene Editing in Cells

In some aspects, provided herein are methods for editing a genomic site of interest (e.g., a target gene as disclosed herein) in cells using any of the gene editing systems disclosed herein. To perform this method, the gene editing system can be delivered to or introduced into a population of cells. In some instances, cells comprising the desired genetic editing may be collected and optionally cultured and expanded in vitro.

The cell described herein can be a variety of cells. In some embodiments, the cell is an isolated cell. In some embodiments, the cell is in cell culture or a co-culture of two or more cell types. In some embodiments, the cell is ex vivo. In some embodiments, the cell is obtained from a living organism and maintained in a cell culture. In some embodiments, the cell is a single-cellular organism.

In some embodiments, the cell is a prokaryotic cell. In some embodiments, the cell is a bacterial cell or derived from a bacterial cell. In some embodiments, the cell is an archaeal cell or derived from an archaeal cell.

In some embodiments, the cell is a eukaryotic cell. In some embodiments, the cell is a plant cell or derived from a plant cell. In some embodiments, the cell is a fungal cell or derived from a fungal cell. In some embodiments, the cell is an animal cell or derived from an animal cell. In some embodiments, the cell is an invertebrate cell or derived from an invertebrate cell. In some embodiments, the cell is a vertebrate cell or derived from a vertebrate cell. In some embodiments, the cell is a mammalian cell or derived from a mammalian cell. In some embodiments, the cell is a human cell. In some embodiments, the cell is a zebra fish cell. In some embodiments, the cell is a primate cell. In some embodiments, the cell is a rodent cell. In some embodiments, the cell is synthetically made, sometimes termed an artificial cell.

In some embodiments, the cell is derived from a cell line. A wide variety of cell lines for tissue culture are known in the art. Examples of cell lines include, but are not limited to, HEK293T, MF7, K562, HeLa, CHO, and transgenic varieties thereof. Cell lines are available from a variety of sources known to those with skill in the art (see, e.g., the American Type Culture Collection (ATCC) (Manassas, Va.)). In some embodiments, the cell is an immortal or immortalized cell. In some embodiments, the cell is a stem cell such as a totipotent stem cell (e.g., omnipotent), a pluripotent stem cell, a multipotent stem cell, an oligopotent stem cell, or an unipotent stem cell. In some embodiments, the cell is an induced pluripotent stem cell (iPSC) or derived from an iPSC. In some embodiments, the cell is a mesenchymal stem cell. In some embodiments, the cell is an embryonic stem cell. In some embodiments, the cell is a hematopoietic stem cell. In some embodiments, the cell is a differentiated cell. For example, in some embodiments, the differentiated cell is a muscle cell (e.g., a myocyte), a fat cell (e.g., an adipocyte), a bone cell (e.g., an osteoblast, osteocyte, osteoclast), a blood cell (e.g., a monocyte, a lymphocyte, a neutrophil, an eosinophil, a basophil, a macrophage, a erythrocyte, or a platelet), a nerve cell (e.g., a neuron), an epithelial cell, an immune cell (e.g., a lymphocyte, a neutrophil, a monocyte, or a macrophage), a liver cell (e.g., a hepatocyte), a fibroblast, or a sex cell. In some embodiments, the cell is a terminally differentiated cell. For example, in some embodiments, the terminally differentiated cell is a neuronal cell, an adipocyte, a cardiomyocyte, a skeletal muscle cell, an epidermal cell, or a gut cell. In some embodiments, the cell is a glial cell. In some embodiments, the cell is a pancreatic islet cell, including an alpha cell, beta cell, delta cell, or enterochromaffin cell. In some embodiments, the cell is an immune cell. In some embodiments, the immune cell is a T cell. In some embodiments, the immune cell is a B cell. In some embodiments, the immune cell is a Natural Killer (NK) cell. In some embodiments, the immune cell is a Tumor Infiltrating Lymphocyte (TIL). In some embodiments, the cell is a mammalian cell, e.g., a human cell or primate cell or a murine cell. In some embodiments, the murine cell is derived from a wild-type mouse, an immunosuppressed mouse, or a disease-specific mouse model. In some embodiments, the cell is a cell within a living tissue, organ, or organism.

In some embodiments, the cell is a primary cell. For example, cultures of primary cells can be passaged 0 times, 1 time, 2 times, 4 times, 5 times, 10 times, 15 times or more. In some embodiments, the primary cells are harvest from an individual by any known method. For example, leukocytes may be harvested by apheresis, leukocytapheresis, density gradient separation, etc. Cells from tissues such as skin, muscle, bone marrow, spleen, liver, pancreas, lung, intestine, stomach, etc. can be harvested by biopsy. An appropriate solution may be used for dispersion or suspension of the harvested cells. Such solution can generally be a balanced salt solution, (e.g., normal saline, phosphate-buffered saline (PBS), Hank's balanced salt solution, etc.), conveniently supplemented with fetal calf serum or other naturally occurring factors, in conjunction with an acceptable buffer at low concentration. Buffers can include HEPES, phosphate buffers, lactate buffers, etc. Cells may be used immediately, or they may be stored (e.g., by freezing). Frozen cells can be thawed and can be capable of being reused. Cells can be frozen in a DMSO, serum, medium buffer (e.g., 10% DMSO, 50% serum, 40% buffered medium), and/or some other such common solution used to preserve cells at freezing temperatures.

In embodiments wherein a gene editing system disclosed herein is introduced into a plurality of cells, at least about 0.5% of the cells comprise the desired edit. In some embodiments, at least about 1% of the cells comprise the desired edit. In some embodiments, at least about 2% of the cells comprise the desired edit. In some embodiments, at least about 3% of the cells comprise the desired edit. In some embodiments, at least about 4% of the cells comprise the desired edit. In some embodiments, at least about 5% of the cells comprise the desired edit. In some embodiments, at least about 10% of the cells comprise the desired edit. In some embodiments, at least about 20% of the cells comprise the desired edit. In some embodiments, at least about 30% of the cells comprise the desired edit. In some embodiments, at least about 40% of the cells comprise the desired edit. In some embodiments, at least about 50% of the cells comprise the desired edit.

The cells carrying the desired genetic edit, e.g., produced by the method disclosed herein using any of the gene editing systems also disclosed herein, are also within the scope of the present disclosure. In some instances, the cells modified by the CRISPR nuclease polypeptide disclosed herein may be useful as an expression system to manufacture biomolecules. For example, the modified cells may be useful to produce biomolecules such as proteins (e.g., cytokines, antibodies, antibody-based molecules), peptides, lipids, carbohydrates, nucleic acids, amino acids, and vitamins. In other embodiments, the modified cell may be useful in the production of a viral vector such as a lentivirus, adenovirus, adeno-associated virus, and oncolytic virus vector. In some embodiments, the modified cell may be useful in cytotoxicity studies. In some embodiments, the modified cell may be useful as a disease model. In some embodiments, the modified cell may be useful in vaccine production. In some embodiments, the modified cell may be useful in therapeutics. For example, in some embodiments, the modified cell may be useful in cellular therapies such as transfusions and transplantations.

In some embodiments, the cells modified by the CRISPR nuclease polypeptide as disclosed herein may be useful to establish a new cell line comprising a modified genomic sequence. In some embodiments, a modified cell of the disclosure is a modified stem cell (e.g., a modified totipotent/omnipotent stem cell, a modified pluripotent stem cell, a modified multipotent stem cell, a modified oligopotent stem cell, or a modified unipotent stem cell) that differentiates into one or more cell lineages comprising the deletion of the modified stem cell. The disclosure further provides organisms (such as animals, plants, or fungi) comprising or produced from a modified cell of the disclosure.

### C. Delivery of Gene Editing Systems to Cells

In some embodiments, any of the gene editing systems or components thereof as disclosed herein may be formulated, for example, including a carrier, such as a carrier and/or a polymeric carrier, e.g., a liposome or lipid nanoparticle, and delivered by known methods to a cell (e.g., a prokaryotic, eukaryotic, plant, mammalian, etc.). Such methods include, but not limited to, transfection (e.g., lipid-mediated, cationic polymers, calcium phosphate, dendrimers); electroporation or other methods of membrane disruption (e.g., nucleofection), viral delivery (e.g., lentivirus, retrovirus, adenovirus, AAV), microinjection, microprojectile bombardment (“gene gun”), fugene, direct sonic loading, cell squeezing, optical transfection, protoplast fusion, impalefection, magnetofection, exosome-mediated transfer, lipid nanoparticle-mediated transfer, and any combination thereof.

In some embodiments, the method comprises delivering one or more nucleic acids (e.g., nucleic acids encoding the CRISPR nuclease polypeptide and/or the gRNAs, and/or a pre-formed ribonucleoprotein to a cell. Exemplary intracellular delivery methods, include, but are not limited to: viruses or virus-like agents; chemical-based transfection methods, such as those using calcium phosphate, dendrimers, liposomes, or cationic polymers (e.g., DEAE-dextran or polyethylenimine); non-chemical methods, such as microinjection, electroporation, cell squeezing, sonoporation, optical transfection, impalefection, protoplast fusion, bacterial conjugation, delivery of plasmids or transposons; particle-based methods, such as using a gene gun, magnetofection or magnet assisted transfection, particle bombardment; and hybrid methods, such as nucleofection. In some embodiments, the present application further provides cells produced by such methods, and organisms (such as animals, plants, or fungi) comprising or produced from such cells. In some embodiments, a composition of the present invention is further delivered with an agent (e.g., compound, molecule, or biomolecule) that affects DNA repair or DNA repair machinery. In some embodiments, a composition of the present invention is further delivered with an agent (e.g., compound, molecule, or biomolecule) that affects the cell cycle.

In some embodiments, a first composition comprising a CRISPR nuclease polypeptide is delivered to a cell. In some embodiments, a second composition comprising a gRNA is delivered to the cell. In some embodiments, the first composition is contacted with a cell before the second composition is contacted with the cell. In some embodiments, the first composition is contacted with a cell at the same time as the second composition is contacted with the cell. In some embodiments, the first composition is contacted with a cell after the second composition is contacted with the cell. In some embodiments, the first composition is delivered by a first delivery method and the second composition is delivered by a second delivery method. In some embodiments, the first delivery method is the same as the second delivery method. For example, in some embodiments, the first composition and the second composition are delivered via viral delivery. In some embodiments, the first delivery method is different than the second delivery method. For example, in some embodiments, the first composition is delivered by viral delivery and the second composition is delivered by lipid nanoparticle-mediated transfer and the second composition is delivered by viral delivery or the first composition is delivered by lipid nanoparticle-mediated transfer and the second composition is delivered by viral delivery.

In some examples, any of the gene editing systems provided herein comprises one or more lipid excipients, which associate with the components of the gene editing system, to facilitate delivery of the gene editing system to host cells. In some instances, the one or more lipid excipients may form lipid nanoparticles (LNPs), which associate with or encapsulating components of the gene editing system (e.g., a mRNA molecule encoding the CRISPR nuclease polypeptide and the gRNA). Such LNP-containing gene editing systems can be brought in contact with host cells (e.g., administering to a subject in need of gene editing) and the LNP can facilitate delivery of the gene editing system into the host cells.

In other examples, the gene editing system provided herein may be delivered to host cells via a viral vector-mediated approach. Such a gene editing system may comprise a viral vector that carries a transgene encoding the CRISPR nuclease polypeptide and optionally a transgene encoding the gRNA. In some instances, the viral vector may be an AAV vector. The viral vector may facilitate delivery of the transgene(s) into host cells where the transgene(s) produces the CRISPR nuclease polypeptide and optionally the gRNA to effect genetic editing of a target gene.

### IV. Therapeutic Applications

Any of the gene editing systems or modified cells generated using such a gene editing system as disclosed herein may be used for treating a disease that may be benefit from the gene edit introduced by the gene editing system or carried by the modified cells. For example, the disease may be a genetic disease and the gene edit fixes the gene mutation associated with the genetic disease. Alternatively, the disease may be associated with abnormal expression of a gene and the gene edit rescues such abnormal expression.

In some embodiments, provided herein is a method for treating a disease comprising administering to a subject (e.g., a human patient) in need of the treatment any of the gene editing system disclosed herein. The gene editing system may be delivered to a specific tissue or specific type of cells where the gene edit is needed. The gene editing system may comprise LNPs encompassing one or more of the components, one or more vectors (e.g., viral vectors) encoding one or more of the components, or a combination thereof. Components of the gene editing system may be formulated to form a pharmaceutical composition, which may further comprise one or more pharmaceutically acceptable carriers.

In some embodiments, modified cells produced using any of the gene editing systems disclosed herein may be administered to a subject (e.g., a human patient) in need of the treatment. The modified cells may comprise a substitution, insertion, and/or deletion described herein. In some examples, the modified cells may include a cell line modified by the CRISPR nuclease polypeptide and the gRNA as disclosed herein. In some instances, the modified cells may be a heterogenous population comprising cells with different types of gene edits. Alternatively, the modified cells may comprise a substantially homogenous cell population (e.g., at least 80% of the cells in the whole population) comprising one particular gene edit. In some examples, the cells can be suspended in a suitable media.

In some embodiments, provided herein is a composition comprising the gene editing system or components thereof or the modified cells. Such a composition can be a pharmaceutical composition. A pharmaceutical composition that is useful may be prepared, packaged, or sold in a formulation suitable for oral, rectal, vaginal, parenteral, topical, pulmonary, intranasal, intra-lesional, buccal, ophthalmic, intravenous, intra-organ or another route of administration. A pharmaceutical composition of the disclosure may be prepared, packaged, or sold in bulk, as a single unit dose, or as a plurality of single unit doses. As used herein, a “unit dose” is discrete amount of the pharmaceutical composition comprising a predetermined number of cells. The number of cells is generally equal to the dosage of the cells which would be administered to a subject or a convenient fraction of such a dosage such as, for example, one-half or one-third of such a dosage.

A formulation of a pharmaceutical composition suitable for parenteral administration may comprise the active agent (e.g., the gene editing system or components thereof or the modified cells) combined with a pharmaceutically acceptable carrier, such as sterile water or sterile isotonic saline. Such a formulation may be prepared, packaged, or sold in a form suitable for bolus administration or for continuous administration. Some injectable formulations may be prepared, packaged, or sold in unit dosage form, such as in ampules or in multi-dose containers containing a preservative. Some formulations for parenteral administration include, but are not limited to, suspensions, solutions, emulsions in oily or aqueous vehicles, pastes, and implantable sustained-release or biodegradable formulations. Some formulations may further comprise one or more additional ingredients including, but not limited to, suspending, stabilizing, or dispersing agents.

The pharmaceutical composition may be in the form of a sterile injectable aqueous or oily suspension or solution. This suspension or solution may be formulated according to the known art, and may comprise, in addition to the cells, additional ingredients such as the dispersing agents, wetting agents, or suspending agents described herein. Such sterile injectable formulation may be prepared using a non-toxic parenterally-acceptable diluent or solvent, such as water or saline. Other acceptable diluents and solvents include, but are not limited to, Ringer's solution, isotonic sodium chloride solution, and fixed oils such as synthetic mono- or di-glycerides. Other parentally-administrable formulations which that are useful include those which may comprise the cells in a packaged form, in a liposomal preparation, or as a component of a biodegradable polymer system. Some compositions for sustained release or implantation may comprise pharmaceutically acceptable polymeric or hydrophobic materials such as an emulsion, an ion exchange resin, a sparingly soluble polymer, or a sparingly soluble salt.

### V. Kits and Uses Thereof

The present disclosure also provides kits or systems that can be used, for example, to carry out a method described herein. In some embodiments, the kits or systems include a CRISPR nuclease polypeptide and optionally a gRNA. In some embodiments, the kits or systems include a polynucleotide that encodes the CRISPR nuclease polypeptide and optionally the gRNA. The gRNA of the kits can be designed to target a sequence of interest.

The CRISPR nuclease polypeptide and the gRNA can be packaged within the same vial or other vessel within a kit or system or can be packaged in separate vials or other vessels, the contents of which can be mixed prior to use. The kits or systems can additionally include, optionally, a buffer and/or instructions for use of the CRISPR nuclease polypeptide and the gRNA.

In some embodiments, the kit comprises a first composition comprising a CRISPR nuclease polypeptide as disclosed herein. In some embodiments, the kit comprises a second composition comprising a gRNA as also disclosed herein. In some embodiments, the first composition and the second composition are packaged within the same vial. In some embodiments, the first composition and the second composition are packaged within different vials.

In some embodiments, the kit may be useful for research purposes. For example, in some embodiments, the kit may be useful to study gene function.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### Example 1: Editing of Human Target Genes in HEK293T Cells by Nuclease A-Derived CRISPR Nuclease Polypeptides

This Example describes the genomic editing of the AAVS1, EMX1, and VEGFA genes by Nuclease A (SEQ ID NO: 1) or variants thereof introduced into cells by lipid-based transient transfection into the HEK293T cell line.

Nuclease A was tagged with N-terminal SV40 nuclear localization signal (NLS) and a C-terminal nucleoplasmin nuclear localization signal (NLS), and its coding sequence was converted to a human codon-optimized DNA sequence, synthesized, and cloned into a pcDNA3.1 vector (Invitrogen), containing a CMV promoter for expression. The reference and NLS-tagged sequences used are in Table 1. Plasmids were purified using a midiprep kit.

RNA guides were designed and cloned and into a pUC19 plasmid following the U6 PolIII promoter and terminated with a 6× polyT sequence. RNA guides were designed to be specific to target sequences within the coding exons of AAVS1, EMX1, and VEGFA with 5′-NGG-3′ PAM sequences (the PAM sequence is on the 3′ end of the target sequence). See all RNA guide sequences in Table 2. The U6 PolIII promoter uses a +1 G at the start of the transcript (i.e., the 5′ end of the RNA) for more efficient transcription that is excluded from the sequences described in Table 2. Plasmids were purified using a midiprep kit.

TABLE 1
Amino Acid Sequences of Nuclease A and Exemplary Variants Thereof

| Component | Amino Acid Sequence | SEQ ID |
|---|---|---|
| Nuclease A | MRNCKKDLYVEVTPMQKELVLGIDYGGKYTGLAVVDRRNNQVL | 1 |
| | YARTVKMRDDVADILEGRREQRGIRRTGQTKKKRLRELKTYLKN | |
| | IGGKYDESIGTFKNEPFMTVYKLCHKRGYDYVEMPKEKTREEIEA | |
| | MSADEKKQWEKEKKEWEETERNSRHRDEVTKDVRKIMSERDAT | |
| | EQQISHVIKIFNKQYRPKRFNNRILTKCKVEDCGVNTPLRKNVRDL | |
| | LIKDIVRFLPLTQSEKAELEAHVLDKEGREKVKTFFRRHRINEFLR | |
| | KQIYDIAFNQKLSGRTVFCKKHILEHEQHTKEERKVFRLAPSLKTK | |
| | INNVLAVVKDEILPQFGINKVIMESNNFDIAARTKGKKRLAKDEY | |
| | GKGHKEAKESLIEVLLRETKNQCVYCGKVITVTTANTDHIFPRKA | |
| | GGINVFANLVACCRECNEKKKGRTPLESGVLPQPEITAHLENDLK | |
| | KKILEDARNMNELNLNSIKYMSYASIGWRHMRDRLRGLIGNEELP | |
| | IERQSGILTAYFRRWWGFRKERGNDIHHALDAVILASRKDYTDDG | |
| | VDMTLKPKSSNGREFDPEKHLPDTISFKRDKSNKGTPLYDKNPLSI | |
| | KNSLITRRWMVTEIEHGKEDTIISDEYRKKLKEAFKHWGTSKGKC | |
| | LTDEEARQTGFYQRKNGNFMSLRCVEKGLGWGQMVKINNNAFK | |
| | TNVHNIGVAVYLDEKGKKKAYEIKNPRLLKHFVEKPPEIKGKKLY | |
| | TLRRGDYVTHESEDSVYRIKKLGTSPVLEAVVKGSIGRIRNSASAT | |
| | KLTKVSKPQKVF | |
| SV40 NLS | KRTADGSEFESPKKKRKV | 3 |
| Nucleoplasmin | KRPAATKKAGQAKKKK | 4 |
| NLS | | |
| XTEN linker | SGGSSGGSSGSETPGTSESATPESSGGSSGGSS | 95 |
| NLS-tagged | KRTADGSEFESPKKKRKVRNCKKDLYVEVTPMQKELVLGIDYGGK | 5 |
| Nuclease A | YTGLAVVDRRNNQVLYARTVKMRDDVADILEGRREQRGIRRTG | |
| | QTKKKRLRELKTYLKNIGGKYDESIGTFKNEPFMTVYKLCHKRG | |
| | YDYVEMPKEKTREEIEAMSADEKKQWEKEKKEWEETERNSRHR | |
| | DEVTKDVRKIMSERDATEQQISHVIKIFNKQYRPKRFNNRILTKCK | |
| | VEDCGVNTPLRKNVRDLLIKDIVRFLPLTQSEKAELEAHVLDKEG | |
| | REKVKTFFRRHRINEFLRKQIYDIAFNQKLSGRTVFCKKHILEHEQ | |
| | HTKEERKVFRLAPSLKTKINNVLAVVKDEILPQFGINKVIMESNNF | |
| | DIAARTKGKKRLAKDEYGKGHKEAKESLIEVLLRETKNQCVYCG | |
| | KVITVTTANTDHIFPRKAGGINVFANLVACCRECNEKKKGRTPLE | |
| | SGVLPQPEITAHLENDLKKKILEDARNMNELNLNSIKYMSYASIG | |
| | WRHMRDRLRGLIGNEELPIERQSGILTAYFRRWWGFRKERGNDIH | |
| | HALDAVILASRKDYTDDGVDMTLKPKSSNGREFDPEKHLPDTISF | |
| | KRDKSNKGTPLYDKNPLSIKNSLITRRWMVTEIEHGKEDTIISDEY | |
| | RKKLKEAFKHWGTSKGKCLTDEEARQTGFYQRKNGNFMSLRCV | |
| | EKGLGWGQMVKINNNAFKTNVHNIGVAVYLDEKGKKKAYEIKN | |
| | PRLLKHFVEKPPEIKGKKLYTLRRGDYVTHESEDSVYRIKKLGTSP | |
| | VLEAVVKGSIGRIRNSASATKLTKVSKPQKVFSGGSSGGSSGSETP | |
| | GTSESATPESSGGSSGGSSKRPAATKKAGQAKKKK | |
| I67R/D568R/ | MRNCKKDLYVEVTPMQKELVLGIDYGGKYTGLAVVDRRNNQVL | 6 |
| E706R variant | YARTVKMRDDVADILEGRREQRGRRRTGQTKKKRLRELKTYLK | |
| | NIGGKYDESIGTFKNEPFMTVYKLCHKRGYDYVEMPKEKTREEIE | |
| | AMSADEKKQWEKEKKEWEETERNSRHRDEVTKDVRKIMSERDA | |
| | TEQQISHVIKIFNKQYRPKRFNNRILTKCKVEDCGVNTPLRKNVRD | |
| | LLIKDIVRFLPLTQSEKAELEAHVLDKEGREKVKTFFRRHRINEFL | |
| | RKQIYDIAFNQKLSGRTVFCKKHILEHEQHTKEERKVFRLAPSLKT | |
| | KINNVLAVVKDEILPQFGINKVIMESNNFDIAARTKGKKRLAKDE | |
| | YGKGHKEAKESLIEVLLRETKNQCVYCGKVITVTTANTDHIFPRK | |
| | AGGINVFANLVACCRECNEKKKGRTPLESGVLPQPEITAHLENDL | |
| | KKKILEDARNMNELNLNSIKYMSYASIGWRHMRDRLRGLIGNEE | |
| | LPIERQSGILTAYFRRWWGFRKERGNDIHHALDAVILASRKDYTD | |
| | DGVDMTLKPKSSNGREFDPEKHLPDTISFKRRKSNKGTPLYDKNP | |
| | LSIKNSLITRRWMVTEIEHGKEDTIISDEYRKKLKEAFKHWGTSKG | |
| | KCLTDEEARQTGFYQRKNGNFMSLRCVEKGLGWGQMVKINNNA | |
| | FKTNVHNIGVAVYLDEKGKKKAYEIKNPRLLKHFVRKPPEIKGKK | |
| | LYTLRRGDYVTHESEDSVYRIKKLGTSPVLEAVVKGSIGRIRNSAS | |
| | ATKLTKVSKPQKVF | |
| I67R/D56R/ | MRNCKKDLYVEVTPMQKELVLGIDYGGKYTGLAVVDRRNNQVL | 7 |
| D568R variant | YARTVKMRDDVARILEGRREQRGRRRTGQTKKKRLRELKTYLK | |
| | NIGGKYDESIGTFKNEPFMTVYKLCHKRGYDYVEMPKEKTREEIE | |
| | AMSADEKKQWEKEKKEWEETERNSRHRDEVTKDVRKIMSERDA | |
| | TEQQISHVIKIFNKQYRPKRFNNRILTKCKVEDCGVNTPLRKNVRD | |
| | LLIKDIVRFLPLTQSEKAELEAHVLDKEGREKVKTFFRRHRINEFL | |
| | RKQIYDIAFNQKLSGRTVFCKKHILEHEQHTKEERKVFRLAPSLKT | |
| | KINNVLAVVKDEILPQFGINKVIMESNNFDIAARTKGKKRLAKDE | |
| | YGKGHKEAKESLIEVLLRETKNQCVYCGKVITVTTANTDHIFPRK | |
| | AGGINVFANLVACCRECNEKKKGRTPLESGVLPQPEITAHLENDL | |
| | KKKILEDARNMNELNLNSIKYMSYASIGWRHMRDRLRGLIGNEE | |
| | LPIERQSGILTAYFRRWWGFRKERGNDIHHALDAVILASRKDYTD | |
| | DGVDMTLKPKSSNGREFDPEKHLPDTISFKRRKSNKGTPLYDKNP | |
| | LSIKNSLITRRWMVTEIEHGKEDTIISDEYRKKLKEAFKHWGTSKG | |
| | KCLTDEEARQTGFYQRKNGNFMSLRCVEKGLGWGQMVKINNNA | |
| | FKTNVHNIGVAVYLDEKGKKKAYEIKNPRLLKHFVEKPPEIKGKK | |
| | LYTLRRGDYVTHESEDSVYRIKKLGTSPVLEAVVKGSIGRIRNSAS | |
| | ATKLTKVSKPQKVF | |
| I67R/T605R/ | MRNCKKDLYVEVTPMQKELVLGIDYGGKYTGLAVVDRRNNQVL | 8 |
| D568R variant | YARTVKMRDDVADILEGRREQRGRRRTGQTKKKRLRELKTYLK | |
| | NIGGKYDESIGTFKNEPFMTVYKLCHKRGYDYVEMPKEKTREEIE | |
| | AMSADEKKQWEKEKKEWEETERNSRHRDEVTKDVRKIMSERDA | |
| | TEQQISHVIKIFNKQYRPKRFNNRILTKCKVEDCGVNTPLRKNVRD | |
| | LLIKDIVRFLPLTQSEKAELEAHVLDKEGREKVKTFFRRHRINEFL | |
| | RKQIYDIAFNQKLSGRTVFCKKHILEHEQHTKEERKVFRLAPSLKT | |
| | KINNVLAVVKDEILPQFGINKVIMESNNFDIAARTKGKKRLAKDE | |
| | YGKGHKEAKESLIEVLLRETKNQCVYCGKVITVTTANTDHIFPRK | |
| | AGGINVFANLVACCRECNEKKKGRTPLESGVLPQPEITAHLENDL | |
| | KKKILEDARNMNELNLNSIKYMSYASIGWRHMRDRLRGLIGNEE | |
| | LPIERQSGILTAYFRRWWGFRKERGNDIHHALDAVILASRKDYTD | |
| | DGVDMTLKPKSSNGREFDPEKHLPDTISFKRRKSNKGTPLYDKNP | |
| | LSIKNSLITRRWMVTEIEHGKEDRIISDEYRKKLKEAFKHWGTSKG | |
| | KCLTDEEARQTGFYQRKNGNFMSLRCVEKGLGWGQMVKINNNA | |
| | FKTNVHNIGVAVYLDEKGKKKAYEIKNPRLLKHFVEKPPEIKGKK | |
| | LYTLRRGDYVTHESEDSVYRIKKLGTSPVLEAVVKGSIGRIRNSAS | |
| | ATKLTKVSKPQKVF | |
| I67R/D56R/ | MRNCKKDLYVEVTPMQKELVLGIDYGGKYTGLAVVDRRNNQVL | 9 |
| E706R variant | YARTVKMRDDVARILEGRREQRGRRRTGQTKKKRLRELKTYLK | |
| | NIGGKYDESIGTFKNEPFMTVYKLCHKRGYDYVEMPKEKTREEIE | |
| | AMSADEKKQWEKEKKEWEETERNSRHRDEVTKDVRKIMSERDA | |
| | TEQQISHVIKIFNKQYRPKRFNNRILTKCKVEDCGVNTPLRKNVRD | |
| | LLIKDIVRFLPLTQSEKAELEAHVLDKEGREKVKTFFRRHRINEFL | |
| | RKQIYDIAFNQKLSGRTVFCKKHILEHEQHTKEERKVFRLAPSLKT | |
| | KINNVLAVVKDEILPQFGINKVIMESNNFDIAARTKGKKRLAKDE | |
| | YGKGHKEAKESLIEVLLRETKNQCVYCGKVITVTTANTDHIFPRK | |
| | AGGINVFANLVACCRECNEKKKGRTPLESGVLPQPEITAHLENDL | |
| | KKKILEDARNMNELNLNSIKYMSYASIGWRHMRDRLRGLIGNEE | |
| | LPIERQSGILTAYFRRWWGFRKERGNDIHHALDAVILASRKDYTD | |
| | DGVDMTLKPKSSNGREFDPEKHLPDTISFKRDKSNKGTPLYDKNP | |
| | LSIKNSLITRRWMVTEIEHGKEDTIISDEYRKKLKEAFKHWGTSKG | |
| | KCLTDEEARQTGFYQRKNGNFMSLRCVEKGLGWGQMVKINNNA | |
| | FKTNVHNIGVAVYLDEKGKKKAYEIKNPRLLKHFVRKPPEIKGKK | |
| | LYTLRRGDYVTHESEDSVYRIKKLGTSPVLEAVVKGSIGRIRNSAS | |
| | ATKLTKVSKPQKVF | |
| I67R/T605R/ | MRNCKKDLYVEVTPMQKELVLGIDYGGKYTGLAVVDRRNNQVL | 10 |
| E706R variant | YARTVKMRDDVADILEGRREQRGRRRTGQTKKKRLRELKTYLK | |
| | NIGGKYDESIGTFKNEPFMTVYKLCHKRGYDYVEMPKEKTREEIE | |
| | AMSADEKKQWEKEKKEWEETERNSRHRDEVTKDVRKIMSERDA | |
| | TEQQISHVIKIFNKQYRPKRFNNRILTKCKVEDCGVNTPLRKNVRD | |
| | LLIKDIVRFLPLTQSEKAELEAHVLDKEGREKVKTFFRRHRINEFL | |
| | RKQIYDIAFNQKLSGRTVFCKKHILEHEQHTKEERKVFRLAPSLKT | |
| | KINNVLAVVKDEILPQFGINKVIMESNNFDIAARTKGKKRLAKDE | |
| | YGKGHKEAKESLIEVLLRETKNQCVYCGKVITVTTANTDHIFPRK | |
| | AGGINVFANLVACCRECNEKKKGRTPLESGVLPQPEITAHLENDL | |
| | KKKILEDARNMNELNLNSIKYMSYASIGWRHMRDRLRGLIGNEE | |
| | LPIERQSGILTAYFRRWWGFRKERGNDIHHALDAVILASRKDYTD | |
| | DGVDMTLKPKSSNGREFDPEKHLPDTISFKRDKSNKGTPLYDKNP | |
| | LSIKNSLITRRWMVTEIEHGKEDRIISDEYRKKLKEAFKHWGTSKG | |
| | KCLTDEEARQTGFYQRKNGNFMSLRCVEKGLGWGQMVKINNNA | |
| | FKTNVHNIGVAVYLDEKGKKKAYEIKNPRLLKHFVRKPPEIKGKK | |
| | LYTLRRGDYVTHESEDSVYRIKKLGTSPVLEAVVKGSIGRIRNSAS | |
| | ATKLTKVSKPQKVF | |
| I67R/D568R/ | MRNCKKDLYVEVTPMQKELVLGIDYGGKYTGLAVVDRRNNQVL | 11 |
| E694R variant | YARTVKMRDDVADILEGRREQRGRRRTGQTKKKRLRELKTYLK | |
| | NIGGKYDESIGTFKNEPFMTVYKLCHKRGYDYVEMPKEKTREEIE | |
| | AMSADEKKQWEKEKKEWEETERNSRHRDEVTKDVRKIMSERDA | |
| | TEQQISHVIKIFNKQYRPKRFNNRILTKCKVEDCGVNTPLRKNVRD | |
| | LLIKDIVRFLPLTQSEKAELEAHVLDKEGREKVKTFFRRHRINEFL | |
| | RKQIYDIAFNQKLSGRTVFCKKHILEHEQHTKEERKVFRLAPSLKT | |
| | KINNVLAVVKDEILPQFGINKVIMESNNFDIAARTKGKKRLAKDE | |
| | YGKGHKEAKESLIEVLLRETKNQCVYCGKVITVTTANTDHIFPRK | |
| | AGGINVFANLVACCRECNEKKKGRTPLESGVLPQPEITAHLENDL | |
| | KKKILEDARNMNELNLNSIKYMSYASIGWRHMRDRLRGLIGNEE | |
| | LPIERQSGILTAYFRRWWGFRKERGNDIHHALDAVILASRKDYTD | |
| | DGVDMTLKPKSSNGREFDPEKHLPDTISFKRRKSNKGTPLYDKNP | |
| | LSIKNSLITRRWMVTEIEHGKEDTIISDEYRKKLKEAFKHWGTSKG | |
| | KCLTDEEARQTGFYQRKNGNFMSLRCVEKGLGWGQMVKINNNA | |
| | FKTNVHNIGVAVYLDEKGKKKAYRIKNPRLLKHFVEKPPEIKGKK | |
| | LYTLRRGDYVTHESEDSVYRIKKLGTSPVLEAVVKGSIGRIRNSAS | |
| | ATKLTKVSKPQKVF | |
| I67R/D56R/ | MRNCKKDLYVEVTPMQKELVLGIDYGGKYTGLAVVDRRNNQVL | 12 |
| E694R/E706R | YARTVKMRDDVARILEGRREQRGRRRTGQTKKKRLRELKTYLK | |
| variant | NIGGKYDESIGTFKNEPFMTVYKLCHKRGYDYVEMPKEKTREEIE | |
| | AMSADEKKQWEKEKKEWEETERNSRHRDEVTKDVRKIMSERDA | |
| | TEQQISHVIKIFNKQYRPKRFNNRILTKCKVEDCGVNTPLRKNVRD | |
| | LLIKDIVRFLPLTQSEKAELEAHVLDKEGREKVKTFFRRHRINEFL | |
| | RKQIYDIAFNQKLSGRTVFCKKHILEHEQHTKEERKVFRLAPSLKT | |
| | KINNVLAVVKDEILPQFGINKVIMESNNFDIAARTKGKKRLAKDE | |
| | YGKGHKEAKESLIEVLLRETKNQCVYCGKVITVTTANTDHIFPRK | |
| | AGGINVFANLVACCRECNEKKKGRTPLESGVLPQPEITAHLENDL | |
| | KKKILEDARNMNELNLNSIKYMSYASIGWRHMRDRLRGLIGNEE | |
| | LPIERQSGILTAYFRRWWGFRKERGNDIHHALDAVILASRKDYTD | |
| | DGVDMTLKPKSSNGREFDPEKHLPDTISFKRDKSNKGTPLYDKNP | |
| | LSIKNSLITRRWMVTEIEHGKEDTIISDEYRKKLKEAFKHWGTSKG | |
| | KCLTDEEARQTGFYQRKNGNFMSLRCVEKGLGWGQMVKINNNA | |
| | FKTNVHNIGVAVYLDEKGKKKAYRIKNPRLLKHFVRKPPEIKGKK | |
| | LYTLRRGDYVTHESEDSVYRIKKLGTSPVLEAVVKGSIGRIRNSAS | |
| | ATKLTKVSKPQKVF | |
| I67R/D56R/ | MRNCKKDLYVEVTPMQKELVLGIDYGGKYTGLAVVDRRNNQVL | 13 |
| T605R/E706R | YARTVKMRDDVARILEGRREQRGRRRTGQTKKKRLRELKTYLK | |
| variant | NIGGKYDESIGTFKNEPFMTVYKLCHKRGYDYVEMPKEKTREEIE | |
| | AMSADEKKQWEKEKKEWEETERNSRHRDEVTKDVRKIMSERDA | |
| | TEQQISHVIKIFNKQYRPKRFNNRILTKCKVEDCGVNTPLRKNVRD | |
| | LLIKDIVRFLPLTQSEKAELEAHVLDKEGREKVKTFFRRHRINEFL | |
| | RKQIYDIAFNQKLSGRTVFCKKHILEHEQHTKEERKVFRLAPSLKT | |
| | KINNVLAVVKDEILPQFGINKVIMESNNFDIAARTKGKKRLAKDE | |
| | YGKGHKEAKESLIEVLLRETKNQCVYCGKVITVTTANTDHIFPRK | |
| | AGGINVFANLVACCRECNEKKKGRTPLESGVLPQPEITAHLENDL | |
| | KKKILEDARNMNELNLNSIKYMSYASIGWRHMRDRLRGLIGNEE | |
| | LPIERQSGILTAYFRRWWGFRKERGNDIHHALDAVILASRKDYTD | |
| | DGVDMTLKPKSSNGREFDPEKHLPDTISFKRDKSNKGTPLYDKNP | |
| | LSIKNSLITRRWMVTEIEHGKEDRIISDEYRKKLKEAFKHWGTSKG | |
| | KCLTDEEARQTGFYQRKNGNFMSLRCVEKGLGWGQMVKINNNA | |
| | FKTNVHNIGVAVYLDEKGKKKAYEIKNPRLLKHFVRKPPEIKGKK | |
| | LYTLRRGDYVTHESEDSVYRIKKLGTSPVLEAVVKGSIGRIRNSAS | |
| | ATKLTKVSKPQKVF | |

TABLE 2
Target and RNA Guide Sequences for Nuclease A-Derived CRISPR Nuclease
Polypeptides

| | | Target | |
|---|---|---|---|
| Guide | PAM | Sequence | RNA Guide Sequence* |
| AAVS1- | AGG | TTGTGAGAAT | UUGUGAGAAUGGUGCGUCCUguuacaguuaaggcucuuuggaaa |
| T3 | | GGTGCGTCCT | caaagaagccuuaauuguaaaacgccuauaugguaaagugauguacguuugggu |
| | | (SEQ ID NO: 14) | auauaucgccagccugaaccucuacgccagaaauggcagcuuuaucauggguuag |
| | | | gacgauauuuaaaaaacuuucugcggcuugcuuacuuuaguaagcuuuguggcu |
| | | | gaggcagaauuccuu (SEQ ID NO: 15) |
| AAVS1- | TGG | CTTTGTAGCC | CUUUGUAGCCUCUCCCGCUCguuacaguuaaggcucuuuggaaac |
| T6 | | TCTCCCGCTC | aaagaagccuuaauuguaaaacgccuauaugguaaagugauguacguuugggua |
| | | (SEQ ID NO: 16) | uauaucgccagccugaaccucuacgccagaaauggcagcuuuaucauggguuagg |
| | | | acgauauuuaaaaaacuuucugcggcuugcuuacuuuaguaagcuuuguggcug |
| | | | aggcagaauuccuu (SEQ ID NO: 17) |
| EMX1- | AGG | TGGATGGCGA | UGGAUGGCGACUUCAGGCACguuacaguuaaggcucuuuggaaa |
| T2 | | CTTCAGGCAC | caaagaagccuuaauuguaaaacgccuauaugguaaagugauguacguuugggu |
| | | (SEQ ID NO: 18) | auauaucgccagccugaaccucuacgccagaaauggcagcuuuaucauggguuag |
| | | | gacgauauuuaaaaaacuuucugcggcuugcuuacuuuaguaagcuuuguggcu |
| | | | gaggcagaauuccuu (SEQ ID NO: 19) |
| EMX1- | GGG | CTTTAGCAAG | CUUUAGCAAGGGACUAUUCAguuacaguuaaggcucuuuggaaa |
| T7 | | GGACTATTCA | caaagaagccuuaauuguaaaacgccuauaugguaaagugauguacguuugggu |
| | | (SEQ ID NO: 20) | auauaucgccagccugaaccucuacgccagaaauggcagcuuuaucauggguuag |
| | | | gacgauauuuaaaaaacuuucugcggcuugcuuacuuuaguaagcuuuguggcu |
| | | | gaggcagaauuccuu (SEQ ID NO: 21) |
| VEGFA- | TGG | TTGTTGACAT | UUGUUGACAUUGUCCACACCguuacaguuaaggcucuuuggaaac |
| T3 | | TGTCCACACC | aaagaagccuuaauuguaaaacgccuauaugguaaagugauguacguuugggua |
| | | (SEQ ID NO: 22) | uauaucgccagccugaaccucuacgccagaaauggcagcuuuaucauggguuagg |
| | | | acgauauuuaaaaaacuuucugeggcuugcuuacuuuaguaagcuuuguggcug |
| | | | aggcagaauuccuu (SEQ ID NO: 23) |
| VEGFA- | AGG | TTTGCCCATA | UUUGCCCAUACUGGGGACCAguuacaguuaaggcucuuuggaaac |
| T6 | | CTGGGGACCA | aaagaagccuuaauuguaaaacgccuauaugguaaagugauguacguuugggua |
| | | (SEQ ID NO: 24) | uauaucgccagccugaaccucuacgccagaaauggcagcuuuaucauggguuagg |
| | | | acgauauuuaaaaaacuuucugcggcuugcuuacuuuaguaagcuuuguggcug |
| | | | aggcagaauuccuu (SEQ ID NO: 25) |

*Spacer in upper case and scaffold (SEQ ID NO: 2) in lower case

Approximately 16 hours prior to transfection, 25,000 HEK293T cells in DMEM/10% FBS+Pen/Strep (D10 media) were plated into each well of a 96-well plate. On the day of transfection, the cells were 50-90% confluent. For each well to be transfected, a mixture of Lipofectamine 2000™ (ThermoFisher Scientific) and Opti-MEM™ (ThermoFisher Scientific) was prepared and incubated at room temperature for 5 minutes (Solution 1). After incubation, the Lipofectamine 2000™: Opti-MEM™ mixture was added to a separate mixture containing the CRISPR nuclease plasmid (NLS-tagged), RNA guide plasmid, and Opti-MEM™ (Solution 2). In the case of negative controls, the CRISPR nuclease plasmid was excluded. Solutions 1 and 2 were mixed by pipetting up and down, then incubated at room temperature for 25 minutes. Following incubation, the Solution 1 and 2 mixture was added dropwise to each well of a 96-well plate containing the cells. Approximately 72 hours post transfection, cells were trypsinized by adding TrypLE™ (ThermoFisher Scientific) to the center of each well and incubating at 37° C. for approximately 5 minutes. D10 media was then added to each well and mixed to resuspend cells. The resuspended cells were centrifuged for 10 minutes to obtain a pellet, and the supernatant was discarded. The cell pellet was then resuspended in QuickExtract™ buffer (Lucigen®), and cells were incubated at 65° C. for 15 minutes, 68° C. for 15 minutes, and 98° C. for 10 minutes.

Next Generation Sequencing (NGS) samples were prepared by two rounds of PCR. Three technical replicates were analyzed per target for the reference and each variant. The first round (PCR1) was used to amplify specific genomic regions depending on the target. Round 2 PCR (PCR2) was performed to add Illumina adapters and indices. Reactions were then pooled and purified by column purification. Sequencing runs were performed using a kit such as a 150 Cycle NextSeq 500/550 Mid or High Output v2.5 Kit.

For NGS analysis, the indel mapping function used a sample's fastq file, the amplicon reference sequence, and the forward primer sequence. For each read, a kmer-scanning algorithm was used to calculate the edit operations (match, mismatch, insertion, deletion) between the read and the reference sequence. In order to remove small amounts of primer dimer present in some samples, the first 30 nt of each read was required to match the reference and reads where over half of the mapping nucleotides are mismatches were filtered out as well. Up to 50,000 reads passing those filters were used for analysis, and reads were counted as an indel read if they contained an insertion or deletion. The % indels was calculated as the number of indel-containing reads divided by the number of reads analyzed (reads passing filters up to 50,000). The QC standard for the minimum number of reads passing filters was 10,000.

For each target, indel ratios, referring to the percentage of NGS reads comprising indels, were calculated for each sample and its cognate no protein control. Targets comprising a higher percentage of indels when the CRISPR nuclease was included in the transfection were indicative of DNA editing outcomes in the cell.

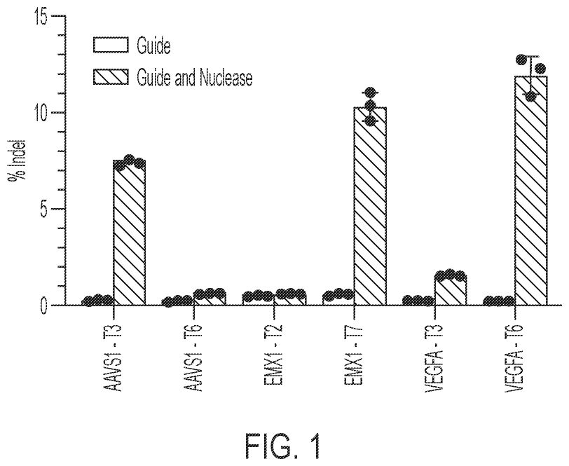

As shown in FIG. 1, four of the six targets tested demonstrated a greater level of indels observed when the Nuclease A (SEQ ID NO: 1) plasmid was present.

### Example 2: Effectiveness of CRISPR Nuclease A Variants for Targeting of Mammalian Genes

This Example describes indel assessment on mammalian targets using variants of CRISPR Nuclease A (SEQ ID NO: 1) transfected into HEK293T cells.

Arginine scanning mutagenesis was performed to individually substitute each non-arginine residue of Nuclease A (SEQ ID NO: 1) to arginine. SEQ ID NO: 1 is referred to herein as the Nuclease A reference sequence. This resulted in 710 single arginine substitution variants. Nucleic acids encoding Nuclease A and each CRISPR nuclease variant were then individually cloned into a pcda3.1 backbone (Invitrogen), and the plasmids were maxi-prepped and diluted. The plasmids comprised a CMV promoter, a first NLS (KRTADGSEFESPKKKRKV; SEQ ID NO: 3) upstream of the coding sequence, an XTEN linker (SGGSSGGSSGSETPGTSESATPESSGGSSGGSS; SEQ ID NO: 95) followed by a second NLS (KRPAATKKAGQAKKKK; SEQ ID NO: 4) downstream of the coding sequence. See also Example 1 above.

The RNA guide and target sequences are shown in Table 3. RNA guides were cloned into a pUC19 backbone (New England Biolabs®) following the U6 PolIII promoter and terminated with a 6× polyT sequence. The U6 PolIII promoter uses a +1 G at the start of the transcript (i.e., the 5′ end of the RNA) for more efficient transcription that is excluded from the sequences described in Table 3. The plasmids were then maxi-prepped and diluted.

TABLE 3
Mammalian Targets and Corresponding crRNAs*

| Target | | PAM | | |
|---|---|---|---|---|
| Identifier | Guide | Sequence | Target Sequence | RNA Guide Sequence |
| AAVS1 | AAVS1-T3 | AGG | SEQ ID NO: 14 | SEQ ID NO: 15 |
| VEGFA | VEGFA-T6 | AGG | SEQ ID NO: 24 | SEQ ID NO: 25 |

*See Table 2 above for the corresponding sequences.

HEK293T cells were transfected with plasmids expressing the CRISPR nuclease polypeptides and the gRNAs provided herein following the methods provided in Example 1 above. Gene editing efficiencies were determined by Next Generation Sequencing following the methods also provided in Example 1.

For each target, indel ratios, referring to the percentage of NGS reads comprising indels, were calculated for the reference and for each variant. The indel ratios used for fold change calculations were the average of two technical replicates. To then calculate fold change in indel ratios at a particular target, the indel ratio for each variant was divided by the indel ratio for the reference. Table 4 shows fold change in indel ratios for each target and as an average of both targets. Numbering is relative to the reference nuclease of SEQ ID NO: 1 (i.e., without an NLS). For example, at the AAVS1 target, the indel ratio for the I67R variant was 7.07 times that of the reference indel ratio, and at the VEGFA target, the indel ratio for the 167R variant was 4.43 times that of the reference indel ratio. The average fold change in indel ratio for the 167R variant at both targets was 5.75 times that of the indel ratio for the reference at both targets.

As shown in Table 4, 21 variants with single arginine substitutions (left column) were characterized as yielding at least a 1.5X increase in indel ratio relative to the reference indel ratio, when averaged across both targets (right column).

TABLE 4
Fold Change in Indel Ratios*

| | | | AAVS1 and |
|---|---|---|---|
| | | | VEGFA Average |
| Substitution | AAVS1 | VEGFA | Fold Change |
| I67R | 7.07 | 4.43 | 5.75 |
| E63R | 7.22 | 3.67 | 5.45 |
| D56R | 3.58 | 3.60 | 3.59 |
| T605R | 2.87 | 3.49 | 3.18 |
| D568R | 2.91 | 2.72 | 2.82 |
| E694R | 2.57 | 2.98 | 2.77 |
| G72R | 3.06 | 1.77 | 2.41 |
| E706R | 2.09 | 2.71 | 2.40 |
| E655R | 1.68 | 2.76 | 2.22 |
| E59R | 2.67 | 1.19 | 1.93 |
| G60R | 2.22 | 1.55 | 1.88 |
| E599R | 1.60 | 2.16 | 1.88 |
| S564R | 1.76 | 1.96 | 1.86 |
| E444R | 2.04 | 1.46 | 1.75 |
| Q72R | 1.84 | 1.49 | 1.67 |
| G661R | 1.15 | 1.99 | 1.57 |
| L588R | 1.73 | 1.40 | 1.56 |
| E264R | 1.64 | 1.48 | 1.56 |
| K656R | 1.62 | 1.47 | 1.55 |
| I598R | 1.23 | 1.85 | 1.54 |
| W593R | 1.60 | 1.47 | 1.53 |

*Variant indel ratio/Reference indel ratio

155 variants were analyzed as having indel ratios 1X-1.5X of the reference indel ratios, when averaged across both targets: E206R, K74R, S280R, A319R, 196R, A354R, D323R, F265R, E120R, H442R, V284R, E364R, C653R, A365R, N586R, E294R, E490R, N459R, E460R, E144R, S95R, N571R, S587R, P708R, K163R, G485R, V387R, I711R, W148R, K100R, G722R, A132R, Q328R, T201R, K322R, Q277R, N87R, K378R, N758R, I563R, G330R, D207R, K86R, G359R, K360R, K49R, T741R, I519R, E248R, E299R, K333R, Q41R, V12R, E152R, V210R, D53R, Q380R, M105R, K145R, S548R, S761R, K28R, V390R, K17R, D537R, K585R, Q772R, K351R, T255R, K287R, K146R, G66R, A441R, E491R, K302R, N340R, T98R, T83R, S752R, E149R, A55R, S650R, K355R, N489R, N457R, I753R, E94R, V774R, K572R, E371R, K566R, N462R, D561R, K366R, L369R, K546R, Y693R, S368R, K773R, Q16R, P771R, G385R, S180R, E11R, K614R, V10R, K422R, E129R, H243R, G209R, Q436R, K770R, K737R, K767R, K579R, K764R, G97R, K224R, P709R, L8R, K298R, K750R, Q177R, D533R, D356R, S170R, T395R, K449R, K252R, Y271R, E251R, W141R, K714R, T726R, K167R, I370R, K184R, K91R, E418R, D173R, Q295R, E131R, K350R, N332R, Y9R, A242R, K288R, E142R, N154R, F775R, H600R, L266R, E438R, V749R, and S134R. The remaining variants (534 variants) resulted in decreased indel ratios relative to the reference indel ratios (fold change in indel ratios of less than 1.0): G488R, E300R, K348R, K424R, P437R, K557R, S729R, K702R, G713R, E128R, V654R, S547R, T233R, A135R, T542R, T766R, K386R, T13R, Q64R, E453R, H293R, L543R, D536R, K75R, K363R, P560R, Q140R, K143R, K467R, P192R, M15R, K123R, L245R, K5R, T297R, K278R, K76R, S235R, H621R, E367R, K689R, D246R, Y534R, I756R, V768R, K268R, L434R, T391R, K139R, S465R, E236R, K6R, K82R, K665R, D7R, K138R, Y29R, V119R, E137R, N39R, E556R, N379R, C4R, K247R, T151R, A238R, S742R, T126R, D446R, V372R, Y471R, E357R, N549R, T392R, Q234R, I130R, L701R, P698R, G538R, D272R, I678R, E710R, N517R, M133R, K613R, D159R, E292R, K626R, E552R, E430R, T562R, G516R, K311R, M594R, D164R, A274R, T535R, P122R, T70R, F275R, N677R, K687R, N263R, A747R, L559R, S570R, S155R, G550R, K237R, D52R, K620R, G751R, K254R, T763R, T440R, P575R, N196R, K109R, E495R, K188R, K216R, F304R, S769R, V205R, Q269R, L291R, G249R, E633R, K628R, E239R, K691R, K644R, K712R, K715R, A760R, N667R, P493R, I226R, E176R, G754R, L684R, H260R, D554R, H727R, N276R, S625R, Q637R, N315R, E160R, A403R, N3R, V433R, K696R, K202R, K402R, D609R, L200R, F229R, V744R, L429R, E18R, T718R, I607R, T73R, H296R, L374R, L78R, D481R, V748R, Q189R, E147R, S583R, H181R, E728R, E150R, E171R, A174R, D220R, K690R, K532R, Y724R, E124R, S759R, Y717R, H703R, P14R, K707R, A681R, L43R, L716R, C629R, L19R, K204R, G740R, T106R, K125R, T175R, E421R, M121R, K113R, K602R, D136R, G361R, 1487R, K193R, E617R, G349R, L326R, G623R, K616R, E610R, I695R, E686R, Q497R, E746R, E597R, H558R, N445R, I168R, K313R, E241R, G688R, Q642R, D518R, V733R, K544R, D93R, H362R, L279R, N187R, S732R, K569R, L214R, D225R, V244R, P308R, N464R, G646R, P231R, L412R, I57R, F565R, W508R, Y504R, T162R, Y190R, E730R, D731R, G573R, H478R, G404R, N668R, Q178R, W622R, D540R, S431R, L221R, 1199R, T590R, L501R, M541R, G627R, D685R, G601R, I439R, H112R, N407R, V705R, D454R, A344R, N647R, W476R, T631R, N217R, D632R, E80R, N101R, V539R, L486R, E324R, I335R, N211R, N645R, L110R, L492R, V253R, S498R, F409R, G639R, L447R, F195R, V20R, P555R, K738R, V161R, I223R, T638R, T283R, S608R, Y92R, E514R, P428R, T624R, L58R, V218R, K448R, I179R, M458R, I88R, I183R, A692R, Y577R, I325R, L222R, A45R, E102R, A670R, G89R, F329R, T427R, T574R, E634R, D117R, G432R, L232R, V303R, C381R, W660R, D36R, G281R, I584R, A307R, D604R, G679R, G510R, P327R, N316R, L318R, I262R, V227R, F256R, Y84R, N580R, L443R, V320R, F257R, D723R, V54R, F553R, D342R, L353R, T212R, M649R, Y116R, G425R, F104R, H157R, Y25R, A503R, 1666R, 1314R, I589R, G405R, L230R, T312R, I500R, I290R, N40R, Y44R, P435R, P103R, L582R, Y468R, V165R, P213R, C419R, G90R, F99R, F704R, F505R, F648R, L306R, A618R, L463R, I388R, N197R, A522R, P545R, H676R, F285R, Y118R, N669R, Y734R, N697R, V680R, H520R, L373R, I273R, K513R, L240R, F671R, C203R, E376R, L461R, N394R, L719R, 1185R, M479R, V682R, A345R, E603R, T47R, F619R, A762R, L576R, D578R, A410R, 1451R, 1494R, L81R, I331R, V408R, C208R, S530R, Y383R, K423R, M469R, F511R, Y108R, V321R, I270R, V382R, S309R, L651R, T377R, N674R, F640R, L658R, G31R, V107R, A455R, K672R, W509R, I23R, P743R, V42R, T596R, L452R, M169R, H289R, M336R, L630R, T30R, V34R, V35R, G22R, G659R, L85R, C111R, G115R, V317R, V725R, Y683R, V595R, M50R, L615R, I736R, V526R, A529R, I606R, Q662R, A414R, C384R, I406R, V48R, L700R, Y611R, I466R, H397R, A635R, L523R, T347R, F399R, 1527R, G657R, C415R, A525R, V664R, L310R, V182R, D524R, F186R, E337R, V675R, L765R, L483R, L32R, K450R, S473R, G499R, P581R, L21R, H521R, D396R, C416R, 1398R, Y641R, T673R, L739R, G27R, V413R, M663R, L745R, P400R, C286R, T389R, N339R, A393R, 1474R, T502R, N411R, A33R, F341R, S338R, G26R, L528R, G475R, N420R, D24R, Y358R, V334R, A472R, 1343R, and S470R.

Based on this experiment, the following substitutions were selected for further engineering relative to Nuclease A of SEQ ID NO: 1: I67R, E63R, D56R, T605R, D568R, E694R, G71R, E706R, and E655R.

### Example 3: Effectiveness of CRISPR Nuclease A Combination Variants for Targeting of Mammalian Genes

This Example describes indel assessment on mammalian targets using Nuclease A variants comprising two or more substitutions identified as increasing indel activity in

### Example 2. 106 Combination Nuclease A Variants were Tested

Each Nuclease A variant and RNA guide (see Table 5) was cloned as described in Example 2. HEK293T cells were further transfected, followed by NGS analysis, as described in Example 2. For each target, indel ratios, referring to the percentage of NGS reads comprising indels, were calculated for Nuclease A (SEQ ID NO: 1) and for each variant of Nuclease A. The indel ratios shown in Table 6 were calculated as the average of two bioreplicates, each of which contained two technical replicates.

TABLE 5
Mammalian Targets and Corresponding crRNAs*

| Target | | PAM | | |
|---|---|---|---|---|
| Identifier | Guide | Sequence | Target Sequence | RNA Guide Sequence |
| AAVS1 | AAVS1-T3 | AGG | SEQ ID NO: 14 | SEQ ID NO: 15 |
| VEGFA | VEGFA-T6 | AGG | SEQ ID NO: 24 | SEQ ID NO: 25 |
| EMX1 | EMX1-T7 | GGG | SEQ ID NO: 20 | SEQ ID NO: 21 |

*See Table 2 for corresponding sequences

TABLE 6
Indel Ratios for Mammalian Targets

| | AAVS1 | EMX1 | VEGFA | |
|---|---|---|---|---|
| | average | average | average | |
| Substitutions | indel ratio | indel ratio | indel ratio | Average indel ratio targets |
| I67R, D568R, E706R | 0.370 | 0.337 | 0.325 | 0.344 |
| I67R, D56R, D568R | 0.369 | 0.344 | 0.296 | 0.336 |
| I67R, T605R, D568R | 0.357 | 0.330 | 0.319 | 0.335 |
| I67R, D56R, E706R | 0.356 | 0.332 | 0.287 | 0.325 |
| I67R, T605R, E706R | 0.339 | 0.319 | 0.303 | 0.320 |
| I67R, D568R, E694R | 0.326 | 0.336 | 0.296 | 0.319 |
| I67R, D56R, E694R, E706R | 0.301 | 0.336 | 0.281 | 0.306 |
| I67R, D56R, T605R, E706R | 0.285 | 0.340 | 0.277 | 0.300 |
| I67R, T605R | 0.317 | 0.297 | 0.273 | 0.296 |
| I67R, D56R, D568R, E706R | 0.293 | 0.312 | 0.281 | 0.295 |
| I67R, T605R, E694R | 0.283 | 0.320 | 0.273 | 0.292 |
| I67R, D56R, E694R | 0.300 | 0.318 | 0.256 | 0.291 |
| T605R, D568R, E706R | 0.268 | 0.289 | 0.315 | 0.291 |
| I67R, D568R | 0.339 | 0.267 | 0.264 | 0.290 |
| I67R, D56R, T605R | 0.302 | 0.308 | 0.260 | 0.290 |
| I67R, D56R, D568R, E694R | 0.273 | 0.320 | 0.268 | 0.287 |
| E63R, T605R | 0.329 | 0.272 | 0.260 | 0.287 |
| I67R, T605R, D568R, E706R | 0.271 | 0.302 | 0.281 | 0.285 |
| I67R, D56R | 0.331 | 0.284 | 0.238 | 0.284 |
| D56R, D568R, E706R | 0.267 | 0.293 | 0.289 | 0.283 |
| I67R, T605R, D568R, E694R | 0.238 | 0.324 | 0.284 | 0.282 |
| E63R, E706R | 0.361 | 0.238 | 0.245 | 0.282 |
| D56R, T605R, D568R | 0.255 | 0.306 | 0.277 | 0.279 |
| T605R, D568R, E694R | 0.233 | 0.293 | 0.288 | 0.271 |
| E63R, E655R | 0.296 | 0.265 | 0.251 | 0.271 |
| E63R, D568R | 0.335 | 0.234 | 0.239 | 0.270 |
| I67R, E694R | 0.289 | 0.279 | 0.240 | 0.270 |
| T605R, D568R | 0.258 | 0.270 | 0.278 | 0.269 |
| I67R, D568R, E694R, E706R | 0.265 | 0.280 | 0.253 | 0.266 |
| D56R, T605R, E706R | 0.227 | 0.299 | 0.268 | 0.264 |
| D56R, T605R | 0.229 | 0.298 | 0.250 | 0.259 |
| I67R, E706R, E655R | 0.226 | 0.286 | 0.259 | 0.257 |
| I67R, T605R, E694R, E706R | 0.239 | 0.284 | 0.247 | 0.257 |
| I67R, D56R, T605R, D568R | 0.241 | 0.279 | 0.248 | 0.256 |
| I67R, E706R | 0.299 | 0.226 | 0.230 | 0.252 |
| D56R, D568R, E694R | 0.232 | 0.263 | 0.259 | 0.251 |
| I67R, E694R, E706R | 0.278 | 0.247 | 0.216 | 0.247 |
| I67R, D568R, E655R | 0.217 | 0.269 | 0.250 | 0.245 |
| E63R, E694R | 0.276 | 0.239 | 0.217 | 0.244 |
| D56R, D568R | 0.245 | 0.257 | 0.222 | 0.241 |
| D56R, T605R, E694R | 0.191 | 0.291 | 0.241 | 0.241 |
| D568R, E706R, E655R | 0.179 | 0.261 | 0.279 | 0.240 |
| I67R, E655R | 0.233 | 0.245 | 0.238 | 0.239 |
| D56R, E694R | 0.205 | 0.280 | 0.228 | 0.238 |
| I67R, D56R, D568R, E655R | 0.180 | 0.292 | 0.237 | 0.236 |
| T605R, E694R | 0.190 | 0.272 | 0.242 | 0.235 |
| I67R, D56R, T605R, E694R | 0.195 | 0.278 | 0.219 | 0.231 |
| G71R, E655R | 0.258 | 0.221 | 0.191 | 0.224 |
| D56R, D568R, E655R | 0.167 | 0.255 | 0.241 | 0.221 |
| D56R, D568R, E694R, E706R | 0.183 | 0.247 | 0.233 | 0.221 |
| D568R, E694R | 0.197 | 0.241 | 0.219 | 0.219 |
| I67R, D56R, E706R, E655R | 0.166 | 0.267 | 0.223 | 0.219 |
| D56R, T605R, D568R, E706R | 0.176 | 0.242 | 0.235 | 0.218 |
| D568R, E706R | 0.219 | 0.204 | 0.227 | 0.217 |
| D56R, E706R | 0.201 | 0.243 | 0.205 | 0.216 |
| I67R, D56R, E655R | 0.210 | 0.242 | 0.196 | 0.216 |
| E694R, G71R | 0.250 | 0.213 | 0.181 | 0.214 |
| I67R, D568R, E706R, E655R | 0.147 | 0.258 | 0.238 | 0.214 |
| I67R, G71R | 0.314 | 0.171 | 0.158 | 0.214 |
| D56R, T605R, E694R, E706R | 0.160 | 0.253 | 0.223 | 0.212 |
| D568R, G71R | 0.290 | 0.160 | 0.186 | 0.212 |
| D56R, G71R | 0.255 | 0.219 | 0.158 | 0.211 |
| D568R, E655R | 0.164 | 0.228 | 0.238 | 0.210 |
| T605R, G71R | 0.224 | 0.198 | 0.205 | 0.209 |
| T605R, E694R, E706R | 0.155 | 0.254 | 0.216 | 0.208 |
| T605R, E706R | 0.171 | 0.225 | 0.225 | 0.207 |
| D56R, E694R, E706R | 0.169 | 0.252 | 0.196 | 0.206 |
| D568R, E694R, E706R, E655R | 0.129 | 0.239 | 0.246 | 0.205 |
| D568R, E694R, E706R | 0.178 | 0.219 | 0.211 | 0.203 |
| I67R, E63R | 0.336 | 0.179 | 0.088 | 0.201 |
| I67R, E694R, E655R | 0.152 | 0.244 | 0.207 | 0.201 |
| D568R, E694R, E655R | 0.126 | 0.245 | 0.231 | 0.201 |
| D56R, E655R | 0.143 | 0.248 | 0.205 | 0.199 |
| E63R, G71R | 0.334 | 0.138 | 0.123 | 0.198 |
| I67R, T605R, D568R, E655R | 0.129 | 0.240 | 0.221 | 0.197 |
| T605R, D568R, E694R, E706R | 0.149 | 0.204 | 0.215 | 0.189 |
| D56R, E706R, E655R | 0.126 | 0.227 | 0.214 | 0.189 |
| I67R, T605R, E655R | 0.143 | 0.220 | 0.192 | 0.185 |
| I67R, D56R, T605R, E655R | 0.123 | 0.237 | 0.178 | 0.179 |
| I67R, E694R, E706R, E655R | 0.120 | 0.205 | 0.188 | 0.171 |
| D56R, D568R, E706R, E655R | 0.110 | 0.199 | 0.204 | 0.171 |
| E694R, E655R | 0.100 | 0.218 | 0.195 | 0.171 |
| D56R, T605R, D568R, E694R | 0.123 | 0.200 | 0.187 | 0.170 |
| G71R, E706R | 0.227 | 0.131 | 0.148 | 0.169 |
| I67R, T605R, E706R, E655R | 0.102 | 0.206 | 0.196 | 0.168 |
| T605R, D568R, E655R | 0.093 | 0.201 | 0.209 | 0.168 |
| I67R, D56R, E694R, E655R | 0.103 | 0.222 | 0.163 | 0.163 |
| E706R, E655R | 0.105 | 0.181 | 0.197 | 0.161 |
| T605R, D568R, E706R, E655R | 0.080 | 0.185 | 0.209 | 0.158 |
| I67R, D568R, E694R, E655R | 0.091 | 0.199 | 0.179 | 0.156 |
| D56R, E694R, E655R | 0.080 | 0.198 | 0.172 | 0.150 |
| E694R, E706R, E655R | 0.077 | 0.186 | 0.173 | 0.145 |
| D56R, E694R, E706R, E655R | 0.080 | 0.187 | 0.165 | 0.144 |
| D56R, T605R, D568R, E655R | 0.076 | 0.166 | 0.174 | 0.139 |
| D56R, T605R, E655R | 0.062 | 0.180 | 0.150 | 0.131 |
| D56R, D568R, E694R, E655R | 0.069 | 0.153 | 0.153 | 0.125 |
| T605R, E706R, E655R | 0.052 | 0.140 | 0.152 | 0.115 |
| D56R, T605R, E706R, E655R | 0.052 | 0.146 | 0.141 | 0.113 |
| T605R, D568R, E694R, E655R | 0.048 | 0.129 | 0.146 | 0.107 |
| I67R, T605R, E694R, E655R | 0.056 | 0.140 | 0.126 | 0.107 |
| T605R, E694R, E655R | 0.044 | 0.137 | 0.124 | 0.102 |
| T605R, E655R | 0.045 | 0.118 | 0.120 | 0.094 |
| T605R, E694R, E706R, E655R | 0.041 | 0.113 | 0.121 | 0.091 |
| E694R, E706R | 0.065 | 0.109 | 0.093 | 0.089 |
| D56R, T605R, E694R, E655R | 0.037 | 0.113 | 0.112 | 0.087 |
| Nuclease A | 0.044 | 0.033 | 0.049 | 0.042 |
| E63R, D56R | 0.001 | 0.004 | 0.001 | 0.002 |

As shown in Table 6, all but one of the variants of Nuclease A with combinations of amino acid substitutions exhibited higher indel activity than Nuclease A (SEQ ID NO: 1). 8 CRISPR nuclease variants resulted in indel ratios of over 0.3 when averaged across the three targets, indicating that over 30% of NGS reads comprised indels. These 8 nuclease variants of Nuclease A comprised the following substitution combinations: a) 167R, D568R, and E706R; b) I67R, D56R, and D568R; c) I67R, T605R, and D568R; d) I67R, D56R, and E706R; e) I67R, T605R, and E706R; f) I67R, D568R, and E694R; g) I67R, D56R, E694R, and E706R; and h) I67R, D56R, T605R, and E706R. 64 variants of Nuclease A resulted in indel ratios of over 0.2 when averaged across the three targets, indicating that over 20% of NGS reads comprised indels. 29 variants of Nuclease A resulted in indel ratios of over 0.1 when averaged across the three targets, indicating that over 10% of NGS reads comprised indels.

Based on this experiment, the Nuclease A variant comprising the following substitutions was selected for further testing: I67R, D568R, and E706R. This Nuclease A variant exhibited an over 8-fold increase in indel activity compared to the reference Nuclease A (SEQ ID NO: 1).

### Example 4-Editing of Human Target Genes by RNA-Guided Nuclease A Variants in HEK293T Cells and Additional RNA Scaffold Sequences

This Example describes the genomic editing of the exemplary target genes by Nuclease A of SEQ ID NO: 1 and the I67R, D568R, E706R Nuclease A variant of SEQ ID NO: 6 when combined with RNA guides comprising alternative scaffold sequences.

RNA guides were designed using the RNA scaffold sequences of Table 7 and cloned into a pUC19 plasmid following the U6 PolIII promoter and terminated with a 6× polyT sequence, unless noted otherwise. RNA guides were designed to be specific to the AAVS1-T3, EMX1-T7, and/or AAVS1-T3b target sequences (see Example 1 and Table 7). See all RNA guide sequences in Table 8. The U6 PolIII uses a +1 G at the start of the transcript (i.e., the 5′ end of the RNA) for more efficient transcription that is excluded from the sequences described in Table 8. Industrial-grade plasmids were received from GenScript.

TABLE 7
RNA Guide Component Sequences

| Component | Sequence |
|---|---|
| AAVS1-T3b | GAGAAAGAGAAAGGGAGUAG (SEQ ID NO: 26) |
| Spacer | |
| Truncated | Guuacaguuaagggaaaccuuaauuguaaaacgccua |
| Reference | uaugguaaagugauguacguuuggguauauaucgcca |
| Scaffold | gccugaaccucuacgccagaaauggcagcuuuaucau |
| | ggguuaggacgauauu (SEQ ID NO: 27) |
| Scaffold 2 | Guuacaguuaagggaaaccuuaauuguaaaacgccua |
| | uacggugaagggauauacgcuuggguuuguccagccu |
| | gagccucuaugccagaaauggcgccuucaucgugggu |
| | uaggaca (SEQ ID NO: 28) |

TABLE 8
RNA Guide Sequences

| Description | Sequence |
|---|---|
| Truncated | UUGUGAGAAUGGUGCGUCCUguuacaguuaagggaaa |
| Reference | ccuuaauuguaaaacgccuauaugguaaagugaugua |
| Scaffold | cguuuggguauauaucgccagccugaaccucuacgcc |
| (AAVS1-T3) | agaaauggcagcuuuaucauggguuaggacgauauu |
| | (SEQ ID NO: 29) |
| Truncated | CUUUAGCAAGGGACUAUUCAguuacaguuaagggaaa |
| Reference | ccuuaauuguaaaacgccuauaugguaaagugaugua |
| Scaffold | cguuuggguauauaucgccagccugaaccucuacgcc |
| (EMX1-T7) | agaaauggcagcuuuaucauggguuaggacgauauu |
| | (SEQ ID NO: 30) |
| Scaffold 2 | GAGAAAGAGAAAGGGAGUAGguuacaguuaagggaaa |
| (AAVS1-T3b) | ccuuaauuguaaaacgccuauacggugaagggauaua |
| | cgcuuggguuuguccagccugagccucuaugccagaa |
| | auggcgccuucaucguggguuaggaca |
| | (SEQ ID NO: 31) |

*Spacer sequences in upper case and scaffold sequences in lower case

The RNA guides of Table 8 were combined with either Nuclease A or the I67R/D568R/E706R variant of Nuclease A and were introduced into HEK293T cells by lipid-based transient transfection as described in Example 1. The AAVS1-T3 and EMX1-T7 RNA guides from Example 1 were further used as controls. Genomic DNA was recovered approximately 72 hours post-transfection, and samples were prepared for NGS and analyzed as described in Example 1. The percentage of NGS reads comprising indels shown in Table 9 were calculated as the average of two biological replicates, which each comprised two technical replicates. The percentage of NGS reads comprising indels shown in Table 10 were calculated as the average of three technical replicates, unless indicated otherwise.

TABLE 9
Indel activity of Nuclease A

| RNA Guide | Average Indel Percentage |
|---|---|
| Reference Scaffold (AAVS1-T3) | 4.85 |
| Reference Scaffold (EMX1-T7) | 4.10 |
| Truncated Reference Scaffold (AAVS1-T3) | 12.89* |
| Truncated Reference Scaffold (EMX1-T7) | 9.93 |
| No guide (AAVS1-T3) | 0.10 |
| No guide (EMX1-T7) | 0.24 |

TABLE 10
I67R/D568R/E706R Variant CRISPR Nuclease Indel Activity

| RNA Guide | Average Indel Percentage |
|---|---|
| Reference Scaffold (AAVS1-T3) | 35.90 |
| Reference Scaffold (EMX1-T7) | 28.97 |
| Scaffold 2 (AAVS1-T3b) | 36.79* |
| No guide (AAVS1-T3) | 0.34 |
| No guide (EMX1-T7) | 0.66 |
| No guide (AAVS1-T3b) | 0.15 |

As shown in Table 9, RNA guides comprising the truncated reference scaffold exhibited higher indel activity than the RNA guides comprising the reference scaffold when paired with the reference Nuclease A. As shown in Table 10, RNA guides comprising the reference scaffold or scaffold 2 each exhibited high indel activity with the I67R, D568R, E706R Nuclease A variant. In all cases, the average percent indel observed was significantly higher than background controls comprising either the reference Nuclease A or the I67R, D568R, E706R Nuclease A variant in the absence of an RNA guide.

This Example thus shows that the truncated reference scaffold, and scaffold 2 are capable of being recognized by Nuclease A (SEQ ID NO: 1) and Nuclease A variant (SEQ ID NO: 6).

### Example 5: Engineering and Effectiveness of Nickase Variants of CRISPR Nuclease A for Targeting Mammalian Genes

This Example describes introducing mutations into Nuclease A of SEQ ID NO: 1 that disrupt either the HNH or RuvC domains to produce a functional nickase. D396, H397, and N420 were identified as putative catalytic residues of the HNH domain. D24, E337, and D524 positions were identified as putative catalytic residues of the RuvC domain. These positions were identified by analyzing models generated with AlphaFold2 (Jumper et al., Nature 596:583-9 (2021)) for structural regions resembling known HNH and RuvC active sites and/or by performing sequence alignments to other nucleases for which candidate positions had been previously identified. Examples of reference structures used to identify the HNH and RuvC active sites are represented with the following PDB IDs: 5h0m, 7eu9, 6ltu, 7odf, 7lys, 8dc2, 4cmp, 4008, 7z4j, 5axw, 5b20, 6kc8, 7utn, 8csz, 8ctl, 8dmb.

The coding sequence of Nuclease A was converted into an E. coli-codon optimized DNA sequence, synthesized, and cloned into a pET-28a (+) vector (Novagen), containing lac and T7 RNA polymerase promoters for gene expression. To test for nickase activity, individual alanine mutants were cloned for each of the positions identified as putative active site residues of the HNH and RuvC domains. A leucine mutant was also cloned for position H397. Research grade plasmids were received from GenScript. The engineered nickase sequences are shown in Table 11. The codon encoding the substituted residue is capitalized, bold, and underlined in the nucleotide sequence, and the substituted residue is shown in bold and underlined in the amino acid sequence. The putative HNH-knockout nickases were anticipated to cleave the non-target strand but not the target strand. The putative RuvC-knockout nickases were anticipated to cleave the target strand but not the non-target strand.

TABLE 11
CRISPR Nuclease and Nickase Sequences

| CRISPR | | |
|---|---|---|
| Nuclease and | | |
| Nickase | Nucleotide sequence | Amino acid sequence |
| Nuclease A | atgaggaattgtaagaaagatctatatgtagaggttaccccgatgca | SEQ ID NO: 1 (see Table 1) |
| | gaaagagttagtcctgggtatcgattacggcggtaagtataccggg | |
| | ctggcggtcgtggatcgtcgcaataaccaagttctgtacgcgcgta | |
| | ccgtcaagatgcgtgacgatgtagctgatattttagagggtcgtcgg | |
| | gagcagcgtggtatccgtcgtaccggccagaccaagaagaaacgt | |
| | ctacgtgaattgaagacctatttgaaaaacattggcggtaagtacga | |
| | cgaaagcatcggtacattcaagaacgaaccgtttatgaccgtgtaca | |
| | aactgtgtcataagcgtggctatgactacgttgagatgccgaaaga | |
| | gaagacccgtgaagagattgaggctatgagcgcggatgagaaga | |
| | agcaatgggaaaaagagaaaaaggagtgggaagagaccgaacg | |
| | caactctcgtcaccgtgacgaggtaacgaaggacgttcgtaaaatc | |
| | atgagcgaacgcgacgcgaccgaacagcagatcagccatgttatt | |
| | aaaatcttcaacaaacaatatcgtccaaagcgttttaacaaccgtatc | |
| | ctgaccaagtgcaaagttgaagattgtggtgtcaataccccgctgcg | |
| | taaaaacgtgcgtgacctgctgattaaggacatcgtgcgttttctgcc | |
| | gctgacgcagagcgaaaaggctgaattggaggcccacgtgctgg | |
| | acaaagaaggccgcgagaaggttaaaaccttcttccgacgccacc | |
| | gcattaacgagttcctgcgcaagcagatttatgacatcgctttcaatc | |
| | agaaactttctggtcgtaccgtcttctgcaaaaagcacattctggagc | |
| | acgaacaacacacgaaggaggaacgcaaggtgttccgcttggcg | |
| | ccaagcttgaaaaccaagatcaataacgtgttggctgtggttaaaga | |
| | tgagatccttccgcaatttggtattaataaagtgattatggagtccaac | |
| | aattttgatatcgccgcacgtaccaagggcaaaaagagattagcaa | |
| | aagacgagtatggtaagggtcataaagaagcgaaggagtccctca | |
| | tcgaggtgctgctgagagaaaccaaaaaccaatgtgtttactgcggt | |
| | aaggtgatcaccgttacgaccgcgaacaccgaccacatctttccgc | |
| | gtaaggcgggtggtatcaacgtctttgcaaatctggttgcctgctgtc | |
| | gtgagtgcaatgaaaaaaaaaaaggccgtaccccgctggagtccg | |
| | gtgtgttgccacaaccggaaattactgcgcacctggagaacgatct | |
| | gaagaagaaaattctggaggacgctcggaacatgaatgagttgaa | |
| | cctgaattcgatcaaatacatgagctatgccagcatcggctggcgtc | |
| | acatgcgtgaccgactccgtggtttaatcggcaacgaagaactgcc | |
| | gattgaacgccaaagcggcatcttgaccgcgtactttcgtcgctggt | |
| | ggggtttccgcaaagaaaggggcaatgacattcatcacgcactgg | |
| | acgcggttattttagcgtctcgcaaggattacaccgacgacggcgtt | |
| | gatatgacgctgaagccgaaatccagcaacggtcgcgaattcgatc | |
| | cggaaaaacacctgccggataccatcagttttaaacgtgacaaaag | |
| | caacaaaggcacgcctctgtacgacaagaatccgctttcgattaag | |
| | aacagcttgattacccgtcgctggatggttactgaaatcgaacatgg | |
| | taaggaggataccattatttcggatgagtacagaaaaaagctaaaa | |
| | gaagcatttaagcattggggcacgagcaaaggcaaatgcctgacg | |
| | gatgaagaggcgcgtcagaccggcttttatcagcgcaaaaacggg | |
| | aacttcatgtcactccgctgcgttgaaaaaggtctgggctggggac | |
| | aaatggtgaaaatcaataacaacgcgttcaaaaccaatgtgcataat | |
| | attggtgtagcagtgtacctggatgagaagggtaagaaaaaggcgt | |
| | atgagatcaagaacccgcgtctgctcaaacacttcgtggaaaaacc | |
| | gccggagataaaaggcaagaagctctacacactgcgtcgtggcga | |
| | ttatgttacgcatgaaagtgaggacagcgtttaccgtatcaaaaagct | |
| | gggtacgtccccggttctcgaggccgtggttaaaggctctattggta | |
| | gaatccgtaatagcgcatcggctaccaagttgactaaggtctccaaa | |
| | ccgcagaaagtgttc (SEQ ID NO: 32) | |
| D396A | atgaggaattgtaagaaagatctatatgtagaggttaccccgatgca | MRNCKKDLYVEVTPMQKE |
| (putative HNH- | gaaagagttagtcctgggtatcgattacggcggtaagtataccggg | LVLGIDYGGKYTGLAVVDR |
| knockout) | ctggcggtcgtggatcgtcgcaataaccaagttctgtacgcgcgta | RNNQVLYARTVKMRDDVA |
| | ccgtcaagatgcgtgacgatgtagctgatattttagagggtcgtcgg | DILEGRREQRGIRRTGQTKK |
| | gagcagcgtggtatccgtcgtaccggccagaccaagaagaaacgt | KRLRELKTYLKNIGGKYDE |
| | ctacgtgaattgaagacctatttgaaaaacattggcggtaagtacga | SIGTFKNEPFMTVYKLCHKR |
| | cgaaagcatcggtacattcaagaacgaaccgtttatgaccgtgtaca | GYDYVEMPKEKTREEIEAM |
| | aactgtgtcataagcgtggctatgactacgttgagatgccgaaaga | SADEKKQWEKEKKEWEET |
| | gaagacccgtgaagagattgaggctatgagcgcggatgagaaga | ERNSRHRDEVTKDVRKIMS |
| | agcaatgggaaaaagagaaaaaggagtgggaagagaccgaacg | ERDATEQQISHVIKIFNKQY |
| | caactctcgtcaccgtgacgaggtaacgaaggacgttcgtaaaatc | RPKRFNNRILTKCKVEDCG |
| | atgagcgaacgcgacgcgaccgaacagcagatcagccatgttatt | VNTPLRKNVRDLLIKDIVRF |
| | aaaatcttcaacaaacaatatcgtccaaagcgttttaacaaccgtatc | LPLTQSEKAELEAHVLDKE |
| | ctgaccaagtgcaaagttgaagattgtggtgtcaataccccgctgcg | GREKVKTFFRRHRINEFLRK |
| | taaaaacgtgcgtgacctgctgattaaggacatcgtgcgttttctgcc | QIYDIAFNQKLSGRTVFCKK |
| | gctgacgcagagcgaaaaggctgaattggaggcccacgtgctgg | HILEHEQHTKEERKVFRLAP |
| | acaaagaaggccgcgagaaggttaaaaccttcttccgacgccacc | SLKTKINNVLAVVKDEILPQ |
| | gcattaacgagttcctgcgcaagcagatttatgacatcgctttcaatc | FGINKVIMESNNFDIAARTK |
| | agaaactttctggtcgtaccgtcttctgcaaaaagcacattctggagc | GKKRLAKDEYGKGHKEAK |
| | acgaacaacacagaaggaggaacgcaaggtgttccgcttggcg | ESLIEVLLRETKNQCVYCGK |
| | ccaagcttgaaaaccaagatcaataacgtgttggctgtggttaaaga | VITVTTANTAHIFPRKAGGI |
| | tgagatccttccgcaatttggtattaataaagtgattatggagtccaac | NVFANLVACCRECNEKKKG |
| | aattttgatatcgccgcacgtaccaagggcaaaaagagattagcaa | RTPLESGVLPQPEITAHLEN |
| | aagacgagtatggtaagggtcataaagaagcgaaggagtccctca | DLKKKILEDARNMNELNLN |
| | tcgaggtgctgctgagagaaaccaaaaaccaatgtgtttactgcggt | SIKYMSYASIGWRHMRDRL |
| | aaggtgatcaccgttacgaccgcgaacaccGCGcacatctttcc | RGLIGNEELPIERQSGILTAY |
| | gcgtaaggcgggtggtatcaacgtctttgcaaatctggttgcctgct | FRRWWGFRKERGNDIHHAL |
| | gtcgtgagtgcaatgaaaaaaaaaaaggccgtaccccgctggagt | DAVILASRKDYTDDGVDMT |
| | ccggtgtgttgccacaaccggaaattactgcgcacctggagaacg | LKPKSSNGREFDPEKHLPDT |
| | atctgaagaagaaaattctggaggacgctcggaacatgaatgagtt | ISFKRDKSNKGTPLYDKNPL |
| | gaacctgaattcgatcaaatacatgagctatgccagcatcggctgg | SIKNSLITRRWMVTEIEHGK |
| | cgtcacatgcgtgaccgactccgtggtttaatcggcaacgaagaac | EDTIISDEYRKKLKEAFKHW |
| | tgccgattgaacgccaaagcggcatcttgaccgcgtactttcgtcgc | GTSKGKCLTDEEARQTGFY |
| | tggtggggtttccgcaaagaaaggggcaatgacattcatcacgcac | QRKNGNFMSLRCVEKGLG |
| | tggacgcggttattttagcgtctcgcaaggattacaccgacgacgg | WGQMVKINNNAFKTNVHNI |
| | cgttgatatgacgctgaagccgaaatccagcaacggtcgcgaattc | GVAVYLDEKGKKKAYEIKN |
| | gatccggaaaaacacctgccggataccatcagttttaaacgtgaca | PRLLKHFVEKPPEIKGKKLY |
| | aaagcaacaaaggcacgcctctgtacgacaagaatccgctttcgat | TLRRGDYVTHESEDSVYRIK |
| | taagaacagcttgattacccgtcgctggatggttactgaaatcgaac | KLGTSPVLEAVVKGSIGRIR |
| | atggtaaggaggataccattatttcggatgagtacagaaaaaagcta | NSASATKLTKVSKPQKVF |
| | aaagaagcatttaagcattggggcacgagcaaaggcaaatgcctg | (SEQ ID NO: 34) |
| | acggatgaagaggcgcgtcagaccggcttttatcagcgcaaaaac | |
| | gggaacttcatgtcactccgctgcgttgaaaaaggtctgggctggg | |
| | gacaaatggtgaaaatcaataacaacgcgttcaaaaccaatgtgca | |
| | taatattggtgtagcagtgtacctggatgagaagggtaagaaaaag | |
| | gcgtatgagatcaagaacccgcgtctgctcaaacacttcgtggaaa | |
| | aaccgccggagataaaaggcaagaagctctacacactgcgtcgtg | |
| | gcgattatgttacgcatgaaagtgaggacagcgtttaccgtatcaaa | |
| | aagctgggtacgtccccggttctcgaggccgtggttaaaggctctat | |
| | tggtagaatccgtaatagcgcatcggctaccaagttgactaaggtct | |
| | ccaaaccgcagaaagtgttc (SEQ ID NO: 33) | |
| H397A | atgaggaattgtaagaaagatctatatgtagaggttaccccgatgca | MRNCKKDLYVEVTPMQKE |
| (putative HNH- | gaaagagttagtcctgggtatcgattacggcggtaagtataccggg | LVLGIDYGGKYTGLAVVDR |
| knockout) | ctggcggtcgtggatcgtcgcaataaccaagttctgtacgcgcgta | RNNQVLYARTVKMRDDVA |
| | ccgtcaagatgcgtgacgatgtagctgatattttagagggtcgtcgg | DILEGRREQRGIRRTGQTKK |
| | gagcagcgtggtatccgtcgtaccggccagaccaagaagaaacgt | KRLRELKTYLKNIGGKYDE |
| | ctacgtgaattgaagacctatttgaaaaacattggcggtaagtacga | SIGTFKNEPFMTVYKLCHKR |
| | cgaaagcatcggtacattcaagaacgaaccgtttatgaccgtgtaca | GYDYVEMPKEKTREEIEAM |
| | aactgtgtcataagcgtggctatgactacgttgagatgccgaaaga | SADEKKQWEKEKKEWEET |
| | gaagacccgtgaagagattgaggctatgagcgcggatgagaaga | ERNSRHRDEVTKDVRKIMS |
| | agcaatgggaaaaagagaaaaaggagtgggaagagaccgaacg | ERDATEQQISHVIKIFNKQY |
| | caactctcgtcaccgtgacgaggtaacgaaggacgttcgtaaaatc | RPKRFNNRILTKCKVEDCG |
| | atgagcgaacgcgacgcgaccgaacagcagatcagccatgttatt | VNTPLRKNVRDLLIKDIVRF |
| | aaaatcttcaacaaacaatatcgtccaaagcgttttaacaaccgtatc | LPLTQSEKAELEAHVLDKE |
| | ctgaccaagtgcaaagttgaagattgtggtgtcaataccccgctgcg | GREKVKTFFRRHRINRFLRK |
| | taaaaacgtgcgtgacctgctgattaaggacatcgtgcgttttctgcc | QIYDIAFNQKLSGRTVFCKK |
| | gctgacgcagagcgaaaaggctgaattggaggcccacgtgctgg | HILEHEQHTKEERKVFRLAP |
| | acaaagaaggccgcgagaaggttaaaaccttcttccgacgccacc | SLKTKINNVLAVVKDEILPQ |
| | gcattaacgagttcctgcgcaagcagatttatgacatcgctttcaatc | FGINKVIMESNNFDIAARTK |
| | agaaactttctggtcgtaccgtcttctgcaaaaagcacattctggagc | GKKRLAKDEYGKGHKEAK |
| | acgaacaacacacgaaggaggaacgcaaggtgttccgcttggcg | ESLIEVLLRETKNQCVYCGK |
| | ccaagcttgaaaaccaagatcaataacgtgttggctgtggttaaaga | VITVTTANTDAIFPRKAGGI |
| | tgagatccttccgcaatttggtattaataaagtgattatggagtccaac | NVFANLVACCRECNEKKKG |
| | aattttgatatcgccgcacgtaccaagggcaaaaagagattagcaa | RTPLESGVLPQPEITAHLEN |
| | aagacgagtatggtaagggtcataaagaagcgaaggagtccctca | DLKKKILEDARNMNELNLN |
| | tcgaggtgctgctgagagaaaccaaaaaccaatgtgtttactgcggt | SIKYMSYASIGWRNHRDRL |
| | aaggtgatcaccgttacgaccgcgaacaccgacGCGatctttcc | RGLIGNEELPIERQSGILTAY |
| | gcgtaaggcgggtggtatcaacgtctttgcaaatctggttgcctgct | FRRWWGFRKERGNDIHHAL |
| | gtcgtgagtgcaatgaaaaaaaaaaaggccgtaccccgctggagt | DAVILASRKDYTDDGVDMT |
| | ccggtgtgttgccacaaccggaaattactgcgcacctggagaacg | LKPKSSNGREFDPEKHLPDT |
| | atctgaagaagaaaattctggaggacgctcggaacatgaatgagtt | ISFKRDKSNKGTPLYDKNPL |
| | gaacctgaattcgatcaaatacatgagctatgccagcatcggctgg | SIKNSLITRRWMVTEIEHGK |
| | cgtcacatgcgtgaccgactccgtggtttaatcggcaacgaagaac | EDTIISDEYRKKLKEAFKHW |
| | tgccgattgaacgccaaagcggcatcttgaccgcgtactttcgtcgc | GTSKGKCLTDEEARQTGFY |
| | tggtggggtttccgcaaagaaaggggcaatgacattcatcacgcac | QRKNGNFMSLRCVEKGLG |
| | tggacgcggttattttagcgtctcgcaaggattacaccgacgacgg | WGQMVKINNNAFKTNVHNI |
| | cgttgatatgacgctgaagccgaaatccagcaacggtcgcgaattc | GVAVYLDEKGKKKAYEIKN |
| | gatccggaaaaacacctgccggataccatcagttttaaacgtgaca | PRLLKHFVEKPPEIKGKKLY |
| | aaagcaacaaaggcacgcctctgtacgacaagaatccgctttcgat | TLRRGDYVTHESEDSVYRIK |
| | taagaacagcttgattacccgtcgctggatggttactgaaatcgaac | KLGTSPVLEAVVKGSIGRIR |
| | atggtaaggaggataccattatttcggatgagtacagaaaaaagcta | NSASATKLTKVSKPQKVF |
| | aaagaagcatttaagcattggggcacgagcaaaggcaaatgcctg | (SEQ ID NO: 36) |
| | acggatgaagaggcgcgtcagaccggcttttatcagcgcaaaaac | |
| | gggaacttcatgtcactccgctgcgttgaaaaaggtctgggctggg | |
| | gacaaatggtgaaaatcaataacaacgcgttcaaaaccaatgtgca | |
| | taatattggtgtagcagtgtacctggatgagaagggtaagaaaaag | |
| | gcgtatgagatcaagaacccgcgtctgctcaaacacttcgtggaaa | |
| | aaccgccggagataaaaggcaagaagctctacacactgcgtcgtg | |
| | gcgattatgttacgcatgaaagtgaggacagcgtttaccgtatcaaa | |
| | aagctgggtacgtccccggttctcgaggccgtggttaaaggctctat | |
| | tggtagaatccgtaatagcgcatcggctaccaagttgactaaggtct | |
| | ccaaaccgcagaaagtgttc (SEQ ID NO: 35) | |
| H397L (putative | atgaggaattgtaagaaagatctatatgtagaggttaccccgatgca | MRNCKKDLYVEVTPMQKE |
| HNH-knockout) | gaaagagttagtcctgggtatcgattacggcggtaagtataccggg | LVLGIDYGGKYTGLAVVDR |
| | ctggcggtcgtggatcgtcgcaataaccaagttctgtacgcgcgta | RNNQVLYARTVKMRDDVA |
| | ccgtcaagatgcgtgacgatgtagctgatattttagagggtcgtcgg | DILEGRREQRGIRRTGQTKK |
| | gagcagcgtggtatccgtcgtaccggccagaccaagaagaaacgt | KRLRELKTYLKNIGGKYDE |
| | ctacgtgaattgaagacctatttgaaaaacattggcggtaagtacga | SIGTFKNEPFMTVYKLCHKR |
| | cgaaagcatcggtacattcaagaacgaaccgtttatgaccgtgtaca | GYDYVEMPKEKTREEIEAM |
| | aactgtgtcataagcgtggctatgactacgttgagatgccgaaaga | SADEKKQWEKEKKEWEET |
| | gaagacccgtgaagagattgaggctatgagcgcggatgagaaga | ERNSRHRDEVTKDVRKIMS |
| | agcaatgggaaaaagagaaaaaggagtgggaagagaccgaacg | ERDATEQQISHVIKIFNKQY |
| | caactctcgtcaccgtgacgaggtaacgaaggacgttcgtaaaatc | RPKRFNNRILTKCKVEDCG |
| | atgagcgaacgcgacgcgaccgaacagcagatcagccatgttatt | VNTPLRKNVRDLLIKDIVRF |
| | aaaatcttcaacaaacaatatcgtccaaagcgttttaacaaccgtatc | LPLTQSEKAELEAHVLDKE |
| | ctgaccaagtgcaaagtggaagattgtggtgtcaataccccgctgcg | GREKVKTFFRRHRINEFLRK |
| | taaaaacgtgcgtgacctgctgattaaggacatcgtgcgttttctgcc | QIYDIAFNQKLSGRTVFCKK |
| | gctgacgcagagcgaaaaggctgaattggaggcccacgtgctgg | HILEHEQHTKEERKVFRLAP |
| | acaaagaaggccgcgagaaggttaaaaccttcttccgacgccacc | SLKTKINNVLAVVKDEILPQ |
| | gcattaacgagttcctgcgcaagcagatttatgacatcgctttcaatc | FGINKVIMESNNFDIAARTK |
| | agaaactttctggtcgtaccgtcttctgcaaaaagcacattctggagc | GKKRLAKDEYGKGHKEAK |
| | acgaacaacacacgaaggaggaacgcaaggtgttccgcttggcg | ESLIEVLLRETKNQCVYCGK |
| | ccaagcttgaaaaccaagatcaataacgtgttggctgtggttaaaga | VITVTTANTDLIFPRKAGGI |
| | tgagatccttccgcaatttggtattaataaagtgattatggagtccaac | NVFANLVACCRECNEKKKG |
| | aattttgatatcgccgcacgtaccaagggcaaaaagagattagcaa | RTPLESGVLPQPEITAHLEN |
| | aagacgagtatggtaagggtcataaagaagcgaaggagtccctca | DLKKKILEDARNMNELNLN |
| | tcgaggtgctgctgagagaaaccaaaaaccaatgtgtttactgcggt | SIKYMSYASIGWRHMRDRL |
| | aaggtgatcaccgttacgaccgcgaacaccgacCTGatctttcc | RGLIGNEELPIERQSGILTAY |
| | gcgtaaggcgggtggtatcaacgtctttgcaaatctggttgcctgct | FRRWWGFRKERGNDIHHAL |
| | gtcgtgagtgcaatgaaaaaaaaaaaggccgtaccccgctggagt | DAVILASRKDYTDDGVDMT |
| | ccggtgtgttgccacaaccggaaattactgcgcacctggagaacg | LKPKSSNGREFDPEKHLPDT |
| | atctgaagaagaaaattctggaggacgctcggaacatgaatgagtt | ISFKRDKSNKGTPLYDKNPL |
| | gaacctgaattcgatcaaatacatgagctatgccagcatcggctgg | SIKNSLITRRWMVTEIEHGK |
| | cgtcacatgcgtgaccgactccgtggtttaatcggcaacgaagaac | EDTIISDEYRKKLKEAFKHW |
| | tgccgattgaacgccaaagcggcatcttgaccgcgtactttcgtcgc | GTSKGKCLTDEEARQTGFY |
| | tggtggggtttccgcaaagaaaggggcaatgacattcatcacgcac | QRKNGNFMSLRCVEKGLG |
| | tggacgcggttattttagcgtctcgcaaggattacaccgacgacgg | WGQMVKINNNAFKTNVHNI |
| | cgttgatatgacgctgaagccgaaatccagcaacggtcgcgaattc | GVAVYLDEKGKKKAYEIKN |
| | gatccggaaaaacacctgccggataccatcagttttaaacgtgaca | PRLLKHFVEKPPEIKGKKLY |
| | aaagcaacaaaggcacgcctctgtacgacaagaatccgctttcgat | TLRRGDYVTHESEDSVYRIK |
| | taagaacagcttgattacccgtcgctggatggttactgaaatcgaac | KLGTSPVLEAVVKGSIGRIR |
| | atggtaaggaggataccattatttcggatgagtacagaaaaaagcta | NSASATKLTKVSKPQKVF |
| | aaagaagcatttaagcattggggcacgagcaaaggcaaatgcctg | (SEQ ID NO: 38) |
| | acggatgaagaggcgcgtcagaccggcttttatcagcgcaaaaac | |
| | gggaacttcatgtcactccgctgcgttgaaaaaggtctgggctggg | |
| | gacaaatggtgaaaatcaataacaacgcgttcaaaaccaatgtgca | |
| | taatattggtgtagcagtgtacctggatgagaagggtaagaaaaag | |
| | gcgtatgagatcaagaacccgcgtctgctcaaacacttcgtggaaa | |
| | aaccgccggagataaaaggcaagaagctctacacactgcgtcgtg | |
| | gcgattatgttacgcatgaaagtgaggacagcgtttaccgtatcaaa | |
| | aagctgggtacgtccccggttctcgaggccgtggttaaaggctctat | |
| | tggtagaatccgtaatagcgcatcggctaccaagttgactaaggtct | |
| | ccaaaccgcagaaagtgttc (SEQ ID NO: 37) | |
| N420A | atgaggaattgtaagaaagatctatatgtagaggttaccccgatgca | MRNCKKDLYVEVTPMQKE |
| (putative HNH- | gaaagagttagtcctgggtatcgattacggcggtaagtataccggg | LVLGIDYGGKYTGLAVVDR |
| knockout) | ctggcggtcgtggatcgtcgcaataaccaagttctgtacgcgcgta | RNNQVLYARTVKMRDDVA |
| | ccgtcaagatgcgtgacgatgtagctgatattttagagggtcgtcgg | DILEGRREQRGIRRTGQTKK |
| | gagcagcgtggtatccgtcgtaccggccagaccaagaagaaacgt | KRLRELKTYLKNIGGKYDE |
| | ctacgtgaattgaagacctatttgaaaaacattggcggtaagtacgca | SIGTFKNEPFMTVYKLCHKR |
| | cgaaagcatcggtacattcaagaacgaaccgtttatgaccgtgtaca | GYDYVEMPKEKTREEIEAM |
| | aactgtgtcataagcgtggctatgactacgttgagatgccgaaaga | SADEKKQWEKEKKEWEET |
| | gaagacccgtgaagagattgaggctatgagcgcggatgagaaga | ERNSRHRDEVTKDVRKIMS |
| | agcaatgggaaaaagagaaaaaggagtgggaagagaccgaacg | ERDATEQQISHVIKIFNKQY |
| | caactctcgtcaccgtgacgaggtaacgaaggacgttcgtaaaatc | RPKRFNNRILTKCKVEDCG |
| | atgagcgaagcgacgcgaccgaacagcagatcagccatgttatt | VNTPLRKNVRDLLIKDIVRF |
| | aaaatcttcaacaaacaatatcgtccaaagcgttttaacaaccgtatc | LPLTQSEKAELEAHVLDKE |
| | ctgaccaagtgcaaagttgaagattgtggtgtcaataccccgctgcg | GREKVKTFFRRHRINEFLRK |
| | taaaaacgtgcgtgacctgctgattaaggacatcgtgcgttttctgcc | QIYDIAFNQKLSGRTVFCKK |
| | gctgacgcagagcgaaaaggctgaattggaggcccacgtgctgg | HILEHEQHTKEERKVFRLAP |
| | acaaagaaggccgcgagaaggttaaaaccttcttccgacgccacc | SLKTKINNVLAVVKDEILPQ |
| | gcattaacgagttcctgcgcaagcagatttatgacatcgctttcaatc | FGINKVIMESNNFDIAARTK |
| | agaaactttctggtcgtaccgtcttctgcaaaaagcacattctggagc | GKKRLAKDEYGKGHKEAK |
| | acgaacaacacacgaaggaggaacgcaaggtgttccgcttggcg | ESLIEVLLRETKNQCVYCGK |
| | ccaagcttgaaaaccaagatcaataacgtgttggctgtggttaaaga | VITVTTANTDHIFPRKAGGI |
| | tgagatccttccgcaatttggtattaataaagtgattatggagtccaac | NVFANLVACCRECAEKKKG |
| | aattttgatatcgccgcacgtaccaagggcaaaaagagattagcaa | RTPLESGVLPQPEITAHLEN |
| | aagacgagtatggtaagggtcataaagaagcgaaggagtccctca | DLKKKILEDARNMNELNLN |
| | tcgaggtgctgctgagagaaaccaaaaaccaatgtgtttactgcggt | SIKYMSYASIGWRHMRDRL |
| | aaggtgatcaccgttacgaccgcgaacaccgaccacatctttccgc | RGLIGNEELPIERQSGILTAY |
| | gtaaggcgggtggtatcaacgtctttgcaaatctggttgcctgctgtc | FRRWWGFRKERGNDIHHAL |
| | gtgagtgcGCGgaaaaaaaaaaaggccgtaccccgctggagt | DAVILASRKDYTDDGVDMT |
| | ccggtgtgttgccacaaccggaaattactgcgcacctggagaacg | LKPKSSNGREFDPEKHLPDT |
| | atctgaagaagaaaattctggaggacgctcggaacatgaatgagtt | ISFKRDKSNKGTPLYDKNPL |
| | gaacctgaattcgatcaaatacatgagctatgccagcatcggctgg | SIKNSLITRRWMVTEIEHGK |
| | cgtcacatgcgtgaccgactccgtggtttaatcggcaacgaagaac | EDTIISDEYRKKLKEAFKHW |
| | tgccgattgaacgccaaagcggcatcttgaccgcgtactttcgtcgc | GTSKGKCLTDEEARQTGFY |
| | tggtggggtttccgcaaagaaaggggcaatgacattcatcacgcac | QRKNGNFMSLRCVEKGLG |
| | tggacgcggttattttagcgtctcgcaaggattacaccgacgacgg | WGQMVKINNNAFKTNVHNI |
| | cgttgatatgacgctgaagccgaaatccagcaacggtcgcgaattc | GVAVYLDEKGKKKAYEIKN |
| | gatccggaaaaacacctgccggataccatcagttttaaacgtgaca | PRLLKHFVEKPPEIKGKKLY |
| | aaagcaacaaaggcacgcctctgtacgacaagaatccgctttcgat | TLRRGDYVTHESEDSVYRIK |
| | taagaacagcttgattacccgtcgctggatggttactgaaatcgaac | KLGTSPVLEAVVKGSIGRIR |
| | atggtaaggaggataccattatttcggatgagtacagaaaaaagcta | NSASATKLTKVSKPQKVF |
| | aaagaagcatttaagcattggggcacgagcaaaggcaaatgcctg | (SEQ ID NO: 40) |
| | acggatgaagaggcgcgtcagaccggcttttatcagcgcaaaaac | |
| | gggaacttcatgtcactccgctgcgttgaaaaaggtctgggctggg | |
| | gacaaatggtgaaaatcaataacaacgcgttcaaaaccaatgtgca | |
| | taatattggtgtagcagtgtacctggatgagaagggtaagaaaaag | |
| | gcgtatgagatcaagaacccgcgtctgctcaaacacttcgtggaaa | |
| | aaccgccggagataaaaggcaagaagctctacacactgcgtcgtg | |
| | gcgattatgttacgcatgaaagtgaggacagcgtttaccgtatcaaa | |
| | aagctgggtacgtccccggttctcgaggccgtggttaaaggctctat | |
| | tggtagaatccgtaatagcgcatcggctaccaagttgactaaggtct | |
| | ccaaaccgcagaaagtgttc (SEQ ID NO: 39) | |
| D24A (putative | atgaggaattgtaagaaagatctatatgtagaggttaccccgatgca | MRNCKKDLYVEVTPMQKE |
| RuvC- | gaaagagttagtcctgggtatcGCGtacggcggtaagtataccg | LVLGIAYGGKYTGLAVVDR |
| knockout) | ggctggcggtcgtggatcgtcgcaataaccaagttctgtacgcgcg | RNNQVLYARTVKMRDDVA |
| | taccgtcaagatgcgtgacgatgtagctgatattttagagggtcgtc | DILEGRREQRGIRRTGQTKK |
| | gggagcagcgtggtatccgtcgtaccggccagaccaagaagaaa | KRLRELKTYLKNIGGKYDE |
| | cgtctacgtgaattgaagacctatttgaaaaacattggcggtaagtac | SIGTFKNEPFMTVYKLCHKR |
| | gacgaaagcatcggtacattcaagaacgaaccgtttatgaccgtgt | GYDYVEMPKEKTREEIEAM |
| | acaaactgtgtcataagcgtggctatgactacgttgagatgccgaaa | SADEKKQWEKEKKEWEET |
| | gagaagacccgtgaagagattgaggctatgagcgcggatgagaa | ERNSRHRDEVTKDVRKIMS |
| | gaagcaatgggaaaaagagaaaaaggagtgggaagagaccgaa | ERDATEQQISHVIKIFNKQY |
| | cgcaactctcgtcaccgtgacgaggtaacgaaggacgttcgtaaaa | RPKRFNNRILTKCKVEDCG |
| | tcatgagcgaacgcgacgcgaccgaacagcagatcagccatgtta | VNTPLRKNVRDLLIKDIVRF |
| | ttaaaatcttcaacaaacaatatcgtccaaagcgttttaacaaccgtat | LPLTQSEKAELEAHVLDKE |
| | cctgaccaagtgcaaagttgaagattgtggtgtcaataccccgctgc | GREKVKTFFRRHRINEFLRK |
| | gtaaaaacgtgcgtgacctgctgattaaggacatcgtgcgttttctgc | QIYDIAFNQKLSGRTVFCKK |
| | cgctgacgcagagcgaaaaggctgaattggaggcccacgtgctg | HILEHEQHTKEERKVFRLAP |
| | gacaaagaaggccgcgagaaggttaaaaccttcttccgacgccac | SLKTKINNVLAVVKDEILPQ |
| | cgcattaacgagttcctgcgcaagcagatttatgacatcgctttcaat | FGINKVIMESNNFDIAARTK |
| | cagaaactttctggtcgtaccgtcttctgcaaaaagcacattctggag | GKKRLAKDEYGKGHKEAK |
| | cacgaacaacacacgaaggaggaacgcaaggtgttccgcttggc | ESLIEVLLRETKNQCVYCGK |
| | gccaagcttgaaaaccaagatcaataacgtgttggctgtggttaaag | VITVTTANTDHIFPRKAGGI |
| | atgagatccttccgcaatttggtattaataaagtgattatggagtccaa | NVFANLVACCRECNEKKKG |
| | caattttgatatcgccgcacgtaccaagggcaaaaagagattagca | RTPLESGVLPQPEITAHLEN |
| | aaagacgagtatggtaagggtcataaagaagcgaaggagtccctc | DLKKKILEDARNMNELNLN |
| | atcgaggtgctgctgagagaaaccaaaaaccaatgtgtttactgcg | SIKYMSYASIGWRHMRDRL |
| | gtaaggtgatcaccgttacgaccgcgaacaccgaccacatctttcc | RGLIGNEELPIERQSGILTAY |
| | gcgtaaggcgggtggtatcaacgtctttgcaaatctggttgcctgct | FRRWWGFRKERGNDIHHAL |
| | gtcgtgagtgcaatgaaaaaaaaaaaggccgtaccccgctggagt | DAVILASRKDYTDDGVDMT |
| | ccggtgtgttgccacaaccggaaattactgcgcacctggagaacg | LKPKSSNGREFDPEKHLPDT |
| | atctgaagaagaaaattctggaggacgctcggaacatgaatgagtt | ISFKRDKSNKGTPLYDKNPL |
| | gaacctgaattcgatcaaatacatgagctatgccagcatcggctgg | SIKNSLITRRWMVTEIEHGK |
| | cgtcacatgcgtgaccgactccgtggtttaatcggcaacgaagaac | EDTIISDEYRKKLKEAFKHW |
| | tgccgattgaacgccaaagcggcatcttgaccgcgtactttcgtcgc | GTSKGKCLTDEEARQTGFY |
| | tggtggggtttccgcaaagaaaggggcaatgacattcatcacgcac | QRKNGNFMSLRCVEKGLG |
| | tggacgcggttattttagcgtctcgcaaggattacaccgacgacgg | WGQMVKINNNAFKTNVHNI |
| | cgttgatatgacgctgaagccgaaatccagcaacggtcgcgaattc | GVAVYLDEKGKKKAYEIKN |
| | gatccggaaaaacacctgccggataccatcagttttaaacgtgaca | PRLLKHFVEKPPEIKGKKLY |
| | aaagcaacaaaggcacgcctctgtacgacaagaatccgctttcgat | TLRRGDYVTHESEDSVYRIK |
| | taagaacagcttgattacccgtcgctggatggttactgaaatcgaac | KLGTSPVLEAVVKGSIGRIR |
| | atggtaaggaggataccattatttcggatgagtacagaaaaaagcta | NSASATKLTKVSKPQKVF |
| | aaagaagcatttaagcattggggcacgagcaaaggcaaatgcctg | (SEQ ID NO: 42) |
| | acggatgaagaggcgcgtcagaccggcttttatcagcgcaaaaac | |
| | gggaacttcatgtcactccgctgcgttgaaaaaggtctgggctggg | |
| | gacaaatggtgaaaatcaataacaacgcgttcaaaaccaatgtgca | |
| | taatattggtgtagcagtgtacctggatgagaagggtaagaaaaag | |
| | gcgtatgagatcaagaacccgcgtctgctcaaacacttcgtggaaa | |
| | aaccgccggagataaaaggcaagaagctctacacactgcgtcgtg | |
| | gcgattatgttacgcatgaaagtgaggacagcgtttaccgtatcaaa | |
| | aagctgggtacgtccccggttctcgaggccgtggttaaaggctctat | |
| | tggtagaatccgtaatagcgcatcggctaccaagttgactaaggtct | |
| | ccaaaccgcagaaagtgttc (SEQ ID NO: 41) | |
| E337A (putative | atgaggaattgtaagaaagatctatatgtagaggttaccccgatgca | MRNCKKDLYVEVTPMQKE |
| RuvC- knockout) | gaaagagttagtcctgggtatcgattacggcggtaagtataccggg | LVLGIDYGGKYTGLAVVDR |
| | ctggcggtcgtggatcgtcgcaataaccaagttctgtacgcgcgta | RNNQVLYARTVKMRDDVA |
| | ccgtcaagatgcgtgacgatgtagctgatattttagagggtcgtcgg | DILEGRREQRGIRRTGQTKK |
| | gagcagcgtggtatccgtcgtaccggccagaccaagaagaaacgt | KRLRELKTYLKNIGGKYDE |
| | ctacgtgaattgaagacctatttgaaaaacattggcggtaagtacga | SIGTFKNEPFMTVYKLCHKR |
| | cgaaagcatcggtacattcaagaacgaaccgtttatgaccgtgtaca | GYDYVEMPKEKTREEIEAM |
| | aactgtgtcataagcgtggctatgactacgttgagatgccgaaaga | SADEKKQWEKEKKEWEET |
| | gaagacccgtgaagagattgaggctatgagcgcggatgagaaga | ERNSRHRDEVTKDVRKIMS |
| | agcaatgggaaaaagagaaaaaggagtgggaagagaccgaacg | ERDATEQQISHVIKIFNKQY |
| | caactctcgtcaccgtgacgaggtaacgaaggacgttcgtaaaatc | RPKRFNNRILTKCKVEDCG |
| | atgagcgaacgcgacgcgaccgaacagcagatcagccatgttatt | VNTPLRKNVRDLLIKDIVRF |
| | aaaatcttcaacaaacaatatcgtccaaagcgttttaacaaccgtatc | LPLTQSEKAELEAHVLDKE |
| | ctgaccaagtgcaaagttgaagattgtggtgtcaataccccgctgcg | GREKVKTFFRRHRINEFLRK |
| | taaaaacgtgcgtgacctgctgattaaggacatcgtgcgttttctgcc | QIYDIAFNQKLSGRTVFCKK |
| | gctgacgcagagcgaaaaggctgaattggaggcccacgtgctgg | HILEHEQHTKEERKVFRLAP |
| | acaaagaaggccgcgagaaggttaaaaccttcttccgacgccacc | SLKTKINNVLAVVKDEILPQ |
| | gcattaacgagttcctgcgcaagcagatttatgacatcgctttcaatc | FGINKVIMASNNFDIAARTK |
| | agaaactttctggtcgtaccgtcttctgcaaaaagcacattctggagc | GKKRLAKDEYGKGHKEAK |
| | acgaacaacacacgaaggaggaacgcaaggtgttccgcttggcg | ESLIEVLLRETKNQCVYCGK |
| | ccaagcttgaaaaccaagatcaataacgtgttggctgtggttaaaga | VITVTTANTDHIFPRKAGGI |
| | tgagatccttccgcaatttggtattaataaagtgattatgGCGtcca | NVFANLVACCRECNEKKKG |
| | acaattttgatatcgccgcacgtaccaagggcaaaaagagattagc | RTPLESGVLPQPEITAHLEN |
| | aaaagacgagtatggtaagggtcataaagaagcgaaggagtccct | DLKKKILEDARNMNELNLN |
| | catcgaggtgctgctgagagaaaccaaaaaccaatgtgtttactgc | SIKYMSYASIGWRHMRDRL |
| | ggtaaggtgatcaccgttacgaccgcgaacaccgaccacatctttc | RGLIGNEELPIERQSGILTAY |
| | cgcgtaaggcgggtggtatcaacgtctttgcaaatctggttgcctgc | FRRWWGFRKERGNDIHHAL |
| | tgtcgtgagtgcaatgaaaaaaaaaaaggccgtaccccgctggag | DAVILASRKDYTDDGVDMT |
| | tccggtgtgttgccacaaccggaaattactgcgcacctggagaacg | LKPKSSNGREFDPEKHLPDT |
| | atctgaagaagaaaattctggaggacgctcggaacatgaatgagtt | ISFKRDKSNKGTPLYDKNPL |
| | gaacctgaattcgatcaaatacatgagctatgccagcatcggctgg | SIKNSLITRRWMVTEIEHGK |
| | cgtcacatgcgtgaccgactccgtggtttaatcggcaacgaagaac | EDTIISDEYRKKLKEAFKHW |
| | tgccgattgaacgccaaagcggcatcttgaccgcgtactttcgtcgc | GTSKGKCLTDEEARQTGFY |
| | tggtggggtttccgcaaagaaaggggcaatgacattcatcacgcac | QRKNGNFMSLRCVEKGLG |
| | tggacgcggttattttagcgtctcgcaaggattacaccgacgacgg | WGQMVKINNNAFKTNVHNI |
| | cgttgatatgacgctgaagccgaaatccagcaacggtcgcgaattc | GVAVYLDEKGKKKAYEIKN |
| | gatccggaaaaacacctgccggataccatcagttttaaacgtgaca | PRLLKHFVEKPPEIKGKKLY |
| | aaagcaacaaaggcacgcctctgtacgacaagaatccgctttcgat | TLRRGDYVTHESEDSVYRIK |
| | taagaacagcttgattacccgtcgctggatggttactgaaatcgaac | KLGTSPVLEAVVKGSIGRIR |
| | atggtaaggaggataccattatttcggatgagtacagaaaaaagcta | NSASATKLTKVSKPQKVF |
| | aaagaagcatttaagcattggggcacgagcaaaggcaaatgcctg | (SEQ ID NO: 44) |
| | acggatgaagaggcgcgtcagaccggcttttatcagcgcaaaaac | |
| | gggaacttcatgtcactccgctgcgttgaaaaaggtctgggctggg | |
| | gacaaatggtgaaaatcaataacaacgcgttcaaaaccaatgtgca | |
| | taatattggtgtagcagtgtacctggatgagaagggtaagaaaaag | |
| | gcgtatgagatcaagaacccgcgtctgctcaaacacttcgtggaaa | |
| | aaccgccggagataaaaggcaagaagctctacacactgcgtcgtg | |
| | gcgattatgttacgcatgaaagtgaggacagcgtttaccgtatcaaa | |
| | aagctgggtacgtccccggttctcgaggccgtggttaaaggctctat | |
| | tggtagaatccgtaatagcgcatcggctaccaagttgactaaggtct | |
| | ccaaaccgcagaaagtgttc (SEQ ID NO: 43) | |
| D524A | atgaggaattgtaagaaagatctatatgtagaggttaccccgatgca | MRNCKKDLYVEVTPMQKE |
| (putative RuvC- | gaaagagttagtcctgggtatcgattacggcggtaagtataccggg | LVLGIDYGGKYTGLAVVDR |
| knockout) | ctggcggtcgtggatcgtcgcaataaccaagttctgtacgcgcgta | RNNQVLYARTVKMRDDVA |
| | ccgtcaagatgcgtgacgatgtagctgatattttagagggtcgtcgg | DILEGRREQRGIRRTGQTKK |
| | gagcagcgtggtatccgtcgtaccggccagaccaagaagaaacgt | KRLRELKTYLKNIGGKYDE |
| | ctacgtgaattgaagacctatttgaaaaacattggcggtaagtacga | SIGTFKNEPFMTVYKLCHKR |
| | cgaaagcatcggtacattcaagaacgaaccgtttatgaccgtgtaca | GYDYVEMPKEKTREEIEAM |
| | aactgtgtcataagcgtggctatgactacgttgagatgccgaaaga | SADEKKQWEKEKKEWEET |
| | gaagacccgtgaagagattgaggctatgagcgcggatgagaaga | ERNSRHRDEVTKDVRKIMS |
| | agcaatgggaaaaagagaaaaaggagtgggaagagaccgaacg | ERDATEQQISHVIKIFNKQY |
| | caactctcgtcaccgtgacgaggtaacgaaggacgttcgtaaaatc | RPKRFNNRILTKCKVEDCG |
| | atgagcgaacgcgacgcgaccgaacagcagatcagccatgttatt | VNTPLRKNVRDLLIKDIVRF |
| | aaaatcttcaacaaacaatatcgtccaaagcgttttaacaaccgtatc | LPLTQSEKAELEAHVLDKE |
| | ctgaccaagtgcaaagttgaagattgtggtgtcaataccccgctgcg | GREKVKTFFRRHRINEFLRK |
| | taaaaacgtgcgtgacctgctgattaaggacatcgtgcgttttctgcc | QIYDIAFNQKLSGRTVFCKK |
| | gctgacgcagagcgaaaaggctgaattggaggcccacgtgctgg | HILEHEQHTKEERKVFRLAP |
| | acaaagaaggccgcgagaaggttaaaaccttcttccgacgccacc | SLKTKINNVLAVVKDEILPQ |
| | gcattaacgagttcctgcgcaagcagatttatgacatcgctttcaatc | FGINKVIMESNNFDIAARTK |
| | agaaactttctggtcgtaccgtcttctgcaaaaagcacattctggagc | GKKRLAKDEYGKGHKEAK |
| | acgaacaacacacgaaggaggaacgcaaggtgttccgcttggcg | ESLIEVLLRETKNQCVYCGK |
| | ccaagcttgaaaaccaagatcaataacgtgttggctgtggttaaaga | VITVTTANTDHIFPRKAGGI |
| | tgagatccttccgcaatttggtattaataaagtgattatggagtccaac | NVFANLVACCRECNEKKKG |
| | aattttgatatcgccgcacgtaccaagggcaaaaagagattagcaa | RTPLESGVLPQPEITAHLEN |
| | aagacgagtatggtaagggtcataaagaagcgaaggagtccctca | DLKKKILEDARNMNELNLN |
| | tcgaggtgctgctgagagaaaccaaaaaccaatgtgtttactgcggt | SIKYMSYASIGWRHMRDRL |
| | aaggtgatcaccgttacgaccgcgaacaccgaccacatctttccgc | RGLIGNEELPIERQSGILTAY |
| | gtaaggcgggtggtatcaacgtctttgcaaatctggttgcctgctgtc | FRRWWGFRKERGNDIHHAL |
| | gtgagtgcaatgaaaaaaaaaaaggccgtaccccgctggagtccg | AAVILASRKDYTDDGVDMT |
| | gtgtgttgccacaaccggaaattactgcgcacctggagaacgatct | LKPKSSNGREFDPEKHLPDT |
| | gaagaagaaaattctggaggacgctcggaacatgaatgagttgaa | ISFKRDKSNKGTPLYDKNPL |
| | cctgaattcgatcaaatacatgagctatgccagcatcggctggcgtc | SIKNSLITRRWMVTEIEHGK |
| | acatgcgtgaccgactccgtggtttaatcggcaacgaagaactgcc | EDTIISDEYRKKLKEAFKHW |
| | gattgaacgccaaagcggcatcttgaccgcgtactttcgtcgctggt | GTSKGKCLTDEEARQTGFY |
| | ggggtttccgcaaagaaaggggcaatgacattcatcacgcatgG | QRKNGNFMSLRCVEKGLG |
| | CGgcggttattttagcgtctcgcaaggattacaccgacgacggcg | WGQMVKINNNAFKTNVHNI |
| | ttgatatgacgctgaagccgaaatccagcaacggtcgcgaattcga | GVAVYLDEKGKKKAYEIKN |
| | tccggaaaaacacctgccggataccatcagttttaaacgtgacaaa | PRLLKHFVEKPPEIKGKKLY |
| | agcaacaaaggcacgcctctgtacgacaagaatccgctttcgatta | TLRRGDYVTHESEDSVYRIK |
| | agaacagcttgattacccgtcgctggatggttactgaaatcgaacat | KLGTSPVLEAVVKGSIGRIR |
| | ggtaaggaggataccattatttcggatgagtacagaaaaaagctaa | NSASATKLTKVSKPQKVF |
| | aagaagcatttaagcattggggcacgagcaaaggcaaatgcctga | (SEQ ID NO: 46) |
| | cggatgaagaggcgcgtcagaccggcttttatcagcgcaaaaacg | |
| | ggaacttcatgtcactccgctgcgttgaaaaaggtctgggctgggg | |
| | acaaatggtgaaaatcaataacaacgcgttcaaaaccaatgtgcata | |
| | atattggtgtagcagtgtacctggatgagaagggtaagaaaaaggc | |
| | gtatgagatcaagaacccgcgtctgctcaaacacttcgtggaaaaa | |
| | ccgccggagataaaaggcaagaagctctacacactgcgtcgtggc | |
| | gattatgttacgcatgaaagtgaggacagcgtttaccgtatcaaaaa | |
| | gctgggtacgtccccggttctcgaggccgtggttaaaggctctattg | |
| | gtagaatccgtaatagcgcatcggctaccaagttgactaaggtctcc | |
| | aaaccgcagaaagtgttc (SEQ ID NO: 45) | |

A linear DNA template sequence encoding an RNA guide was designed and ordered (IDT) with a T7 promoter upstream and a T7Te terminator sequence downstream of the guide. The RNA guide was designed to be specific to a previously tested target sequence, described in Example 1, within the coding exon of AAVS1 with a 5′-NGG-3′ PAM sequence (the PAM is 3′ of the target sequence). The sequence of the encoded RNA guide and its individual components are shown in Table 12. The T7 promoter uses a +1 G at the start of the transcript (i.e., the 5′ end of the RNA) for more efficient transcription. This +1 G is shown for SEQ ID NO: 49.

TABLE 12
RNA Sequences

| Components | RNA sequence | SEQ ID NO |
|---|---|---|
| Reference | See Table 2 | 2 |
| sgRNA scaffold | | |
| AAVS1-T3 | UUGUGAGAAUGGUGCGUCCU | 47 |
| spacer | | |
| T7Te | ggcucaccuucgggugggccuuucugcg | 48 |
| terminator | | |
| T7 transcribed | gUUGUGAGAAUGGUGCGUCCUguuacaguuaaggcucuu | 49 |
| AAVS1-T3 | uggaaacaaagaagccuuaauuguaaaacgccuauaugguaaagugaugu | |
| sgRNA | acguuuggguauauaucgccagccugaaccucuacgccagaaauggcagc | |
| | uuuaucauggguuaggacgauauuuaaaaaacuuucugeggcuugcuua | |
| | cuuuaguaagcuuuguggcugaggcagaauuccuuggcucaccuucggg | |
| | ugggccuuucugcg | |

A DNA target was designed and ordered as a synthesized linear DNA fragment. The target sequence from AAVS1 and 10 bases upstream and downstream within the exon was flanked by 100 bases of unrelated sequence upstream and 200 bases of unrelated sequence downstream. The extra sequence was added so that the cleaved and uncleaved products would separate well on a gel. The target and non-target strands were labelled with 5′ IR800 and 5′ IR700 labels, respectively, through PCR amplification using labelled primers. The sequences of the DNA target, the individual components of the DNA target, and the labelled PCR primers are in Table 13.

TABLE 13
Target gBlock and Primer Sequences

| Components | DNA sequence | SEQ ID NO |
|---|---|---|
| AAVS1-T3 target | See Table 2 | 14 |
| AAVS1-T3 | AAAACTCCCT | 50 |
| upstream 10 nt | | |
| AAVS1-T3 | AGGTGTTCAC | 51 |
| downstream 10 nt | | |
| 100 nt upstream | gctcaaagctttagactggaggtaccgtagtactatgagcttaatcgcgctgag | 52 |
| sequence | ggtgatttgggtgttcgtctcatatcttcgattatcggacagtagt | |
| 200 nt | agtagtatcggaccggttggcgaaccacagcgtataacgagacatggactcg | 53 |
| downstream | cattccctaccttaagcgggcggttttggagctcggaattcatgtaaactcgccc | |
| sequence | gtttaacataatctagatggatcttgcgcgcaccgcccgaacaccactcgccac | |
| | aaaaaaccgccggaacgtccaaaagtacgggttttgctg | |
| Full target gblock | gctcaaagctttagactggaggtaccgtagtactatgagcttaatcgcgctgag | 54 |
| | ggtgatttgggtgttcgtctcatatcttcgattatcggacagtagtAAAACTC | |
| | CCTTTGTGAGAATGGTGCGTCCTAGGTGTTCACag | |
| | tagtatcggaccggttggcgaaccacagcgtataacgagacatggactcgcat | |
| | tccctaccttaagcgggcggttttggagctcggaattcatgtaaactcgcccgtt | |
| | taacataatctagatggatcttgcgcgcaccgcccgaacaccactcgccacaa | |
| | aaaaccgccggaacgtccaaaagtacgggttttgctg | |
| Forward primer | /5IRD700/GCTCAAAGCTTTAGACTGGAGGTACCG | 55 |
| Reverse primer | /5IRD800/CAGCAAAACCCGTACTTTTGGACG | 56 |

Cleavage activity of Nuclease A (SEQ ID NO: 1) and each of the putative nickases was assessed using in vitro cleavage assays. Each polypeptide was individually co-expressed with the RNA guide in vitro by incubating the plasmid encoding the protein of interest from Table 11 and linear DNA template for the T7 transcribed AAVS1-T3 sgRNA from Table 12 in a PURExpress® solution (NEB) containing SUPERase. In™ RNase Inhibitor (Invitrogen) for 2 hours at 37° C. The unpurified polypeptide/RNA solution was then diluted into a solution of 1X NEB Buffer 2 (NEB) containing approximately 1 ng/μl of the labelled DNA target amplicon. The solution was then incubated for 1 hour at 37° C. Reactions were stopped by incubating with RNase Cocktail™ (Invitrogen; approximately 1 U/μl final concentration) at 37° C. for 15 minutes, followed by incubating with Proteinase K (NEB; approximately 0.04 U/μl final concentration) at 55° C. for 30 minutes. The DNA was then purified using CleanNGS DNA & RNA Clean-Up Magnetic Beads (Bulldog Bio).

The cleaved and uncleaved products of the target and non-target strands were separated by running the samples on a 10% TBE-Urea PAGE gel. The gel was imaged using a LI-COR Odysssey M imaging system using the 800 nm and 700 nm channels to visualize the 5′ IR800 and 5′ IR700 labels on the target and non-target strands of the target DNA substrate. Band intensities were quantified using ImageJ software.

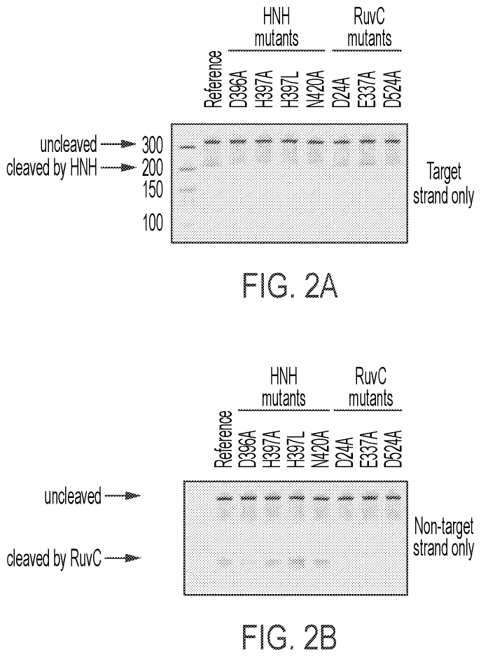

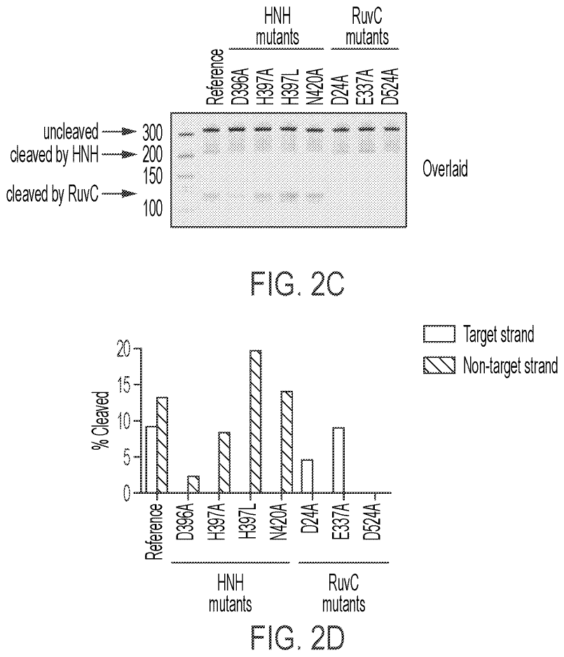

Gel images are shown in FIGS. 2A-2C, and quantification of the percent of cleaved target and non-target strands are shown in FIG. 2D. The uncleaved, HNH-cleaved, and RuvC-cleaved strands are indicated. FIG. 2A is a gel image captured using the 800 nm channel showing cleavage of the target strand. FIG. 2B is a gel image captured using the 700 nm channel showing cleavage of the non-target strand. FIG. 2C is an overlay of the gel images from FIG. 2A and FIG. 2B. As shown in FIGS. 2A-2D, Nuclease A (SEQ ID NO: 1) cleaved both the target strand and the non-target strand, as expected. Each of the four HNH-knockout nickase constructs (D396A, H397A, H397L, and N420A) showed significantly decreased activity on the target strand while retaining activity on the non-target strand. Two of the three RuvC-knockout nickase constructs (D24A and E337A) showed significantly decreased activity on the non-target strand while retaining activity on the target strand (FIGS. 2A-2D).

This Example thus shows that HNH-knockout nickases and RuvC-knockout nickases were successfully engineered.

Additional variant nuclease polypeptide derived from Nuclease A are provided in Table 14 below, each of which is also within the scope of the present disclosure.

TABLE 14
Variant CRISPR Nuclease Polypeptides

| Nuclease | | |
|---|---|---|
| polypeptide | Nucleotide Sequence | Amino Acid Sequence |
| I67R, D568R, | atgaaacggacagccgacggaagcgagttcgagtcaccaa | MKRTADGSEFESPKKKR |
| W593R, E706R | agaagaagcggaaagtccggaactgcaagaaggacctgta | KVRNCKKDLYVEVTPM |
| | cgtggaagtgacccctatgcagaaagagctggtgctgggc | QKELVLGIDYGGKYTGL |
| | attgactacggcggaaagtacacaggcctggccgtggtgg | AVVDRRNNQVLYARTV |
| | accggagaaacaaccaggtgctgtacgccagaaccgtcaa | KMRDDVADILEGRREQR |
| | gatgagagacgacgtggccgatatcctcgaaggccggagg | GRRRTGQTKKKRLRELK |
| | gaacagcgaggcAGAagaagaacaggccagacaaag | TYLKNIGGKYDESIGTFK |
| | aagaagagactgagagaactgaaaacctacctgaagaaca | NEPFMTVYKLCHKRGYD |
| | tcggaggaaaatatgacgagtctatcggcacattcaaaaac | YVEMPKEKTREEIEAMS |
| | gagcctttcatgacagtgtacaagctgtgtcacaagagaggc | ADEKKQWEKEKKEWEE |
| | tacgactacgtggaaatgcctaaggaaaagaccagagaag | TERNSRHRDEVTKDVRKI |
| | aaatcgaggccatgtctgccgacgagaaaaagcagtggga | MSERDATEQQISHVIKIF |
| | aaaagagaaaaaggagtgggaggaaaccgagcggaactc | NKQYRPKRFNNRILTKC |
| | cagacaccgggatgaggtgaccaaggacgtgcggaagat | KVEDCGVNTPLRKNVRD |
| | catgagcgagagagatgccaccgagcagcagatcagcca | LLIKDIVRFLPLTQSEKAE |
| | cgtgatcaagatcttcaacaagcagtaccggcccaagcggt | LEAHVLDKEGREKVKTF |
| | tcaataatagaatcctgacaaaatgtaaggttgaggattgcg | FRRHRINEFLRKQIYDIAF |
| | gcgtgaacacacctctgagaaagaacgtcagagacctgctg | NQKLSGRTVFCKKHILEH |
| | atcaaggacatcgtgagattcctgcccctgacccagagcga | EQHTKEERKVFRLAPSLK |
| | gaaagccgagctggaagctcatgtgctggataaggaagga | TKINNVLAVVKDEILPQF |
| | agagagaaggtgaagaccttcttcagacggcacagaatcaa | GINKVIMESNNFDIAART |
| | cgagttcctgcgcaagcagatctacgacatcgcctttaacca | KGKKRLAKDEYGKGHK |
| | aaagctgagcggcagaaccgtgttctgcaagaagcacatcc | EAKESLIEVLLRETKNQC |
| | tggagcacgagcaacacaccaaagaggaacgcaaggtctt | VYCGKVITVTTANTDHIF |
| | tagactggcccctagcctgaaaactaagattaacaacgtgct | PRKAGGINVFANLVACC |
| | ggctgtggttaaggacgagatcctgccccagttcggcatca | RECNEKKKGRTPLESGV |
| | acaaggtcatcatggaatccaacaacttcgatattgccgcta | LPQPEITAHLENDLKKKI |
| | gaaccaagggcaagaagcggctggccaaggatgagtacg | LEDARNMNELNLNSIKY |
| | gcaagggccacaaggaggccaaagagagcctgatcgagg | MSYASIGWRHMRDRLRG |
| | tgctgctgcgggaaaccaagaaccagtgcgtgtactgcgga | LIGNEELPIERQSGILTAY |
| | aaggtgatcaccgtgaccaccgccaataccgaccacatctt | FRRWWGFRKERGNDIHH |
| | ccccagaaaggcgggggcatcaacgttttcgccaacctg | ALDAVILASRKDYTDDG |
| | gtggcctgttgtagagagtgcaacgagaagaagaagggca | VDMTLKPKSSNGREFDP |
| | gaacacctctggaatccggcgtgcttccacagcctgagatc | EKHLPDTISFKRRKSNKG |
| | accgcccacctggaaaacgacctcaaaaagaagatccttga | TPLYDKNPLSIKNSLITRR |
| | ggacgccagaaatatgaacgagctgaatctgaacagcatca | RMVTEIEHGKEDTIISDE |
| | agtacatgagctacgccagtatcggatggcggcacatgaga | YRKKLKEAFKHWGTSKG |
| | gatagactgaggggccttatcggtaatgaagaactgcctatc | KCLTDEEARQTGFYQRK |
| | gagcgccaaagcggcatcctgacagcttacttcagacggtg | NGNFMSLRCVEKGLGW |
| | gtggggatttcggaaggaacggggaaatgacattcaccac | GQMVKINNNAFKTNVHN |
| | gccctggacgctgtgatcctggccagcagaaaagactatac | IGVAVYLDEKGKKKAYE |
| | agatgacggcgtggatatgaccctgaagcccaaaagcagc | IKNPRLLKHFVRKPPEIK |
| | aacggccgagaattcgaccccgagaaacatctgcctgatac | GKKLYTLRRGDYVTHES |
| | aatcagcttcaaaagaAGAaagtccaacaaaggaacccc | EDSVYRIKKLGTSPVLEA |
| | tctgtatgataagaacccattgtctatcaagaattctctgatcac | VVKGSIGRIRNSASATKL |
| | aagaaggCGGatggtgacagagatcgaacacggcaag | TKVSKPQKVFSGGSSGGS |
| | gaggacaccatcatcagcgacgagtacagaaagaagctga | SGSETPGTSESATPESSGG |
| | aggaggctttcaaacactggggcaccagcaagggcaagtg | SSGGSSKRPAATKKAGQ |
| | cctgaccgacgaggaagccagacagaccggcttctaccag | AKKKK |
| | agaaagaatggcaacttcatgagcctgcggtgcgtggagaa | (SEQ ID NO: 58) |
| | gggcctgggctggggccagatggtgaagatcaataacaac | |
| | gcctttaagacgaacgtgcacaacatcggggtggctgtttac | |
| | ctggacgagaagggcaagaagaaggcctacgaaattaaga | |
| | accccagactgctgaaacatttcgtgAGAaagccacctg | |
| | agatcaagggaaagaagctgtacaccctgcggagaggcg | |
| | actacgtgacccacgagagcgaggactccgtgtacagaatc | |
| | aaaaaactgggcacaagccctgtgctggaagctgtggtcaa | |
| | gggctctatcggccgcatccggaacagcgccagcgccaca | |
| | aaactgaccaaggtgtccaagcctcagaaggtgttttctgga | |
| | ggatctagcggaggatcctctggcagcgagacaccaggaa | |
| | caagcgagtcagcaacaccagagagcagtggcggcagca | |
| | gcggcggcagcagcaaaaggccggcggccacgaaaaag | |
| | gccggccaggcaaaaaagaaaaag | |
| | (SEQ ID NO: 57) | |
| Truncated (16- | atgaaacggacagccgacggaagcgagttcgagtcaccaa | MKRTADGSEFESPKKKR |
| 775) Reference | agaagaagcggaaagtccagaaagagctggtgctgggcat | KVQKELVLGIDYGGKYT |
| | tgactacggcggaaagtacacaggcctggccgtggtggac | GLAVVDRRNNQVLYART |
| | cggagaaacaaccaggtgctgtacgccagaaccgtcaaga | VKMRDDVADILEGRREQ |
| | tgagagacgacgtggccgatatcctcgaaggccggaggga | RGIRRTGQTKKKRLRELK |
| | acagcgaggcattagaagaacaggccagacaaagaagaa | TYLKNIGGKYDESIGTFK |
| | gagactgagagaactgaaaacctacctgaagaacatcgga | NEPFMTVYKLCHKRGYD |
| | ggaaaatatgacgagtctatcggcacattcaaaaacgagcct | YVEMPKEKTREEIEAMS |
| | ttcatgacagtgtacaagctgtgtcacaagagaggctacgac | ADEKKQWEKEKKEWEE |
| | tacgtggaaatgcctaaggaaaagaccagagaagaaatcg | TERNSRHRDEVTKDVRKI |
| | aggccatgtctgccgacgagaaaaagcagtgggaaaaaga | MSERDATEQQISHVIKIF |
| | gaaaaaggagtgggaggaaaccgagcggaactccagaca | NKQYRPKRFNNRILTKC |
| | ccgggatgaggtgaccaaggacgtgcggaagatcatgagc | KVEDCGVNTPLRKNVRD |
| | gagagagatgccaccgagcagcagatcagccacgtgatca | LLIKDIVRFLPLTQSEKAE |
| | agatcttcaacaagcagtaccggcccaagcggttcaataata | LEAHVLDKEGREKVKTF |
| | gaatcctgacaaaatgtaaggttgaggattgcggcgtgaac | FRRHRINEFLRKQIYDIAF |
| | acacctctgagaaagaacgtcagagacctgctgatcaagga | NQKLSGRTVFCKKHILEH |
| | catcgtgagattcctgcccctgacccagagcgagaaagccg | EQHTKEERKVFRLAPSLK |
| | agctggaagctcatgtgctggataaggaaggaagagagaa | TKINNVLAVVKDEILPQF |
| | ggtgaagaccttcttcagacggcacagaatcaacgagttcct | GINKVIMESNNFDIAART |
| | gcgcaagcagatctacgacatcgcctttaaccaaaagctga | KGKKRLAKDEYGKGHK |
| | gcggcagaaccgtgttctgcaagaagcacatcctggagca | EAKESLIEVLLRETKNQC |
| | cgagcaacacaccaaagaggaacgcaaggtctttagactg | VYCGKVITVTTANTDHIF |
| | gcccctagcctgaaaactaagattaacaacgtgctggctgtg | PRKAGGINVFANLVACC |
| | gttaaggacgagatcctgccccagttcggcatcaacaaggt | RECNEKKKGRTPLESGV |
| | catcatggaatccaacaacttcgatattgccgctagaaccaa | LPQPEITAHLENDLKKKI |
| | gggcaagaagcggctggccaaggatgagtacggcaaggg | LEDARNMNELNLNSIKY |
| | ccacaaggaggccaaagagagcctgatcgaggtgctgctg | MSYASIGWRHMRDRLRG |
| | cgggaaaccaagaaccagtgcgtgtactgcggaaaggtga | LIGNEELPIERQSGILTAY |
| | tcaccgtgaccaccgccaataccgaccacatcttccccaga | FRRWWGFRKERGNDIHH |
| | aagggggcggcatcaacgttttcgccaacctggtggcctg | ALDAVILASRKDYTDDG |
| | ttgtagagagtgcaacgagaagaagaagggcagaacacct | VDMTLKPKSSNGREFDP |
| | ctggaatccggcgtgcttccacagcctgagatcaccgccca | EKHLPDTISFKRDKSNKG |
| | cctggaaaacgacctcaaaaagaagatccttgaggacgcc | TPLYDKNPLSIKNSLITRR |
| | agaaatatgaacgagctgaatctgaacagcatcaagtacatg | WMVTEIEHGKEDTIISDE |
| | agctacgccagtatcggatggcggcacatgagagatagact | YRKKLKEAFKHWGTSKG |
| | gaggggccttatcggtaatgaagaactgcctatcgagcgcc | KCLTDEEARQTGFYQRK |
| | aaagcggcatcctgacagcttacttcagacggtggtgggga | NGNFMSLRCVEKGLGW |
| | tttcggaaggaacggggaaatgacattcaccacgccctgga | GQMVKINNNAFKTNVHN |
| | cgctgtgatcctggccagcagaaaagactatacagatgacg | IGVAVYLDEKGKKKAYE |
| | gcgtggatatgaccctgaagcccaaaagcagcaacggccg | IKNPRLLKHFVEKPPEIKG |
| | agaattcgaccccgagaaacatctgcctgatacaatcagctt | KKLYTLRRGDYVTHESE |
| | caaaagagataagtccaacaaaggaacccctctgtatgata | DSVYRIKKLGTSPVLEAV |
| | agaacccattgtctatcaagaattctctgatcacaagaaggtg | VKGSIGRIRNSASATKLT |
| | gatggtgacagagatcgaacacggcaaggaggacaccatc | KVSKPQKVFSGGSSGGSS |
| | atcagcgacgagtacagaaagaagctgaaggaggctttca | GSETPGTSESATPESSGGS |
| | aacactggggcaccagcaagggcaagtgcctgaccgacg | SGGSSKRPAATKKAGQA |
| | aggaagccagacagaccggcttctaccagagaaagaatgg | KKKK |
| | caacttcatgagcctgcggtgcgtggagaagggcctgggct | (SEQ ID NO: 60) |
| | ggggccagatggtgaagatcaataacaacgcctttaagacg | |
| | aacgtgcacaacatcggggggctgtttacctggacgagaa | |
| | gggcaagaagaaggcctacgaaattaagaaccccagactg | |
| | ctgaaacatttcgtggaaaagccacctgagatcaagggaaa | |
| | gaagctgtacaccctgcggagaggcgactacgtgacccac | |
| | gagagcgaggactccgtgtacagaatcaaaaaactgggca | |
| | caagccctgtgctggaagctgtggtcaagggctctatcggc | |
| | cgcatccggaacagcgccagcgccacaaaactgaccaag | |
| | gtgtccaagcctcagaaggtgttttctggaggatctagcgga | |
| | ggatcctctggcagcgagacaccaggaacaagcgagtcag | |
| | caacaccagagagcagtggcggcagcagcggcggcagc | |
| | agcaaaaggccggcggccacgaaaaaggccggccaggc | |
| | aaaaaagaaaaag | |
| | (SEQ ID NO: 59) | |
| Truncated (16- | atgaaacggacagccgacggaagcgagttcgagtcaccaa | MKRTADGSEFESPKKKR |
| 775) I67R, | agaagaagcggaaagtccagaaagagctggtgctgggcat | KVQKELVLGIDYGGKYT |
| D568R, E706R | tgactacggcggaaagtacacaggcctggccgtggtggac | GLAVVDRRNNQVLYART |
| | cggagaaacaaccaggtgctgtacgccagaaccgtcaaga | VKMRDDVADILEGRREQ |
| | tgagagacgacgtggccgatatcctcgaaggccggaggga | RGRRRTGQTKKKRLREL |
| | acagcgaggcAGAagaagaacaggccagacaaagaa | KTYLKNIGGKYDESIGTF |
| | gaagagactgagagaactgaaaacctacctgaagaacatc | KNEPFMTVYKLCHKRGY |
| | ggaggaaaatatgacgagtctatcggcacattcaaaaacga | DYVEMPKEKTREEIEAM |
| | gcctttcatgacagtgtacaagctgtgtcacaagagaggcta | SADEKKQWEKEKKEWE |
| | cgactacgtggaaatgcctaaggaaaagaccagagaagaa | ETERNSRHRDEVTKDVR |
| | atcgaggccatgtctgccgacgagaaaaagcagtgggaaa | KIMSERDATEQQISHVIKI |
| | aagagaaaaaggagtgggaggaaaccgagcggaactcca | FNKQYRPKRFNNRILTKC |
| | gacaccgggatgaggtgaccaaggacgtgcggaagatcat | KVEDCGVNTPLRKNVRD |
| | gagcgagagagatgccaccgagcagcagatcagccacgt | LLIKDIVRFLPLTQSEKAE |
| | gatcaagatcttcaacaagcagtaccggcccaagcggttca | LEAHVLDKEGREKVKTF |
| | ataatagaatcctgacaaaatgtaaggttgaggattgcggcg | FRRHRINEFLRKQIYDIAF |
| | tgaacacacctctgagaaagaacgtcagagacctgctgatc | NQKLSGRTVFCKKHILEH |
| | aaggacatcgtgagattcctgcccctgacccagagcgagaa | EQHTKEERKVFRLAPSLK |
| | agccgagctggaagctcatgtgctggataaggaaggaaga | TKINNVLAVVKDEILPQF |
| | gagaaggtgaagaccttcttcagacggcacagaatcaacga | GINKVIMESNNFDIAART |
| | gttcctgcgcaagcagatctacgacatcgcctttaaccaaaa | KGKKRLAKDEYGKGHK |
| | gctgagcggcagaaccgtgttctgcaagaagcacatcctgg | EAKESLIEVLLRETKNQC |
| | agcacgagcaacacaccaaagaggaacgcaaggtctttag | VYCGKVITVTTANTDHIF |
| | actggcccctagcctgaaaactaagattaacaacgtgctggc | PRKAGGINVFANLVACC |
| | tgtggttaaggacgagatcctgccccagttcggcatcaacaa | RECNEKKKGRTPLESGV |
| | ggtcatcatggaatccaacaacttcgatattgccgctagaac | LPQPEITAHLENDLKKKI |
| | caagggcaagaagcggctggccaaggatgagtacggcaa | LEDARNMNELNLNSIKY |
| | gggccacaaggaggccaaagagagcctgatcgaggtgct | MSYASIGWRHMRDRLRG |
| | gctgcgggaaaccaagaaccagtgcgtgtactgcggaaag | LIGNEELPIERQSGILTAY |
| | gtgatcaccgtgaccaccgccaataccgaccacatcttcccc | FRRWWGFRKERGNDIHH |
| | agaaaggcgggcggcatcaacgttttcgccaacctggtggc | ALDAVILASRKDYTDDG |
| | ctgttgtagagagtgcaacgagaagaagaagggcagaaca | VDMTLKPKSSNGREFDP |
| | cctctggaatccggcgtgcttccacagcctgagatcaccgc | EKHLPDTISFKRRKSNKG |
| | ccacctggaaaacgacctcaaaaagaagatccttgaggac | TPLYDKNPLSIKNSLITRR |
| | gccagaaatatgaacgagctgaatctgaacagcatcaagta | WMVTEIEHGKEDTIISDE |
| | catgagctacgccagtatcggatggcggcacatgagagata | YRKKLKEAFKHWGTSKG |
| | gactgaggggccttatcggtaatgaagaactgcctatcgag | KCLTDEEARQTGFYQRK |
| | cgccaaagcggcatcctgacagcttacttcagacggtggtg | NGNFMSLRCVEKGLGW |
| | gggatttcggaaggaacggggaaatgacattcaccacgcc | GQMVKINNNAFKTNVHN |
| | ctggacgctgtgatcctggccagcagaaaagactatacaga | IGVAVYLDEKGKKKAYE |
| | tgacggcgtggatatgaccctgaagcccaaaagcagcaac | IKNPRLLKHFVRKPPEIK |
| | ggccgagaattcgaccccgagaaacatctgcctgatacaat | GKKLYTLRRGDYVTHES |
| | cagcttcaaaagaAGAaagtccaacaaaggaacccctct | EDSVYRIKKLGTSPVLEA |
| | gtatgataagaacccattgtctatcaagaattctctgatcacaa | VVKGSIGRIRNSASATKL |
| | gaaggtggatggtgacagagatcgaacacggcaaggagg | TKVSKPQKVFSGGSSGGS |
| | acaccatcatcagcgacgagtacagaaagaagctgaagga | SGSETPGTSESATPESSGG |
| | ggctttcaaacactggggcaccagcaagggcaagtgcctg | SSGGSSKRPAATKKAGQ |
| | accgacgaggaagccagacagaccggcttctaccagaga | AKKKK |
| | aagaatggcaacttcatgagcctgcggtgcgtggagaagg | (SEQ ID NO: 62) |
| | gcctgggctggggccagatggtgaagatcaataacaacgc | |
| | ctttaagacgaacgtgcacaacatcggggggctgtttacct | |
| | ggacgagaagggcaagaagaaggcctacgaaattaagaa | |
| | ccccagactgctgaaacatttcgtgAGAaagccacctga | |
| | gatcaagggaaagaagctgtacaccctgcggagaggcga | |
| | ctacgtgacccacgagagcgaggactccgtgtacagaatca | |
| | aaaaactgggcacaagccctgtgctggaagctgtggtcaag | |
| | ggctctatcggccgcatccggaacagcgccagcgccacaa | |
| | aactgaccaaggtgtccaagcctcagaaggtgttttctggag | |
| | gatctagcggaggatcctctggcagcgagacaccaggaac | |
| | aagcgagtcagcaacaccagagagcagtggcggcagcag | |
| | cggcggcagcagcaaaaggccggcggccacgaaaaagg | |
| | ccggccaggcaaaaaagaaaaag | |
| | (SEQ ID NO: 61) | |
| Truncated (16- | atgaaacggacagccgacggaagcgagttcgagtcaccaa | MKRTADGSEFESPKKKR |
| 775) I67R, | agaagaagcggaaagtccagaaagagctggtgctgggcat | KVQKELVLGIDYGGKYT |
| D568R, | tgactacggcggaaagtacacaggcctggccgtggtggac | GLAVVDRRNNQVLYART |
| W593R, E706R | cggagaaacaaccaggtgctgtacgccagaaccgtcaaga | VKMRDDVADILEGRREQ |
| | tgagagacgacgtggccgatatcctcgaaggccggaggga | RGRRRTGQTKKKRLREL |
| | acagcgaggcAGAagaagaacaggccagacaaagaa | KTYLKNIGGKYDESIGTF |
| | gaagagactgagagaactgaaaacctacctgaagaacatc | KNEPFMTVYKLCHKRGY |
| | ggaggaaaatatgacgagtctatcggcacattcaaaaacga | DYVEMPKEKTREEIEAM |
| | gcctttcatgacagtgtacaagctgtgtcacaagagaggcta | SADEKKQWEKEKKEWE |
| | cgactacgtggaaatgcctaaggaaaagaccagagaagaa | ETERNSRHRDEVTKDVR |
| | atcgaggccatgtctgccgacgagaaaaagcagtgggaaa | KIMSERDATEQQISHVIKI |
| | aagagaaaaaggagtgggaggaaaccgagcggaactcca | FNKQYRPKRFNNRILTKC |
| | gacaccgggatgaggtgaccaaggacgtgcggaagatcat | KVEDCGVNTPLRKNVRD |
| | gagcgagagagatgccaccgagcagcagatcagccacgt | LLIKDIVRFLPLTQSEKAE |
| | gatcaagatcttcaacaagcagtaccggcccaagcggttca | LEAHVLDKEGREKVKTF |
| | ataatagaatcctgacaaaatgtaaggttgaggattgcggcg | FRRHRINEFLRKQIYDIAF |
| | tgaacacacctctgagaaagaacgtcagagacctgctgatc | NQKLSGRTVFCKKHILEH |
| | aaggacatcgtgagattcctgcccctgacccagagcgagaa | EQHTKEERKVFRLAPSLK |
| | agccgagctggaagctcatgtgctggataaggaaggaaga | TKINNVLAVVKDEILPQF |
| | gagaaggtgaagaccttcttcagacggcacagaatcaacga | GINKVIMESNNFDIAART |
| | gttcctgcgcaagcagatctacgacatcgcctttaaccaaaa | KGKKRLAKDEYGKGHK |
| | gctgagcggcagaaccgtgttctgcaagaagcacatcctgg | EAKESLIEVLLRETKNQC |
| | agcacgagcaacacaccaaagaggaacgcaaggtctttag | VYCGKVITVTTANTDHIF |
| | actggcccctagcctgaaaactaagattaacaacgtgctggc | PRKAGGINVFANLVACC |
| | tgtggttaaggacgagatcctgccccagttcggcatcaacaa | RECNEKKKGRTPLESGV |
| | ggtcatcatggaatccaacaacttcgatattgccgctagaac | LPQPEITAHLENDLKKKI |
| | caagggcaagaagcggctggccaaggatgagtacggcaa | LEDARNMNELNLNSIKY |
| | gggccacaaggaggccaaagagagcctgatcgaggtgct | MSYASIGWRHMRDRLRG |
| | gctgcgggaaaccaagaaccagtgcgtgtactgcggaaag | LIGNEELPIERQSGILTAY |
| | gtgatcaccgtgaccaccgccaataccgaccacatcttcccc | FRRWWGFRKERGNDIHH |
| | agaaaggcgggcggcatcaacgttttcgccaacctggtggc | ALDAVILASRKDYTDDG |
| | ctgttgtagagagtgcaacgagaagaagaagggcagaaca | VDMTLKPKSSNGREFDP |
| | cctctggaatccggcgtgcttccacagcctgagatcaccgc | EKHLPDTISFKRRKSNKG |
| | ccacctggaaaacgacctcaaaaagaagatccttgaggac | TPLYDKNPLSIKNSLITRR |
| | gccagaaatatgaacgagctgaatctgaacagcatcaagta | RMVTEIEHGKEDTIISDE |
| | catgagctacgccagtatcggatggcggcacatgagagata | YRKKLKEAFKHWGTSKG |
| | gactgaggggccttatcggtaatgaagaactgcctatcgag | KCLTDEEARQTGFYQRK |
| | cgccaaagcggcatcctgacagcttacttcagacggtggtg | NGNFMSLRCVEKGLGW |
| | gggatttcggaaggaacggggaaatgacattcaccacgcc | GQMVKINNNAFKTNVHN |
| | ctggacgctgtgatcctggccagcagaaaagactatacaga | IGVAVYLDEKGKKKAYE |
| | tgacggcgtggatatgaccctgaagcccaaaagcagcaac | IKNPRLLKHFVRKPPEIK |
| | ggccgagaattcgaccccgagaaacatctgcctgatacaat | GKKLYTLRRGDYVTHES |
| | cagcttcaaaagaAGAaagtccaacaaaggaacccctct | EDSVYRIKKLGTSPVLEA |
| | gtatgataagaacccattgtctatcaagaattctctgatcacaa | VVKGSIGRIRNSASATKL |
| | gaaggCGGatggtgacagagatcgaacacggcaagga | TKVSKPQKVFSGGSSGGS |
| | ggacaccatcatcagcgacgagtacagaaagaagctgaag | SGSETPGTSESATPESSGG |
| | gaggctttcaaacactggggcaccagcaagggcaagtgcc | SSGGSSKRPAATKKAGQ |
| | tgaccgacgaggaagccagacagaccggcttctaccagag | AKKKK |
| | aaagaatggcaacttcatgagcctgcggtgcgtggagaag | (SEQ ID NO: 64) |
| | ggcctgggctggggccagatggtgaagatcaataacaacg | |
| | cctttaagacgaacgtgcacaacatcggggtggctgtttacc | |
| | tggacgagaagggcaagaagaaggcctacgaaattaagaa | |
| | ccccagactgctgaaacatttcgtgAGAaagccacctga | |
| | gatcaagggaaagaagctgtacaccctgcggagaggcga | |
| | ctacgtgacccacgagagcgaggactccgtgtacagaatca | |
| | aaaaactgggcacaagccctgtgctggaagctgtggtcaag | |
| | ggctctatcggccgcatccggaacagcgccagcgccacaa | |
| | aactgaccaaggtgtccaagcctcagaaggtgttttctggag | |
| | gatctagcggaggatcctctggcagcgagacaccaggaac | |
| | aagcgagtcagcaacaccagagagcagtggcggcagcag | |
| | cggcggcagcagcaaaaggccggcggccacgaaaaagg | |
| | ccggccaggcaaaaaagaaaaag | |
| | (SEQ ID NO: 63) | |

### Example 6. Editing of Human Genes in HEK293T Cells Using Nuclease A-Derived CRISPR Nuclease Polypeptides

This Example shows genetic modification of human genes utilizing the nuclease polypeptides derived from Nuclease A listed in Tables 1 and 14 constructed following the descriptions provided in Examples 1, and 3. Specifically, the polypeptides are used to indels into the human target genes when combined with RNA guides.

RNA guides, including EMX1-T3 with reference scaffold, truncated reference scaffold, or Scaffold 2, EMX1-T7 with Scaffold 2, and AAVS-T3 with Scaffold 2, are shown in Table 2 of Example 1 or Table 8 of Example 4. Samples are prepared for NGS and analyzed as described in Example 1.

It is anticipated that the nuclease polypeptides derived from Nuclease A described in this Example are capable of creating indels at target sites encoded by RNA guides, such as those described in this Example, into human genes.

### Example 7: Editing of Human Target Genes in HEK293T Cells by CRISPR Nuclease K or Variants Thereof

This Example describes the genomic editing of the AAVS1, EMX1, and VEGFA genes by Nuclease K of SEQ ID NO: 65 introduced into cells by lipid-based transient transfection into the HEK293T cell line.

Nuclease K was tagged with N-terminal SV40 nuclear localization sequence (NLS) and a C-terminal nucleoplasmin nuclear localization sequence (NLS), and its coding sequence was converted to a human codon-optimized DNA sequence, synthesized, and cloned into a pcDNA3.1 vector (Invitrogen), containing a CMV promoter for expression. The reference and NLS-tagged sequences used are in Table 15. The U6 PolIII promoter uses a +1 G at the start of the transcript (i.e., the 5′ end of the RNA) for more efficient transcription that is excluded from the sequences described in Table 16. Plasmids were purified using a midiprep kit.

RNA guides were designed and cloned and into a pUC19 plasmid following the U6 PolIII promoter and terminated with a 6× polyT sequence. RNA guides were designed to be specific to target sequences within the coding exons of AAVS1, EMX1, and VEGFA with 5′-NGG-3′ PAM sequences (the PAM sequence is on the 3′ end of the target sequence). See all RNA guide sequences in Table 16. Plasmids were purified using a midiprep kit.

TABLE 15
Amino Acid Sequences for Exemplary CRISPR Nuclease Polypeptides
Derived from Nuclease K

| Components | Amino acid sequence | SEQ ID NO |
|---|---|---|
| Nuclease K | MERELVLGIDYGGKYTGLAVVDRRHNQVLYANRLKMRDDVAGI | 65 |
| | LKDRRKQRGIRRTAQTKKKRLRELKNYLKSIGYNESTATFETVYS | |
| | LAHKRGYDYADMPEEKTSEEIEAMDVEERKQWEKEKQEWEETK | |
| | RNSRHRKEVVKDVHKAMIEGRATEEQIKRVERIFNKQYRPKRFN | |
| | NRILTKCKVEDCGVNTPLRKNVRDLLIENIVRFFPIEQSEKDNLKD | |
| | AVLDKNRREEVKSFFRKHKTDEHIRKQVYDIADNKLSGRTVFCKE | |
| | HILEHTEHSKEERKVFRLAPSLKTKIENVLAVIKDEILPKFTVNKV | |
| | VMESNNFDIAAKTQGKKRLAKEEYGKGPREGKETRKEALLRETD | |
| | GRCIYCGKSIDISNAHDDHIFPRKAGGLNIFANLVACCAVCNENK | |
| | KGRTPLESGISPKPEIIAFMKNDLKKKILEDARNINTVDFNKYMSH | |
| | ASIGWRYMRDRLRESAGNKKLPIERQSGIYTAYFRRWWGFKKER | |
| | GNTLHHALDAVILASRKGYSDDGLVDMTLKPKYNKGGGFDSEK | |
| | HLPEPIEFKRDKGSKGSALHDRNPLSYKKGIITRRFMVTEIECGKE | |
| | DDVISETYREKLKEAFKRFDTKKGKCLTDKEAKEAGFCIKKNELV | |
| | MSLKCSIKGTGPGQMIRINNNVFKTNVHNVGVDVYLDEKGKKKA | |
| | YERKNPRLSKHFIEPPPQPNGRVSFTLKRRDMVTVEGEDAIYRIKK | |
| | LGTSPTIEAVVGSDGKTRTVSATKLTKANSAE | |
| SV40 NLS | KRTADGSEFESPKKKRKV | 3 |
| Nucleoplasmin | KRPAATKKAGQAKKKK | 4 |
| NLS | | |
| XTEN linker | SGGSSGGSSGSETPGTSESATPESSGGSSGGSS | 95 |
| NLS-tagged | MKRTADGSEFESPKKKRKVERELVLGIDYGGKYTGLAVVDRRHNQ | 66 |
| Nuclease K | VLYANRLKMRDDVAGILKDRRKQRGIRRTAQTKKKRLRELKNYL | |
| | KSIGYNESTATFETVYSLAHKRGYDYADMPEEKTSEEIEAMDVEE | |
| | RKQWEKEKQEWEETKRNSRHRKEVVKDVHKAMIEGRATEEQIK | |
| | RVERIFNKQYRPKRFNNRILTKCKVEDCGVNTPLRKNVRDLLIENI | |
| | VRFFPIEQSEKDNLKDAVLDKNRREEVKSFFRKHKTDEHIRKQVY | |
| | DIADNKLSGRTVFCKEHILEHTEHSKEERKVFRLAPSLKTKIENVL | |
| | AVIKDEILPKFTVNKVVMESNNFDIAAKTQGKKRLAKEEYGKGPR | |
| | EGKETRKEALLRETDGRCIYCGKSIDISNAHDDHIFPRKAGGLNIF | |
| | ANLVACCAVCNENKKGRTPLESGISPKPEIIAFMKNDLKKKILEDA | |
| | RNINTVDFNKYMSHASIGWRYMRDRLRESAGNKKLPIERQSGIYT | |
| | AYFRRWWGFKKERGNTLHHALDAVILASRKGYSDDGLVDMTLK | |
| | PKYNKGGGFDSEKHLPEPIEFKRDKGSKGSALHDRNPLSYKKGIIT | |
| | RRFMVTEIECGKEDDVISETYREKLKEAFKRFDTKKGKCLTDKEA | |
| | KEAGFCIKKNELVMSLKCSIKGTGPGQMIRINNNVFKTNVHNVGV | |
| | DVYLDEKGKKKAYERKNPRLSKHFIEPPPQPNGRVSFTLKRRDM | |
| | VTVEGEDAIYRIKKLGTSPTIEAVVGSDGKTRTVSATKLTKANSAE | |
| | KRPAATKKAGQAKKKK | |

TABLE 16
Target and RNA Guide Sequences

| | | Target | |
|---|---|---|---|
| Guide | PAM | Sequence | RNA Guide Sequence* |
| AAVS1- | AGG | TTGTGAGAAT | UUGUGAGAAUGGUGCGUCCUguuacaguuaaggcucugaaaaga |
| T3 | | GGTGCGTCCT | gccuuaauuguaaaacgccuauacagugaagggauauacgcuuggguuugucca |
| | | (SEQ ID NO: 67) | gccugagccucuaugccagaaauggcgccuucaucguggguuaggacauuuaaa |
| | | | uuuaaaaacuauucagcacuguuugcucuugucagcuugguggcaga (SEQ |
| | | | ID NO: 68) |
| AAVS1- | TGG | CTTTGTAGCC | CUUUGUAGCCUCUCCCGCUCguuacaguuaaggcucugaaaagag |
| T6 | | TCTCCCGCTC | ccuuaauuguaaaacgccuauacagugaagggauauacgcuuggguuuguccag |
| | | (SEQ ID NO: 69) | ccugagccucuaugccagaaauggcgccuucaucguggguuaggacauuuaaau |
| | | | uuaaaaacuauucagcacuguuugcucuugucagcuugguggcaga (SEQ ID |
| | | | NO: 70) |
| EMX1- | AGG | TGGATGGCGA | UGGAUGGCGACUUCAGGCACguuacaguuaaggcucugaaaagag |
| T2 | | CTTCAGGCAC | ccuuaauuguaaaacgccuauacagugaagggauauacgcuuggguuuguccag |
| | | (SEQ ID NO: 71) | ccugagccucuaugccagaaauggcgccuucaucguggguuaggacauuuaaau |
| | | | uuaaaaacuauucagcacuguuugcucuugucagcuugguggcaga (SEQ ID |
| | | | NO: 72) |
| EMX1- | GGG | CTTTAGCAAG | CUUUAGCAAGGGACUAUUCAguuacaguuaaggcucugaaaaga |
| T7 | | GGACTATTCA | gccuuaauuguaaaacgccuauacagugaagggauauacgcuuggguuugucca |
| | | (SEQ ID NO: 73) | gccugagccucuaugccagaaauggcgccuucaucguggguuaggacauuuaaa |
| | | | uuuaaaaacuauucagcacuguuugcucuugucagcuugguggcaga (SEQ |
| | | | ID NO: 74) |
| VEGFA- | TGG | TTGTTGACAT | UUGUUGACAUUGUCCACACCguuacaguuaaggcucugaaaagag |
| T3 | | TGTCCACACC | ccuuaauuguaaaacgccuauacagugaagggauauacgcuuggguuuguccag |
| | | (SEQ ID NO: 75) | ccugagccucuaugccagaaauggcgccuucaucguggguuaggacauuuaaau |
| | | | uuaaaaacuauucagcacuguuugcucuugucagcuugguggcaga (SEQ ID |
| | | | NO: 76) |
| VEGFA- | AGG | TTTGCCCATA | UUUGCCCAUACUGGGGACCAguuacaguuaaggcucugaaaagag |
| T6 | | CTGGGGACCA | ccuuaauuguaaaacgccuauacagugaagggauauacgcuuggguuuguccag |
| | | (SEQ ID NO: 77) | ccugagccucuaugccagaaauggcgccuucaucguggguuaggacauuuaaau |
| | | | uuaaaaacuauucagcacuguuugcucuugucagcuugguggcaga (SEQ ID |
| | | | NO: 78) |

*Spacer in upper case and scaffold (SEQ ID NO: 79) in lower case

HEK293T cells were transfected with plasmids expressing the nuclease polypeptides derived from Nuclease K and the gRNAs provided herein following the methods provided in Example 1 above. Gene editing efficiencies were determined by NGS following the methods also provided in Example 1. The NGS results were analyzed also following the methods provided in Example 1 above.

For each target, indel ratios, referring to the percentage of NGS reads comprising indels, were calculated for each sample and its cognate no protein control. Targets comprising a higher percentage of indels when Nuclease K was included in the transfection were indicative of DNA editing outcomes in the cell.

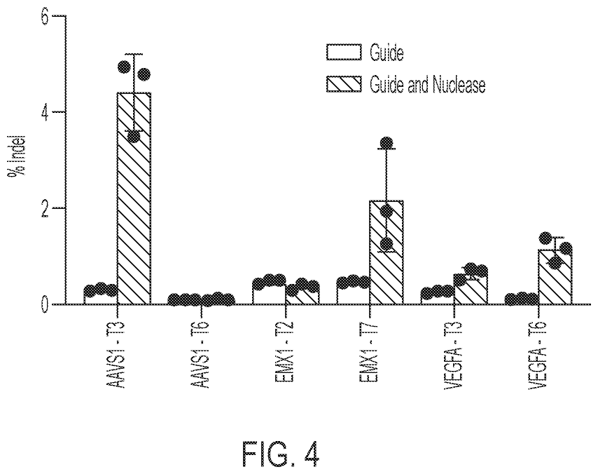

As shown in FIG. 4, four of the six targets tested demonstrated a greater level of indels observed when the Nuclease K (SEQ ID NO: 65) plasmid was present.

### Example 8: Effectiveness CRISPR Nuclease K Variants for Targeting of Mammalian Genes

This Example describes indel assessment on mammalian targets using variants of Nuclease K transfected into HEK293T cells.

Arginine scanning mutagenesis was performed to individually substitute each non-arginine residue of the reference Nuclease K (SEQ ID NO: 65) to arginine. This resulted in 682 single arginine substitution variants. Nucleic acids encoding Nuclease K and each variant thereof were then individually cloned into a pcda3.1 backbone (Invitrogen), and the plasmids were maxi-prepped and diluted. The plasmids comprised a CMV promoter, a first NLS (KRTADGSEFESPKKKRKV; SEQ ID NO: 3) upstream of the coding sequence, an XTEN linker (SGGSSGGSSGSETPGTSESATPESSGGSSGGSS; SEQ ID NO: 95) followed by a second NLS (KRPAATKKAGQAKKKK; SEQ ID NO: 4) downstream of the coding sequence. See also Example 7 above.

The RNA guide and target sequences are shown in Table 17. RNA guides were cloned into a pUC19 backbone (New England Biolabs®) following the U6 PolIII promoter and terminated with a 6× polyT sequence. The U6 PolIII uses a +1 G at the start of the transcript (i.e., the 5′ end of the RNA) for more efficient transcription that is excluded from the sequences described in Table 17. The plasmids were then maxi-prepped and diluted.

TABLE 17
Mammalian Targets and Corresponding crRNAs.

| Target | PAM | | |
|---|---|---|---|
| identifier | sequence | Target sequence | RNA guide sequence |
| AAVS1-T3 | AGG | SEQ ID NO: 67 (see, Table 16 | SEQ ID NO: 68 (see, Table 16 above) |
| | | above) | |
| EMX1-T7 | GGG | SEQ ID NO: 73 (see, Table 16 | SEQ ID NO: 74 (see, Table 16 above) |
| | | above) | |

HEK293T cells were transfected with plasmids expressing the nuclease polypeptides derived from Nuclease K and the gRNAs provided herein following the methods provided in Example 1 above. Gene editing efficiencies were determined by NGS following the methods also provided in Example 1. The NGS results were analyzed also following the methods provided in Example 1 above.

For each target, indel ratios, referring to the percentage of NGS reads comprising indels, were calculated for the reference and for each variant. The indel ratios used for fold change calculations were the average of two technical replicates. To then calculate fold change in indel ratios at a particular target, the indel ratio for each variant was divided by the indel ratio for the reference. Table 4 shows fold change in indel ratios for each target and as an average of both targets. Numbering is relative to the reference nuclease of SEQ ID NO: 1 (i.e., without an NLS). For example, at the AAVS1 target, the indel ratio for the D582R variant was 3.68 times that of the reference indel ratio, and at the EMX1 target, the indel ratio for the D582R variant was 31.80 times that of the reference indel ratio. The average fold change in indel ratio for the D582R variant at both targets was 17.74 times that of the indel ratio for the reference at both targets.

As shown in Table 18, 29 variants with single arginine substitutions (left column) were characterized as yielding at least a 1.5X increase in indel ratio relative to the reference indel ratio, when averaged across both targets (right column).

TABLE 18
Fold Change in Indel Ratios*

| | | | AAVS1 and EMX1 |
|---|---|---|---|
| Substitution | AAVS1 | EMX1 | average fold change |
| D582R | 3.68 | 31.80 | 17.74 |
| D46R | 6.71 | 9.86 | 8.29 |
| I631R | 2.47 | 9.96 | 6.22 |
| F570R | 2.81 | 8.15 | 5.48 |
| I53R | 6.66 | 3.35 | 5.01 |
| G42R | 3.94 | 5.30 | 4.62 |
| E576R | 2.70 | 5.18 | 3.94 |
| T734R | 3.64 | 1.81 | 2.72 |
| C630R | 1.81 | 2.84 | 2.33 |
| E683R | 1.82 | 2.34 | 2.08 |
| S719R | 2.03 | 1.89 | 1.96 |
| E128R | 1.91 | 1.94 | 1.93 |
| G638R | 1.60 | 2.20 | 1.90 |
| S631R | 1.91 | 1.79 | 1.85 |
| D99R | 1.54 | 2.10 | 1.82 |
| A332R | 1.87 | 1.70 | 1.78 |
| E84R | 2.11 | 1.38 | 1.75 |
| E270R | 1.71 | 1.78 | 1.74 |
| I368R | 1.51 | 1.93 | 1.72 |
| E273R | 1.69 | 1.64 | 1.66 |
| A552R | 2.00 | 1.30 | 1.65 |
| D373R | 1.44 | 1.80 | 1.62 |
| D600R | 1.90 | 1.31 | 1.60 |
| A57R | 1.88 | 1.25 | 1.57 |
| L515R | 1.81 | 1.28 | 1.55 |
| E541R | 1.32 | 1.77 | 1.55 |
| A746R | 1.73 | 1.30 | 1.51 |
| T718R | 1.60 | 1.42 | 1.51 |
| E243R | 1.49 | 1.53 | 1.51 |

*Variant indel ratio/Reference indel ratio

177 variants were analyzed as having indel ratios 1X-1.5X of the reference indel ratios, when averaged across both targets: S548R, S365R, S134R, D729R, E747R, A724R, D581R, D301R, E623R, W127R, T180R, V114R, A395R, E123R, T703R, K68R, S258R, K60R, K324R, A297R, A381R, K402R, D217R, D356R, A98R, E266R, T85R, N218R, K311R, V396R, N400R, K131R, K633R, V692R, 1565R, E671R, I109R, E110R, A709R, Q27R, K300R, Q125R, H25R, S214R, T438R, V726R, K605R, D186R, K563R, L495R, P637R, E121R, T741R, K306R, E107R, N689R, K521R, L269R, V262R, Q58R, K333R, S79R, K414R, E212R, D367R, K380R, K549R, D512R, K629R, K276R, P101R, E334R, G730R, M112R, K14R, F420R, L407R, D708R, P685R, I149R, K238R, E594R, E203R, K428R, K603R, W120R, K697R, E108R, E102R, S745R, G492R, N437R, H244R, E115R, K45R, E538R, K364R, K427R, S88R, K159R, T308R, V189R, K35R, K328R, D113R, N744R, K666R, K602R, Q50R, K49R, Q326R, K72R, E116R, G343R, K543R, E590R, T105R, K124R, E103R, K142R, K731R, G464R, A322R, K329R, M701R, P686R, S316R, K534R, E230R, D242R, M571R, K610R, A111R, K664R, K62R, Q687R, E349R, P684R, K344R, E342R, E431R, V184R, K679R, G337R, G727R, E150R, L193R, K146R, K265R, A419R, C606R, S275R, E705R, S511R, L624R, K338R, K240R, D221R, T519R, P469R, K104R, N133R, H239R, S678R, P210R, H145R, K466R, I584R, and K422R.

The remaining variants (483 variants) resulted in decreased indel ratios relative to the reference indel ratios (fold change in indel ratios of less than 1.0): G357R, K216R, N644R, E586R, M100R, D424R, D199R, D545R, K443R, G527R, S106R, G529R, K739R, K562R, K620R, H274R, L200R, E155R, T272R, S73R, S627R, N69R, E345R, K597R, K667R, K122R, K233R, P305R, Q119R, P415R, T346R, S369R, T130R, K488R, K579R, K613R, N525R, S736R, E2R, E185R, K523R, K673R, E614R, Q213R, S412R, K220R, K61R, K668R, K280R, K591R, P171R, N175R, K714R, K118R, K138R, K289R, T587R, N370R, F208R, K593R, T732R, L661R, I540R, S728R, I268R, E277R, G528R, K467R, K226R, Y95R, N318R, P688R, E533R, K526R, I710R, E215R, E126R, G604R, D513R, K181R, A743R, D254R, I619R, G188R, E723R, E278R, K742R, E399R, K195R, L224R, P340R, K247R, N310R, S693R, G564R, N77R, N227R, I158R, Y250R, C577R, N435R, N622R, T154R, K546R, D225R, G509R, A147R, E156R, T695R, N255R, D531R, K256R, Y670R, K92R, E471R, L390R, Y660R, T738R, G52R, D251R, K621R, 1411R, G363R, M518R, H535R, A253R, V735R, P539R, E707R, L179R, T567R, V725R, P537R, E408R, N654R, S532R, K167R, E490R, E78R, Q248R, Q473R, N204R, K172R, E4R, 1245R, T241R, G578R, N645R, G547R, T608R, V625R, F282R, E663R, F694R, E129R, I43R, L257R, C618R, E416R, S560R, E461R, E335R, Y524R, E302R, E162R, I476R, A350R, G656R, E139R, Q168R, N423R, Y510R, E574R, G550R, V197R, G616R, D143R, K183R, A222R, N465R, G514R, T601R, K348R, D440R, L384R, E231R, H271R, F599R, F174R, A41R, G665R, G690R, I178R, G706R, D609R, Y480R, D517R, V704R, G75R, T59R, G486R, I418R, K508R, K715R, A615R, D457R, Y76R, Y336R, L5R, T56R, L304R, S462R, H680R, P675R, E293R, I211R, L536R, N385R, 1417R, F209R, G151R, F542R, F530R, L29R, E611R, S409R, T721R, H372R, V655R, H91R, G327R, 1436R, W484R, L89R, V232R, I205R, D662R, G382R, T494R, D658R, V141R, G339R, T80R, V652R, H136R, G717R, Y561R, A463R, G403R, A433R, L64R, N651R, N166R, Y588R, A371R, Q157R, D432R, H653R, T405R, L520R, N196R, L696R, Y169R, K401R, L425R, P192R, P406R, S585R, L352R, N442R, A285R, V313R, F387R, V281R, I74R, A388R, Y454R, V140R, G410R, I682R, N190R, F263R, Y477R, I566R, D96R, I643R, M626R, V647R, N493R, V516R, L44R, S551R, D22R, T290R, N557R, T635R, L677R, I366R, L351R, A669R, F377R, E354R, A31R, F681R, N294R, K291R, G383R, E580R, C264R, G634R, N317R, F617R, A323R, V249R, L331R, F307R, D39R, K426R, V659R, F235R, E66R, P522R, G259R, N646R, D700R, H267R, S474R, L201R, C394R, V161R, G636R, N674R, F648R, L288R, Y15R, L296R, P286R, N32R, T261R, H447R, P413R, A612R, V6R, I386R, Q639R, T82R, T325R, F236R, I292R, C359R, D38R, K649R, 1575R, M640R, V295R, I164R, V40R, S446R, N176R, Y711R, Y97R, T191R, D320R, V583R, V298R, T650R, N389R, I252R, I641R, V657R, I360R, A153R, I722R, L284R, 1321R, V206R, 1376R, V28R, M148R, C187R, Y30R, A737R, V391R, N398R, F83R, E315R, I202R, L628R, V309R, L219R, V144R, M421R, F481R, V21R, L553R, M455R, A505R, P378R, I303R, P720R, I713R, A498R, Y70R, G12R, C182R, V312R, V86R, L499R, S287R, A19R, M314R, T573R, L740R, L7R, T355R, I299R, L18R, I503R, Y361R, V20R, Y87R, Y11R, G94R, F441R, N26R, F487R, M445R, L716R, F319R, L468R, H375R, V223R, G8R, A448R, D374R, V439R, A501R, V502R, M36R, L559R, F165R, G17R, L607R, V702R, A595R, L34R, K489R, L592R, S506R, W452R, F596R, C362R, L504R, T16R, L430R, S449R, D555R, H496R, DIOR, H554R, G13R, G451R, A479R, L67R, T478R, P558R, D500R, I429R, I470R, Y444R, A392R, V572R, I450R, H497R, C393R, C397R, G475R, I9R, W485R, L71R, A90R, L459R, and A81R.

Based on this experiment, the following variants were selected for further engineering relative to Nuclease K of SEQ ID NO: 65: D582R, D46R, 1631R, F570R, 153R, G42R, E576R, T734R, C630R, E683R, S719R, and E128R.

### Example 9: Effectiveness of Combination CRISPR Nuclease K Variants for Targeting of Mammalian Genes

This Example describes indel assessment on mammalian targets using nuclease variants of Nuclease K comprising two or more substitutions identified as increasing indel activity in Example 8. 71 combination Nuclease K variants were tested.

Each Nuclease K variant and RNA guide (see Table 19) was cloned as described in Example 8. HEK293T cells were further transfected, followed by NGS analysis, as described in Example 8. For each target, indel ratios, referring to the percentage of NGS reads comprising indels, were calculated for the reference Nuclease K (SEQ ID NO: 65) and for each Nuclease K variant. The indel ratios shown in Table 20 were calculated as the average of two bioreplicates, each of which contained two technical replicates.

TABLE 19
Mammalian Targets and Corresponding crRNAs

| Target | PAM | | |
|---|---|---|---|
| identifier | sequence | Target sequence | RNA guide sequence |
| AAVS1- | AGG | SEQ ID NO: 67 (see, Table | SEQ ID NO: 68 (see, Table 16 |
| T3 | | 16 above) | above) |
| EMX1- | GGG | SEQ ID NO: 73 (see, Table | SEQ ID NO: 74 (see, Table 16 |
| T7 | | 16 above) | above) |
| VEGFA- | AGG | SEQ ID NO: 77 (see, Table | SEQ ID NO: 78 (see, Table 16 |
| T6 | | 16 above) | above) |

TABLE 20
Indel Ratios for Mammalian Targets

| | AAVS1 | EMX1 | VEGFA | |
|---|---|---|---|---|
| | average | average | average | |
| Substitutions | indel ratio | indel ratio | indel ratio | Average indel ratio targets |
| D582R, I53R, T734R | 0.270 | 0.250 | 0.097 | 0.205 |
| D582R, G42R | 0.221 | 0.231 | 0.153 | 0.202 |
| D582R, I53R | 0.257 | 0.224 | 0.096 | 0.192 |
| D582R, D46R | 0.238 | 0.207 | 0.120 | 0.188 |
| I632R, G42R | 0.157 | 0.234 | 0.139 | 0.176 |
| I53R, G42R | 0.286 | 0.142 | 0.089 | 0.173 |
| F570R, G42R | 0.194 | 0.204 | 0.103 | 0.167 |
| F570R, I53R, T734R | 0.249 | 0.192 | 0.057 | 0.166 |
| D582R, D46R, T734R | 0.228 | 0.171 | 0.099 | 0.166 |
| D582R, D46R, I53R | 0.194 | 0.222 | 0.079 | 0.165 |
| D582R, T734R | 0.259 | 0.147 | 0.083 | 0.163 |
| D582R, F570R, T734R | 0.222 | 0.171 | 0.068 | 0.154 |
| D46R, F570R | 0.200 | 0.189 | 0.071 | 0.153 |
| D46R, I53R, T734R | 0.286 | 0.112 | 0.061 | 0.153 |
| D46R, F570R, T734R | 0.223 | 0.167 | 0.068 | 0.153 |
| D582R, F570R, I53R, T734R | 0.188 | 0.207 | 0.049 | 0.148 |
| D46R, T734R | 0.285 | 0.099 | 0.054 | 0.146 |
| D582R, F570R, I53R | 0.188 | 0.199 | 0.051 | 0.146 |
| D46R, E576R | 0.224 | 0.143 | 0.071 | 0.146 |
| I632R, E576R | 0.125 | 0.165 | 0.140 | 0.143 |
| D582R, E576R | 0.182 | 0.129 | 0.119 | 0.143 |
| G42R, E576R | 0.178 | 0.142 | 0.094 | 0.138 |
| F570R, I53R | 0.203 | 0.154 | 0.050 | 0.136 |
| D46R, F570R, I53R | 0.171 | 0.184 | 0.050 | 0.135 |
| D582R, D46R, F570R | 0.168 | 0.167 | 0.070 | 0.135 |
| I632R, I53R, T734R | 0.154 | 0.192 | 0.058 | 0.134 |
| I632R, T734R | 0.168 | 0.152 | 0.077 | 0.132 |
| D46R, I632R | 0.132 | 0.164 | 0.089 | 0.128 |
| I632R, I53R | 0.146 | 0.177 | 0.058 | 0.127 |
| I53R, E576R | 0.236 | 0.081 | 0.061 | 0.126 |
| D582R, 1632R, 153R, T734R | 0.147 | 0.162 | 0.061 | 0.123 |
| D582R, I632R, I53R | 0.146 | 0.161 | 0.061 | 0.123 |
| I53R, T734R | 0.280 | 0.040 | 0.043 | 0.121 |
| D582R, D46R, I632R | 0.122 | 0.157 | 0.081 | 0.120 |
| F570R, E576R | 0.163 | 0.105 | 0.090 | 0.119 |
| D582R, F570R | 0.149 | 0.146 | 0.059 | 0.118 |
| D582R, D46R, I53R, T734R | 0.139 | 0.157 | 0.054 | 0.117 |
| D46R, F570R, I53R, T734R | 0.140 | 0.154 | 0.042 | 0.112 |
| I632R, F570R, T734R | 0.128 | 0.143 | 0.061 | 0.110 |
| F570R, T734R | 0.214 | 0.076 | 0.038 | 0.109 |
| G42R, T734R | 0.212 | 0.065 | 0.049 | 0.109 |
| D46R, I53R | 0.211 | 0.065 | 0.042 | 0.106 |
| D582R, I632R, T734R | 0.141 | 0.109 | 0.064 | 0.105 |
| D582R, D46R, I632R, T734R | 0.117 | 0.118 | 0.073 | 0.103 |
| D46R, I632R, T734R | 0.114 | 0.120 | 0.064 | 0.100 |
| E576R, T734R | 0.203 | 0.050 | 0.042 | 0.099 |
| I632R, F570R, I53R, T734R | 0.101 | 0.135 | 0.036 | 0.091 |
| D582R, D46R, I632R, I53R | 0.096 | 0.121 | 0.047 | 0.088 |
| I632R, F570R, I53R | 0.094 | 0.131 | 0.036 | 0.087 |
| D46R, I632R, F570R | 0.089 | 0.113 | 0.057 | 0.086 |
| D582R, D46R, F570R, T734R | 0.111 | 0.090 | 0.043 | 0.081 |
| D582R, D46R, F570R, I53R | 0.095 | 0.108 | 0.030 | 0.078 |
| D582R, D46R, 1632R, F570R | 0.074 | 0.097 | 0.054 | 0.075 |
| D46R, I632R, I53R | 0.073 | 0.102 | 0.034 | 0.070 |
| D46R, I632R, F570R, T734R | 0.081 | 0.078 | 0.040 | 0.066 |
| D46R, G42R | 0.147 | 0.031 | 0.019 | 0.066 |
| I632R, F570R | 0.061 | 0.084 | 0.042 | 0.062 |
| D582R, I632R | 0.070 | 0.069 | 0.046 | 0.062 |
| D46R, I632R, I53R, T734R | 0.068 | 0.080 | 0.028 | 0.058 |
| D46R, I632R, F570R, I53R | 0.059 | 0.069 | 0.026 | 0.051 |
| D582R, I632R, F570R, I53R | 0.065 | 0.053 | 0.031 | 0.050 |
| D46R, C630S | 0.064 | 0.033 | 0.023 | 0.040 |
| D582R, I632R, F570R, T734R | 0.057 | 0.035 | 0.026 | 0.039 |
| D582R, C630S | 0.045 | 0.042 | 0.028 | 0.038 |
| I53R, C630S | 0.073 | 0.016 | 0.014 | 0.034 |
| D582R, I632R, F570R | 0.030 | 0.024 | 0.022 | 0.025 |
| G42R, C630S | 0.025 | 0.020 | 0.016 | 0.021 |
| F570R, C630S | 0.035 | 0.014 | 0.010 | 0.020 |
| Reference (SEQ ID NO: 1) | 0.037 | 0.011 | 0.009 | 0.019 |
| E576R, C630S | 0.021 | 0.019 | 0.015 | 0.018 |
| T734R, C630S | 0.028 | 0.009 | 0.005 | 0.014 |
| I632R, C630S | 0.016 | 0.012 | 0.011 | 0.013 |

As shown in Table 20, all but three of the combination Nuclease K variants exhibited higher indel activity than the reference Nuclease K (SEQ ID NO: 65). Two Nuclease K variants resulted in indel ratios of over 0.2 when averaged across the three targets, indicating that over 20% of NGS reads comprised indels. These 2 Nuclease K variants comprised the following substitutions: a) D582R, 153R, and T734R and b) D582R and G42R. 42 Nuclease K variants resulted in indel ratios of over 0.1 when averaged across the three targets, indicating that over 10% of NGS reads comprised indels.

Based on this experiment, the Nuclease K variant comprising the following substitutions was selected for further testing: D582R and G42R. This Nuclease K variant exhibited an over 8-fold increase in indel activity compared to the reference Nuclease K (SEQ ID NO: 65).

### Example 10: Editing of Human Target Sequences in HEK293T Cells Mediated by Nuclease M

This Example describes the genomic editing of the AAVS1, EMX1, and VEGFA genes by Nuclease M (SEQ ID NO: 80) introduced into cells by lipid-based transient transfection into the HEK293T cell line.

Nuclease M was tagged with N-terminal SV40 nuclear localization sequence and a C-terminal nucleoplasmin nuclear localization sequence and converted to a human codon-optimized DNA sequence, synthesized, and cloned into a pcDNA3.1 vector (Invitrogen), containing a CMV promoter for expression. The reference and NLS-tagged sequences used are in Table 21. The U6 PolIII promoter uses a +1 G at the start of the transcript (i.e., the 5′ end of the RNA) for more efficient transcription that is excluded from the sequences described in Table 22. Plasmids were purified using a midiprep kit.

RNA guides were designed and cloned and into a pUC19 plasmid following the U6 PolIII promoter and terminated with a 6× polyT sequence. RNA guides were designed to be specific to target sequences within the coding exons of AAVS1, EMX1, and VEGFA with 5′-WTAAH-3′ PAM sequences (the PAM sequence is on the 3′ end of the target sequence). See all RNA guide sequences in Table 22. Plasmids were purified using a midiprep kit.

TABLE 21
Amino Acid Sequences

| Components | Amino acid sequence | SEQ ID NO |
|---|---|---|
| Nuclease M | MVYVISKNGKPLMPTKRHGLVRILLKEKKARVVQRKPFTIQLL | 80 |
| | YDSTTYTQDITAGFDTGRAYQSITAVNARTGEVLYSSVLETRN | |
| | KEVPKLMKSRKMYRAIRRHNRRMKKVRRAVRNKTYFRAPKK | |
| | VVWPGAKEPITAKYIKPKEARFNNRKRPEGWLTPTAVHLLRTH | |
| | LNYFKKVRKILPIKTLVLEYGKFDIQKLENPDIKGAEYQRGKLY | |
| | GYNNLREYVIAVQQGKCLLCEKKPIEHLHHVMPGSKEGSDTY | |
| | KNIAGLCSKCHAKVHTSPKAKEKLAEKAAGTAKEYADTSILNII | |
| | MPYLYSELKSMLGTENIALCYGYETEAARKSFGLAKSHSNDSY | |
| | AMALMAIGQASRIEKIEPYRYKQYRRHNRAFCDAQRDRLYKK | |
| | DGKIAARNRRRRTEQEGISLAEYRSELITALGKKIATREISKLKV | |
| | YRAVKKKSTSIKDVLFPPGCAVNYKGQRAVVKSFFNKGSSLIL | |
| | EGISGYVPAKDCQLITKKAGIVCL | |
| SV40 NLS | KRTADGSEFESPKKKRKV | 3 |
| Nucleoplasmin | KRPAATKKAGQAKKKK | 4 |
| NLS | | |
| XTEN linker | SGGSSGGSSGSETPGTSESATPESSGGSSGGSS | 95 |
| NLS-tagged | MKRTADGSEFESPKKKRKVVYVISKNGKPLMPTKRHGLVRILLK | 81 |
| Nuclease M | EKKARVVQRKPFTIQLLYDSTTYTQDITAGFDTGRAYQSITAVN | |
| | ARTGEVLYSSVLETRNKEVPKLMKSRKMYRAIRRHNRRMKKV | |
| | RRAVRNKTYFRAPKKVVWPGAKEPITAKYIKPKEARFNNRKRP | |
| | EGWLTPTAVHLLRTHLNYFKKVRKILPIKTLVLEYGKFDIQKLE | |
| | NPDIKGAEYQRGKLYGYNNLREYVIAVQQGKCLLCEKKPIEHL | |
| | HHVMPGSKEGSDTYKNIAGLCSKCHAKVHTSPKAKEKLAEKA | |
| | AGTAKEYADTSILNIIMPYLYSELKSMLGTENIALCYGYETEAA | |
| | RKSFGLAKSHSNDSYAMALMAIGQASRIEKIEPYRYKQYRRHN | |
| | RAFCDAQRDRLYKKDGKIAARNRRRRTEQEGISLAEYRSELITA | |
| | LGKKIATREISKLKVYRAVKKKSTSIKDVLFPPGCAVNYKGQR | |
| | AVVKSFFNKGSSLILEGISGYVPAKDCQLITKKAGIVCLSGGSSG | |
| | GSSGSETPGTSESATPESSGGSSGGSSKRPAATKKAGQAKKKK | |

TABLE 22
Target and RNA Guide Sequences

| | | Target/Spacer | |
|---|---|---|---|
| Guide | PAM | Sequence | RNA Guide Sequence* |
| AAVS1- | TTAAA | GCTGCCAA | GCUGCCAAAGCCAGGGCCAGgucaacuacccccgucuaaaga |
| T5 | | AGCCAGGG | cggaggcaugagguuucguaaccaaguguuguaccugcggguacaguaguu |
| | | CCAG (SEQ | gaacaggcggcgaugcggcugggcacuccaggaugccacucccagucccgga |
| | | ID NO: 82) | cacugccgacgagccgcaucaagccgggggagaccaaccggcuaacgauagc |
| | | | cgagcaauuaccuaaaaagaggugcaaaggaaaugguau (SEQ ID NO: |
| | | | 83) |
| AAVS1- | ATAAT | CAAAGCTG | CAAAGCUGGGGCCUUCGGCUgucaacuacccccgucuaaaga |
| T7 | | GGGCCTTC | cggaggcaugagguuucguaaccaaguguuguaccugcggguacaguaguu |
| | | GGCT (SEQ | gaacaggcggcgaugcggcugggcacuccaggaugccacucccagucccgga |
| | | ID NO: 84) | cacugccgacgagccgcaucaagccgggggagaccaaccggcuaacgauagc |
| | | | cgagcaauuaccuaaaaagaggugcaaaggaaaugguau (SEQ ID NO: |
| | | | 85) |
| EMX1-T5 | TTAAA | AGTGGTGC | AGUGGUGCCUGGAAAAUAAAgucaacuacccccgucuaaag |
| | | CTGGAAAA | acggaggcaugagguuucguaaccaaguguuguaccugcggguacaguagu |
| | | TAAA (SEQ | ugaacaggcggcgaugcggcugggcacuccaggaugccacucccagucccg |
| | | ID NO: 86) | gacacugccgacgagccgcaucaagccgggggagaccaaccggcuaacgaua |
| | | | gccgagcaauuaccuaaaaagaggugcaaaggaaaugguau (SEQ ID |
| | | | NO: 87) |
| EMX1-T7 | ATAAA | CTTGCTAA | CUUGCUAAAGAAACAUGUGUgucaacuacccccgucuaaaga |
| | | AGAAACAT | cggaggcaugagguuucguaaccaaguguuguaccugcggguacaguaguu |
| | | GTGT (SEQ | gaacaggcggcgaugcggcugggcacuccaggaugccacucccagucccgga |
| | | ID NO: 88) | cacugccgacgagccgcaucaagccgggggagaccaaccggcuaacgauagc |
| | | | cgagcaauuaccuaaaaagaggugcaaaggaaaugguau (SEQ ID NO: |
| | | | 89) |
| VEGFA- | TTAAA | TCACCATG | UCACCAUGACUGGUGGAGGAgucaacuacccccgucuaaaga |
| T2 | | ACTGGTGG | cggaggcaugagguuucguaaccaaguguuguaccugcggguacaguaguu |
| | | AGGA (SEQ | gaacaggcggcgaugcggcugggcacuccaggaugccacucccagucccgga |
| | | ID NO: 90) | cacugccgacgagccgcaucaagccgggggagaccaaccggcuaacgauagc |
| | | | cgagcaauuaccuaaaaagaggugcaaaggaaaugguau (SEQ ID NO: |
| | | | 91) |
| VEGFA- | TTAAA | GAGCCTGG | GAGCCUGGUCCAUGGAGAGUgucaacuacccccgucuaaaga |
| T5 | | TCCATGGA | cggaggcaugagguuucguaaccaaguguuguaccugcggguacaguaguu |
| | | GAGT (SEQ | gaacaggcggcgaugcggcugggcacuccaggaugccacucccagucccgga |
| | | ID NO: 92) | cacugccgacgagccgcaucaagccgggggagaccaaccggcuaacgauagc |
| | | | cgagcaauuaccuaaaaagaggugcaaaggaaaugguau (SEQ ID NO: |
| | | | 93) |

*Spacer in upper case and scaffold (SEQ ID NO: 94) in lower case

HEK293T cells were transfected with plasmids expressing the CRISPR nuclease polypeptides and the gRNAs provided herein following the methods provided in Example 1 above. Gene editing efficiencies were determined by Next Generation Sequencing following the methods also provided in Example 1. The NGS results were analyzed also following the methods provided in Example 1 above.

For each target, indel ratios, referring to the percentage of NGS reads comprising indels, were calculated for each sample and its cognate no protein control. Targets comprising a higher percentage of indels when Nuclease M was included in the transfection were indicative of DNA editing outcomes in the cell.

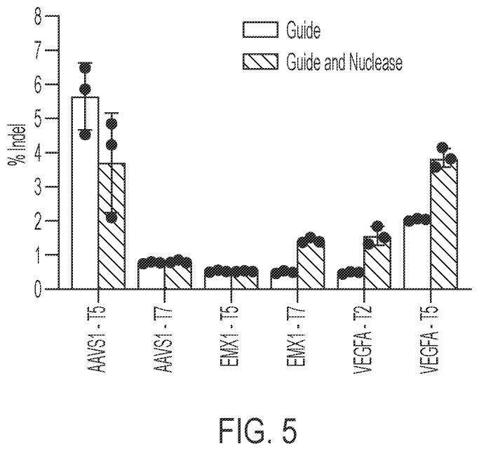

As shown in FIG. 5, three of the six targets tested demonstrated a greater level of indels observed when the Nuclease M (SEQ ID NO: 80) plasmid was present.

### Example 11: Effectiveness of Variant CRISPR Nucleases for Targeting of Mammalian Genes

This Example describes indel assessment on mammalian targets using CRISPR nuclease variants derived from Nuclease M (SEQ ID NO: 80) transfected into HEK293T cells.

Arginine scanning mutagenesis was performed to individually substitute select non-arginine residues of Nuclease M (SEQ ID NO: 80) to arginine. This resulted in 450 single arginine substitution variants. Nucleic acids encoding the reference CRISPR nuclease and each CRISPR nuclease variant were then individually cloned into a pcDNA3.1 backbone (Invitrogen™), and the plasmids were maxi-prepped and diluted. The plasmids comprised a CMV promoter, a first NLS (KRTADGSEFESPKKKRKV; SEQ ID NO: 3) upstream of the coding sequence, an XTEN linker (SGGSSGGSSGSETPGTSESATPESSGGSSGGSS; SEQ ID NO: 95) followed by a second NLS (KRPAATKKAGQAKKKK; SEQ ID NO: 4) downstream of the coding sequence. See also Example 10 above.

The RNA guide and target sequence are shown in Table 23. The RNA guide was cloned into a pUC19 backbone (New England Biolabs®) following the U6 PolIII promoter and terminated with a 6× polyT sequence. The U6 PolIII uses a +1 G at the start of the transcript (i.e., the 5′ end of the RNA) for more efficient transcription that is excluded from the sequences described in Table 23. The plasmid was then maxi-prepped and diluted.

TABLE 23
Mammalian Targets and Corresponding crRNAs*

| Target | | PAM | Target | RNA Guide |
|---|---|---|---|---|
| Identifier | gRNA | Sequence | Sequence | Sequence* |
| VEGFA | VEGFA-t5 | TTAAA | SEQ ID | SEQ ID |
| | | | NO: 92 | NO: 93 |

*: Sec Table 22 above for sequence information

HEK293T cells were transfected with plasmids expressing the CRISPR nuclease polypeptides and the gRNAs provided herein following the methods provided in Example 1 above. Gene editing efficiencies were determined by NGS following the methods also provided in Example 10. The NGS results were analyzed also following the methods provided in Example 10 above.

Indel ratios, referring to the percentage of NGS reads comprising indels, were calculated for the reference and for each variant. The indel ratios used for fold change calculations were the average of two technical replicates. To then calculate fold change in indel ratios, the indel ratio for each variant was divided by the indel ratio for the reference. Table 4 shows fold change in indel ratios. Numbering is relative to the reference Nuclease M of SEQ ID NO: 80 (i.e., without an NLS).

As shown in Table 24, 8 variants of Nuclease M with single arginine substitutions (left column) were characterized as yielding at least a 1.5X increase in indel ratio relative to the reference indel ratio.

TABLE 24
Fold Change in Indel Ratios*

| Substitution | VEGFA |
|---|---|
| E88R | 2.09 |
| S95R | 2.05 |
| L92R | 1.81 |
| E401R | 1.77 |
| E83R | 1.71 |
| N371R | 1.65 |
| P481R | 1.60 |
| A373R | 1.53 |

*Variant indel ratio/Reference indel ratio

173 variants of Nuclease M were analyzed as having indel ratios 1-1.5X of the reference indel ratios: G211R, D52R, T54R, K237R, E207R, A327R, K283R, A133R, K212R, E282R, K126R, L412R, K365R, K236R, S46R, F57R, A328R, V112R, L312R, N106R, Y141R, K143R, K277R, T414R, V116R, K10R, I102R, V81R, E27R, Y256R, E75R, G132R, G215R, V245R, A353R, Y454R, G333R, V270R, E315R, H18R, T73R, L171R, S310R, T138R, K7R, V33R, V128R, L232R, K26R, I180R, V430R, Y49R, G351R, T294R, I356R, L219R, A225R, A335R, T398R, P11R, P182R, S79R, K140R, S253R, V4R, Q378R, Y367R, K436R, K257R, L487R, D386R, V32R, W130R, A318R, L167R, T50R, 1137R, S470R, K28R, S249R, K127R, K29R, K110R, K309R, P37R, P447R, S6R, L24R, D443R, Y343R, G9R, D194R, N218R, L25R, Y362R, L20R, K119R, N340R, V177R, Y321R, H370R, N172R, T314R, I488R, K111R, L347R, Y408R, E146R, I359R, L242R, P131R, I195R, A492R, M301R, S337R, F174R, P136R, G402R, K145R, A451R, I5R, E474R, Y222R, S331R, L198R, T39R, N217R, T489R, K330R, E324R, F446R, L280R, N118R, S438R, D45R, V69R, W158R, Q400R, Y3R, Q227R, S339R, I296R, F122R, L12R, Y364R, G417R, S295R, I183R, C485R, T160R, I239R, E407R, S426R, 1472R, Y121R, Y78R, A163R, M13R, P361R, T15R, Y44R, V434R, F38R, V2R, T169R, F149R, P125R, P144R, C375R, I317R, F374R, N151R, P14R, L186R, Q64R, C263R, C266R, N86R, M93R, Y431R, L334R, L308R, L297R, Y479R, Q366R, G252R, G60R, T162R, I389R, H244R, H271R, D376R, P161R, A377R, V444R, K384R, V89R, A30R, A115R, A55R, L166R, T120R, C450R, E189R, T67R, A147R, V460R, V21R, H267R, A68R, D58R, I66R, C234R, L181R, I300R, I40R, Y323R, T84R, N258R, S65R, G191R, H165R, T59R, I142R, A139R, E307R, S463R, H243R, S80R, L471R, L188R, F465R, G56R, H170R, L428R, T439R, A421R, N466R, S404R, I53R, 1425R, Y173R, L82R, F464R, D380R, G261R, L304R, S342R, K336R, L262R, G322R, L382R, L405R, A433R, A391R, A346R, G449R, V452R, V461R, G468R, N393R, C231R, F193R, D341R, T325R, Y383R, M345R, H338R, and A344R.

The remaining variants of Nuclease M (269 variants) resulted in decreased indel ratios relative to Nuclease M indel ratios (fold change in indel ratios of less than 1.0).

Based on this experiment, variants of Nuclease M (SEQ ID NO: 80) having one or more of the following substitutions exhibited enhanced activities: E88R, S95R, L92R, E401R, E83R, N371R, P481R, and A373R.

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. An CRISPR nuclease polypeptide, comprising a RuvC nuclease domain and an HNH nuclease domain, wherein the CRISPR nuclease polypeptide comprises an amino acid sequence at least 90% identical to SEQ ID NO: 1; optionally wherein the CRISPR nuclease polypeptide is a variant comprising at least one mutation relative to SEQ ID NO: 1.

2. The CRISPR nuclease polypeptide of claim 1, wherein the CRISPR nuclease polypeptide is the variant comprising the at least one mutation, which comprises:
(a) one or more arginine and/or lysine substitutions, optionally one or more arginine substitutions, relative to SEQ ID NO: 1;
(b) one or more nickase mutations in the HNH nuclease domain or in the RuvC nuclease domain of SEQ ID NO: 1;
(c) an N-terminal truncation;
(d) a C-terminal truncation; or
(e) a combination of (a), (b), (c) and/or (d).

3. The CRISPR nuclease polypeptide of claim 1, wherein the at least one mutation comprises (a), which is located in a bridge helix (BH) domain, in a phosphate lock loop (PLL) domain, in a wedge (WED) domain, in a PAM-interacting (PID) domain, or a combination thereof.

4. The CRISPR nuclease polypeptide of claim 2, wherein at least one mutation comprises (a), and wherein the one or more arginine and/or lysine substitutions, optionally one or more arginine substitutions, are located at one or more of positions D56, E59, G60, E63, I67, G71, S564, D568, T605, E655, E694, and E706 in SEQ ID NO: 1.

5. The CRISPR nuclease polypeptide of claim 4, wherein the CRISPR nuclease polypeptide comprises arginine and/or lysine substitutions at the following positions relative to SEQ ID NO: 1:
a) I67, D568, and E706;
b) I67, D56, and D568;
c) I67, T605, and D568;
d) I67, D56, and E706;
e) I67, T605, and E706;
f) I67, D568, and E694;
g) I67, D56, E694, and E706;
h) I67, D56, T605, and E706; or
i) I67, D568, W593, and E706.

6. The CRISPR nuclease polypeptide of claim 1, wherein the CRISPR nuclease polypeptide has an N-terminal truncation relative to SEQ ID NO: 1, optionally wherein the N-terminal truncation is a deletion within residues 1-15 of SEQ ID NO: 1; preferably wherein the N-terminal truncation is a deletion of residues 1-14 or residues 1-15 of SEQ ID NO: 1.

7. The CRISPR nuclease of claim 2, wherein the CRISPR nuclease polypeptide contains up to 20 arginine and/or lysine substitutions, optionally up to 20 arginine substitutions, relative to SEQ ID NO: 1; optionally wherein the CRISPR nuclease polypeptide contains up to 15 arginine and/or lysine substitutions, optionally up to 15 arginine substitutions, relative to SEQ ID NO: 1.

8. The CRISPR nuclease of claim 7, wherein the CRISPR nuclease polypeptide comprises the following combination of arginine substitutions:
a) I67R, D568R, and E706R;
b) I67R, D56R, and D568R;
c) I67R, T605R, and D568R;
d) I67R, D56R, and E706R;
e) I67R, T605R, and E706R;
f) I67R, D568R, and E694R;
g) I67R, D56R, E694R, and E706R;
h) I67R, D56R, T605R, and E706R; or
i) I67R, D568R, W593R, and E706R.

9. The CRISPR nuclease of claim 8, wherein the CRISPR nuclease polypeptide comprises the arginine substitutions of I67R, D568R, and E706R.

10. The CRISPR nuclease of claim 2, wherein the at least one mutation in the CRISPR nuclease polypeptide comprises (b), which comprises one or more mutations at position H397, D396, N420, D24, E337, and/or D524 of SEQ ID NO: 1.

11. The CRISPR nuclease of claim 10, wherein the at least one mutation comprises a mutation at position H397, optionally wherein the mutation is amino acid substitution of H397A or H397L.

12. The CRISPR nuclease polypeptide of claim 2, wherein the variant comprises:
(a) the one or more nickase mutations in the HNH nuclease domain, which optionally are at positions D396, H397, and/or N420 of SEQ ID NO: 1; optionally wherein the nickase mutation is at position H397; and
(b) the one or more arginine and/or lysine substitutions, which optionally are at positions I67, D568, and E706 of SEQ ID NO: 1.

13. The CRISPR nuclease polypeptide of claim 2, wherein the variant comprises:
(a) the one or more nickase mutations in the HNH nuclease domain, which optionally are at positions D396, H397, and/or N420 of SEQ ID NO: 1;
(b) the one or more arginine and/or lysine substitutions, which optionally are at positions I67, D568, and E706 of SEQ ID NO: 1; and
(c) the N-terminal truncation within residues 1-15 of SEQ ID NO: 1, optionally wherein the N-terminal truncation is the deletion of residues 1-14 or 1-15 of SEQ ID NO: 1.

14. The CRISPR nuclease polypeptide of claim 13, wherein the variant comprises:
(a) the nickase mutation of H397A;
(b) the arginine substitutions of I67R, D568R, and E706R; and
(c) the N-terminal truncation comprising deletion of residues 1-14 or 1-15 of SEQ ID NO: 1.

15. The CRISPR nuclease polypeptide of claim 1, wherein the CRISPR nuclease polypeptide is listed in Table 1, Table 11, or Table 14.

16. The CRISPR nuclease polypeptide of claim 1, wherein the CRISPR nuclease polypeptide comprises an amino acid sequence at least 95% identical to SEQ ID NO: 1.

17. The CRISPR nuclease polypeptide of claim 16, wherein the CRISPR nuclease polypeptide comprises an amino acid sequence at least 98% identical to SEQ ID NO: 1.

18. The CRISPR nuclease polypeptide of claim 1, which is a fusion polypeptide further comprising one or more functional fragments.

19. The CRISPR nuclease polypeptide of claim 18, wherein the one or more functional fragments comprise one or more nuclear localization signals (NLSs), one or more peptide linkers, or a combination thereof.

20. A nucleic acid, comprising a nucleotide sequence encoding the CRISPR nuclease polypeptide of claim 1.

21. The nucleic acid of claim 20, wherein the nucleic acid is a messenger RNA (mRNA).

22. The nucleic acid of claim 20, wherein the nucleic acid is an expression vector, in which the nucleotide sequence encoding the CRISPR nuclease polypeptide is in operable linkage to a promoter; optionally wherein the expression vector is a viral vector.

23. A host cell comprising the nucleic acid of claim 21.

24. A gene editing system, comprising:
(a) a CRISPR nuclease polypeptide or a first nucleic acid encoding the CRISPR nuclease, wherein the CRISPR nuclease polypeptide is set forth in claim 1; and
(b) a guide RNA (gRNA) or a second nucleic acid encoding the gRNA, wherein the gRNA comprises a scaffold sequence recognizable by the CRISPR nuclease and a spacer specific to a target sequence in a genomic site of interest, wherein the target sequence is adjacent to a protospacer adjacent motif (PAM).

25. The gene editing system of claim 24, wherein the scaffold sequence comprises a nucleotide sequence at least 85% identical to SEQ ID NO: 2;
optionally wherein the scaffold sequence comprises one or more deletions, one or more nucleotide substitutions, or a combination thereof, as compared with SEQ ID NO: 2.

26. The gene editing system of claim 25, wherein the scaffold sequence is a truncated variant of SEQ ID NO: 2, which has a 3′ truncation relative to SEQ ID NO: 2, and wherein the truncated variant is about 110-140-nt in length, optionally wherein the 3′ truncation comprises deletions within residues 143-202 of SEQ ID NO: 2.

27. The gene editing system of claim 26, wherein the truncated variant further comprises one or more deletions within residues 10-40 of SEQ ID NO: 2; optionally wherein the deletions comprise residues 14-20 and/or 25-32 of SEQ ID NO: 2.

28. The gene editing system of claim 26, wherein the truncated variant further comprises one or more mutations relative to SEQ ID NO: 2; optionally wherein the one or more mutations comprise a deletion within residues 82-85 of SEQ ID NO: 2.

29. The gene editing system of claim 26, wherein the scaffold sequence comprises the nucleotide sequence of SEQ ID NO: 27, or SEQ ID NO: 28; and wherein the scaffold sequence is about 115-130-nt in length.

30. The gene editing system of claim 29, wherein in the gene editing system:
(a) the CRISPR nuclease polypeptide comprises the amino acid sequence of SEQ ID NO: 1; and the scaffold comprises the nucleotide sequence of SEQ ID NO: 27;
(b) the CRISPR nuclease polypeptide is a variant of SEQ ID NO: 1 comprising mutations at positions 167, D568, and E706 of SEQ ID NO: 1, which optionally are arginine substitutions I67R, D568R, and E706R; and the scaffold comprises the nucleotide sequence of SEQ ID NO: 28; or
(c) the CRISPR nuclease polypeptide is a variant of SEQ ID NO: 1 comprising a deletion within residues 1-15 of SEQ ID NO: 1; optionally a deletion of residues 1-14 or 1-15 of SEQ ID NO: 1; and the scaffold comprises the nucleotide sequence of SEQ ID NO: 28.

31. The gene editing system of claim 24, wherein the target sequence is adjacent to the PAM of 5′-NGG-3′, in which N represents any nucleotide.

32. The gene editing system of claim 24, which further comprises one or more lipid excipients associated with the element (a) and/or element (b) of the gene editing system; optionally wherein the one or more lipid excipients form lipid nanoparticles, which are associated with or encapsulate the element (a) and/or element (b) of the gene editing system.

33. A gene editing method, comprising delivering the gene editing system of claim 24 to a host cell to edit a genomic site targeted by the gRNA of the gene editing system.

34. A guide RNA, comprising a spacer sequence and a scaffold sequence, wherein the scaffold sequence is a variant of SEQ ID NO: 2 comprising one or more deletions, one or more nucleotide substitutions, or a combination thereof, as compared with SEQ ID NO: 2, and wherein the scaffold sequence is recognizable by a CRISPR nuclease polypeptide set forth in claim 1.

35. The guide RNA of claim 34, wherein the scaffold sequence is a truncated variant of SEQ ID NO: 2, which has a 3′ truncation relative to SEQ ID NO: 2, and wherein the truncated variant is about 110-140-nt in length.

36. The guide RNA of claim 35, wherein the 3′ truncation comprises deletions within residues 143-202 of SEQ ID NO: 2.

37. The guide RNA of claim 35, wherein the truncated variant further comprises one or more deletions within residues 10-40 of SEQ ID NO: 2.

38. The guide RNA of claim 37, wherein the deletions comprise residues 14-20 and/or 25-32 of SEQ ID NO: 2.

39. The guide RNA of claim 37, wherein the truncated variant further comprises one or more mutations relative to SEQ ID NO: 2; optionally the one or more mutations comprise a deletion within residues 81-85 of SEQ ID NO: 2.

40. The guide RNA of claim 35, wherein the scaffold sequence comprises the nucleotide sequence of SEQ ID NO: 27, or SEQ ID NO: 28; and wherein the scaffold is about 115-130-nt in length.

41. A CRISPR nuclease polypeptide, comprising a RuvC nuclease domain and an HNH nuclease domain, wherein the CRISPR nuclease polypeptide comprises an amino acid sequence at least 90% identical to SEQ ID NO: 65; optionally wherein the CRISPR nuclease polypeptide is a variant comprising at least one mutation relative to SEQ ID NO: 65.

42. The CRISPR nuclease polypeptide of claim 41, wherein the CRISPR nuclease polypeptide is the variant comprising at least one mutation, which comprises:
(a) one or more arginine and/or lysine substitutions, optionally one or more arginine substitutions, relative to SEQ ID NO: 65;
(b) one or more nickase mutations in the HNH nuclease domain or in the RuvC nuclease domain of SEQ ID NO: 65; or
(c) a combination of (a) and (b).

43. The CRISPR nuclease polypeptide of claim 42, wherein the CRISPR nuclease polypeptide comprises a bridge helix (BH) domain, a wedge (WED) domain, and a PAM-interacting (PID) domain, and wherein the CRISPR nuclease polypeptide is a variant comprising the one or more arginine and/or lysine substitutions of (a), which are located in the BH domain, in the WED domain, in the PID domain, or a combination thereof.

44. The CRISPR nuclease polypeptide of claim 43, wherein the one or more arginine and/or lysine substitutions are located at one or more of positions G42, D46, 153, F83, E128, E541, F570, E576, D582, C630, 1631, E683, S719, T734 in SEQ ID NO: 65.

45. The CRISPR nuclease polypeptide of claim 44, wherein the CRISPR nuclease polypeptide comprises arginine and/or lysine substitutions at the following positions relative to SEQ ID NO: 65:
(i) D582 and G42; or
(ii) D582, 153, and T734R.

46. The CRISPR nuclease polypeptide of claim 45, wherein the CRISPR nuclease polypeptide comprises the following arginine substitutions:
a) D582R and G42R; or
b) D582R, I53R, and T734R.

47. The CRISPR nuclease polypeptide of claim 42, wherein the CRISPR nuclease polypeptide contains up to 20 arginine and/or lysine substitutions, optionally up to 20 arginine substitutions, relative to SEQ ID NO: 65; optionally wherein the CRISPR nuclease polypeptide contains up to 15 arginine and/or lysine substitutions, optionally up to 15 arginine substitutions, relative to SEQ ID NO: 65.

48. The CRISPR nuclease polypeptide of claim 42, wherein the CRISPR nuclease polypeptide comprises the one or more nickase mutations of (b), which are at position D374, H375, and/or N398 in SEQ ID NO: 65.

49. The CRISPR nuclease polypeptide of claim 48, wherein the nickase mutation is at position H375; optionally wherein the mutation is an amino acid substitution of H375A.

50. The CRISPR nuclease polypeptide of claim 41, wherein the CRISPR nuclease polypeptide comprises an amino acid sequence at least 95% identical to SEQ ID NO: 65.

51. The CRISPR nuclease polypeptide of claim 50, wherein the CRISPR nuclease polypeptide comprises an amino acid sequence at least 98% identical to SEQ ID NO: 65.

52. The CRISPR nuclease polypeptide of claim 41, which is a fusion polypeptide comprising one or more additional functional elements.

53. The CRISPR nuclease polypeptide of claim 52, wherein the one or more additional functional elements comprise one or more nuclear localization signals (NLSs), one or more peptide linkers, or a combination thereof.

54. A nucleic acid, comprising a nucleotide sequence encoding the CRISPR nuclease polypeptide of claim 41.

55. The nucleic acid of claim 54, wherein the nucleic acid is an expression vector, in which the nucleotide sequence encoding the CRISPR nuclease polypeptide is in operable linkage to a promoter; optionally wherein the expression vector is a viral vector.

56. The nucleic acid of claim 54, wherein the nucleic acid is a messenger RNA (mRNA).

57. A host cell comprising the nucleic acid of claim 54.

58. A gene editing system, comprising:
(a) the CRISPR nuclease polypeptide of claim 41 or a first nucleic acid encoding the CRISPR nuclease polypeptide; and
(b) a guide RNA (gRNA) or a second nucleic acid encoding the gRNA, wherein the gRNA comprises a scaffold sequence recognizable by the CRISPR nuclease polypeptide and a spacer sequence specific to a target sequence within a genomic site of interest, wherein the target sequence is adjacent to a protospacer adjacent motif (PAM).

59. The gene editing system of claim 58, wherein the scaffold sequence comprises a nucleotide sequence at least 85% identical to SEQ ID NO: 79.

60. The gene editing system of claim 59, wherein the scaffold sequence is a fragment of SEQ ID NO: 79.

61. The gene editing system of claim 58, wherein the scaffold sequence comprises one or more deletions, one or more nucleotide substitutions, or a combination thereof, as compared with SEQ ID NO: 79.

62. The gene editing system of claim 58, wherein the target sequence is upstream to the PAM of 5′-NGG-3′, in which N represents any nucleotide.

63. The gene editing system of claim 58, which further comprises one or more lipid excipients associated with the element (a) and/or element (b) of the gene editing system; optionally wherein the one or more lipid excipients form lipid nanoparticles, which are associated with or encapsulate the element (a) and/or element (b) of the gene editing system.

64. A gene editing method, comprising delivering the gene editing system of claim 58 to a host cell to edit a genomic site targeted by the gRNA of the gene editing system.

65. A nuclease polypeptide, comprising an RuvC nuclease domain and an HNH nuclease domain, wherein the nuclease polypeptide comprises an amino acid sequence at least 90% identical to SEQ ID NO: 80; optionally wherein the nuclease polypeptide is a variant of SEQ ID NO: 80 comprising at least one mutation relative to SEQ ID NO: 80.

66. The nuclease polypeptide of claim 65, wherein the nuclease polypeptide is the variant comprising the at least one mutation, which comprises:
(a) one or more arginine and/or lysine substitutions, optionally one or more arginine substitutions, relative to SEQ ID NO: 80;
(b) one or more nickase mutations in the HNH nuclease domain or in the RuvC nuclease domain of SEQ ID NO: 80; or
(c) a combination of (a) and (b).

67. The nuclease polypeptide of claim 66, wherein the at least one mutation comprises (a), which is located in a bridge helix (BH) domain, in a nucleic acid recognition (REC) domain, in a phosphate lock loop (PLL) domain, in a wedge (WED) domain, in a PAM-interacting (PID) domain, in a nuclease domain, or a combination thereof.

68. The nuclease polypeptide of claim 66, wherein the nuclease polypeptide contains up to 20 arginine and/or lysine substitutions, optionally up to 20 arginine substitutions, relative to SEQ ID NO: 80.

69. The nuclease polypeptide of claim 68, wherein the nuclease polypeptide contains up to 15 arginine and/or lysine substitutions, optionally up to 15 arginine substitutions, relative to SEQ ID NO: 80.

70. The nuclease polypeptide of claim 67, wherein the one or more arginine and/or lysine substitutions, optionally arginine substitutions, are at positions E88, S95, L92, E401, E83, N371, P481, and/or A373 in SEQ ID NO: 80.

71. The nuclease polypeptide of claim 66, wherein the at least one mutation comprises (b), and wherein the one or more nickase mutations are at one or more of positions D58, E189, D341, H243, H244, H267, R329, and/or H338 in SEQ ID NO: 80.

72. The nuclease polypeptide of claim 65, wherein the nuclease polypeptide comprises an amino acid sequence at least 95% identical to SEQ ID NO: 80.

73. The nuclease polypeptide of claim 72, wherein the nuclease polypeptide comprises an amino acid sequence at least 98% identical to SEQ ID NO: 80.

74. The nuclease polypeptide of claim 65, wherein the nuclease polypeptide is a fusion polypeptide, which further comprises one or more functional fragments that are heterologous to the nuclease moiety in the fusion polypeptide.

75. The nuclease polypeptide of claim 74, wherein the one or more functional fragments comprise one or more nuclear localization signal(s) (NLSs), one or more peptide linker, or a combination thereof.

76. A nucleic acid, comprising a nucleotide sequence encoding the nuclease polypeptide of claim 65.

77. The nucleic acid of claim 76, wherein the nucleic acid is an expression vector, in which the nucleotide sequence encoding the nuclease is in operable linkage to a promoter; optionally wherein the expression vector is a viral vector.

78. The nucleic acid of claim 77, wherein the nucleic acid is a messenger RNA (mRNA).

79. A host cell comprising the nucleic acid of claim 76.

80. A gene editing system, comprising:
(a) a nuclease polypeptide or a first nucleic acid encoding the nuclease, wherein the nuclease polypeptide is set forth in claim 65; and
(b) a guide RNA (gRNA) or a second nucleic acid encoding the gRNA; wherein the gRNA comprises a scaffold sequence recognizable by the nuclease polypeptide and a spacer sequence, which is specific to a target sequence in a genomic site of interest, wherein the target sequence is adjacent to a protospacer adjacent motif (PAM).

81. The gene editing system of claim 80, wherein the scaffold sequence comprises a nucleotide sequence at least 85% identical to SEQ ID NO: 94.

82. The gene editing system of claim 81, wherein the scaffold sequence is a fragment of SEQ ID NO: 94.

83. The gene editing system of claim 82, wherein the scaffold sequence comprises one or more deletions, one or more nucleotide substitutions, or a combination thereof, as compared with SEQ ID NO: 94.

84. The gene editing system of claim 80, wherein the PAM is 5′-WTAAH-3′, in which W is A or T and His A, C, or T; optionally wherein the PAM is 5′-TTAAA-3′.

85. The gene editing system of claim 80, which further comprises one or more lipid excipients associated with the element (a) and/or element (b) of the gene editing system; optionally wherein the one or more lipid excipients form lipid nanoparticles, which are associated with or encapsulate the element (a) and/or element (b) of the gene editing system.

86. A gene editing method, comprising delivering the gene editing system of claim 80 to a host cell to edit a genomic site targeted by the gRNA of the gene editing system.
